SEQUENCE LISTING (95 sequences)

SEQ ID NO: 1 - DNA, 4166 bp, synthetic construct
  source 1..4166: mol_type = other DNA
gtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggcgcgcgccaggcggggcggggcggggcgaggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgcatcgatattctgagccgccaccatggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggcggcaccggtagtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaaagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggg

SEQ ID NO: 2 - DNA, 500 bp, synthetic construct
  source 1..500: mol_type = other DNA
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 3 - DNA, 609 bp, synthetic construct
  source 1..609: mol_type = other DNA
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 4 - DNA, 4457 bp, synthetic construct
  source 1..4457: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggcggcaccggtagtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaaagaagcttgcctcgagcagcgcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccggctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 3745 bp, synthetic construct
  source 1..3745: mol_type = other DNA
tcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgctttcttcgcctcttggggcatgtacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtgaccgcggccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgc

SEQ ID NO: 6 - DNA, 3626 bp, synthetic construct
  source 1..3626: mol_type = other DNA
gcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggcccacgcccccggcaccgaccagatgttctacgtgggcaccatggacggctggtacctggacaccaagctgaacagcgtggccatcggcgcccactggagctgcttcatcgtgctgaccatcaccaccttctacctgggctacgagagctggaccagcaggggccccagcaagaggaccagcttctacgccggctaccaggaggagcagaacctggccctgttcgtgaacttcttcgccatgctgagctacttcggcaagatcgtggccgacaccctgggccacaacttcggcgacgtgggccccttcatcatcggcttcggcaactacaggtacgccgactacatgctgacctgccccatgctggtgtacgacctgctgtaccagctgagggccccctacagggtgagctgcagcgccatcatcttcgccatcctgatgagcggcgtgctggccgagttctacgccgagggcgaccccaggctgaggaacggcgcctacgcctggtacggcttcggctgcttctggttcatcttcgcctacagcatcgtgatgagcatcgtggccaagcagtacagcaggctggcccagctggcccaggacaccggcgccgagcacagcctgcacgtgctgaagttcgccgtgttcaccttcagcatgctgtggattctgttccccctggtgtgggccatctgccccaggggcttcggctggatcgacgacaactggaccgaggtggcccactgcgtgtgcgacatcgtggccaagagctgctacggcttcgccctggccaggttcaggaagacctacgacgaggagctgttcaggctgctggagcagctgggccacgacgaggacgagttccagaagctggagctggacatgaggctgagcagcaacggcgagaggctgaggaggctgagcgcggcaccggtagtagcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcc

SEQ ID NO: 7 - DNA, 3741 bp, synthetic construct
  source 1..3741: mol_type = other DNA
gtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggg

SEQ ID NO: 8 - DNA, 593 bp, synthetic construct
  source 1..593: mol_type = other DNA
aatcaacctctggattacaaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaggtctgctgagactcggggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 9 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 10 - DNA, 1000 bp, synthetic construct
  source 1..1000: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 11 - DNA, 1500 bp, synthetic construct
  source 1..1500: mol_type = other DNA
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 12 - DNA, 1185 bp, synthetic construct
  source 1..1185: mol_type = other DNA
gttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 13 - DNA, 1500 bp, synthetic construct
  source 1..1500: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcccatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 14 - DNA, 1185 bp, synthetic construct
  source 1..1185: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 15 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 16 - DNA, 1056 bp, synthetic construct
  source 1..1056: mol_type = other DNA
atggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgag

SEQ ID NO: 17 - DNA, 3021 bp, synthetic construct
  source 1..3021: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 3706 bp, synthetic construct
  source 1..3706: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 3706 bp, synthetic construct
  source 1..3706: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 4235 bp, synthetic construct
  source 1..4235: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 4293 bp, synthetic construct
  source 1..4293: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttgacaggtatttgccactaagcccagctaattgttttttatttagtagaaacggggtttcaccatgttagtcaggctggtcgggaactcctgacctcaggagatctacccgccttggcctcccaaagtgctgggattacaggcgtgtgccactgtgcccagccacttttttttagacagagtcttggtctgttgcccaggctagagttcagtggcgccatctcagctcactgcaacctccgcctcccagattcaagcgattctcctgcctcgacctcccagtagctgggattacaggtttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccatctagagctgccgtcggggacagggctttccatagcccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 1724 bp, synthetic construct
  source 1..1724: mol_type = other DNA
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcc

SEQ ID NO: 23 - DNA, 1782 bp, synthetic construct
  source 1..1782: mol_type = other DNA
taggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttgacaggtatttgccactaagcccagctaattgttttttatttagtagaaacggggtttcaccatgttagtcaggctggtcgggaactcctgacctcaggagatctacccgccttggcctcccaaagtgctgggattacaggcgtgtgccactgtgcccagccacttttttttagacagagtcttggtctgttgcccaggctagagttcagtggcgccatctcagctcactgcaacctccgcctcccagattcaagcgattctcctgcctcgacctcccagtagctgggattacaggtttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccatctagagctgccgtcggggacagggctttccatagcc

SEQ ID NO: 24 - DNA, 395 bp, synthetic construct
  source 1..395: mol_type = other DNA
atctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 25 - DNA, 290 bp, synthetic construct
  source 1..290: mol_type = other DNA
ggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 26 - DNA, 185 bp, synthetic construct
  source 1..185: mol_type = other DNA
gttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 27 - DNA, 150 bp, synthetic construct
  source 1..150: mol_type = other DNA
ttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 28 - DNA, 115 bp, synthetic construct
  source 1..115: mol_type = other DNA
aggggacaatccccggattaggaaggagctctccagctcgggtaaggagtctcaaggcaaggtaggcaagcaccaccggtccgcactctcgcccagcttttacgggaagaagaga

SEQ ID NO: 29 - DNA, 80 bp, synthetic construct
  source 1..80: mol_type = other DNA
gctcgggtaaggagtctcaaggcaaggtaggcaagcaccaccggtccgcactctcgcccagcttttacgggaagaagaga

SEQ ID NO: 30 - DNA, 395 bp, synthetic construct
  source 1..395: mol_type = other DNA
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaat

SEQ ID NO: 31 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - protein, 352 aa, synthetic construct
MVSRRPWLLALALAVALAAGSAGASTGSDATVPVATQDGPDYVFHRAHERMLFQTSYTLENNGSVICIPNNGQCFCLAWLKSNGTNAEKLAANILQWVVFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESSLE

SEQ ID NO: 33 - DNA, 1056 bp, synthetic construct
  source 1..1056: mol_type = other DNA
atggtgtctagaagaccttggctgctggctctggctctggcagtggccctggctgcaggttctgccggcgccagcaccggcagcgacgccacagtgcctgtggccacacaggacggccctgattacgtgttccacagagcccacgagagaatgctgtttcagacctcttataccctggaaaacaatggcagcgtgatctgcatccccaacaacggccaatgtttctgcctggcctggctcaagagcaacggcaccaacgccgagaagctggccgccaacatcctgcagtgggtcgtgtttgccctgagcgtggcttgtctgggctggtacgcctaccaggcctggcgggccacatgcggctgggaggaagtgtacgtcgccctgatcgagatgatgaaaagcatcatcgaagccttccacgagttcgactctccagccaccctgtggctgagctctggcaacggcgtggtgtggatgcgctacggcgagtggctgctcacctgccctgtgatcctgatccacctgtccaacctgaccggcctgaaggacgactatagcaagcggaccatgggcctgctggtgtccgatgtgggctgtatcgtgtggggcgctaccagcgccatgtgcaccggatggaccaagattctgtttttcctgattagcctgagctacggcatgtacacctacttccacgccgctaaagtgtacatcgaggccttccatacagtgcctaagggcctgtgcagacagctggtgcgggccatggcctggctgttcttcgtgagctggggaatgttccccgtgctgttcctgcttggacctgagggcttcggccacatcagcccctacgggtcagccatcggccacagcatcctggacctgatcgccaagaacatgtggggcgtgctgggaaattacctgagagtgaagatccacgaacacatcctgctgtacggcgacatcagaaagaagcagaaaataacaatcgccggccaggagatggaagtcgagacactggttgctgaagaggaagataagtacgagagcagcctggaa

SEQ ID NO: 34 - DNA, 1056 bp, synthetic construct
  source 1..1056: mol_type = other DNA
atggtgtcaagacgaccctggcttctggcactcgcgcttgcagtcgctcttgctgccgggtctgcaggcgccagcacaggtagtgatgccaccgttcccgtggcaacgcaggatggcccggattacgtgtttcatagagcacacgaaagaatgcttttccagacgagctacactctggaaaacaatggtagcgttatttgtatacccaacaacggacagtgcttttgcttggcttggcttaaatctaatgggactaacgctgagaagctcgcagcaaacatactccaatgggtggtgtttgctttgtccgttgcgtgtcttggttggtacgcttatcaagcctggcgagcgacatgcggatgggaagaggtttatgttgccctcatagagatgatgaagtcaataatcgaggcttttcatgagttcgatagtcccgcgactctgtggttgagtagtggtaatggtgttgtgtggatgcggtatggggagtggctccttacatgcccagttattttgattcacctgtcaaatttgactggtcttaaagacgactacagcaaacgcacgatgggtttgttggtcagtgatgtcggttgcatcgtttggggggctacgtccgctatgtgtaccgggtggacgaaaattctgttctttctgattagcttgtcttacgggatgtatacatatttccacgccgctaaggtctacatcgaagcctttcatactgtgccgaagggattgtgtcgccaattggtccgcgcgatggcgtggctgttttttgtcagctggggaatgtttcctgttttgttcttgttgggtcctgaaggatttgggcatatttccccttatggttcagccattggacactctattctggatctgattgcaaaaaatatgtggggcgtgctgggaaactacttgagggtgaaaatccacgagcacatactgctctatggtgacatccgaaagaagcaaaaaattactatagctgggcaggagatggaggtggagaccttggttgcagaggaggaggacaagtacgaatcttccctggag

SEQ ID NO: 35 - DNA, 3738 bp, synthetic construct
  source 1..3738: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 36 - DNA, 895 bp, synthetic construct
  source 1..895: mol_type = other DNA
aaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 37 - DNA, 790 bp, synthetic construct
  source 1..790: mol_type = other DNA
acagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 38 - DNA, 685 bp, synthetic construct
  source 1..685: mol_type = other DNA
agctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 39 - DNA, 580 bp, synthetic construct
  source 1..580: mol_type = other DNA
cccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 40 - DNA, 475 bp, synthetic construct
  source 1..475: mol_type = other DNA
ccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 41 - DNA, 370 bp, synthetic construct
  source 1..370: mol_type = other DNA
acctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 42 - DNA, 265 bp, synthetic construct
  source 1..265: mol_type = other DNA
ctagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 43 - DNA, 160 bp, synthetic construct
  source 1..160: mol_type = other DNA
aatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagcc

SEQ ID NO: 44 - DNA, 55 bp, synthetic construct
  source 1..55: mol_type = other DNA
attggaaggcctccgtcccagtccttctacagagtagccagtgggattcccagcc

SEQ ID NO: 45 - DNA, 895 bp, synthetic construct
  source 1..895: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcaga

SEQ ID NO: 46 - DNA, 790 bp, synthetic construct
  source 1..790: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatct

SEQ ID NO: 47 - DNA, 685 bp, synthetic construct
  source 1..685: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggca

SEQ ID NO: 48 - DNA, 580 bp, synthetic construct
  source 1..580: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtca

SEQ ID NO: 49 - DNA, 475 bp, synthetic construct
  source 1..475: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccgg

SEQ ID NO: 50 - DNA, 370 bp, synthetic construct
  source 1..370: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccaga

SEQ ID NO: 51 - DNA, 265 bp, synthetic construct
  source 1..265: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgt

SEQ ID NO: 52 - DNA, 160 bp, synthetic construct
  source 1..160: mol_type = other DNA
aacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctc

SEQ ID NO: 53 - DNA, 55 bp, synthetic construct
  source 1..55: mol_type = other DNA
aacccaagaaattacaggctgaaaccagaaaagaacacattaaagcaccaagaga

SEQ ID NO: 54 - DNA, 265 bp, synthetic construct
  source 1..265: mol_type = other DNA
acctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcaga

SEQ ID NO: 55 - DNA, 4423 bp, synthetic construct
  source 1..4423: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 4423 bp, synthetic construct
  source 1..4423: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggctgctggccctggccctggccgtggccctggccgccggcagcgccggcgccagcaccggcagcgacgccaccgtgcccgtggccacccaggacggccccgactacgtgttccacagagcccacgagagaatgctgttccagaccagctacaccctggagaacaacggcagcgtgatctgcatccccaacaacggccagtgcttctgcctggcctggctgaagagtaacggcaccaacgccgagaagctggccgccaacatcctgcagtgggtggtgtttgcgctgagcgtggcgtgcctgggctggtatgcgtatcaggcgtggcgcgcgacctgcggctgggaagaagtgtatgtggcgctgattgaaatgatgaaaagcattattgaagcgtttcatgaatttgatagcccggcgaccctgtggctgagcagcggcaacggcgtggtgtggatgcgctatggcgaatggctgctgacctgccccgtgattctgattcatctgagcaacctgaccggcctgaaagatgattatagcaaacgcaccatgggcctgctggtgagcgacgtgggctgcattgtgtggggcgcgaccagcgcgatgtgcaccggctggaccaaaattctgttttttctgattagcctgagctatggcatgtatacctattttcatgcggccaaagtgtatattgaagcgtttcataccgtgccgaaaggcctgtgcagacagctggtgagagccatggcctggctgttcttcgtgagctggggcatgttccccgtgctgttcctgctgggccccgagggcttcggccatattagcccgtatggcagcgcgattggccatagcattctggatctgattgcgaagaacatgtggggcgtgctgggcaactatctgcgcgtgaaaattcatgaacatattctgctgtatggcgatattcgcaaaaaacagaaaattaccattgcgggccaggaaatggaagtggaaaccctggtggcggaagaagaagataagtacgagagcagcctcgagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 57 - protein, 95 aa, synthetic construct
MVSRRPWLLALALAVALAAGSAGASTGSDATVPVATQDGPDYVFHRAHERMLFQTSYTLENNGSVICIPNNGQCFCLAWLKSNGTNAEKLAANIL

SEQ ID NO: 58 - DNA, 1056 bp, synthetic construct
  source 1..1056: mol_type = other DNA
atggtgtctagaagaccttggctgctggccctggctctggctgtggcccttgctgctggatctgctggagccagcacaggctcagatgccacagtgccagtggccacacaggacggcccagattatgtgtttcacagagcccatgagagaatgctgttccaaacaagctatacactggaaaacaacggatctgtgatctgcatccccaacaacggccagtgcttctgcctggcttggcttaagagtaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctgggaatggagtggtgtggatgaggtatggagagtggctcctgacctgtcctgtgatcctgatccacctgtctaatctgacaggactgaaagacgactacagcaaaaggactatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaaaaacatgtggggagtgctgggaaattacctgagagtgaagatccatgagcacattctgctgtatggagacatcagaaagaaacagaagatcaccattgcaggccaggaaatggaagttgagacactggtggctgaagaagaagataagtatgagagcagcctggaa

SEQ ID NO: 59 - DNA, 1056 bp, synthetic construct
  source 1..1056: mol_type = other DNA
atggtgagcagaagaccctggcttttggccttggccctggctgtggcccttgctgctggatctgctggggcatctacaggctctgatgctacagttccagttgcaacccaggacggccctgactatgtgttccacagagcccatgagagaatgctgttccagaccagctacaccctggagaacaacggctctgtgatctgcatccccaacaacggccagtgcttctgccttgcatggctgaagagtaatggcaccaatgctgagaagctggctgccaacatcctgcagtgggtggtgtttgcactgtctgtggcatgcctgggctggtatgcatatcaggcttggagggcaacctgtgggtgggaagaagtgtatgtggcactgattgaaatgatgaaaagcattattgaagccttccatgaatttgatagcccagccaccctgtggctgagctctggcaacggagtggtgtggatgaggtatggagagtggctgctgacctgccctgtgattctgattcatctgagcaacctgacaggcctgaaagatgattatagcaaaaggactatgggcctgctggtgtctgatgtgggctgcattgtgtggggagcaacctctgcaatgtgcacaggctggaccaaaattctgttcttcctgattagcctgtcttatggcatgtatacctattttcatgcagccaaagtgtatattgaagcatttcatacagtgccaaaaggcctgtgcagacagctggtgagagctatggcctggctgttctttgtgagctggggaatgttccctgtgctgttcctgctgggccctgagggctttggccatattagcccttatggctctgcaattggccatagcattctggatctgattgcaaagaatatgtggggagtgctgggcaactatctgagggtgaagattcatgaacacattctgctgtatggagatattaggaaaaaacagaaaattaccattgcaggccaggagatggaagtggaaaccctggtggcagaagaagaagataagtatgagagcagcctggag

SEQ ID NO: 60 - DNA, 3634 bp, synthetic construct
  source 1..3634: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccttgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaccgccaccatggtgtctagaagaccttggctgctggccctggctctggctgtggcccttgctgctggatctgctggagccagcacaggctcagatgccacagtgccagtggccacacaggacggcccagattatgtgtttcacagagcccatgagagaatgctgttccaaacaagctatacactggaaaacaacggatctgtgatctgcatccccaacaacggccagtgcttctgcctggcttggcttaagagtaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctgggaatggagtggtgtggatgaggtatggagagtggctcctgacctgtcctgtgatcctgatccacctgtctaatctgacaggactgaaagacgactacagcaaaaggactatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaaaaacatgtggggagtgctgggaaattacctgagagtgaagatccatgagcacattctgctgtatggagacatcagaaagaaacagaagatcaccattgcaggccaggaaatggaagttgagacactggtggctgaagaagaagataagtatgagagcagcctggaataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 61 - DNA, 1056 bp, synthetic construct; identical to SEQ ID NO: 16
  source 1..1056: mol_type = other DNA

SEQ ID NO: 62 - DNA, 1056 bp, synthetic construct
  source 1..1056: mol_type = other DNA
atggtgtctagaagaccttggctgctggccctggctctggccgtggcccttgccgctggaagcgctggcgccagcaccggctccgacgccacagtgccagtggccacacaggacggccctgattacgtgtttcacagagcccacgagagaatgctgttccaaacaagctatacactggaaaacaacggcagcgtgatctgcatccccaacaacggccagtgcttctgcctggcctggctgaagagcaacggcaccaacgccgagaagctggctgccaacatcctgcagtgggtcgtgttcgccctgtctgttgcttgcctgggctggtacgcctaccaggcctggcgcgctacctgcggctgggaagaggtgtatgtggccctgatcgagatgatgaaaagcatcatcgaggccttccacgagttcgacagccctgccaccctgtggctgtccagcggcaacggcgtggtgtggatgcggtacggagaatggctcctgacctgtcctgtgatcctgatccacctgtctaatctgacaggactgaaggacgactacagcaagcggaccatgggcctgctggtctctgatgtgggatgtatcgtgtggggagccaccagcgccatgtgcaccggctggaccaagatcctgtttttcctgatttccctgagctacggcatgtacacctacttccatgccgcaaaagtgtacatcgaggctttccacaccgtgcctaagggtctgtgtagacagctggtgcgggccatggcctggctgttcttcgtgtcctggggcatgttccccgtgctgtttctgctgggccccgagggcttcggccacatctctccttacggcagcgccatcggccacagcatcctggacctgatcgccaagaacatgtggggcgtgctgggaaattacctgagagtgaagatccacgagcacattctgctgtacggcgacatcagaaagaaacagaagatcaccatcgccggccaggagatggaagtcgagacactggtggccgaagaagaagataagtacgagagcagcctggaa

SEQ ID NO: 63 - DNA, 1056 bp, synthetic construct
  source 1..1056: mol_type = other DNA
atggtgagcagaagaccctggctgctggccctggccctggctgtggccctggctgctggctctgctggagccagcacaggctctgatgccacagtgcctgtggccacccaggatggccctgactatgtgttccacagagcccatgagagaatgctgttccagaccagctacaccctggagaacaatggctctgtgatctgcatccccaacaatggccagtgcttctgcctggcctggctgaagagtaatggcaccaatgctgagaagctggctgccaacatcctgcagtgggtggtgtttgcactgtctgtggcatgcctgggctggtatgcatatcaggcatggagggcaacctgtggctgggaagaagtgtatgtggcactgattgaaatgatgaaaagcattattgaagcatttcatgaatttgatagcccagccaccctgtggctgagctctggcaatggagtggtgtggatgaggtatggagaatggctgctgacctgccctgtgattctgattcatctgagcaacctgacaggcctgaaagatgattatagcaaaaggaccatgggcctgctggtgtctgatgtgggctgcattgtgtggggagcaacctctgcaatgtgcacaggctggaccaaaattctgttttttctgattagcctgagctatggcatgtatacctattttcatgcagccaaagtgtatattgaagcatttcatacagtgccaaaaggcctgtgcagacagctggtgagagccatggcctggctgttctttgtgagctggggcatgttccctgtgctgttcctgctgggccctgagggctttggccatattagcccttatggctctgcaattggccatagcattctggatctgattgcaaagaacatgtggggagtgctgggcaactatctgagggtgaaaattcatgaacatattctgctgtatggagatattaggaaaaaacagaaaattaccattgcaggccaggaaatggaagtggaaaccctggtggcagaagaagaagataagtatgagagcagcctggag

SEQ ID NO: 64 - DNA, 1056 bp, synthetic construct
  source 1..1056: mol_type = other DNA
atggtgtctagaagaccttggctgctggccctggctctggctgtggcccttgctgctggatctgctggagccagcacaggctcagatgccacagtgccagtggccacacaggatggccctgattatgtgtttcacagagcccatgagagaatgctgttccaaacaagctatacactggaaaacaatggatctgtgatctgcatccccaacaatggccagtgcttctgcctggcctggctgaagagcaatggcaccaatgcagagaagctggctgccaacatcctgcagtgggttgtgtttgccctgtctgttgcttgcctgggctggtatgcctaccaggcctggagggctacctgtggctgggaagaggtgtatgtggccctgattgagatgatgaaaagcatcattgaggccttccatgagtttgacagccctgccaccctgtggctgtcctctggcaatggagtggtgtggatgaggtatggagaatggctcctgacctgtcctgtgatcctgatccacctgtctaatctgacaggactgaaggatgactacagcaaaaggaccatgggcctgctggtctctgatgtgggatgtattgtgtggggagccacctctgccatgtgcacaggctggaccaagatcctgtttttcctgatttccctgagctatggcatgtacacctacttccatgctgcaaaagtgtacattgaggctttccacacagtgcctaagggtctgtgtagacagctggtgagggccatggcctggctgttctttgtgtcctggggcatgttccctgtgctgtttctgctgggccctgagggctttggccacatctctccttatggctctgccattggccacagcatcctggacctgatagccaagaacatgtggggagtgctgggaaattacctgagagtgaagatccatgagcacattctgctgtatggagacatcagaaagaaacagaagatcaccattgcaggccaggagatggaagttgagacactggtggctgaagaagaagataagtatgagagcagcctggaa

SEQ ID NO: 65 - DNA, 1056 bp, synthetic construct
  source 1..1056: mol_type = other DNA
atggtgagcagaagaccctggcttttggccttggccctggctgtggcccttgctgctggatctgctggggcatctacaggctctgatgctacagttccagttgcaacccaggatggccctgactatgtgttccacagagcccatgagagaatgctgttccagaccagctacaccctggagaacaatggctctgtgatctgcatccccaacaatggccagtgcttctgccttgcatggctgaagagtaatggcaccaatgctgagaagctggctgccaacatcctgcagtgggtggtgtttgcactgtctgtggcatgcctgggctggtatgcatatcaggcatggagggcaacctgtgggtgggaagaagtgtatgtggcactgattgaaatgatgaaaagcattattgaagccttccatgaatttgatagcccagccaccctgtggctgagctctggcaatggagtggtgtggatgaggtatggagaatggctgctgacctgccctgtgattctgattcatctgagcaacctgacaggcctgaaagatgattatagcaaaaggaccatgggcctgctggtgtctgatgtgggctgcattgtgtggggagcaacctctgcaatgtgcacaggctggaccaaaattctgttcttcctgattagcctgagctatggcatgtatacctattttcatgcagccaaagtgtatattgaagcatttcatacagtgccaaaaggcctgtgcagacagctggtgagagccatggcctggctgttctttgtgagctggggaatgttccctgtgctgttcctgctgggccctgagggctttggccatattagcccttatggctctgcaattggccatagcattctggatctgattgcaaagaacatgtggggagtgctgggcaactatctgagggtgaagattcatgaacacattctgctgtatggagatattaggaaaaaacagaaaattaccattgcaggccaggaaatggaagtggaaaccctggtggcagaagaagaagataagtatgagagcagcctggag

SEQ ID NO: 66 - DNA, 1056 bp, synthetic construct
  source 1..1056: mol_type = other DNA
atggtgtctagaagaccttggctgctggccctggctctggctgtggcccttgctgctggatctgctggagccagcacaggctcagatgccacagtgccagtggccacacaggatggcccagattatgtgtttcacagagcccatgagagaatgctgttccaaacaagctatacactggaaaacaatggatctgtgatctgcatccccaacaatggccagtgcttctgcctggcatggcttaagagcaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctggcaatggagtggtgtggatgaggtatggagaatggctcctgacctgtcctgtgatcctgatccacctgtctaatctgacaggactgaaggatgactacagcaaaaggaccatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaagaacatgtggggagtgctgggaaattacctgagagtgaagatccatgagcacattctgctgtatggagacatcagaaagaaacagaagatcaccattgcaggccaggagatggaagttgagacactggtggctgaagaagaagataagtatgagagcagcctggaa

SEQ ID NO: 67 - DNA, 3711 bp, synthetic construct
  source 1..3711: mol_type = other DNA
gtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggcttttggccttggccctggctgtggcccttgctgctggatctgctggggcatctacaggctctgatgctacagttccagttgcaacccaggatggccctgactatgtgttccacagagcccatgagagaatgctgttccagaccagctacaccctggagaacaatggctctgtgatctgcatccccaacaatggccagtgcttctgccttgcatggctgaagagtaatggcaccaatgctgagaagctggctgccaacatcctgcagtgggtggtgtttgcactgtctgtggcatgcctgggctggtatgcatatcaggcatggagggcaacctgtgggtgggaagaagtgtatgtggcactgattgaaatgatgaaaagcattattgaagccttccatgaatttgatagcccagccaccctgtggctgagctctggcaatggagtggtgtggatgaggtatggagaatggctgctgacctgccctgtgattctgattcatctgagcaacctgacaggcctgaaagatgattatagcaaaaggaccatgggcctgctggtgtctgatgtgggctgcattgtgtggggagcaacctctgcaatgtgcacaggctggaccaaaattctgttcttcctgattagcctgagctatggcatgtatacctattttcatgcagccaaagtgtatattgaagcatttcatacagtgccaaaaggcctgtgcagacagctggtgagagccatggcctggctgttctttgtgagctggggaatgttccctgtgctgttcctgctgggccctgagggctttggccatattagcccttatggctctgcaattggccatagcattctggatctgattgcaaagaacatgtggggagtgctgggcaactatctgagggtgaagattcatgaacacattctgctgtatggagatattaggaaaaaacagaaaattaccattgcaggccaggaaatggaagtggaaaccctggtggcagaagaagaagataagtatgagagcagcctggagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggc

SEQ ID NO: 68 - DNA, 3706 bp, synthetic construct
  source 1..3706: mol_type = other DNA
tgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgagcagaagaccctggcttttggccttggccctggctgtggcccttgctgctggatctgctggggcatctacaggctctgatgctacagttccagttgcaacccaggacggccctgactatgtgttccacagagcccatgagagaatgctgttccagaccagctacaccctggagaacaacggctctgtgatctgcatccccaacaacggccagtgcttctgccttgcatggctgaagagtaatggcaccaatgctgagaagctggctgccaacatcctgcagtgggtggtgtttgcactgtctgtggcatgcctgggctggtatgcatatcaggcttggagggcaacctgtgggtgggaagaagtgtatgtggcactgattgaaatgatgaaaagcattattgaagccttccatgaatttgatagcccagccaccctgtggctgagctctggcaacggagtggtgtggatgaggtatggagagtggctgctgacctgccctgtgattctgattcatctgagcaacctgacaggcctgaaagatgattatagcaaaaggactatgggcctgctggtgtctgatgtgggctgcattgtgtggggagcaacctctgcaatgtgcacaggctggaccaaaattctgttcttcctgattagcctgtcttatggcatgtatacctattttcatgcagccaaagtgtatattgaagcatttcatacagtgccaaaaggcctgtgcagacagctggtgagagctatggcctggctgttctttgtgagctggggaatgttccctgtgctgttcctgctgggccctgagggctttggccatattagcccttatggctctgcaattggccatagcattctggatctgattgcaaagaatatgtggggagtgctgggcaactatctgagggtgaagattcatgaacacattctgctgtatggagatattaggaaaaaacagaaaattaccattgcaggccaggagatggaagtggaaaccctggtggcagaagaagaagataagtatgagagcagcctggagtaaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgc

SEQ ID NO: 69 - DNA, 1056 bp, synthetic construct; identical to SEQ ID NO: 59
  source 1..1056: mol_type = other DNA

SEQ ID NO: 70 - protein, 352 aa, synthetic construct; identical to SEQ ID NO: 32

SEQ ID NO: 71 - DNA, 3694 bp, synthetic construct
  source 1..3694: mol_type = other DNA
gcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgtctagaagaccttggctgctggccctggctctggctgtggcccttgctgctggatctgctggagccagcacaggctcagatgccacagtgccagtggccacacaggatggcccagattatgtgtttcacagagcccatgagagaatgctgttccaaacaagctatacactggaaaacaatggatctgtgatctgcatccccaacaatggccagtgcttctgcctggcatggcttaagagcaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctggcaatggagtggtgtggatgaggtatggagaatggctcctgacctgtcctgtgatcctgatccacctgtctaatctgacaggactgaaggatgactacagcaaaaggaccatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaagaacatgtggggagtgctgggaaattacctgagagtgaagatccatgagcacattctgctgtatggagacatcagaaagaaacagaagatcaccattgcaggccaggagatggaagttgagacactggtggctgaagaagaagataagtatgagagcagcctggaataaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagct

SEQ ID NO: 72 - DNA, 3707 bp, synthetic construct
  source 1..3707: mol_type = other DNA
gcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaggatccgccaccatggtgtctagaagaccttggctgctggccctggctctggctgtggcccttgctgctggatctgctggagccagcacaggctcagatgccacagtgccagtggccacacaggacggcccagattatgtgtttcacagagcccatgagagaatgctgttccaaacaagctatacactggaaaacaacggatctgtgatctgcatccccaacaacggccagtgcttctgcctggcttggcttaagagtaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctgggaatggagtggtgtggatgaggtatggagagtggctcctgacctgtcctgtgatcctgatccacctgtctaatctgacaggactgaaagacgactacagcaaaaggactatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaaaaacatgtggggagtgctgggaaattacctgagagtgaagatccatgagcacattctgctgtatggagacatcagaaagaaacagaagatcaccattgcaggccaggaaatggaagttgagacactggtggctgaagaagaagataagtatgagagcagcctggaataaagcggccgccggcaagtaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcg

SEQ ID NO: 73 - DNA, 1056 bp, synthetic construct; identical to SEQ ID NO: 58
  source 1..1056: mol_type = other DNA

SEQ ID NO: 74 - protein, 352 aa, synthetic construct; identical to SEQ ID NO: 32

SEQ ID NO: 75 - DNA, 3634 bp, synthetic construct
  source 1..3634: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccttgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaccgccaccatggtgagcagaagaccctggcttttggccttggccctggctgtggcccttgctgctggatctgctggggcatctacaggctctgatgctacagttccagttgcaacccaggacggccctgactatgtgttccacagagcccatgagagaatgctgttccagaccagctacaccctggagaacaacggctctgtgatctgcatccccaacaacggccagtgcttctgccttgcatggctgaagagtaatggcaccaatgctgagaagctggctgccaacatcctgcagtgggtggtgtttgcactgtctgtggcatgcctgggctggtatgcatatcaggcttggagggcaacctgtgggtgggaagaagtgtatgtggcactgattgaaatgatgaaaagcattattgaagccttccatgaatttgatagcccagccaccctgtggctgagctctggcaacggagtggtgtggatgaggtatggagagtggctgctgacctgccctgtgattctgattcatctgagcaacctgacaggcctgaaagatgattatagcaaaaggactatgggcctgctggtgtctgatgtgggctgcattgtgtggggagcaacctctgcaatgtgcacaggctggaccaaaattctgttcttcctgattagcctgtcttatggcatgtatacctattttcatgcagccaaagtgtatattgaagcatttcatacagtgccaaaaggcctgtgcagacagctggtgagagctatggcctggctgttctttgtgagctggggaatgttccctgtgctgttcctgctgggccctgagggctttggccatattagcccttatggctctgcaattggccatagcattctggatctgattgcaaagaatatgtggggagtgctgggcaactatctgagggtgaagattcatgaacacattctgctgtatggagatattaggaaaaaacagaaaattaccattgcaggccaggagatggaagtggaaaccctggtggcagaagaagaagataagtatgagagcagcctggagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 76 - DNA, 3634 bp, synthetic construct
  source 1..3634: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaccgccaccatggtgtctagaagaccttggctgctggccctggctctggctgtggcccttgctgctggatctgctggagccagcacaggctcagatgccacagtgccagtggccacacaggacggcccagattatgtgtttcacagagcccatgagagaatgctgttccaaacaagctatacactggaaaacaacggatctgtgatctgcatccccaacaacggccagtgcttctgcctggcttggcttaagagtaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctgggaatggagtggtgtggatgaggtatggagagtggctcctgacctgtcctgtgatcctgatccacctgtctaatctgacaggactgaaagacgactacagcaaaaggactatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaaaaacatgtggggagtgctgggaaattacctgagagtgaagatccatgagcacattctgctgtatggagacatcagaaagaaacagaagatcaccattgcaggccaggaaatggaagttgagacactggtggctgaagaagaagataagtatgagagcagcctggaataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 77 - DNA, 3634 bp, synthetic construct
  source 1..3634: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcaacccaagaaattacaggctgaaaccagaaaagaacacattaaagcaccaagagaaagttggagtgggttgaagggaaacagatttttaaagttaaggctctgtgaaatgggtagaattaactacaggttaaaaataaaatgttaactaaaggttgcctctgagtaacaggattatgggtgattttaattgtcttctttgtgtatgttcaacagtgactataatatgtattacttttggaataaaggaaaacctgaaaggtgtgttgttttataagggcccttaggttgccaaaattagagtcattgaaatctaaagctgataaaaactttagtgcaaagattgtgacatgggagactacacataccagatccataatgtacatgaggacagtaggccgaggggccctgcacattgaaagcccacatgggagaagcccttgggaaggggagtggaaggatgaggcaaggggccggggggatgcagaggctggcaggcagtcatttctcagcttcagccattcccgccatgggggaatgtggacagagaagccaaacaaatctcctaaacagtaaatgtcagtcttctgtgtcagatatttaagaaaactaacagaggtcagagaagacacacctacagcaagtagactgtccctgtgctgcctttttgcaacccctgctttggcagtgctcaagcccacctcctgctctgtgcagacatctcttctttgctcttactagaccaaggtgaaagaaaactctcaccttctcccatctggccccacagcatctggaacacactgatcctcataatccttgttcttgagaaatattaatgacttaatctcccaagcttgctccctctcctgtgcaggccatctcagtatgttttgcagacaagacccagagaagtccagactggacttgttgcagactgcaaaactgccattggaaggcctccgtcccagtccttctacagagtagccagtgggattcccagccgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaccgccaccatggtgagcagaagaccctggcttttggccttggccctggctgtggcccttgctgctggatctgctggggcatctacaggctctgatgctacagttccagttgcaacccaggacggccctgactatgtgttccacagagcccatgagagaatgctgttccagaccagctacaccctggagaacaacggctctgtgatctgcatccccaacaacggccagtgcttctgccttgcatggctgaagagtaatggcaccaatgctgagaagctggctgccaacatcctgcagtgggtggtgtttgcactgtctgtggcatgcctgggctggtatgcatatcaggcttggagggcaacctgtgggtgggaagaagtgtatgtggcactgattgaaatgatgaaaagcattattgaagccttccatgaatttgatagcccagccaccctgtggctgagctctggcaacggagtggtgtggatgaggtatggagagtggctgctgacctgccctgtgattctgattcatctgagcaacctgacaggcctgaaagatgattatagcaaaaggactatgggcctgctggtgtctgatgtgggctgcattgtgtggggagcaacctctgcaatgtgcacaggctggaccaaaattctgttcttcctgattagcctgtcttatggcatgtatacctattttcatgcagccaaagtgtatattgaagcatttcatacagtgccaaaaggcctgtgcagacagctggtgagagctatggcctggctgttctttgtgagctggggaatgttccctgtgctgttcctgctgggccctgagggctttggccatattagcccttatggctctgcaattggccatagcattctggatctgattgcaaagaatatgtggggagtgctgggcaactatctgagggtgaagattcatgaacacattctgctgtatggagatattaggaaaaaacagaaaattaccattgcaggccaggagatggaagtggaaaccctggtggcagaagaagaagataagtatgagagcagcctggagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 78 - DNA, 46 bp, Adeno-associated virus
  source 1..46: mol_type = genomic DNA
ggaggggtggagtcgtgacgtgaattacgtcatagggttagggagg

SEQ ID NO: 79 - DNA, 37 bp, Adeno-associated virus
  source 1..37: mol_type = genomic DNA
gtagataagtagcatggcgggttaatcattaactaca

SEQ ID NO: 80 - DNA, 2949 bp, synthetic construct
  source 1..2949: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactccatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaccgccaccatggtgtctagaagaccttggctgctggccctggctctggctgtggcccttgctgctggatctgctggagccagcacaggctcagatgccacagtgccagtggccacacaggacggcccagattatgtgtttcacagagcccatgagagaatgctgttccaaacaagctatacactggaaaacaacggatctgtgatctgcatccccaacaacggccagtgcttctgcctggcttggcttaagagtaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctgggaatggagtggtgtggatgaggtatggagagtggctcctgacctgtcctgtgatcctgatccacctgtctaatctgacaggactgaaagacgactacagcaaaaggactatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaaaaacatgtggggagtgctgggaaattacctgagagtgaagatccatgagcacattctgctgtatggagacatcagaaagaaacagaagatcaccattgcaggccaggaaatggaagttgagacactggtggctgaagaagaagataagtatgagagcagcctggaataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 2949 bp, synthetic construct
  source 1..2949: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactccatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaccgccaccatggtgagcagaagaccctggcttttggccttggccctggctgtggcccttgctgctggatctgctggggcatctacaggctctgatgctacagttccagttgcaacccaggacggccctgactatgtgttccacagagcccatgagagaatgctgttccagaccagctacaccctggagaacaacggctctgtgatctgcatccccaacaacggccagtgcttctgccttgcatggctgaagagtaatggcaccaatgctgagaagctggctgccaacatcctgcagtgggtggtgtttgcactgtctgtggcatgcctgggctggtatgcatatcaggcttggagggcaacctgtgggtgggaagaagtgtatgtggcactgattgaaatgatgaaaagcattattgaagccttccatgaatttgatagcccagccaccctgtggctgagctctggcaacggagtggtgtggatgaggtatggagagtggctgctgacctgccctgtgattctgattcatctgagcaacctgacaggcctgaaagatgattatagcaaaaggactatgggcctgctggtgtctgatgtgggctgcattgtgtggggagcaacctctgcaatgtgcacaggctggaccaaaattctgttcttcctgattagcctgtcttatggcatgtatacctattttcatgcagccaaagtgtatattgaagcatttcatacagtgccaaaaggcctgtgcagacagctggtgagagctatggcctggctgttctttgtgagctggggaatgttccctgtgctgttcctgctgggccctgagggctttggccatattagcccttatggctctgcaattggccatagcattctggatctgattgcaaagaatatgtggggagtgctgggcaactatctgagggtgaagattcatgaacacattctgctgtatggagatattaggaaaaaacagaaaattaccattgcaggccaggagatggaagtggaaaccctggtggcagaagaagaagataagtatgagagcagcctggagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 82 - DNA, 3190 bp, synthetic construct
  source 1..3190: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagccgccaccatggtgtctagaagaccttggctgctggccctggctctggctgtggcccttgctgctggatctgctggagccagcacaggctcagatgccacagtgccagtggccacacaggacggcccagattatgtgtttcacagagcccatgagagaatgctgttccaaacaagctatacactggaaaacaacggatctgtgatctgcatccccaacaacggccagtgcttctgcctggcttggcttaagagtaatggcaccaatgcagagaagctggctgccaacatcctgcagtgggttgtgtttgccctgtctgttgcttgcctgggctggtatgcctaccaagcctggagggctacctgtgggtgggaagaggtgtatgtggccctgattgagatgatgaaaagcatcattgaggccttccatgagtttgacagccctgccaccctgtggctgtcctctgggaatggagtggtgtggatgaggtatggagagtggctcctgacctgtcctgtgatcctgatccacctgtctaatctgacaggactgaaagacgactacagcaaaaggactatgggcctgctggtctctgatgtgggatgtattgtgtggggagccacctctgccatgtgcacaggctggaccaagatcctgtttttcctgatttccctgagctatggaatgtacacctacttccatgctgcaaaagtgtacattgaggctttccacacagtgcctaagggtctgtgtagacagctggtgagggctatggcctggcttttctttgtgtcctggggcatgttccctgtgctgtttctgctgggccctgagggctttggccacatctctccttatggctctgccattggccacagcatcctggacctgatagccaaaaacatgtggggagtgctgggaaattacctgagagtgaagatccatgagcacattctgctgtatggagacatcagaaagaaacagaagatcaccattgcaggccaggaaatggaagttgagacactggtggctgaagaagaagataagtatgagagcagcctggaataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 83 - DNA, 3190 bp, synthetic construct
  source 1..3190: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgactcgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagccgccaccatggtgagcagaagaccctggcttttggccttggccctggctgtggcccttgctgctggatctgctggggcatctacaggctctgatgctacagttccagttgcaacccaggacggccctgactatgtgttccacagagcccatgagagaatgctgttccagaccagctacaccctggagaacaacggctctgtgatctgcatccccaacaacggccagtgcttctgccttgcatggctgaagagtaatggcaccaatgctgagaagctggctgccaacatcctgcagtgggtggtgtttgcactgtctgtggcatgcctgggctggtatgcatatcaggcttggagggcaacctgtgggtgggaagaagtgtatgtggcactgattgaaatgatgaaaagcattattgaagccttccatgaatttgatagcccagccaccctgtggctgagctctggcaacggagtggtgtggatgaggtatggagagtggctgctgacctgccctgtgattctgattcatctgagcaacctgacaggcctgaaagatgattatagcaaaaggactatgggcctgctggtgtctgatgtgggctgcattgtgtggggagcaacctctgcaatgtgcacaggctggaccaaaattctgttcttcctgattagcctgtcttatggcatgtatacctattttcatgcagccaaagtgtatattgaagcatttcatacagtgccaaaaggcctgtgcagacagctggtgagagctatggcctggctgttctttgtgagctggggaatgttccctgtgctgttcctgctgggccctgagggctttggccatattagcccttatggctctgcaattggccatagcattctggatctgattgcaaagaatatgtggggagtgctgggcaactatctgagggtgaagattcatgaacacattctgctgtatggagatattaggaaaaaacagaaaattaccattgcaggccaggagatggaagtggaaaccctggtggcagaagaagaagataagtatgagagcagcctggagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgagtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 84 - DNA, 2175 bp, synthetic construct
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 85 - DNA, 741 bp, synthetic construct
  source 1..741: mol_type = other DNA
gttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagag

SEQ ID NO: 86 - DNA, 590 bp, synthetic construct
  source 1..590: mol_type = other DNA
taatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 87 - DNA, 485 bp, synthetic construct
  source 1..485: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgatttt

SEQ ID NO: 88 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 89 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 90 - DNA, 21 bp, synthetic construct
  source 1..21: mol_type = other DNA
ggaacccctagtgatggagtt

SEQ ID NO: 91 - DNA, 16 bp, synthetic construct
  source 1..16: mol_type = other DNA
cggcctcagtgagcga

SEQ ID NO: 92 - DNA, 21 bp, synthetic construct
  source 1..21: mol_type = other DNA
cactccctctctgcgcgctcg

SEQ ID NO: 93 - DNA, 19 bp, synthetic construct
  source 1..19: mol_type = other DNA
ggctgttgggcactgacaa

SEQ ID NO: 94 - DNA, 22 bp, synthetic construct
  source 1..22: mol_type = other DNA
ccaaggaaaggacgatgatttc

SEQ ID NO: 95 - DNA, 15 bp, synthetic construct
  source 1..15: mol_type = other DNA
tccgtggtgttgtcg

(19) United States
(12) Patent Application Publication
(10) Pub. No.: US 2025/0250311 A1
(43) Pub. Date: Aug. 7, 2025

(54) HUMAN CONE PHOTORECEPTOR OPTOGENETIC CONSTRUCTS

(71) Applicant: INSTITUTE OF MOLECULAR AND CLINICAL OPHTHALMOLOGY BASEL (IOB), BASEL (CH)

(72) Inventors: Bence György, Riehen (CH); Botond Roska, Oberwil (CH); Hendrik P.N. Scholl, Binningen (CH)

(21) Appl. No.: 19/044,053

(22) Filed: Feb. 3, 2025

Related U.S. Application Data
(63) Continuation of application No. PCT/IB2023/058059, filed on Aug. 9, 2023.
(60) Provisional application No. 63/397,180, filed on Aug. 11, 2022.
(60) Provisional application No. 63/400,095, filed on Aug. 23, 2022.

(51) Int. Cl. C07K 14/47; A61K 48/00; A61P 27/02; C12N 15/86

(52) U.S. Cl. CPC C07K 14/4702; A61K 48/0066; A61P 27/02; C12N 15/86; C12N 2750/14143; C12N 2830/48; C12N 2830/50

(57) ABSTRACT

The disclosure relates to nucleic acids comprising a synthetic cone-specific promoter, a depolarizing optogenetic protein and optionally a reporter molecule; and a Woodchuck Hepatitis Virus Posttranscriptional Regulatory element (WPRE). The nucleic acid can be used in vectors or host cells to drive expression of the depolarizing optogenetic protein in retinal cells, particularly cone photoreceptors.

The present application is a continuation of International Application No. PCT/IB2023/058059, filed on Aug. 9, 2023, which designated the International Bureau, which claims the benefit of U.S. Provisional Application No. 63/397,180, filed on Aug. 11, 2022, and U.S. Provisional Application No. 63/400,095, filed on Aug. 23, 2022, each of which are incorporated herein by reference in their entirety.

The instant application contains a Sequence Listing which has been submitted electronically in XML format and is hereby incorporated by reference in its entirety. Said XML copy, created on Aug. 3, 2023, is named 761131_500010_SL.xml and is 217,974 bytes in size.

### 1. BACKGROUND

Blindness is the complete or nearly complete loss of vision and in most forms are incurable. It is estimated that 43 million people were blind in 2020 (GBD 2019 Blindness and Vision Impairment Collaborators, Lancet Glob Health, (2021), 9(2):e130-e143). One of the most common cause of blindness is retinal disease, such as degeneration or dysfunction of retinal photoreceptors cells and the consequent loss of light sensitivity. In a certain subset of blind patients, studies have shown that cone photoreceptors remain alive in a dormant stage (Cideciyan and Jacobson, Invest Ophthalmol. Vis. Sci. (2019), 60(5):1680-1695).

Photoreceptors are responsible for the detection and transduction of light signals (Osakada et al., (2008), Nat Biotechnol., 26(2):215-224). Photoreceptor cells involved in vision are rods and cones. These cells contain a chromophore (a molecule that absorbs light), such as retinal, bound to cell membrane proteins referred to as opsins (e.g., rhodopsin). Rods primarily contribute to night-time vision (scotopic conditions) whereas cones primarily contribute to day-time vision (photopic conditions). When light hits the photoreceptor, it causes a shape change in the chromophore, for example, converting retinal from the 11-cis isomer that predominates in dark conditions to the all-trans isomer. The isomerization of the chromophore causes structural changes in the opsin protein. This photoactivation causes a cascade of events that leads to hyperpolarization of the photoreceptor cell membrane potential. Thus, unlike most sensory neurons, which become depolarized by exposure to a stimulus, photoreceptors become hyperpolarized upon photostimulation. Choung et al., (2014), Nature Neuroscience, 17, 1123-1129; Carter and Lecea (2011), Trends Mol. Med., 17(4):197-206; Simon et al., (2020), BBRC, 527(2):325-330.

“Optogenetics” is a term of art that refers to methods in which cells are typically genetically modified to express light sensors to make the cells responsive to light. Accordingly, optogenetic approaches have potential for treating various types of blindness. For example, some optogenetic approaches in patients with dormant cone photoreceptors focus on recapitulating the normal healthy human cone cell by resensitizing these dysfunctional cone cells to light by genetically expressing light-activated hyperpolarizing ion pumps in the cone photoreceptors, which in their health state hyperpolarize in response to optical stimulation. Choung et al., (2014), Nature Neuroscience, 17, 1123-1129; Carter and Lecea (2011), Trends Mol. Med., 17(4):197-206; Simon et al., (2020), BBRC, 527(2):325-330. Khabou et al., JCI Insight, (2018) 3(2):e96029, Nikonov et al., (2022) TVST, 11(5):24.

Another optogenetic approach has focused on transducing ganglion cells with an optogenetic protein. Sahel et al., (2021), Nature Medicine, 27, 1223-1229. Ganglion cells are the final output cells of the retina, which collect information about the visual world from bipolar cells and amacrine cells in the form of chemical messengers sensed by receptors that trigger an electrical signal. These electrical signals create a retinal computation by generating a diversity of spikes that are transmitted through the optical nerve to trigger vision. However, transducing ganglion cells with light sensitive optogenetic proteins, transforms the ganglion cells into artificial photoreceptor cells and all transduced cells have substantially the same light-sensing capabilities. In other words, the transduced ganglion cells sense and respond to light to generate substantially the same electrical signals and thereby eliminates the retinal computational effect normally present in the retina, which is important for vision. Furthermore, ganglion and bipolar cells are on a ring around the fovea and not located like pixels on a camera, therefore, optogenetic approaches targeting these cells would lead to major image distortion.

These efforts have not succeeded in restoring vision that is not facilitated using optical devices and few therapies have been evaluated clinically. Sahel et al., Nature Medicine, 27, 1223-1229 (2021). Further, there are well-known obstacles to the effective use of optogenetics for treating blindness, such as insufficient light sensitivity of optogenetically transduced proteins. In addition, targeting the optogenetic proteins selectively to cone cells and achieving sufficient expression levels in cone cells remains a major technical challenge. Thus, it remains largely an experimental approach. An additional challenge is that studies conducted on animal models typically do not translate to human cells and this necessitates vector development directly on human retinas. No previous studies have demonstrated expression and function of optogenetic constructs in human cone cells using optogenetic technology. Thus, translating experimental approaches to humans using optogenetics is highly unpredictable.

There is a need for therapeutic approaches for restoring vision.

### 2. SUMMARY

The disclosure relates to novel optogenetic constructs comprising a depolarizing optogenetic protein, which is preferably a light-gated ion channel polypeptide that is selectively expressed in human cone cells. The optogenetic constructs can restore light sensitivity of human cone cells and retinal information processing when the construct is introduced into and expressed in human cone cells.

As noted above, photoreceptor cells hyperpolarize in response to optical stimulation. Based on this well-known phenomena, it was believed that for any potential optogenetic approach to be successful, hyperpolarization of the photoreceptor cell membrane using a hyperpolarizing protein (e.g., a halorhodopsin) would be essential. Choung et al., (2014), Nature Neuroscience, 17, 1123-1129; Carter and Lecea (2011), Trends Mol. Med., 17(4):197-206; Simon et al., (2020), BBRC, 527(2):325-330. The inventors unexpectedly and surprisingly found that rather than recapitulating the normal healthy human cone photoreceptor cells, human cone cells can be re-sensitized using a depolarizing optogenetic protein that depolarizes the human cone cell membranes in response to optical stimulation. The depolarizing optogenetic protein can generate a depolarization current that depolarizes the human cone cell at sufficient magnitude to generate spiking of ganglion cells. This is opposite to the normal functioning of photoreceptors in a healthy state. Ganglion cells are the final output cells of the retina. Their axons gather at the optic disk, where they become myelinated and form the optic nerve. Activation of ganglion cells triggers vision. Moreover, without wishing to be bound by theory, the inventors expected that all depolarizing optogenetic proteins were expected to function. However, the inventors surprisingly discovered that only a subset of depolarizing optogenetic proteins were functional in human cones, and thus suitable for the optogenetic constructs described herein.

In addition, the inventors surprisingly discovered that a specific combination of regulatory elements and specific depolarizing optogenetic proteins and optionally a reporter molecule and a synthetic intron are necessary to both achieve sufficient expression and restoration of light sensitivity in human cone cells.

Further, the inventors discovered that an identifiable subset of patients with retinal dystrophies and geographic atrophy due to age-related macular degeneration are particularly suitable for therapy with the optogenetic construct disclosed herein (See, Janeschitz-Kriegl et al., (2022), Investigative Opthalmology & Visual Science, 63(7):455). The inventors have found that patients with inherited retinal dystrophies and low vision are suitable candidates for treatment when they have a preserved cone photoreceptor layer in the central retina. Without being bound by theory, patients that are particularly suitable candidates for therapy have at least some remaining cones that appear stable overtime, the cone cells are likely connected to ganglion cells and the patients have a normal appearing optic nerve.

Accordingly, the optogenetic constructs disclosed herein comprise a promoter or a cone-specific promoter, a depolarizing optogenetic protein, and a Woodchuck Hepatitis Virus Posttranscriptional Regulatory element (WPRE). The optogenetic constructs can optionally comprise a reporter molecule. The optogenetic constructs preferably express the depolarizing optogenetic protein in human cone cells, such as dormant human cone cells that are normally not responsive to photostimulation. It is desirable, that other types of cell types, including, but not limited to, rod cells, amacrine cells, or ganglion cells do not express the depolarizing optogenetic protein when the optogenetic construct is introduced into the cell.

In particular, this disclosure relates to isolated nucleic acids that comprise a promoter that is a hybrid promoter comprising a first nucleotide sequence comprising at least a portion of a cone-specific promoter and a second sequence comprising at least a portion of a rod specific promoter, where the first and second nucleotide sequences are operably linked and function as a single promoter for expression in cone photoreceptors, or a cone specific promoter, or an active variant, fragment or truncation of either of the foregoing. The isolated nucleic acid also comprises a nucleotide sequence encoding a depolarizing optogenetic protein and optionally a reporter molecule, and a WPRE. The promoter, the nucleotide sequence encoding a depolarizing optogenetic protein and optionally a reporter molecule, and a WPRE are operably linked. A preferred promoter is Pro573.2 having SEQ ID NO: 14. Another preferred promoter is ProA7 promoter having SEQ ID NO: 2. Another preferred promoter is 4×ProSc having SEQ ID NO: 85.

The isolated nucleic acid can comprise a promoter that is a hybrid promoter, such as a promoter that contains SEQ ID NO: 14, SEQ ID NO: 13, SEQ ID NO: 12, or SEQ ID NO: 11 or active variant, fragment, or truncation of any of the foregoing sequences.

The isolated nucleic acid can further comprise a hybrid promoter and the first nucleotide sequence can include at least about 150 consecutive nucleotides of SEQ ID NO: 2 and the second nucleotide sequence can include at least about 264 consecutive nucleotides of SEQ ID NO: 10, or an active variant, fragment or truncation of any of the foregoing sequences. For example, the first nucleotide sequence can be selected from the group consisting of SEQ ID NO:24, SEQ ID NO:25, SEQ ID NO:26, SEQ ID NO:27, and a sequence that contains at least about 150 continuous nucleotides from the 3′ terminus of SEQ ID NO:2, and the second nucleic acid can include SEQ ID NO:54, or an active variant of any of the foregoing.

The isolated nucleic acid can comprise a hybrid promoter in which the first nucleotide sequence is selected from the group consisting of SEQ ID NO:24, SEQ ID NO:25, SEQ ID NO:26, SEQ ID NO:27, and a sequence that contains at least about 150 continuous nucleotides from the 3′ terminus of SEQ ID NO:2, and the second nucleic acid comprises SEQ ID NO: 10, or an active variant, fragment or truncation thereof.

The isolated nucleic acid can further comprise a nucleotide sequence encoding a polyadenylation signal (PolyA) that is 3′ of the nucleotide sequence encoding the WPRE. The nucleotide sequence encoding the PolyA and the nucleotide sequence encoding the WPRE can be operably linked.

The nucleic acid can include a cone specific promoter comprising at least about 150 consecutive nucleotides of SEQ ID NO:2 or an active variant thereof. In examples, cone-specific promoter is selected from the group consisting of SEQ ID NO:24, SEQ ID NO:25, SEQ ID NO:26, SEQ ID NO:27, and a sequence that contains at least about 150 continuous nucleotides from the 3′ terminus of SEQ ID NO:2, or an active variant thereof.

The nucleic acid can include a hybrid promoter or cone specific promoter comprising one to about ten copies of a sequence selected from the group consisting of SEQ ID NO:24, SEQ ID NO:25, SEQ ID NO:26, SEQ ID NO:27, a sequence that contains at least about 150 continuous nucleotides from the 3′ terminus of SEQ ID NO:2, an active variant of any of the foregoing, and combinations thereof. The hybrid promoter can contain one to about ten copies of SEQ ID NO: 54, or an active variant thereof.

The WPRE in the nucleic acids disclosed herein, can comprise a nucleotide sequence having at least 70% identity to SEQ ID NO: 3 (e.g. SEQ ID NO:8). The isolated nucleic acid can further comprise a nucleotide sequence encoding a polyadenylation signal (PolyA) that is 3′ of the nucleotide sequence encoding the WPRE. The nucleotide sequence encoding the PolyA and the nucleotide sequence encoding the WPRE can be operably linked. The WPRE in the nucleic acid disclosed herein can comprise a nucleotide sequence having at least 70% identity to SEQ ID NO: 86. When the nucleic acids disclosed herein comprises a PolyA, the PolyA can comprise a nucleotide sequence having SEQ ID NO: 87.

The isolated nucleic acid can further comprise an AAV inverted terminal repeat sequence (ITR). The isolated nucleic acid can comprise a first AAV ITR that is 5′ of the promoter and a second AAV ITR that is 3′ of the WPRE and preferably 3′ of the PolyA signal. The depolarizing optogenetic protein can be a light responsive polypeptide. The light responsive polypeptide can be a light-gated ion channel polypeptide. The light-gated ion channel polypeptide can be a channelrhodopsin or a functional variant thereof. The light-gated ion channel polypeptide can be a channelrhodopsin. The channelrhodopsin can be a ReaChR polypeptide or a functional variant thereof.

The isolated nucleic acid preferably comprises a nucleotide sequence encoding ReaChR or a functional variant thereof. The nucleotide sequence encoding ReaChR or a functional variant thereof can be selected from the group consisting of SEQ ID NO:16, SEQ ID NO:32, SEQ ID NO:33, SEQ ID NO:34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or an amino acid sequence with at least about 70% identity to any of the foregoing.

The isolated nucleic acid can comprise a nucleic acid comprising SEQ ID NO: 18, SEQ ID NO: 17, SEQ ID NO: 19, SEQ ID NO: 20, SEQ ID NO: 21, SEQ ID NO: 35, SEQ ID NO: 55, SEQ ID NO: 56, SEQ ID NO: 60, SEQ ID NO: 67, SEQ ID NO: 68, SEQ ID NO: 71, SEQ ID NO: 72, SEQ ID NO: 75, SEQ ID NO: 76, SEQ ID NO: 77, SEQ ID NO: 80, SEQ ID NO: 81, SEQ ID NO: 82, or SEQ ID NO: 83.

Some preferred optogenetic constructs comprise a nucleotide sequence comprising SEQ ID NO: 60, SEQ ID NO: 75, SEQ ID NO:76, SEQ ID NO:77, SEQ ID NO:80, SEQ ID NO:81, SEQ ID NO:82, or SEQ ID NO: 83. A preferred optogenetic construct comprises a nucleotide sequence comprising SEQ ID NO: 60.

The isolated nucleic acid can further comprise a channelrhodopsin or functional variant thereof engineered to enhance membrane localization when expressed in human cone photoreceptors. The engineered channelrhodopsin can comprise SEQ ID NO: 57 or a functional variant thereof.

In some embodiments, the isolated nucleic acid does not encode the optional molecule.

The promoter can comprise two or more first nucleotide sequences; two or more second nucleotide sequences; two or more cone-specific promoters; or two or more thereof. The nucleic acid can further comprise an intron.

The nucleic acid can contain a nucleotide sequence comprising SEQ ID NO: 4, SEQ ID NO: 5, SEQ ID NO: 6, SEQ ID NO: 7, SEQ ID NO: 17, SEQ ID NO: 18, SEQ ID NO: 19, SEQ ID NO: 20, SEQ ID NO: 21, SEQ ID NO: 35, SEQ ID NO: 55, SEQ ID NO: 56, SEQ ID NO: 60, SEQ ID NO: 67, SEQ ID NO: 68, SEQ ID NO: 71, SEQ ID NO: 72, SEQ ID NO: 75, SEQ ID NO: 76, SEQ ID NO: 77, SEQ ID NO: 80, SEQ ID NO: 81, SEQ ID NO: 82, or SEQ ID NO: 83, or a sequence with at least about 70% identity to any of the foregoing. In certain embodiments, the nucleic acid can contain a nucleotide sequence comprising SEQ ID NO: 3, SEQ ID NO: 15, SEQ ID NO: 16, SEQ ID NO: 32, SEQ ID NO: 70, SEQ ID NO: 74, SEQ ID NO: 9, SEQ ID NO: 15, and/or SEQ ID NO: 87, or a sequence with at least about 70% identity to any of the foregoing.

The nucleic acid can further comprise an active variant having at least about 70% identity to the corresponding reference sequence. The active variant can contain one or more of codon optimization, CpG-reduction or elimination, alternative start site removal, repeat removal, hairpin removal, unwanted splice donor and acceptor site removal, ITR terminal resolution site deletion, stuffer sequence addition, or miRNA addition.

The present disclosure also relates to a viral particle comprising a nucleic acid as described herein. The viral particle can be an AAV particle. The present disclosure further relates to a host cell comprising a nucleic acid or a viral particle as described herein.

The disclosure additionally relates to an AAV vector, comprising a nucleic acid as described herein and an AAV capsid. In the AAV vector, the AAV capsid can be an AAV5 capsid, a PHP.eB capsid, an NHP26 capsid, an AAV8 capsid, an AAV8-BP2 capsid, an AAV9 capsid, or a PHP.B capsid. A host cell can comprise an AAV vector described herein.

Also provided herein are pharmaceutical compositions comprising the nucleic acid, the viral particle, the AAV vector, or a host cell as described herein. The pharmaceutical composition can also comprise a pharmaceutically acceptable excipient.

The disclosure also relates to methods of delivering depolarizing optogenetic proteins to a human cone cell in a subject in need thereof. The method comprises administering to the subject the nucleic acid, the viral particle, the AAV vector, the host cell, or the pharmaceutical composition as described herein.

The disclosure also relates to a method for treating a retinal disease, the method comprising administering to a subject in need thereof an effective amount of a nucleic acid, a viral particle, an AAV vector, a host cell, or a pharmaceutical composition as described herein.

The disclosure also relates to methods for treating a retinal disease, for example restoring vision or restoring sensitivity to light in human cone photoreceptor cells, or treating retinal degeneration, comprising administering to a subject in need thereof an effective amount of the nucleic acid disclosed herein, the, viral particle, the AAV vector, the host cell, or a the pharmaceutical composition as described herein. The nucleic acid, AAV vector or a pharmaceutical composition thereof can be administered by subretinal, intravitreal, or suprachoroideal injection. The nucleic acid, AAV vector, or pharmaceutical composition may be administered before, after, or initiation of photoreceptor loss or dysfunction.

The depolarizing optogenetic protein is capable of mediating a depolarizing current that depolarizes a human cone cell when exposed to light when the nucleic acid sequence encoding the depolarizing optogenetic protein is introduced into the human cone cell. The depolarizing current is potent enough to generate an electrical current that is transmitted to the ganglion cells and generates spiking of light-driven ganglion cells. This depolarization is preferably comparable or stronger than light-driven ganglion cell spiking in a functional human cone cell. The light-driven ganglion cell spiking can be assessed using any suitable assay. For example, light-driven ganglion cell spiking can be measured using a multi-electrode array.

Any retinal disease may be suitable for treatment according to the methods and with the compositions disclosed herein. Exemplary retinal disorders that may be suitable for treating include, but are not limited to, retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, post-retinal detachment, cone dysfunction, a tapetoretinal degeneration, retinal vein occlusion and geographic atrophy. Treatment according to the methods and with the compositions disclosed herein is suitable for a subject that has a disorder impairing vision in which the optic nerve retains at least some function.

In some embodiments, the depolarizing optogenetic protein can be expressed in a cell membrane of a human cone cell. In some embodiments, the depolarizing optogenetic protein can be capable of mediating a depolarizing current that depolarizes a human cone cell when exposed to light. In some embodiments, depolarization of the human cone cell induces light-driven ganglion cell spiking that can be comparable to light-driven ganglion cell spiking in a functional human cone cell. In some embodiments, the light-driven ganglion cell spiking can be assessed using a multi-electrode array.

The nucleic acid can be capable of restoring light sensitivity when introduced into a human cone cell, wherein restoration of light sensitivity occurs when (i) the depolarizing optogenetic protein is expressed in a cell membrane of the human cone cell, (ii) the depolarizing optogenetic protein is capable of mediating a depolarizing current that depolarizes a human cone cell when exposed to light, and (iii) depolarization of the human cone cell induces light-driven ganglion cell spiking.

In particular, this disclosure relates to isolated nucleic acids that comprises a promoter comprising (a) a first nucleotide sequence selected from the group consisting of a nucleotide sequence of at least 150 nucleotides which has at least 70% identity to a sequence of equal length from SEQ ID NO: 2, a nucleotide sequence which has at least 70% identity to SEQ ID NO: 22, a nucleotide sequence which has at least 70% identity to SEQ ID NO: 23, and combinations thereof; and a second nucleotide sequence of at least about 370 nucleotides having at least 70% identity to a sequence of equal length from the sequence of SEQ ID NO: 10; or (b) a cone-specific promoter of at least about 150 nucleotides and no more than 499 nucleotides which has at least 70% identity to a sequence of equal length from the sequence of SEQ ID NO: 2. The isolated nucleic acid also comprises a nucleotide sequence encoding a depolarizing optogenetic protein and optionally a reporter molecule, and a Woodchuck Hepatitis Virus Posttranscriptional Regulatory element (WPRE). The promoter, the nucleotide sequence encoding a depolarizing optogenetic protein and optionally a reporter molecule, and the WPRE are operably linked.

The isolated nucleic acid can further comprise a nucleotide sequence encoding a polyadenylation signal (PolyA) that is 3′ of the nucleotide sequence encoding the WPRE. The nucleotide sequence encoding the PolyA and the nucleotide sequence encoding the WPRE can be operably linked.

The isolated nucleic acid can further comprise a nucleotide sequence encoding an AAV inverted terminal repeat sequence (ITR). The isolated nucleic acid can comprise a first AAV ITR that is 5′ of the promoter and a second AAV ITR that is 3′ of the WPRE and preferably 3′ of the PolyA signal. The depolarizing optogenetic protein can be a light responsive polypeptide. The light responsive polypeptide can be a light-gated ion channel polypeptide. The light-gated ion channel polypeptide can be a channelrhodopsin or a functional variant thereof.

The light-gated ion channel polypeptide can be a channelrhodopsin. The channelrhodopsin can be a ReaChR polypeptide or a functional variant thereof.

In some embodiments, the isolated nucleic acid does not encode the optional reporter molecule.

The WPRE can comprise a nucleotide sequence having at least 70% identity to SEQ ID NO: 3. The WPRE can comprise a nucleotide sequence having at least 70% identity to SEQ ID NO: 86.

The promoter can comprise two or more first nucleotide sequences; two or more second nucleotide sequences; two or more cone-specific promoters; or two or more thereof. The nucleic acid can further comprise an intron. The promoter can comprise one or more sequences selected from the group consisting of SEQ ID NO: 2, SEQ ID NO: 10; SEQ ID NO: 11, SEQ ID NO: 12, SEQ ID NO: 13, SEQ ID NO: 14, SEQ ID NO: 22, SEQ ID NO: 23; SEQ ID NO: 24, SEQ ID NO: 25, SEQ ID NO: 26, SEQ ID NO: 27, SEQ ID NO: 30; SEQ ID NO: 36; SEQ ID NO: 37; SEQ ID NO: 38; SEQ ID NO: 39; SEQ ID NO: 40; SEQ ID NO: 41; and SEQ ID NO: 45.

The present disclosure also relates to a viral particle comprising a nucleic acid as described herein.

The viral particle can be an AAV particle. The viral particle can be capable of transducing about 10% of degenerated human cone cells. The present disclosure further relates to a host cell comprising a nucleic acid or a viral particle as described herein.

The disclosure additionally relates to an AAV vector, comprising a nucleic acid as described herein and an AAV capsid. In the AAV vector, the AAV capsid can be an AAV5 capsid, a PHP.eB capsid, an NHP26 capsid, an AAV8 capsid, an AAV8-BP2 capsid, an AAV9 capsid, or a PHP.B capsid; preferably an AAV5 capsid, a PHP.eB capsid, or an NHP26 capsid; more preferably an AAV5 capsid. The promoter of the AAV vector can comprise SEQ ID NO: 2, the depolarizing optogenetic protein can be ReaChR, the optional reporter molecule can be absent, the PolyA can be present and has SEQ ID NO: 9, and the AAV capsid can be an AAV5.

The promoter of the AAV vector can comprise SEQ ID NO: 14, the depolarizing optogenetic protein can be ReaChR, the optional reporter molecule can be absent, the PolyA can be present and has SEQ ID NO: 9, and the AAV capsid can be an AAV5. The promoter of the AAV vector can comprise SEQ ID NO: 12, the depolarizing optogenetic protein can be ReaChR, the optional reporter molecule can be absent, the PolyA can be present and has SEQ ID NO: 9, and the AAV capsid can be an AAV5. A host cell can comprise the AAV vector described herein. The promoter of the AAV can comprise SEQ ID NO: 85, the depolarizing optogenetic protein can be ReaChR, the optional reporter molecule can be absent, the PolyA can be present and has SEQ ID NO: 9, and the AAV capsid can be an AAV5. The promoter of the AAV can comprise SEQ ID NO: 14, the depolarizing optogenetic protein can be ReaChR, the optional reporter molecule can be absent, the PolyA can be present and has SEQ ID NO: 87, and the AAV capsid can be an AAV5. The promoter of the AAV can comprise SEQ ID NO: 12, the depolarizing optogenetic protein can be ReaChR, the optional reporter molecule can be absent, the PolyA can be present and has SEQ ID NO: 87, and the AAV capsid can be an AAV5. The promoter of the AAV can comprise SEQ ID NO: 85, the depolarizing optogenetic protein can be ReaChR, the optional reporter molecule can be absent, the PolyA can be present and has SEQ ID NO: 87, and the AAV capsid can be an AAV5.

The promoter of the AAV vector can comprise SEQ ID NO: 85, the depolarizing optogenetic protein can be ReaChR, the optional reporter molecule can be absent, the PolyA can be present and has SEQ ID NO: 9, and the AAV capsid can be an AAV5 and has SEQ ID NO: 84. The promoter of the AAV vector can comprise SEQ ID NO: 14, the depolarizing optogenetic protein can be ReaChR, the optional reporter molecule can be absent, the PolyA can be present and has SEQ ID NO: 9, and the AAV capsid can be an AAV5 and has SEQ ID NO: 84. The promoter of the AAV vector can comprise SEQ ID NO: 12, the depolarizing optogenetic protein can be ReaChR, the optional reporter molecule can be absent, the PolyA can be present and has SEQ ID NO: 9, and the AAV capsid can be an AAV5 and has SEQ ID NO: 84.

The promoter of the AAV vector can comprise SEQ ID NO: 12, the depolarizing optogenetic protein can be ReaChR, the optional reporter molecule can be absent, the PolyA can be present and has SEQ ID NO: 87, and the AAV capsid can be an AAV5 and has SEQ ID NO: 84. The promoter of the AAV vector can comprise SEQ ID NO: 85, the depolarizing optogenetic protein can be ReaChR, the optional reporter molecule can be absent, the PolyA can be present and has SEQ ID NO: 87, and the AAV capsid can be an AAV5 and has SEQ ID NO: 84. The promoter of the AAV vector can comprise SEQ ID NO: 14, the depolarizing optogenetic protein can be ReaChR, the optional reporter molecule can be absent, the PolyA can be present and has SEQ ID NO: 87, and the AAV capsid can be an AAV5 and has SEQ ID NO: 84.

Also provided herein are pharmaceutical compositions comprising the nucleic acid, the viral particle, the AAV vector, or a host cell as described herein. The pharmaceutical composition can also comprise a pharmaceutically acceptable excipient.

The disclosure also relates to methods of delivering depolarizing optogenetic proteins to a human cone cell in a subject in need thereof. The method comprises administering to the subject the nucleic acid, the viral particle, the AAV vector, the host cell, or the pharmaceutical composition as described herein.

The disclosure also relates to a method for treating a retinal disease, the method comprising administering to a subject in need thereof an effective amount of a nucleic acid, a viral particle, an AAV vector, a host cell, or a pharmaceutical composition as described herein.

The disclosure also relates to methods for treating a retinal disease, for example restoring vision or restoring sensitivity to light in human cone photoreceptor cells, or treating retinal degeneration, comprising administering to a subject in need thereof an effective amount of the nucleic acid disclosed herein, the, viral particle, the AAV vector, the host cell, or the pharmaceutical composition as described herein. The nucleic acid, AAV vector or a pharmaceutical composition thereof can be administered by subretinal injection. The nucleic acid, AAV vector, or pharmaceutical composition may be administered before, after, or initiation of photoreceptor loss or dysfunction.

The depolarizing optogenetic protein is capable of mediating a depolarizing current that depolarizes a human cone cell when exposed to light when the nucleic acid sequence encoding the depolarizing optogenetic protein is introduced into the human cone cell. The depolarizing current is potent enough to generate an electrical current that is transmitted to the ganglion cells and generates spiking of light-driven ganglion cells. This depolarization is preferably comparable or stronger than light-driven ganglion cell spiking in a functional human cone cell. The light-driven ganglion cell spiking can be assessed using any suitable assay. For example, light-driven ganglion cell spiking can be measured using a multi-electrode array.

Any retinal disease may be suitable for treatment according to the methods and with the compositions disclosed herein. Exemplary retinal disorders that may be suitable for treating include, but are not limited to, retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, post-retinal detachment, cone dysfunction, a tapetoretinal degeneration, retinal vein occlusion and geographic atrophy. Treatment according to the methods and with the compositions disclosed herein is suitable for a subject that has a disorder impairing vision in which the optic nerve retains at least some function.

In some embodiments, the depolarizing optogenetic protein can be expressed in a cell membrane of a human cone cell. In some embodiments, the depolarizing optogenetic protein can be capable of mediating a depolarizing current that depolarizes a human cone cell when exposed to light. In some embodiments, depolarization of the human cone cell induces light-driven ganglion cell spiking that can be comparable to light-driven ganglion cell spiking in a functional human cone cell. In some embodiments, the light-driven ganglion cell spiking can be assessed using a multi-electrode array.

The nucleic acid can be capable of restoring light sensitivity when introduced into a human cone cell, wherein restoration of light sensitivity occurs when (i) the depolarizing optogenetic protein is expressed in a cell membrane of the human cone cell, (ii) the depolarizing optogenetic protein is capable of mediating a depolarizing current that depolarizes a human cone cell when exposed to light, and (iii) depolarization of the human cone cell induces light-driven ganglion cell spiking.

### 3. BRIEF DESCRIPTION OF THE DRAWINGS

FIGS. 1A-1E are graphs depicting the expression of the AAV8-BP2-ProA7-ChrimsonR-tdTomato (no WPRE), AAV8-BP2-CAG-ChrimsonR-tdTomato (no WPRE), AAV8-BP2-ProA7-ChrimsonR-tdTomato-WPRE and AAV8-BP2-ProA7-vfChrimson-EYFP-WPRE in different model systems. The percentage of cone transduction was determined based on a cone marker (cone arrestin) in mouse, human and non-human primate (NHP) cones. No WPRE element is required for expression of the construct in mouse retina and human retinal organoids when using the ProA7 promoter (FIG. 1A, FIG. 1B). However, when no WPRE is present in the ProA7 construct, there is no cone expression in mature, human retinal cones in retinal explants (FIG. 1C). The non-selective CAG promoter does not need a WPRE element in order for the optogenetic protein to be expressed in cones. Addition of the WPRE element to the AAV8-BP2-ProA7-ChrimsonR-tdT construct leads to expression in human cones (FIG. 1C). Therefore in human retina, the requirement of the WPRE element is promoter dependent, as it is required for the ProA7 promoter, but not for the CAG promoter (FIG. 1C). The AAV8-BP2-ProA7-vfChrimson-EYFP-WPRE vector construct also expressed in cone cells in non-human primate (macaque) retina after in vivo injection (FIG. 1D). ProA7 led to mostly cone-specific expression in the mouse retina, and specificity for cones was almost 100% in human retinal cones and non-human primate cones (FIG. 1E). CAG-driven expression of ChrimsonR was not specific to cones as other cells have been targeted as well (FIG. 1E). (All vectors in FIGS. 1A-1E have the hGH polyA sequence).

FIGS. 2A and 2B are graphs depicting the percentage of cones transduced with the AAV-ProA7-vfChrimson-EYFP-WPRE-hGH polyA construct with different AAV capsid serotypes AAV8-BP2, AAV-PHP.B, AAV-PHP.eB, AAV-NHP26, and AAV-44.9 (E531D). FIG. 2A shows the percentage of cones transduced in human retinal organoids at a dose of 8.5E11 vector genomes (v.g.). FIG. 2B shows the percentage of cones transduced in the human retina explants at a high dose (2.8E12 v.g.) and low dose (3.8E11 v.g.), respectively using the same vector with different capsids. Vector genome counts were determined by digital droplet PCR for WPRE to allow for titer matching between the different constructs.

FIGS. 3A-3B are graphs depicting the percentage of cones transduced with a vector construct comprising AAV8-BP2, a ProA7 promoter, WPRE, hGH polyA, different transgenes and a reporter tag. The following transgenes were tested: vfChrimson, ChrimsonR, CatCh, ChrMine, ReaChR, fChrimson-C174, UBI-fChrimson, fChrimson and Jaws. Either EYFP, Citrine or tdTomato (tdT) reporter tags were included in the constructs. FIG. 3A shows the percentage of cones transduced in retinal organoids at a dose of 8.5E11 v.g. per organoid. FIG. 3B shows the percentage of cones transduced in macular human retinal explants at a high dose (2.8E12 v.g.). and low dose (3.8E11 v.g.), respectively.

FIGS. 4A and 4B are graphs depicting the results of a combination screen using AAV8-BP2, AAV-PHP.eB, and AAV-NHP26 capsids and vfChrimson-EYFP, ChrimsonR-tdTomato, ReaChR-Citrine and ChrMine-EYFP transgenes in all combinations. FIG. 4A shows the percentage of cones transduced in human retinal organoids at a dose of 8.5E11 v.g. FIG. 4B shows the percentage of cones transduced in the human retina at a low dose (3.8E11 v.g.). The ProA7-ReaChR-Citrine-WPRE-hGH polyA and ProA7-ChrMine-EYFP-WPRE-hGH polyA led to very high level of expression even when using different capsids in both model systems.

FIGS. 5A and 5B are graphs depicting the results of a capsid screen using AAV5, AAV8, or AAV9 capsids and the ProA7-ReaChR-Citrine-WPRE-hGH poly A transgene. FIG. 5A shows the percentage of cones transduced in human retinal organoids at a dose of 8.5E11 v.g. FIG. 5B shows the percentage of cones transduced in the human retina at a low dose (3.8E11 v.g.). AAV5, AAV8 and AAV9 led to efficient expression of ProA7-ReaChR-Citrine-WPRE-hGH poly A both in retinal organoids and in human retinal explants.

FIGS. 6A-6D are graphs showing light responsiveness and membrane localization of optogenetic constructs in cultured retinas. In FIGS. 6A-6C, all constructs tested included ProA7 promoter, a fluorescent reporter, WPRE, and hGH polyA. Vectors tested were: AAV8-BP2-ProA7-ReaChR-Citrine-WPRE-hGH polyA, AAV9-PHP.eB-ProA7-ReaChR-Citrine-WPRE-hGH polyA, AAV9-PHP.eB-ProA7-ChrMine-EYFP-WPRE-hGH polyA, AAV5-ProA7-ReaChR-Citrine-WPRE-hGH poly A, AAV8-BP2-ProA7-Jaws-EYFP-WPRE-hGH polyA, AAV8-BP2-ProA7-ChrimsonR-EYFP-WPRE-hGH polyA, and AAV-NHP26-ProA7-ReaChR-Citrine-WPRE-hGH polyA. Untreated human retinal explants were used as a control. Untreated human retinal explants lose light sensitivity within a few hours and only optogenetically treated retinas show light responses several weeks after explantation. All recordings were done 4-8 weeks post-AAV treatment. FIG. 6A shows the correlation index (a measure for light responsiveness) for all the constructs and the negative control. FIG. 6B shows the absolute modulation index (another measure for light responsiveness) for the constructs and negative control. Only ProA7-ReaChR-Citrine-WPRE-hGH polyA resulted in light responses in human retinas, with all capsids tested. FIG. 6C shows the percentage of cells in which the optogenetic protein was exclusively localized to the cell membrane. Though not to be bound by theory, the observed light responses with ReaChR may be explained by its superior membrane localization (FIG. 6C). FIG. 6D shows additional testing with hyperpolarising channels (eGTACR1 and HcKCR1, with AAV5-ProA7 construct with WPRE and hGH polyA) on human retina. In contrast to ReaChR (construct was using the ProX573.2 promoter), these hyperpolarising optogenetic channels do not evoke light responses.

FIGS. 7A-7C are graphs showing light-driven activity in human retinal explants transduced with AAV vectors containing ProA7-ReaChR-WPRE-hGH poly A or ProA7-ReaChR-Citrine-WPRE-hGH poly A. FIG. 7A shows multi-electrode array recordings of light-driven ganglion cell spiking from human retinal explants transduced with AAV5 capsid containing ProA7-ReaChR-Citrine-WPRE-hGH polyA. FIG. 7A shows different optogenetically driven light responsive of major cell types in human retina (sustained ON cell, transient ON cell, ON/OFF cell, transient OFF cell, sustained OFF cell) to a 2 seconds white light flash. This diversity in responses indicate optogenetically re-established retinal information processing. These responses are identical to normal human light responses, as depicted in Cowan C et al., (2020) Cell 182(6):1623-1640). FIG. 7B shows light responsiveness as function of photon flux for human retinal explants transduced with AAV5 capsid containing ProA7-ReaChR-Citrine-WPRE-hGH polyA or an identical construct lacking the fluorescent tag, citrine. FIG. 7C shows frequency modulation index in retinas transduced with ProA7-ReaChR-Citrine-WPRE-hGH polyA, and that modulation was seen at a frequency stimulation up to 23.2 Hz. This suggest that the optogenetically treated retina responds to a ˜23 frames per second alternating stimulus.

FIGS. 8A-8C show light responsiveness for AAV5-ProA7-ReaChR-citrine-WPRE-hGH poly A vector transduced and non-transduced areas (control) and cone transduction in transduced areas (‘bleb’) of explanted retinas of in vivo sub-retinally injected, non-human primates (Cynomolgus macaques). Light responses were detected on acute recordings shortly after explantation suggesting good viability of the tissue (“ControlAcute” on FIG. 8A). Retinas then were cultured 1-2 days to abolish endogenous light responses. In the non-treated area (non-bleb), no light responses were detected after culture, as expected (Control (non-bleb) on FIG. 8A). In the treated area (bleb), optogenetic light responses were obvious at the two highest dose levels, with transient ON-, sustained ON-, ON/OFF cells, and ON/OFF- and OFF suppression cells (FIG. 8A and FIG. 8B). At the lowest dose (1.5e10 v.g.) we did not observe light responses (FIG. 8A). FIG. 8C shows the cone transduction efficiency at different dose levels (n=2 eyes for each dose). All dose levels included n=2 per dose except for the 1.5E10 dose, where a single eye was used. The points represent different images taken from different locations within the bleb.

FIGS. 9A-9B show cone transduction efficiency and light responsiveness by AAV5-ProA7-ReaChR-citrine-WPRE-hGH poly A vector in explanted retinas, including the fovea. Immunostaining was performed against GFP to detect the citrine tag in transduced tissue. Cone transduction is highly efficient across donor ages from 18 to 58 years. FIG. 9A shows cone transduction in retinal explants from four human donors. FIG. 9B presents optogenetic light responses measured as light responsivity index (R, correlation between trials) following AAV5-ProA7-ReaChR-citrine-WPRE-hGH poly A vector transduction of explanted retinas from the four additional human donors, ages ranging from 18 to 58 years. FIG. 9C shows cone transduction efficiency by the AAV5-ProA7-ReaChr-citrine-WPRE-hGH polyA vector in explanted retinas from twelve human donors in which the donor age ranged from 18 to 80 years old. FIG. 9D shows the light responsivity index (R, correlation between trials) in the explanted retinas from the twelve donors. The ‘control’ represents untreated, cultured retinas that do not show light sensitivity. The ‘acute’ represents normal light responses from a freshly isolated, non-cultured retina piece. Donor A from FIGS. 9A and 9B corresponds with donor 5 in FIGS. 9C and 9D. Donor B from FIGS. 9A and 9B corresponds with donor 7 in FIGS. 9C and 9D. Donor C from FIGS. 9A and 9B corresponds with donor 6 in the FIGS. 9C and 9D. Quantification of cone transduction was performed for all 12 donors using the same background threshold.

FIG. 10 are schematics showing the structures of three vectors containing ReaChR-citrine, WPRE, and hGH poly A, and differing by promoter: ProA7, Pro572.2, and Pro573.2. ITR: inverted terminal repeat.

FIGS. 11A-11E are graphs showing transduction and light responsiveness of optogenetic constructs under the control of promoters ProA7, Pro572.2 or Pro573.2 (Pro572.2 and Pro573.2 are also referred to as “ProX572.2” and “ProX573.2,” respectively) in human retinal explants. FIGS. 11A-11B are graphs showing cone transduction percentage (FIG. 11A) in human retinal explants and function (light responsiveness) (FIG. 11B) in human retinal explants for ProA7 and Pro572.2-driven optogenetic vectors. FIG. 11C shows that the Pro573.2 driven construct resulted in significantly stronger light responses in human retina, compared to the ProA7 driven construct. FIG. 11D shows that the Pro573.2 driven construct resulted in a higher percentage of light responsive cells. FIG. 11E shows that the percentage of cones that were transduced with optogenetic vectors that contained ProA7 or Pro573.2 were similar.

FIGS. 12A and 12B show light responsivity and the percentage of light responsive cells in human retinal explants from three donors transduced with AAV5-Pro573.2-ReaChR-WPRE-hGH polyA. FIG. 12A is a graph showing the light responsivity index. FIG. 12B is a graph showing the percentage of light responsive cells in the explant.

FIGS. 13A and 13B show that transduction of human retinal explants with AAV5-Pro573.2-ReaChR-WPRE-hGH polyA AAV resulted in better light responsivity compared with AAV5-ProA7-ReaChR-citrine-WPRE-hGH polyA. The Pro573.2-driven construct drives significantly stronger light responses (FIG. 13A) and is more sensitive (curve is shifted to the left with Pro573.2 promoter-driven construct) (FIG. 13B).

FIGS. 14A-14B show light responses and the percentage of light responsive cells in control, acute and bleb tissue samples from macaque retina following subretinal injection of the AAV5-Pro573.2-ReaChR-WPRE-hGH polyA vector. “Acute” means freshly dissected retina, where normal light responses can be measured. The “control” and “bleb” tissue pieces were cultured to eliminate normal light responses prior to analysis. The “control” is from a non-treated area of the retina and as expected showed almost no light responsivity. The “bleb” is from a treated area of the retina, and showed light responsiveness. FIG. 14C shows that the full diversity of light responses in the treated bleb sample (5 classes of cells) were detected following transduction of the AAV5-Pro573.2-ReaChR-WPRE-hGH polyA vector.

FIG. 15. shows light responses from blind rd1 mice injected with AAV5-ProA7-ReaChR-citrine-WPRE-hGH poly A. The few light responsive cells in the control may represent intrinsically photosensitive cells in the retina.

FIG. 16. is a graph showing GFP expression levels in human retinal organoids following transduction with AAV5 and AAVPhP.eB vectors encoding GFP under the control of different promoters and at a dose of 1E10 or 1E11 viral genomes (v.g) per well. Vectors tested were: AAVPhP.eB-ProA7-EGFP-WPRE, AAVPhP.eB-ProSC-EGFP-WPRE, AAVPhP.eB-2xProSC-EGFP-WPRE, AAVPhP.eB-3xProSC-EGFP-WPRE, AAVPhP.eB-4xProSC-EGFP-WPRE, AAV5-ProA330-EGFP-WPRE, AAVPhP.eB-2xmin330-EGFP-WPRE, AAVPhP.eB-3xmin330-EGFP-WPRE, AAVPhP.eB-4xmin330-EGFP-WPRE, AAVPhP.eB-330-3del1del5-EGFP-WPRE, and AAVPhP.eB-3del1del6-EGFP-WPRE. Negative controls include no AAV (Ctrl-noAAV) and AAV5-noPro-EGFP-WPRE (Ctrl-noP).

FIGS. 17A and 17B show cone labeling and cone-specificity for constructs under the control of the ProA7 or 4xProSC promoters. FIG. 17A is a quantification plot for expression in cross-sections of human retinal organoids transduced with AAVPhP.eB-ProA7-EGFP-WPRE or AAVPhP.eB-4xProSC-EGFP-WPRE. Quantification of GFP+ cell density as a percentage of cone photoreceptor density is shown. FIG. 17B is a quantification plot of AAV-targeting specificity shown as percentage of major (circle) and minor (square) cell types or classes among cells expressing GFP in organoids transduced by AAVPhP.eB-ProA7-EGFP-WPRE or AAVPhP.eB-4xProSC-EGFP-WPRE.

FIG. 18 shows spinning disk confocal microscope images of cross-sections of human retinal organoids transduced with AAVPhP.eB-ProSC-EGFP-WPRE and AAVPhP.eB-4xProSC-EGFP-WPRE. Left: GFP (green). Middle-left, immunostaining with cone marker CAR (magenta). Middle-right, immunostaining with a rod marker NRL (blue), GFP, and cone marker (signal overlap visible in white). Right: GFP and cone marker and nuclear stain (Hoechst, white).

### DETAILED DESCRIPTION

The disclosure relates to an optogenetic approach that restores photosensitivity in dormant human cone cells. The approach disclosed herein is contrary to the natural photostimulation mechanism in human cone cells, which become hyperpolarized upon photostimulation. As described and exemplified herein, optogenetic constructs that cause depolarization upon photostimulation of cone cells have been developed and unexpectedly shown to restore photosensitivity in dormant cone cells. In fact, the degree of photosensitivity and depolarization of the cone cells is sufficient to also cause light-modulated current spikes in the down-stream retinal ganglion cells (RGC), which are well-known to transmit visual information to the brain through action potentials. Accordingly, this disclosure relates to nucleic acid constructs and viral vectors comprising a depolarizing optogenetic constructs comprising a depolarizing optogenetic protein, preferably a light-gated ion channel protein. The nucleic acids include expression control elements (e.g., promoter, cis-regulatory elements) that causes the depolarizing optogenetic protein to be specifically expressed in transduced human cone cells at a level sufficient to restore light sensitivity in human cone cells. Preferred combinations of expression control elements, optogenetic proteins and viral vectors, e.g., AAV capsids, that provide selective delivery and expression of the optogenetic proteins in human cone cells are also disclosed.

As described above, conventional wisdom in the field holds that an optogenetic approach for restoring photosensitivity of photoreceptors, such as cone cells, should focus on recreating the normal physiological process of photoactivation of cone cells, which in their normal and healthy state hyperpolarize upon photostimulation. Choung et al., (2014), Nature Neuroscience, 17, 1123-1129; Carter and Lecea (2011), Trends Mol. Med., 17(4):197-206; Simon et al., (2020), BBRC, 527(2):325-330. The inventors proceeded contrary to the conventional wisdom and surprisingly found that instead of recapitulating the light-induced hyperpolarization mechanism of normal healthy cone cells, photosensitivity, and light-induced signal transduction through the RGC can be achieved in human cone cells using a light-activated depolarizing ion channel protein. The inventors found that all types of ganglion cell responses that are present in normal human retina (Cowan C et al. (2020) Cell 182(6) 1623-1640) are recapitulated in optogenetically treated retinas using depolarizing channels. Importantly, and unexpectedly, the depolarizing optogenetic protein can generate a depolarizing current that is sufficiently potent to generate current spiking of RGCs, which can result in restoration of vision in humans. The depolarizing approach described herein is different from and opposite of the hyperpolarizing mechanism for photo-perception in healthy human photoreceptors.

As described herein, the inventors also surprisingly discovered that combining certain regulatory elements and specific depolarizing optogenetic protein (and optionally a reporter molecule) provide improved or superior expression and light sensitivity in transduced human cones.

This disclosure relates to nucleic acids comprising i) a promoter as described herein, ii) a nucleotide sequence encoding a depolarizing optogenetic protein, preferably ReaChR, and optionally a reporter molecule, preferably Citrine, and iii) a viral posttranscriptional regulatory element (PRE), preferably the woodchuck hepatis virus PRE (WPRE) and iv) optionally a synthetic intron. The promoter, nucleotide sequence encoding a depolarizing optogenetic protein and optionally a reporter molecule, and PRE are operably linked.

Generally, the promoter, in the context of the nucleic acids disclosed herein, provide for selective expression in cone photoreceptors, preferably human cone photoreceptors.

In some embodiments, the promoter can be a ProA7 promoter having SEQ ID NO: 2, or a variant thereof, including a functional fragment thereof. The functional fragment can be a truncated version of ProA7 comprising from about 150 to 499 nucleotides from SEQ ID NO: 2, such as from about 150 to 499 nucleotides from the 3′ end of SEQ ID NO: 2.

In some embodiments, the promoter can be a PR1.7 promoter having SEQ ID NO: 22, or a variant thereof, including a functional fragment thereof.

In some embodiments, the promoter can be a hG1.7 promoter having SEQ ID NO: 23, or a variant thereof, including a functional fragment thereof.

In some embodiments, the promoter can be a hybrid promoter comprising a ProA7-derived component having SEQ ID NO: 2, or a variant thereof, including a functional fragment thereof, such as a truncated version of SEQ ID NO: 2 comprising from about 150 to 499 nucleotides, such as from about 150 to 499 nucleotides from the 3′ end of SEQ ID NO: 2. The hybrid promoter also comprises a rod-specific-promoter-derived component having SEQ ID NO: 10, or a variant thereof, including a functional fragment thereof. The functional fragment can comprise from about 370 to 999 nucleotides from SEQ ID NO: 10, such as from about 370 to 999 nucleotides from the 3′ end of SEQ ID NO: 10 or about 895 to 999 nucleotides from the 5′ end of SEQ ID NO: 10. Similarly, the hybrid promoter can include other cone specific promoters or a variant thereof, including a functional fragment thereof, if desired, and a rod-specific-promoter-derived component, such as SEQ ID NO:10 or a variant, including a functional fragment thereof.

A preferred nucleic acid comprises a ProA7 promoter, a ReaChR optogenetic protein and optionally Citrine as a reporter molecule, and WPRE. Preferably, the nucleic acid also includes a nucleotide sequence that encodes a suitable polyadenylation signal (PolyA, such as the human growth hormone (hGH) polyA) that is 3′ of the WPRE. Each of the nucleotide sequence are operably linked. The nucleic acid can further comprise an inverted terminal repeat sequence (ITR), for example, an AAV ITR. For example, the nucleic acid can include an AAV ITR 5′ of the promoter and an AAV ITR 3′ of the WPRE or preferably 3′ of the PolyA signal. If desired the nucleic acid can further encode an AAV capsid protein.

The nucleic acid sequence encoding the depolarizing optogenetic protein can be expressed (transcribed and translated) and the encoded protein localized to the cell membrane of the human cone cell when introduced into a human cone cell. The depolarizing optogenetic protein can mediate a depolarizing current that depolarizes a human cone cell when exposed to light. Depolarization of the human cone cells can induce light-driven current spikes in RGC. The spiking can be similar or comparable to light-driven RGC spiking in a functional human retina with health cone cells. The light-driven RGC spiking can be measuring using, for example, a multi-electrode array.

This disclosure further relates to methods of using the nucleic acid in the treatment of disease, conditions, and disorders associated with retinal disease and restoring vision.

Also provided herein are pharmaceutical compositions comprising the nucleic acid that encode the optogenetic construct as well as recombinant expression vectors and host cells for making the optogenetic constructs disclosed here.

Certain illustrative and preferred embodiments are described in detail herein. The embodiments within the specification should not be construed to limit the scope of the disclosure.

### A. Promoter

a. ProA7 and Variants, Fragments, and Truncations Thereof

As disclosed herein, the nucleic acid can comprise a nucleic acid sequence encoding a ProA7 promoter, or a variant, fragment, or truncation thereof.

The ProA7 promoter is known to be useful for driving high expression of desired genes in a variety of species, tissues and cell types. For instance, the inventors showed that the ProA7 promoter alone can drive expression in mouse photoreceptor cone cells and a variety of human cell types, including preferentially driving expression in human cone cells and not other retinal cells. See, e.g., Juttner et al. Nature Neuroscience 22, 1345-1356 (2019). However, the inventors surprisingly discovered that the ProA7 promoter alone did not drive expression of depolarizing optogenetic protein in human photoreceptor cone cells. The inventors surprisingly discovered that the ProA7 promoter in combination with a posttranscriptional regulatory element (PRE), preferably the woodchuck hepatis virus PRE (WPRE), can drive expression in human photoreceptor cone cells. It was also found that the particular depolarizing optogenetic protein, in combination with ProA7 and the PRE (e.g., WPRE) influences expression levels.

The ProA7 promoter disclosed herein has specificity for human cone cells, and drives high levels of expression in cone cells, for example, cone cells that are dormant and no longer responsive to light. The ProA7 promoter can have promoter activity in human cone cells in retinal explants. In some instances, the ProA7 promoter does not have substantial promoter activity in other cells of the human retina, such as rod cells (e.g., less than about 10% of promoter activity is seen in non-cone cells).

This disclosure relates to synthetic promoters that are cone-specific and can drive expression of a desired nucleic acid (e.g., a transgene) in cone photoreceptor cells. The synthetic promoters can include a first component (a first nucleotide sequence) that is derived from a cone-specific promoter, such as SEQ ID NO:2, and a second component (a second nucleotide sequence) that is derived from a rod-specific promoter, such as SEQ ID NO:10, with the first and second components operably linked. The inventors have surprisingly discovered that such synthetic promoters are unexpectedly cone-specific, and have augmented promoter activity in human cone cells relative to the cone-specific promoter from which the first component (first nucleotide sequence) is derived (e.g., SEQ ID NO:2).

The term “cone-specific” as used herein describes a promoter that has promoter activity in human cone photoreceptors that is at least about 90% selective for human cone cells over human rod cells. For example, the promoter can have least about 90% selectivity, at least about 91% selectivity, at least about 92% selectivity, at least about 93% selectivity, at least about 94% selectivity, at least about 95% selectivity, at least about 96% selectivity, at least about 97% selectivity, at least about 98% selectivity, at least about 99% selectivity, or 100% selectivity for cone cells over rod cells. The selectivity of a promoter in human cone photoreceptors relative to other cells, such as human rod photoreceptors, can be determined using suitable methods that are well-known to a person of ordinary skill in the art. In one preferred method, a retinal organoid or retinal tissue containing both cone and rod cells is transduced with a nucleic acid construct containing the promoter operably linked to a nucleic acid sequence encoding a reporter molecule, such as tdTomato, enhanced yellow fluorescent protein (EYFP), Citrine, green fluorescent protein (GFP), cyan fluorescent protein, red fluorescent protein, or a functional variant thereof, the organoids are sectioned and immunostained to identify cone cells and/or rod cells using a cell-type-specific antibody (e.g., anti-human cone-arrestin for cone cells, anti-human rhodopsin for rod cells); promoter activity is assessed by counting cells expressing the reporter molecule; and fractions of cells expressing the reporter that are co-labeled with each cell-type-specific antibody are determined. Cone-specificity is calculated as the fraction or percentage of total cells expressing the reporter that are cone cells.

The inventors have observed that the cone-specific synthetic promoters disclosed herein are cone-specific in humans and at least one species of non-human primate (macaques). The promoters may have lower selectivity for cone cells of non-primate mammals.

The term “rod-specific” as used herein describes a promoter that has promoter activity in human rod photoreceptors that is at least about 90% selective for human rod cells over human cone cells. For example, the promoter can have at least about 90%, at least about 91% selectivity, at least about 92% selectivity, at least about 93% selectivity, at least about 94% selectivity, at least about 95% selectivity, at least about 96% selectivity, at least about 97% selectivity, at least about 98% selectivity, at least about 99% selectivity, or 100% selectivity for rod cells over cone cells. The selectivity of a promoter in human rod photoreceptors relative to other cells, such as human cone photoreceptors, can be determined using suitable methods that are well-known to a person of ordinary skill in the art. In one preferred method, a retinal organoid or retinal tissue containing both rod and cone cells is transduced with a nucleic acid construct containing the promoter operably linked to a nucleic acid sequence encoding a reporter molecule, such as tdTomato, enhanced yellow fluorescent protein (EYFP), Citrine, green fluorescent protein (GFP), cyan fluorescent protein, red fluorescent protein, or a functional variant thereof, the organoids are sectioned and immunostained to identify rod cells and/or cone cells by using a cell-type-specific antibody (e.g., anti-human cone-arrestin for cone cells, anti-human rhodopsin for rod cells); promoter activity is assessed by counting cells expressing the reporter molecule; and fractions of cells expressing the reporter that are co-labeled with each cell-type-specific antibody are determined. Rod-specificity is calculated as the fraction or percentage of total cells expressing the reporter that are rod cells.

The level of promoter activity in a cell type of interest can be determined by transducing a suitable population of cells (e.g. a human retinal organoid) with a nucleic acid construct containing the promoter operably linked to a nucleic acid sequence encoding a reporter molecule, such as tdTomato, enhanced yellow fluorescent protein (EYFP), Citrine, green fluorescent protein (GFP), cyan fluorescent protein, red fluorescent protein, or a functional variant thereof, the organoids are sectioned and immunostained to identify cells of the cell type of interest by using a cell-type-specific antibody (e.g., anti-human cone-arrestin for cone cells, anti-human rhodopsin for rod cells); counting the number of cells of the cell type of interest; and counting the number of cells of the cell type of interest that express the reporter molecule. Promoter activity in cells of the cell type of interest is calculated as the fraction or percentage of cells of the cell type of interest expressing the reporter molecule.

A promoter comprising the ProA7 promoter or a variant, fragment, or truncation thereof can be operably linked to the nucleotide sequence encoding the depolarizing optogenetic protein. The ProA7 promoter or a variant, fragment, or truncation thereof can be operably linked to a reporter molecule, if desired. The ProA7 promoter or a variant, fragment, or truncation thereof can contain transcriptional sequences that mediate the expression of the depolarizing optogenetic protein.

The ProA7 promoter has been described in International Publication No. WO2017046084. Generally, the ProA7 promoter has a nucleotide sequence comprising SEQ ID NO: 2. The ProA7 promoter may comprise a nucleic acid sequence having at least 70% sequence identity to SEQ ID NO: 2. The ProA7 promoter can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, a at least bout 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or greater sequence identity to SEQ ID NO:2.

If desired, the promoter can be a ProA7 variant of SEQ ID NO: 2 that retains promoter activity. The variant ProA7 promoter may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, compared to SEQ ID NO: 2.

The ProA7 variant can be a truncated form of the ProA7 promoter (e.g. can contain fewer than the 500 nt of ProA7, due to deletion of nucleotides at the 5′ end). A truncated ProA7 promoter can comprise a sequence of at least about 150 nucleotides, and preferably will have at least about 70% or more sequence identity with SEQ ID NO: 2 over the length of the variant sequence. For example, the ProA7 variant can include a 5′ truncation of ProA7, the sequence of the variant can be substantially the same as SEQ ID NO:2 over the length of the variant, or at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO:2 over the length of the variant.

A truncated ProA7 promoter with a 5′ truncation of ProA7 can comprise any number of nucleotides from about 150 to 499 (i.e., one less than the length of SEQ ID NO: 2). For example, the nucleotide sequence of a 5′ truncated Pro7 variant can be the same as nucleotides 386-500 of SEQ ID NO:2, or can be the same as nucleotides 2-500 of SEQ ID NO:2.

In some embodiments, the 5′ truncated ProA7 promoter can contain about 150 nt, about 151 nt, about 152 nt, about 153 nt, about 154 nt, about 155 nt, about 156 nt, about 157 nt, about 158 nt, about 159 nt, about 160 nt, about 161 nt, about 162 nt, about 163 nt, about 164 nt, about 165 nt, about 166 nt, about 167 nt, about 168 nt, about 169 nt, about 170 nt, about 171 nt, about 172 nt, about 173 nt, about 174 nt, about 175 nt, about 176 nt, about 177 nt, about 178 nt, about 179 nt, about 180 nt, about 181 nt, about 182 nt, about 183 nt, about 184 nt, about 185 nt, about 186 nt, about 187 nt, about 188 nt, about 189 nt, about 190 nt, about 191 nt, about 192 nt, about 193 nt, about 194 nt, about 195 nt, about 196 nt, about 197 nt, about 198 nt, about 199 nt, about 200 nt, about 201 nt, about 202 nt, about 203 nt, about 204 nt, about 205 nt, about 206 nt, about 207 nt, about 208 nt, about 209 nt, about 210 nt, about 211 nt, about 212 nt, about 213 nt, about 214 nt, about 215 nt, about 216 nt, about 217 nt, about 218 nt, about 219 nt, about 220 nt, about 221 nt, about 222 nt, about 223 nt, about 224 nt, about 225 nt, about 226 nt, about 227 nt, about 228 nt, about 229 nt, about 230 nt, about 231 nt, about 232 nt, about 233 nt, about 234 nt, about 235 nt, about 236 nt, about 237 nt, about 238 nt, about 239 nt, about 240 nt, about 241 nt, about 242 nt, about 243 nt, about 244 nt, about 245 nt, about 246 nt, about 247 nt, about 248 nt, about 249 nt, about 250 nt, about 251 nt, about 252 nt, about 253 nt, about 254 nt, about 255 nt, about 256 nt, about 257 nt, about 258 nt, about 259 nt, about 260 nt, about 261 nt, about 262 nt, about 263 nt, about 264 nt, about 265 nt, about 266 nt, about 267 nt, about 268 nt, about 269 nt, about 270 nt, about 271 nt, about 272 nt, about 273 nt, about 274 nt, about 275 nt, about 276 nt, about 277 nt, about 278 nt, about 279 nt, about 280 nt, about 281 nt, about 282 nt, about 283 nt, about 284 nt, about 285 nt, about 286 nt, about 287 nt, about 288 nt, about 289 nt, about 290 nt, about 291 nt, about 292 nt, about 293 nt, about 294 nt, about 295 nt, about 296 nt, about 297 nt, about 298 nt, about 299 nt, about 300 nt, about 301 nt, about 302 nt, about 303 nt, about 304 nt, about 305 nt, about 306 nt, about 307 nt, about 308 nt, about 309 nt, about 310 nt, about 311 nt, about 312 nt, about 313 nt, about 314 nt, about 315 nt, about 316 nt, about 317 nt, about 318 nt, about 319 nt, about 320 nt, about 321 nt, about 322 nt, about 323 nt, about 324 nt, about 325 nt, about 326 nt, about 327 nt, about 328 nt, about 329 nt, about 330 nt, about 331 nt, about 332 nt, about 333 nt, about 334 nt, about 335 nt, about 336 nt, about 337 nt, about 338 nt, about 339 nt, about 340 nt, about 341 nt, about 342 nt, about 343 nt, about 344 nt, about 345 nt, about 346 nt, about 347 nt, about 348 nt, about 349 nt, about 350 nt, about 351 nt, about 352 nt, about 353 nt, about 354 nt, about 355 nt, about 356 nt, about 357 nt, about 358 nt, about 359 nt, about 360 nt, about 361 nt, about 362 nt, about 363 nt, about 364 nt, about 365 nt, about 366 nt, about 367 nt, about 368 nt, about 369 nt, about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, or about 499 nt from SEQ ID NO: 2, such as from the 3′ end of SEQ ID NO: 2.

The truncated ProA7 promoter (e.g., 5′ truncated) may comprise a nucleic acid sequence having at least 70% or more sequence identity with SEQ ID NO: 2 over the full length of the truncated ProA7 promoter. For example, the ProA7 variant can include a 5′ truncation, and the sequence of the variant can be substantially the same as SEQ ID NO:2 over the length of the variant, or at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO:2 over the length of the variant.

The truncated ProA7 promoter may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 2.

An exemplary truncated ProA7 promoter suitable for use in the constructs of this disclosure can comprise a sequence of about 395 nt having at least about 70% or greater identity to the 3′ end of SEQ ID NO: 2 over the 395 nt. For example, the truncated ProA7 promoter can comprise SEQ ID NO: 24.

An exemplary truncated ProA7 promoter suitable for use in the constructs of this disclosure can comprise a sequence of about 290 nt having at least about 70% or greater identity to the 3′ end of SEQ ID NO: 2 over the 290 nt. For example, the truncated ProA7 promoter can comprise SEQ ID NO: 25.

An exemplary truncated ProA7 promoter suitable for use in the constructs of this disclosure can comprise a sequence of about 185 nt having at least 70% or greater identity to the 3′ end of SEQ ID NO: 2 over the 185 nt. For example, the truncated ProA7 promoter can comprise SEQ ID NO: 26. The promoter of SEQ ID NO:26 is referred to as ProA7 5′3 or ProSC.

An exemplary truncated ProA7 promoter suitable for use in the constructs of this disclosure can comprise a sequence of about 150 nt having at least 70% or greater identity to the 3′ end of SEQ ID NO: 2 over the 150 nt. For example, the truncated ProA7 promoter can comprise SEQ ID NO: 27.

In some embodiments, the promoter contains a ProA7 or truncated ProA7 that comprises SEQ ID NO: 2, SEQ ID NO: 24, SEQ ID NO: 25, SEQ ID NO: 26, or SEQ ID NO: 27. As described further herein, if desired, the ProA7 promoter, fragment or variant can be used as a single copy or as multiple copies of the promoter sequence. In some embodiments, the promoter can comprise two or more ProA7-derived components, that are the same or different, for example from 1 to about 10 copies of the same or different sequence. Such as one to about 10 copies (i.e., one, two, three, four, five, six, seven, eight, nine or about 10 copies) of SEQ ID NO: 24, SEQ ID NO: 25, SEQ ID NO: 26, or SEQ ID NO: 27 or any combination thereof. In embodiments, the cone specific promoter contains one, two, three, four, five, six, seven, eight, nine or about 10 copies of SEQ ID NO:26.

ProA7 and truncated ProA7 promoters are suitable for inclusion in a nucleic acid of the disclosure.

b. PR1.7, hG1.7, and Variants, Fragments, and Truncations Thereof.

Other suitable promotors include a PR1.7 promoter comprising SEQ ID NO:22, or a sequence which has at least 70% identity to SEQ ID NO: 22. The PR1.7 promoter can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or greater sequence identity to SEQ ID NO:22.

The PR1.7 promoter may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO:22.

The PR1.7 variant can be a truncated form of the PR1.7 promoter (e.g. can contain fewer than the 1724 nt of PR1.7, due to deletion of nucleotides at the 5′ end). A truncated PR1.7 promoter can comprise a sequence of at least about 150 nucleotides, and preferably will have at least about 70% or more sequence identity with SEQ ID NO:22 over the length of the variant sequence. For example, the PR1.7 variant can include a truncation of PR1.7, the sequence of the variant can be substantially the same as SEQ ID NO:22 over the length of the variant, or at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO:22 over the length of the variant.

A truncated PR1.7 promoter can comprise any number of nucleotides from about 150 to 1723 (i.e., one less than the length of SEQ ID NO:22).

In some embodiments, the truncated PR1.7 promoter can contain about 150 nt, about 151 nt, about 152 nt, about 153 nt, about 154 nt, about 155 nt, about 156 nt, about 157 nt, about 158 nt, about 159 nt, about 160 nt, about 161 nt, about 162 nt, about 163 nt, about 164 nt, about 165 nt, about 166 nt, about 167 nt, about 168 nt, about 169 nt, about 170 nt, about 171 nt, about 172 nt, about 173 nt, about 174 nt, about 175 nt, about 176 nt, about 177 nt, about 178 nt, about 179 nt, about 180 nt, about 181 nt, about 182 nt, about 183 nt, about 184 nt, about 185 nt, about 186 nt, about 187 nt, about 188 nt, about 189 nt, about 190 nt, about 191 nt, about 192 nt, about 193 nt, about 194 nt, about 195 nt, about 196 nt, about 197 nt, about 198 nt, about 199 nt, about 200 nt, about 201 nt, about 202 nt, about 203 nt, about 204 nt, about 205 nt, about 206 nt, about 207 nt, about 208 nt, about 209 nt, about 210 nt, about 211 nt, about 212 nt, about 213 nt, about 214 nt, about 215 nt, about 216 nt, about 217 nt, about 218 nt, about 219 nt, about 220 nt, about 221 nt, about 222 nt, about 223 nt, about 224 nt, about 225 nt, about 226 nt, about 227 nt, about 228 nt, about 229 nt, about 230 nt, about 231 nt, about 232 nt, about 233 nt, about 234 nt, about 235 nt, about 236 nt, about 237 nt, about 238 nt, about 239 nt, about 240 nt, about 241 nt, about 242 nt, about 243 nt, about 244 nt, about 245 nt, about 246 nt, about 247 nt, about 248 nt, about 249 nt, about 250 nt, about 251 nt, about 252 nt, about 253 nt, about 254 nt, about 255 nt, about 256 nt, about 257 nt, about 258 nt, about 259 nt, about 260 nt, about 261 nt, about 262 nt, about 263 nt, about 264 nt, about 265 nt, about 266 nt, about 267 nt, about 268 nt, about 269 nt, about 270 nt, about 271 nt, about 272 nt, about 273 nt, about 274 nt, about 275 nt, about 276 nt, about 277 nt, about 278 nt, about 279 nt, about 280 nt, about 281 nt, about 282 nt, about 283 nt, about 284 nt, about 285 nt, about 286 nt, about 287 nt, about 288 nt, about 289 nt, about 290 nt, about 291 nt, about 292 nt, about 293 nt, about 294 nt, about 295 nt, about 296 nt, about 297 nt, about 298 nt, about 299 nt, about 300 nt, about 301 nt, about 302 nt, about 303 nt, about 304 nt, about 305 nt, about 306 nt, about 307 nt, about 308 nt, about 309 nt, about 310 nt, about 311 nt, about 312 nt, about 313 nt, about 314 nt, about 315 nt, about 316 nt, about 317 nt, about 318 nt, about 319 nt, about 320 nt, about 321 nt, about 322 nt, about 323 nt, about 324 nt, about 325 nt, about 326 nt, about 327 nt, about 328 nt, about 329 nt, about 330 nt, about 331 nt, about 332 nt, about 333 nt, about 334 nt, about 335 nt, about 336 nt, about 337 nt, about 338 nt, about 339 nt, about 340 nt, about 341 nt, about 342 nt, about 343 nt, about 344 nt, about 345 nt, about 346 nt, about 347 nt, about 348 nt, about 349 nt, about 350 nt, about 351 nt, about 352 nt, about 353 nt, about 354 nt, about 355 nt, about 356 nt, about 357 nt, about 358 nt, about 359 nt, about 360 nt, about 361 nt, about 362 nt, about 363 nt, about 364 nt, about 365 nt, about 366 nt, about 367 nt, about 368 nt, about 369 nt, about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, about 499 nt, about 500 nt, about 501 nt, about 502 nt, about 503 nt, about 504 nt, about 505 nt, about 506 nt, about 507 nt, about 508 nt, about 509 nt, about 510 nt, about 511 nt, about 512 nt, about 513 nt, about 514 nt, about 515 nt, about 516 nt, about 517 nt, about 518 nt, about 519 nt, about 520 nt, about 521 nt, about 522 nt, about 523 nt, about 524 nt, about 525 nt, about 526 nt, about 527 nt, about 528 nt, about 529 nt, about 530 nt, about 531 nt, about 532 nt, about 533 nt, about 534 nt, about 535 nt, about 536 nt, about 537 nt, about 538 nt, about 539 nt, about 540 nt, about 541 nt, about 542 nt, about 543 nt, about 544 nt, about 545 nt, about 546 nt, about 547 nt, about 548 nt, about 549 nt, about 550 nt, about 551 nt, about 552 nt, about 553 nt, about 554 nt, about 555 nt, about 556 nt, about 557 nt, about 558 nt, about 559 nt, about 560 nt, about 561 nt, about 562 nt, about 563 nt, about 564 nt, about 565 nt, about 566 nt, about 567 nt, about 568 nt, about 569 nt, about 570 nt, about 571 nt, about 572 nt, about 573 nt, about 574 nt, about 575 nt, about 576 nt, about 577 nt, about 578 nt, about 579 nt, about 580 nt, about 581 nt, about 582 nt, about 583 nt, about 584 nt, about 585 nt, about 586 nt, about 587 nt, about 588 nt, about 589 nt, about 590 nt, about 591 nt, about 592 nt, about 593 nt, about 594 nt, about 595 nt, about 596 nt, about 597 nt, about 598 nt, about 599 nt, about 600 nt, about 601 nt, about 602 nt, about 603 nt, about 604 nt, about 605 nt, about 606 nt, about 607 nt, about 608 nt, about 609 nt, about 610 nt, about 611 nt, about 612 nt, about 613 nt, about 614 nt, about 615 nt, about 616 nt, about 617 nt, about 618 nt, about 619 nt, about 620 nt, about 621 nt, about 622 nt, about 623 nt, about 624 nt, about 625 nt, about 626 nt, about 627 nt, about 628 nt, about 629 nt, about 630 nt, about 631 nt, about 632 nt, about 633 nt, about 634 nt, about 635 nt, about 636 nt, about 637 nt, about 638 nt, about 639 nt, about 640 nt, about 641 nt, about 642 nt, about 643 nt, about 644 nt, about 645 nt, about 646 nt, about 647 nt, about 648 nt, about 649 nt, about 650 nt, about 651 nt, about 652 nt, about 653 nt, about 654 nt, about 655 nt, about 656 nt, about 657 nt, about 658 nt, about 659 nt, about 660 nt, about 661 nt, about 662 nt, about 663 nt, about 664 nt, about 665 nt, about 666 nt, about 667 nt, about 668 nt, about 669 nt, about 670 nt, about 671 nt, about 672 nt, about 673 nt, about 674 nt, about 675 nt, about 676 nt, about 677 nt, about 678 nt, about 679 nt, about 680 nt, about 681 nt, about 682 nt, about 683 nt, about 684 nt, about 685 nt, about 686 nt, about 687 nt, about 688 nt, about 689 nt, about 690 nt, about 691 nt, about 692 nt, about 693 nt, about 694 nt, about 695 nt, about 696 nt, about 697 nt, about 698 nt, about 699 nt, about 700 nt, about 701 nt, about 702 nt, about 703 nt, about 704 nt, about 705 nt, about 706 nt, about 707 nt, about 708 nt, about 709 nt, about 710 nt, about 711 nt, about 712 nt, about 713 nt, about 714 nt, about 715 nt, about 716 nt, about 717 nt, about 718 nt, about 719 nt, about 720 nt, about 721 nt, about 722 nt, about 723 nt, about 724 nt, about 725 nt, about 726 nt, about 727 nt, about 728 nt, about 729 nt, about 730 nt, about 731 nt, about 732 nt, about 733 nt, about 734 nt, about 735 nt, about 736 nt, about 737 nt, about 738 nt, about 739 nt, about 740 nt, about 741 nt, about 742 nt, about 743 nt, about 744 nt, about 745 nt, about 746 nt, about 747 nt, about 748 nt, about 749 nt, about 750 nt, about 751 nt, about 752 nt, about 753 nt, about 754 nt, about 755 nt, about 756 nt, about 757 nt, about 758 nt, about 759 nt, about 760 nt, about 761 nt, about 762 nt, about 763 nt, about 764 nt, about 765 nt, about 766 nt, about 767 nt, about 768 nt, about 769 nt, about 770 nt, about 771 nt, about 772 nt, about 773 nt, about 774 nt, about 775 nt, about 776 nt, about 777 nt, about 778 nt, about 779 nt, about 780 nt, about 781 nt, about 782 nt, about 783 nt, about 784 nt, about 785 nt, about 786 nt, about 787 nt, about 788 nt, about 789 nt, about 790 nt, about 791 nt, about 792 nt, about 793 nt, about 794 nt, about 795 nt, about 796 nt, about 797 nt, about 798 nt, about 799 nt, about 800 nt, about 801 nt, about 802 nt, about 803 nt, about 804 nt, about 805 nt, about 806 nt, about 807 nt, about 808 nt, about 809 nt, about 810 nt, about 811 nt, about 812 nt, about 813 nt, about 814 nt, about 815 nt, about 816 nt, about 817 nt, about 818 nt, about 819 nt, about 820 nt, about 821 nt, about 822 nt, about 823 nt, about 824 nt, about 825 nt, about 826 nt, about 827 nt, about 828 nt, about 829 nt, about 830 nt, about 831 nt, about 832 nt, about 833 nt, about 834 nt, about 835 nt, about 836 nt, about 837 nt, about 838 nt, about 839 nt, about 840 nt, about 841 nt, about 842 nt, about 843 nt, about 844 nt, about 845 nt, about 846 nt, about 847 nt, about 848 nt, about 849 nt, about 850 nt, about 851 nt, about 852 nt, about 853 nt, about 854 nt, about 855 nt, about 856 nt, about 857 nt, about 858 nt, about 859 nt, about 860 nt, about 861 nt, about 862 nt, about 863 nt, about 864 nt, about 865 nt, about 866 nt, about 867 nt, about 868 nt, about 869 nt, about 870 nt, about 871 nt, about 872 nt, about 873 nt, about 874 nt, about 875 nt, about 876 nt, about 877 nt, about 878 nt, about 879 nt, about 880 nt, about 881 nt, about 882 nt, about 883 nt, about 884 nt, about 885 nt, about 886 nt, about 887 nt, about 888 nt, about 889 nt, about 890 nt, about 891 nt, about 892 nt, about 893 nt, about 894 nt, about 895 nt, about 896 nt, about 897 nt, about 898 nt, about 899 nt, about 900 nt, about 901 nt, about 902 nt, about 903 nt, about 904 nt, about 905 nt, about 906 nt, about 907 nt, about 908 nt, about 909 nt, about 910 nt, about 911 nt, about 912 nt, about 913 nt, about 914 nt, about 915 nt, about 916 nt, about 917 nt, about 918 nt, about 919 nt, about 920 nt, about 921 nt, about 922 nt, about 923 nt, about 924 nt, about 925 nt, about 926 nt, about 927 nt, about 928 nt, about 929 nt, about 930 nt, about 931 nt, about 932 nt, about 933 nt, about 934 nt, about 935 nt, about 936 nt, about 937 nt, about 938 nt, about 939 nt, about 940 nt, about 941 nt, about 942 nt, about 943 nt, about 944 nt, about 945 nt, about 946 nt, about 947 nt, about 948 nt, about 949 nt, about 950 nt, about 951 nt, about 952 nt, about 953 nt, about 954 nt, about 955 nt, about 956 nt, about 957 nt, about 958 nt, about 959 nt, about 960 nt, about 961 nt, about 962 nt, about 963 nt, about 964 nt, about 965 nt, about 966 nt, about 967 nt, about 968 nt, about 969 nt, about 970 nt, about 971 nt, about 972 nt, about 973 nt, about 974 nt, about 975 nt, about 976 nt, about 977 nt, about 978 nt, about 979 nt, about 980 nt, about 981 nt, about 982 nt, about 983 nt, about 984 nt, about 985 nt, about 986 nt, about 987 nt, about 988 nt, about 989 nt, about 990 nt, about 991 nt, about 992 nt, about 993 nt, about 994 nt, about 995 nt, about 996 nt, about 997 nt, about 998 nt, about 999 nt, about 1000 nt, about 1001 nt, about 1002 nt, about 1003 nt, about 1004 nt, about 1005 nt, about 1006 nt, about 1007 nt, about 1008 nt, about 1009 nt, about 1010 nt, about 1011 nt, about 1012 nt, about 1013 nt, about 1014 nt, about 1015 nt, about 1016 nt, about 1017 nt, about 1018 nt, about 1019 nt, about 1020 nt, about 1021 nt, about 1022 nt, about 1023 nt, about 1024 nt, about 1025 nt, about 1026 nt, about 1027 nt, about 1028 nt, about 1029 nt, about 1030 nt, about 1031 nt, about 1032 nt, about 1033 nt, about 1034 nt, about 1035 nt, about 1036 nt, about 1037 nt, about 1038 nt, about 1039 nt, about 1040 nt, about 1041 nt, about 1042 nt, about 1043 nt, about 1044 nt, about 1045 nt, about 1046 nt, about 1047 nt, about 1048 nt, about 1049 nt, about 1050 nt, about 1051 nt, about 1052 nt, about 1053 nt, about 1054 nt, about 1055 nt, about 1056 nt, about 1057 nt, about 1058 nt, about 1059 nt, about 1060 nt, about 1061 nt, about 1062 nt, about 1063 nt, about 1064 nt, about 1065 nt, about 1066 nt, about 1067 nt, about 1068 nt, about 1069 nt, about 1070 nt, about 1071 nt, about 1072 nt, about 1073 nt, about 1074 nt, about 1075 nt, about 1076 nt, about 1077 nt, about 1078 nt, about 1079 nt, about 1080 nt, about 1081 nt, about 1082 nt, about 1083 nt, about 1084 nt, about 1085 nt, about 1086 nt, about 1087 nt, about 1088 nt, about 1089 nt, about 1090 nt, about 1091 nt, about 1092 nt, about 1093 nt, about 1094 nt, about 1095 nt, about 1096 nt, about 1097 nt, about 1098 nt, about 1099 nt, about 1100 nt, about 1101 nt, about 1102 nt, about 1103 nt, about 1104 nt, about 1105 nt, about 1106 nt, about 1107 nt, about 1108 nt, about 1109 nt, about 1110 nt, about 1111 nt, about 1112 nt, about 1113 nt, about 1114 nt, about 1115 nt, about 1116 nt, about 1117 nt, about 1118 nt, about 1119 nt, about 1120 nt, about 1121 nt, about 1122 nt, about 1123 nt, about 1124 nt, about 1125 nt, about 1126 nt, about 1127 nt, about 1128 nt, about 1129 nt, about 1130 nt, about 1131 nt, about 1132 nt, about 1133 nt, about 1134 nt, about 1135 nt, about 1136 nt, about 1137 nt, about 1138 nt, about 1139 nt, about 1140 nt, about 1141 nt, about 1142 nt, about 1143 nt, about 1144 nt, about 1145 nt, about 1146 nt, about 1147 nt, about 1148 nt, about 1149 nt, about 1150 nt, about 1151 nt, about 1152 nt, about 1153 nt, about 1154 nt, about 1155 nt, about 1156 nt, about 1157 nt, about 1158 nt, about 1159 nt, about 1160 nt, about 1161 nt, about 1162 nt, about 1163 nt, about 1164 nt, about 1165 nt, about 1166 nt, about 1167 nt, about 1168 nt, about 1169 nt, about 1170 nt, about 1171 nt, about 1172 nt, about 1173 nt, about 1174 nt, about 1175 nt, about 1176 nt, about 1177 nt, about 1178 nt, about 1179 nt, about 1180 nt, about 1181 nt, about 1182 nt, about 1183 nt, about 1184 nt, about 1185 nt, about 1186 nt, about 1187 nt, about 1188 nt, about 1189 nt, about 1190 nt, about 1191 nt, about 1192 nt, about 1193 nt, about 1194 nt, about 1195 nt, about 1196 nt, about 1197 nt, about 1198 nt, about 1199 nt, about 1200 nt, about 1201 nt, about 1202 nt, about 1203 nt, about 1204 nt, about 1205 nt, about 1206 nt, about 1207 nt, about 1208 nt, about 1209 nt, about 1210 nt, about 1211 nt, about 1212 nt, about 1213 nt, about 1214 nt, about 1215 nt, about 1216 nt, about 1217 nt, about 1218 nt, about 1219 nt, about 1220 nt, about 1221 nt, about 1222 nt, about 1223 nt, about 1224 nt, about 1225 nt, about 1226 nt, about 1227 nt, about 1228 nt, about 1229 nt, about 1230 nt, about 1231 nt, about 1232 nt, about 1233 nt, about 1234 nt, about 1235 nt, about 1236 nt, about 1237 nt, about 1238 nt, about 1239 nt, about 1240 nt, about 1241 nt, about 1242 nt, about 1243 nt, about 1244 nt, about 1245 nt, about 1246 nt, about 1247 nt, about 1248 nt, about 1249 nt, about 1250 nt, about 1251 nt, about 1252 nt, about 1253 nt, about 1254 nt, about 1255 nt, about 1256 nt, about 1257 nt, about 1258 nt, about 1259 nt, about 1260 nt, about 1261 nt, about 1262 nt, about 1263 nt, about 1264 nt, about 1265 nt, about 1266 nt, about 1267 nt, about 1268 nt, about 1269 nt, about 1270 nt, about 1271 nt, about 1272 nt, about 1273 nt, about 1274 nt, about 1275 nt, about 1276 nt, about 1277 nt, about 1278 nt, about 1279 nt, about 1280 nt, about 1281 nt, about 1282 nt, about 1283 nt, about 1284 nt, about 1285 nt, about 1286 nt, about 1287 nt, about 1288 nt, about 1289 nt, about 1290 nt, about 1291 nt, about 1292 nt, about 1293 nt, about 1294 nt, about 1295 nt, about 1296 nt, about 1297 nt, about 1298 nt, about 1299 nt, about 1300 nt, about 1301 nt, about 1302 nt, about 1303 nt, about 1304 nt, about 1305 nt, about 1306 nt, about 1307 nt, about 1308 nt, about 1309 nt, about 1310 nt, about 1311 nt, about 1312 nt, about 1313 nt, about 1314 nt, about 1315 nt, about 1316 nt, about 1317 nt, about 1318 nt, about 1319 nt, about 1320 nt, about 1321 nt, about 1322 nt, about 1323 nt, about 1324 nt, about 1325 nt, about 1326 nt, about 1327 nt, about 1328 nt, about 1329 nt, about 1330 nt, about 1331 nt, about 1332 nt, about 1333 nt, about 1334 nt, about 1335 nt, about 1336 nt, about 1337 nt, about 1338 nt, about 1339 nt, about 1340 nt, about 1341 nt, about 1342 nt, about 1343 nt, about 1344 nt, about 1345 nt, about 1346 nt, about 1347 nt, about 1348 nt, about 1349 nt, about 1350 nt, about 1351 nt, about 1352 nt, about 1353 nt, about 1354 nt, about 1355 nt, about 1356 nt, about 1357 nt, about 1358 nt, about 1359 nt, about 1360 nt, about 1361 nt, about 1362 nt, about 1363 nt, about 1364 nt, about 1365 nt, about 1366 nt, about 1367 nt, about 1368 nt, about 1369 nt, about 1370 nt, about 1371 nt, about 1372 nt, about 1373 nt, about 1374 nt, about 1375 nt, about 1376 nt, about 1377 nt, about 1378 nt, about 1379 nt, about 1380 nt, about 1381 nt, about 1382 nt, about 1383 nt, about 1384 nt, about 1385 nt, about 1386 nt, about 1387 nt, about 1388 nt, about 1389 nt, about 1390 nt, about 1391 nt, about 1392 nt, about 1393 nt, about 1394 nt, about 1395 nt, about 1396 nt, about 1397 nt, about 1398 nt, about 1399 nt, about 1400 nt, about 1401 nt, about 1402 nt, about 1403 nt, about 1404 nt, about 1405 nt, about 1406 nt, about 1407 nt, about 1408 nt, about 1409 nt, about 1410 nt, about 1411 nt, about 1412 nt, about 1413 nt, about 1414 nt, about 1415 nt, about 1416 nt, about 1417 nt, about 1418 nt, about 1419 nt, about 1420 nt, about 1421 nt, about 1422 nt, about 1423 nt, about 1424 nt, about 1425 nt, about 1426 nt, about 1427 nt, about 1428 nt, about 1429 nt, about 1430 nt, about 1431 nt, about 1432 nt, about 1433 nt, about 1434 nt, about 1435 nt, about 1436 nt, about 1437 nt, about 1438 nt, about 1439 nt, about 1440 nt, about 1441 nt, about 1442 nt, about 1443 nt, about 1444 nt, about 1445 nt, about 1446 nt, about 1447 nt, about 1448 nt, about 1449 nt, about 1450 nt, about 1451 nt, about 1452 nt, about 1453 nt, about 1454 nt, about 1455 nt, about 1456 nt, about 1457 nt, about 1458 nt, about 1459 nt, about 1460 nt, about 1461 nt, about 1462 nt, about 1463 nt, about 1464 nt, about 1465 nt, about 1466 nt, about 1467 nt, about 1468 nt, about 1469 nt, about 1470 nt, about 1471 nt, about 1472 nt, about 1473 nt, about 1474 nt, about 1475 nt, about 1476 nt, about 1477 nt, about 1478 nt, about 1479 nt, about 1480 nt, about 1481 nt, about 1482 nt, about 1483 nt, about 1484 nt, about 1485 nt, about 1486 nt, about 1487 nt, about 1488 nt, about 1489 nt, about 1490 nt, about 1491 nt, about 1492 nt, about 1493 nt, about 1494 nt, about 1495 nt, about 1496 nt, about 1497 nt, about 1498 nt, about 1499 nt, about 1500 nt, about 1501 nt, about 1502 nt, about 1503 nt, about 1504 nt, about 1505 nt, about 1506 nt, about 1507 nt, about 1508 nt, about 1509 nt, about 1510 nt, about 1511 nt, about 1512 nt, about 1513 nt, about 1514 nt, about 1515 nt, about 1516 nt, about 1517 nt, about 1518 nt, about 1519 nt, about 1520 nt, about 1521 nt, about 1522 nt, about 1523 nt, about 1524 nt, about 1525 nt, about 1526 nt, about 1527 nt, about 1528 nt, about 1529 nt, about 1530 nt, about 1531 nt, about 1532 nt, about 1533 nt, about 1534 nt, about 1535 nt, about 1536 nt, about 1537 nt, about 1538 nt, about 1539 nt, about 1540 nt, about 1541 nt, about 1542 nt, about 1543 nt, about 1544 nt, about 1545 nt, about 1546 nt, about 1547 nt, about 1548 nt, about 1549 nt, about 1550 nt, about 1551 nt, about 1552 nt, about 1553 nt, about 1554 nt, about 1555 nt, about 1556 nt, about 1557 nt, about 1558 nt, about 1559 nt, about 1560 nt, about 1561 nt, about 1562 nt, about 1563 nt, about 1564 nt, about 1565 nt, about 1566 nt, about 1567 nt, about 1568 nt, about 1569 nt, about 1570 nt, about 1571 nt, about 1572 nt, about 1573 nt, about 1574 nt, about 1575 nt, about 1576 nt, about 1577 nt, about 1578 nt, about 1579 nt, about 1580 nt, about 1581 nt, about 1582 nt, about 1583 nt, about 1584 nt, about 1585 nt, about 1586 nt, about 1587 nt, about 1588 nt, about 1589 nt, about 1590 nt, about 1591 nt, about 1592 nt, about 1593 nt, about 1594 nt, about 1595 nt, about 1596 nt, about 1597 nt, about 1598 nt, about 1599 nt, about 1600 nt, about 1601 nt, about 1602 nt, about 1603 nt, about 1604 nt, about 1605 nt, about 1606 nt, about 1607 nt, about 1608 nt, about 1609 nt, about 1610 nt, about 1611 nt, about 1612 nt, about 1613 nt, about 1614 nt, about 1615 nt, about 1616 nt, about 1617 nt, about 1618 nt, about 1619 nt, about 1620 nt, about 1621 nt, about 1622 nt, about 1623 nt, about 1624 nt, about 1625 nt, about 1626 nt, about 1627 nt, about 1628 nt, about 1629 nt, about 1630 nt, about 1631 nt, about 1632 nt, about 1633 nt, about 1634 nt, about 1635 nt, about 1636 nt, about 1637 nt, about 1638 nt, about 1639 nt, about 1640 nt, about 1641 nt, about 1642 nt, about 1643 nt, about 1644 nt, about 1645 nt, about 1646 nt, about 1647 nt, about 1648 nt, about 1649 nt, about 1650 nt, about 1651 nt, about 1652 nt, about 1653 nt, about 1654 nt, about 1655 nt, about 1656 nt, about 1657 nt, about 1658 nt, about 1659 nt, about 1660 nt, about 1661 nt, about 1662 nt, about 1663 nt, about 1664 nt, about 1665 nt, about 1666 nt, about 1667 nt, about 1668 nt, about 1669 nt, about 1670 nt, about 1671 nt, about 1672 nt, about 1673 nt, about 1674 nt, about 1675 nt, about 1676 nt, about 1677 nt, about 1678 nt, about 1679 nt, about 1680 nt, about 1681 nt, about 1682 nt, about 1683 nt, about 1684 nt, about 1685 nt, about 1686 nt, about 1687 nt, about 1688 nt, about 1689 nt, about 1690 nt, about 1691 nt, about 1692 nt, about 1693 nt, about 1694 nt, about 1695 nt, about 1696 nt, about 1697 nt, about 1698 nt, about 1699 nt, about 1700 nt, about 1701 nt, about 1702 nt, about 1703 nt, about 1704 nt, about 1705 nt, about 1706 nt, about 1707 nt, about 1708 nt, about 1709 nt, about 1710 nt, about 1711 nt, about 1712 nt, about 1713 nt, about 1714 nt, about 1715 nt, about 1716 nt, about 1717 nt, about 1718 nt, about 1719 nt, about 1720 nt, about 1721 nt, about 1722 nt, or about 1723 nt from SEQ ID NO:22.

Another suitable promoter is a hG1.7 promoter comprising SEQ ID NO:23 or a sequence which has at least 70% identity to SEQ ID NO: 23. The hG1.7 promoter can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or greater sequence identity to a sequence of equal length from SEQ ID NO:23.

The hG1.7 promoter may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 23.

The hG1.7 variant can be a truncated form of the hG1.7 promoter (e.g. can contain fewer than the 1782 nt of hG1.7). A truncated hG1.7 promoter can comprise a sequence of at least about 150 nucleotides, and preferably will have at least about 70% or more sequence identity with SEQ ID NO:23 over the length of the variant sequence. For example, the hG1.7 variant can include a truncation of hG1.7, the sequence of the variant can be substantially the same as SEQ ID NO:23 over the length of the variant, or at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO:23 over the length of the variant.

A truncated hG1.7 promoter can comprise any number of nucleotides from about 150 to 1781 (i.e., one less than the length of SEQ ID NO:23).

In some embodiments, the truncated hG1.7 promoter can contain about 150 nt, about 151 nt, about 152 nt, about 153 nt, about 154 nt, about 155 nt, about 156 nt, about 157 nt, about 158 nt, about 159 nt, about 160 nt, about 161 nt, about 162 nt, about 163 nt, about 164 nt, about 165 nt, about 166 nt, about 167 nt, about 168 nt, about 169 nt, about 170 nt, about 171 nt, about 172 nt, about 173 nt, about 174 nt, about 175 nt, about 176 nt, about 177 nt, about 178 nt, about 179 nt, about 180 nt, about 181 nt, about 182 nt, about 183 nt, about 184 nt, about 185 nt, about 186 nt, about 187 nt, about 188 nt, about 189 nt, about 190 nt, about 191 nt, about 192 nt, about 193 nt, about 194 nt, about 195 nt, about 196 nt, about 197 nt, about 198 nt, about 199 nt, about 200 nt, about 201 nt, about 202 nt, about 203 nt, about 204 nt, about 205 nt, about 206 nt, about 207 nt, about 208 nt, about 209 nt, about 210 nt, about 211 nt, about 212 nt, about 213 nt, about 214 nt, about 215 nt, about 216 nt, about 217 nt, about 218 nt, about 219 nt, about 220 nt, about 221 nt, about 222 nt, about 223 nt, about 224 nt, about 225 nt, about 226 nt, about 227 nt, about 228 nt, about 229 nt, about 230 nt, about 231 nt, about 232 nt, about 233 nt, about 234 nt, about 235 nt, about 236 nt, about 237 nt, about 238 nt, about 239 nt, about 240 nt, about 241 nt, about 242 nt, about 243 nt, about 244 nt, about 245 nt, about 246 nt, about 247 nt, about 248 nt, about 249 nt, about 250 nt, about 251 nt, about 252 nt, about 253 nt, about 254 nt, about 255 nt, about 256 nt, about 257 nt, about 258 nt, about 259 nt, about 260 nt, about 261 nt, about 262 nt, about 263 nt, about 264 nt, about 265 nt, about 266 nt, about 267 nt, about 268 nt, about 269 nt, about 270 nt, about 271 nt, about 272 nt, about 273 nt, about 274 nt, about 275 nt, about 276 nt, about 277 nt, about 278 nt, about 279 nt, about 280 nt, about 281 nt, about 282 nt, about 283 nt, about 284 nt, about 285 nt, about 286 nt, about 287 nt, about 288 nt, about 289 nt, about 290 nt, about 291 nt, about 292 nt, about 293 nt, about 294 nt, about 295 nt, about 296 nt, about 297 nt, about 298 nt, about 299 nt, about 300 nt, about 301 nt, about 302 nt, about 303 nt, about 304 nt, about 305 nt, about 306 nt, about 307 nt, about 308 nt, about 309 nt, about 310 nt, about 311 nt, about 312 nt, about 313 nt, about 314 nt, about 315 nt, about 316 nt, about 317 nt, about 318 nt, about 319 nt, about 320 nt, about 321 nt, about 322 nt, about 323 nt, about 324 nt, about 325 nt, about 326 nt, about 327 nt, about 328 nt, about 329 nt, about 330 nt, about 331 nt, about 332 nt, about 333 nt, about 334 nt, about 335 nt, about 336 nt, about 337 nt, about 338 nt, about 339 nt, about 340 nt, about 341 nt, about 342 nt, about 343 nt, about 344 nt, about 345 nt, about 346 nt, about 347 nt, about 348 nt, about 349 nt, about 350 nt, about 351 nt, about 352 nt, about 353 nt, about 354 nt, about 355 nt, about 356 nt, about 357 nt, about 358 nt, about 359 nt, about 360 nt, about 361 nt, about 362 nt, about 363 nt, about 364 nt, about 365 nt, about 366 nt, about 367 nt, about 368 nt, about 369 nt, about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, about 499 nt, about 500 nt, about 501 nt, about 502 nt, about 503 nt, about 504 nt, about 505 nt, about 506 nt, about 507 nt, about 508 nt, about 509 nt, about 510 nt, about 511 nt, about 512 nt, about 513 nt, about 514 nt, about 515 nt, about 516 nt, about 517 nt, about 518 nt, about 519 nt, about 520 nt, about 521 nt, about 522 nt, about 523 nt, about 524 nt, about 525 nt, about 526 nt, about 527 nt, about 528 nt, about 529 nt, about 530 nt, about 531 nt, about 532 nt, about 533 nt, about 534 nt, about 535 nt, about 536 nt, about 537 nt, about 538 nt, about 539 nt, about 540 nt, about 541 nt, about 542 nt, about 543 nt, about 544 nt, about 545 nt, about 546 nt, about 547 nt, about 548 nt, about 549 nt, about 550 nt, about 551 nt, about 552 nt, about 553 nt, about 554 nt, about 555 nt, about 556 nt, about 557 nt, about 558 nt, about 559 nt, about 560 nt, about 561 nt, about 562 nt, about 563 nt, about 564 nt, about 565 nt, about 566 nt, about 567 nt, about 568 nt, about 569 nt, about 570 nt, about 571 nt, about 572 nt, about 573 nt, about 574 nt, about 575 nt, about 576 nt, about 577 nt, about 578 nt, about 579 nt, about 580 nt, about 581 nt, about 582 nt, about 583 nt, about 584 nt, about 585 nt, about 586 nt, about 587 nt, about 588 nt, about 589 nt, about 590 nt, about 591 nt, about 592 nt, about 593 nt, about 594 nt, about 595 nt, about 596 nt, about 597 nt, about 598 nt, about 599 nt, about 600 nt, about 601 nt, about 602 nt, about 603 nt, about 604 nt, about 605 nt, about 606 nt, about 607 nt, about 608 nt, about 609 nt, about 610 nt, about 611 nt, about 612 nt, about 613 nt, about 614 nt, about 615 nt, about 616 nt, about 617 nt, about 618 nt, about 619 nt, about 620 nt, about 621 nt, about 622 nt, about 623 nt, about 624 nt, about 625 nt, about 626 nt, about 627 nt, about 628 nt, about 629 nt, about 630 nt, about 631 nt, about 632 nt, about 633 nt, about 634 nt, about 635 nt, about 636 nt, about 637 nt, about 638 nt, about 639 nt, about 640 nt, about 641 nt, about 642 nt, about 643 nt, about 644 nt, about 645 nt, about 646 nt, about 647 nt, about 648 nt, about 649 nt, about 650 nt, about 651 nt, about 652 nt, about 653 nt, about 654 nt, about 655 nt, about 656 nt, about 657 nt, about 658 nt, about 659 nt, about 660 nt, about 661 nt, about 662 nt, about 663 nt, about 664 nt, about 665 nt, about 666 nt, about 667 nt, about 668 nt, about 669 nt, about 670 nt, about 671 nt, about 672 nt, about 673 nt, about 674 nt, about 675 nt, about 676 nt, about 677 nt, about 678 nt, about 679 nt, about 680 nt, about 681 nt, about 682 nt, about 683 nt, about 684 nt, about 685 nt, about 686 nt, about 687 nt, about 688 nt, about 689 nt, about 690 nt, about 691 nt, about 692 nt, about 693 nt, about 694 nt, about 695 nt, about 696 nt, about 697 nt, about 698 nt, about 699 nt, about 700 nt, about 701 nt, about 702 nt, about 703 nt, about 704 nt, about 705 nt, about 706 nt, about 707 nt, about 708 nt, about 709 nt, about 710 nt, about 711 nt, about 712 nt, about 713 nt, about 714 nt, about 715 nt, about 716 nt, about 717 nt, about 718 nt, about 719 nt, about 720 nt, about 721 nt, about 722 nt, about 723 nt, about 724 nt, about 725 nt, about 726 nt, about 727 nt, about 728 nt, about 729 nt, about 730 nt, about 731 nt, about 732 nt, about 733 nt, about 734 nt, about 735 nt, about 736 nt, about 737 nt, about 738 nt, about 739 nt, about 740 nt, about 741 nt, about 742 nt, about 743 nt, about 744 nt, about 745 nt, about 746 nt, about 747 nt, about 748 nt, about 749 nt, about 750 nt, about 751 nt, about 752 nt, about 753 nt, about 754 nt, about 755 nt, about 756 nt, about 757 nt, about 758 nt, about 759 nt, about 760 nt, about 761 nt, about 762 nt, about 763 nt, about 764 nt, about 765 nt, about 766 nt, about 767 nt, about 768 nt, about 769 nt, about 770 nt, about 771 nt, about 772 nt, about 773 nt, about 774 nt, about 775 nt, about 776 nt, about 777 nt, about 778 nt, about 779 nt, about 780 nt, about 781 nt, about 782 nt, about 783 nt, about 784 nt, about 785 nt, about 786 nt, about 787 nt, about 788 nt, about 789 nt, about 790 nt, about 791 nt, about 792 nt, about 793 nt, about 794 nt, about 795 nt, about 796 nt, about 797 nt, about 798 nt, about 799 nt, about 800 nt, about 801 nt, about 802 nt, about 803 nt, about 804 nt, about 805 nt, about 806 nt, about 807 nt, about 808 nt, about 809 nt, about 810 nt, about 811 nt, about 812 nt, about 813 nt, about 814 nt, about 815 nt, about 816 nt, about 817 nt, about 818 nt, about 819 nt, about 820 nt, about 821 nt, about 822 nt, about 823 nt, about 824 nt, about 825 nt, about 826 nt, about 827 nt, about 828 nt, about 829 nt, about 830 nt, about 831 nt, about 832 nt, about 833 nt, about 834 nt, about 835 nt, about 836 nt, about 837 nt, about 838 nt, about 839 nt, about 840 nt, about 841 nt, about 842 nt, about 843 nt, about 844 nt, about 845 nt, about 846 nt, about 847 nt, about 848 nt, about 849 nt, about 850 nt, about 851 nt, about 852 nt, about 853 nt, about 854 nt, about 855 nt, about 856 nt, about 857 nt, about 858 nt, about 859 nt, about 860 nt, about 861 nt, about 862 nt, about 863 nt, about 864 nt, about 865 nt, about 866 nt, about 867 nt, about 868 nt, about 869 nt, about 870 nt, about 871 nt, about 872 nt, about 873 nt, about 874 nt, about 875 nt, about 876 nt, about 877 nt, about 878 nt, about 879 nt, about 880 nt, about 881 nt, about 882 nt, about 883 nt, about 884 nt, about 885 nt, about 886 nt, about 887 nt, about 888 nt, about 889 nt, about 890 nt, about 891 nt, about 892 nt, about 893 nt, about 894 nt, about 895 nt, about 896 nt, about 897 nt, about 898 nt, about 899 nt, about 900 nt, about 901 nt, about 902 nt, about 903 nt, about 904 nt, about 905 nt, about 906 nt, about 907 nt, about 908 nt, about 909 nt, about 910 nt, about 911 nt, about 912 nt, about 913 nt, about 914 nt, about 915 nt, about 916 nt, about 917 nt, about 918 nt, about 919 nt, about 920 nt, about 921 nt, about 922 nt, about 923 nt, about 924 nt, about 925 nt, about 926 nt, about 927 nt, about 928 nt, about 929 nt, about 930 nt, about 931 nt, about 932 nt, about 933 nt, about 934 nt, about 935 nt, about 936 nt, about 937 nt, about 938 nt, about 939 nt, about 940 nt, about 941 nt, about 942 nt, about 943 nt, about 944 nt, about 945 nt, about 946 nt, about 947 nt, about 948 nt, about 949 nt, about 950 nt, about 951 nt, about 952 nt, about 953 nt, about 954 nt, about 955 nt, about 956 nt, about 957 nt, about 958 nt, about 959 nt, about 960 nt, about 961 nt, about 962 nt, about 963 nt, about 964 nt, about 965 nt, about 966 nt, about 967 nt, about 968 nt, about 969 nt, about 970 nt, about 971 nt, about 972 nt, about 973 nt, about 974 nt, about 975 nt, about 976 nt, about 977 nt, about 978 nt, about 979 nt, about 980 nt, about 981 nt, about 982 nt, about 983 nt, about 984 nt, about 985 nt, about 986 nt, about 987 nt, about 988 nt, about 989 nt, about 990 nt, about 991 nt, about 992 nt, about 993 nt, about 994 nt, about 995 nt, about 996 nt, about 997 nt, about 998 nt, about 999 nt, about 1000 nt, about 1001 nt, about 1002 nt, about 1003 nt, about 1004 nt, about 1005 nt, about 1006 nt, about 1007 nt, about 1008 nt, about 1009 nt, about 1010 nt, about 1011 nt, about 1012 nt, about 1013 nt, about 1014 nt, about 1015 nt, about 1016 nt, about 1017 nt, about 1018 nt, about 1019 nt, about 1020 nt, about 1021 nt, about 1022 nt, about 1023 nt, about 1024 nt, about 1025 nt, about 1026 nt, about 1027 nt, about 1028 nt, about 1029 nt, about 1030 nt, about 1031 nt, about 1032 nt, about 1033 nt, about 1034 nt, about 1035 nt, about 1036 nt, about 1037 nt, about 1038 nt, about 1039 nt, about 1040 nt, about 1041 nt, about 1042 nt, about 1043 nt, about 1044 nt, about 1045 nt, about 1046 nt, about 1047 nt, about 1048 nt, about 1049 nt, about 1050 nt, about 1051 nt, about 1052 nt, about 1053 nt, about 1054 nt, about 1055 nt, about 1056 nt, about 1057 nt, about 1058 nt, about 1059 nt, about 1060 nt, about 1061 nt, about 1062 nt, about 1063 nt, about 1064 nt, about 1065 nt, about 1066 nt, about 1067 nt, about 1068 nt, about 1069 nt, about 1070 nt, about 1071 nt, about 1072 nt, about 1073 nt, about 1074 nt, about 1075 nt, about 1076 nt, about 1077 nt, about 1078 nt, about 1079 nt, about 1080 nt, about 1081 nt, about 1082 nt, about 1083 nt, about 1084 nt, about 1085 nt, about 1086 nt, about 1087 nt, about 1088 nt, about 1089 nt, about 1090 nt, about 1091 nt, about 1092 nt, about 1093 nt, about 1094 nt, about 1095 nt, about 1096 nt, about 1097 nt, about 1098 nt, about 1099 nt, about 1100 nt, about 1101 nt, about 1102 nt, about 1103 nt, about 1104 nt, about 1105 nt, about 1106 nt, about 1107 nt, about 1108 nt, about 1109 nt, about 1110 nt, about 1111 nt, about 1112 nt, about 1113 nt, about 1114 nt, about 1115 nt, about 1116 nt, about 1117 nt, about 1118 nt, about 1119 nt, about 1120 nt, about 1121 nt, about 1122 nt, about 1123 nt, about 1124 nt, about 1125 nt, about 1126 nt, about 1127 nt, about 1128 nt, about 1129 nt, about 1130 nt, about 1131 nt, about 1132 nt, about 1133 nt, about 1134 nt, about 1135 nt, about 1136 nt, about 1137 nt, about 1138 nt, about 1139 nt, about 1140 nt, about 1141 nt, about 1142 nt, about 1143 nt, about 1144 nt, about 1145 nt, about 1146 nt, about 1147 nt, about 1148 nt, about 1149 nt, about 1150 nt, about 1151 nt, about 1152 nt, about 1153 nt, about 1154 nt, about 1155 nt, about 1156 nt, about 1157 nt, about 1158 nt, about 1159 nt, about 1160 nt, about 1161 nt, about 1162 nt, about 1163 nt, about 1164 nt, about 1165 nt, about 1166 nt, about 1167 nt, about 1168 nt, about 1169 nt, about 1170 nt, about 1171 nt, about 1172 nt, about 1173 nt, about 1174 nt, about 1175 nt, about 1176 nt, about 1177 nt, about 1178 nt, about 1179 nt, about 1180 nt, about 1181 nt, about 1182 nt, about 1183 nt, about 1184 nt, about 1185 nt, about 1186 nt, about 1187 nt, about 1188 nt, about 1189 nt, about 1190 nt, about 1191 nt, about 1192 nt, about 1193 nt, about 1194 nt, about 1195 nt, about 1196 nt, about 1197 nt, about 1198 nt, about 1199 nt, about 1200 nt, about 1201 nt, about 1202 nt, about 1203 nt, about 1204 nt, about 1205 nt, about 1206 nt, about 1207 nt, about 1208 nt, about 1209 nt, about 1210 nt, about 1211 nt, about 1212 nt, about 1213 nt, about 1214 nt, about 1215 nt, about 1216 nt, about 1217 nt, about 1218 nt, about 1219 nt, about 1220 nt, about 1221 nt, about 1222 nt, about 1223 nt, about 1224 nt, about 1225 nt, about 1226 nt, about 1227 nt, about 1228 nt, about 1229 nt, about 1230 nt, about 1231 nt, about 1232 nt, about 1233 nt, about 1234 nt, about 1235 nt, about 1236 nt, about 1237 nt, about 1238 nt, about 1239 nt, about 1240 nt, about 1241 nt, about 1242 nt, about 1243 nt, about 1244 nt, about 1245 nt, about 1246 nt, about 1247 nt, about 1248 nt, about 1249 nt, about 1250 nt, about 1251 nt, about 1252 nt, about 1253 nt, about 1254 nt, about 1255 nt, about 1256 nt, about 1257 nt, about 1258 nt, about 1259 nt, about 1260 nt, about 1261 nt, about 1262 nt, about 1263 nt, about 1264 nt, about 1265 nt, about 1266 nt, about 1267 nt, about 1268 nt, about 1269 nt, about 1270 nt, about 1271 nt, about 1272 nt, about 1273 nt, about 1274 nt, about 1275 nt, about 1276 nt, about 1277 nt, about 1278 nt, about 1279 nt, about 1280 nt, about 1281 nt, about 1282 nt, about 1283 nt, about 1284 nt, about 1285 nt, about 1286 nt, about 1287 nt, about 1288 nt, about 1289 nt, about 1290 nt, about 1291 nt, about 1292 nt, about 1293 nt, about 1294 nt, about 1295 nt, about 1296 nt, about 1297 nt, about 1298 nt, about 1299 nt, about 1300 nt, about 1301 nt, about 1302 nt, about 1303 nt, about 1304 nt, about 1305 nt, about 1306 nt, about 1307 nt, about 1308 nt, about 1309 nt, about 1310 nt, about 1311 nt, about 1312 nt, about 1313 nt, about 1314 nt, about 1315 nt, about 1316 nt, about 1317 nt, about 1318 nt, about 1319 nt, about 1320 nt, about 1321 nt, about 1322 nt, about 1323 nt, about 1324 nt, about 1325 nt, about 1326 nt, about 1327 nt, about 1328 nt, about 1329 nt, about 1330 nt, about 1331 nt, about 1332 nt, about 1333 nt, about 1334 nt, about 1335 nt, about 1336 nt, about 1337 nt, about 1338 nt, about 1339 nt, about 1340 nt, about 1341 nt, about 1342 nt, about 1343 nt, about 1344 nt, about 1345 nt, about 1346 nt, about 1347 nt, about 1348 nt, about 1349 nt, about 1350 nt, about 1351 nt, about 1352 nt, about 1353 nt, about 1354 nt, about 1355 nt, about 1356 nt, about 1357 nt, about 1358 nt, about 1359 nt, about 1360 nt, about 1361 nt, about 1362 nt, about 1363 nt, about 1364 nt, about 1365 nt, about 1366 nt, about 1367 nt, about 1368 nt, about 1369 nt, about 1370 nt, about 1371 nt, about 1372 nt, about 1373 nt, about 1374 nt, about 1375 nt, about 1376 nt, about 1377 nt, about 1378 nt, about 1379 nt, about 1380 nt, about 1381 nt, about 1382 nt, about 1383 nt, about 1384 nt, about 1385 nt, about 1386 nt, about 1387 nt, about 1388 nt, about 1389 nt, about 1390 nt, about 1391 nt, about 1392 nt, about 1393 nt, about 1394 nt, about 1395 nt, about 1396 nt, about 1397 nt, about 1398 nt, about 1399 nt, about 1400 nt, about 1401 nt, about 1402 nt, about 1403 nt, about 1404 nt, about 1405 nt, about 1406 nt, about 1407 nt, about 1408 nt, about 1409 nt, about 1410 nt, about 1411 nt, about 1412 nt, about 1413 nt, about 1414 nt, about 1415 nt, about 1416 nt, about 1417 nt, about 1418 nt, about 1419 nt, about 1420 nt, about 1421 nt, about 1422 nt, about 1423 nt, about 1424 nt, about 1425 nt, about 1426 nt, about 1427 nt, about 1428 nt, about 1429 nt, about 1430 nt, about 1431 nt, about 1432 nt, about 1433 nt, about 1434 nt, about 1435 nt, about 1436 nt, about 1437 nt, about 1438 nt, about 1439 nt, about 1440 nt, about 1441 nt, about 1442 nt, about 1443 nt, about 1444 nt, about 1445 nt, about 1446 nt, about 1447 nt, about 1448 nt, about 1449 nt, about 1450 nt, about 1451 nt, about 1452 nt, about 1453 nt, about 1454 nt, about 1455 nt, about 1456 nt, about 1457 nt, about 1458 nt, about 1459 nt, about 1460 nt, about 1461 nt, about 1462 nt, about 1463 nt, about 1464 nt, about 1465 nt, about 1466 nt, about 1467 nt, about 1468 nt, about 1469 nt, about 1470 nt, about 1471 nt, about 1472 nt, about 1473 nt, about 1474 nt, about 1475 nt, about 1476 nt, about 1477 nt, about 1478 nt, about 1479 nt, about 1480 nt, about 1481 nt, about 1482 nt, about 1483 nt, about 1484 nt, about 1485 nt, about 1486 nt, about 1487 nt, about 1488 nt, about 1489 nt, about 1490 nt, about 1491 nt, about 1492 nt, about 1493 nt, about 1494 nt, about 1495 nt, about 1496 nt, about 1497 nt, about 1498 nt, about 1499 nt, about 1500 nt, about 1501 nt, about 1502 nt, about 1503 nt, about 1504 nt, about 1505 nt, about 1506 nt, about 1507 nt, about 1508 nt, about 1509 nt, about 1510 nt, about 1511 nt, about 1512 nt, about 1513 nt, about 1514 nt, about 1515 nt, about 1516 nt, about 1517 nt, about 1518 nt, about 1519 nt, about 1520 nt, about 1521 nt, about 1522 nt, about 1523 nt, about 1524 nt, about 1525 nt, about 1526 nt, about 1527 nt, about 1528 nt, about 1529 nt, about 1530 nt, about 1531 nt, about 1532 nt, about 1533 nt, about 1534 nt, about 1535 nt, about 1536 nt, about 1537 nt, about 1538 nt, about 1539 nt, about 1540 nt, about 1541 nt, about 1542 nt, about 1543 nt, about 1544 nt, about 1545 nt, about 1546 nt, about 1547 nt, about 1548 nt, about 1549 nt, about 1550 nt, about 1551 nt, about 1552 nt, about 1553 nt, about 1554 nt, about 1555 nt, about 1556 nt, about 1557 nt, about 1558 nt, about 1559 nt, about 1560 nt, about 1561 nt, about 1562 nt, about 1563 nt, about 1564 nt, about 1565 nt, about 1566 nt, about 1567 nt, about 1568 nt, about 1569 nt, about 1570 nt, about 1571 nt, about 1572 nt, about 1573 nt, about 1574 nt, about 1575 nt, about 1576 nt, about 1577 nt, about 1578 nt, about 1579 nt, about 1580 nt, about 1581 nt, about 1582 nt, about 1583 nt, about 1584 nt, about 1585 nt, about 1586 nt, about 1587 nt, about 1588 nt, about 1589 nt, about 1590 nt, about 1591 nt, about 1592 nt, about 1593 nt, about 1594 nt, about 1595 nt, about 1596 nt, about 1597 nt, about 1598 nt, about 1599 nt, about 1600 nt, about 1601 nt, about 1602 nt, about 1603 nt, about 1604 nt, about 1605 nt, about 1606 nt, about 1607 nt, about 1608 nt, about 1609 nt, about 1610 nt, about 1611 nt, about 1612 nt, about 1613 nt, about 1614 nt, about 1615 nt, about 1616 nt, about 1617 nt, about 1618 nt, about 1619 nt, about 1620 nt, about 1621 nt, about 1622 nt, about 1623 nt, about 1624 nt, about 1625 nt, about 1626 nt, about 1627 nt, about 1628 nt, about 1629 nt, about 1630 nt, about 1631 nt, about 1632 nt, about 1633 nt, about 1634 nt, about 1635 nt, about 1636 nt, about 1637 nt, about 1638 nt, about 1639 nt, about 1640 nt, about 1641 nt, about 1642 nt, about 1643 nt, about 1644 nt, about 1645 nt, about 1646 nt, about 1647 nt, about 1648 nt, about 1649 nt, about 1650 nt, about 1651 nt, about 1652 nt, about 1653 nt, about 1654 nt, about 1655 nt, about 1656 nt, about 1657 nt, about 1658 nt, about 1659 nt, about 1660 nt, about 1661 nt, about 1662 nt, about 1663 nt, about 1664 nt, about 1665 nt, about 1666 nt, about 1667 nt, about 1668 nt, about 1669 nt, about 1670 nt, about 1671 nt, about 1672 nt, about 1673 nt, about 1674 nt, about 1675 nt, about 1676 nt, about 1677 nt, about 1678 nt, about 1679 nt, about 1680 nt, about 1681 nt, about 1682 nt, about 1683 nt, about 1684 nt, about 1685 nt, about 1686 nt, about 1687 nt, about 1688 nt, about 1689 nt, about 1690 nt, about 1691 nt, about 1692 nt, about 1693 nt, about 1694 nt, about 1695 nt, about 1696 nt, about 1697 nt, about 1698 nt, about 1699 nt, about 1700 nt, about 1701 nt, about 1702 nt, about 1703 nt, about 1704 nt, about 1705 nt, about 1706 nt, about 1707 nt, about 1708 nt, about 1709 nt, about 1710 nt, about 1711 nt, about 1712 nt, about 1713 nt, about 1714 nt, about 1715 nt, about 1716 nt, about 1717 nt, about 1718 nt, about 1719 nt, about 1720 nt, about 1721 nt, about 1722 nt, about 1723 nt, about 1724 nt, about 1725 nt, about 1726 nt, about 1727 nt, about 1728 nt, about 1729 nt, about 1730 nt, about 1731 nt, about 1732 nt, about 1733 nt, about 1734 nt, about 1735 nt, about 1736 nt, about 1737 nt, about 1738 nt, about 1739 nt, about 1740 nt, about 1741 nt, about 1742 nt, about 1743 nt, about 1744 nt, about 1745 nt, about 1746 nt, about 1747 nt, about 1748 nt, about 1749 nt, about 1750 nt, about 1751 nt, about 1752 nt, about 1753 nt, about 1754 nt, about 1755 nt, about 1756 nt, about 1757 nt, about 1758 nt, about 1759 nt, about 1760 nt, about 1761 nt, about 1762 nt, about 1763 nt, about 1764 nt, about 1765 nt, about 1766 nt, about 1767 nt, about 1768 nt, about 1769 nt, about 1770 nt, about 1771 nt, about 1772 nt, about 1773 nt, about 1774 nt, about 1775 nt, about 1776 nt, about 1777 nt, about 1778 nt, about 1779 nt, about 1780 nt, or about 1781 nt from SEQ ID NO:23.

As described herein, if desired, the RP1.7 promoter, the hG1.7 promoter, and fragment or variant of either can be used as a single copy or as multiple copies, such as one to about 10 copies (i.e., one, two, three, four, five, six, seven, eight, nine or about 10 copies).

c. Hybrid Promoters

The promoter can be a synthetic hybrid promoter. A preferred hybrid promoter herein refers to a promoter comprising a ProA7-derived component having SEQ ID NO: 2, or a variant thereof, including a functional fragment thereof, and a rod-specific-promoter-derived component having SEQ ID NO: 10, or a variant thereof, including a functional fragment thereof, wherein the two components are operably linked to function as a single promoter.

The inventors have surprisingly discovered that such synthetic promoters are unexpectedly cone-specific, and have augmented promoter activity in human cone cells relative to the cone-specific promoter from which the ProA7 component is derived (e.g., SEQ ID NO:2).

The ProA7-derived component, including variants, fragments, and truncations thereof, has been described elsewhere herein.

The rod-specific promoter component can have a nucleotide sequence comprising SEQ ID NO:10. The rod-specific promoter component may comprise a nucleic acid sequence having at least 70% sequence identity to SEQ ID NO:10. The rod-specific promoter component can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, a at least bout 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or greater sequence identity to SEQ ID NO:10.

If desired, the promoter can comprise a rod-specific promoter component variant of SEQ ID NO:10 that retains promoter activity. The variant rod-specific promoter component may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, compared to SEQ ID NO:10.

The functional fragment can comprise from about 370 to 999 nucleotides from SEQ ID NO: 10, such as from about 370 to 999 nucleotides from the 3′ end of SEQ ID NO: 10 or about 895 to 999 nucleotides from the 5′ end of SEQ ID NO: 10.

Preferably, the rod-specific promoter component comprises at least about 370 nucleotides and has at least 70% identity to a sequence of equal length from SEQ ID NO: 10. A promoter consisting of SEQ ID NO: 10 may herein be referred to as ProA330. For example, a rod-specific promoter component can have a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 10 over the length of the rod-specific promoter component. Preferably, the rod-specific promoter component can have at least 90% identity to SEQ ID NO: 10 over the length of the rod-specific promoter component.

These embodiments include, but are not limited to, embodiments wherein the ProA7-derived component has at least 90% identity to a sequence of equal length from the 3′ end of SEQ ID NO: 2.

Exemplary rod-specific promoter components that can be used in the promoters of this disclosure include SEQ ID NO: 10 and a nucleotide sequence that has at least about 90% identity to SEQ ID NO: 10 over the length of the rod-specific promoter component.

The rod-specific promoter component may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 10.

The rod-specific promoter component can comprise a sequence having at least 70% identity to a sequence of equal length from the 3′ end of SEQ ID NO: 10. A rod-specific promoter component according to this paragraph can be considered to be a 5′ truncation of SEQ ID NO: 10, because nucleotides from the 5′ end of SEQ ID NO: 10 have been removed to arrive at the rod-specific promoter component.

The rod-specific promoter component can comprise a sequence having at least 70% identity to a sequence of equal length from the 5′ end of SEQ ID NO: 10. A rod-specific promoter component according to this paragraph can be considered to be a 3′ truncation of SEQ ID NO: 10, because nucleotides from the 3′ end of SEQ ID NO: 10 have been removed to arrive at the rod-specific promoter component.

A rod-specific promoter component can be a 5′ truncation, a 3′ truncation, or both of SEQ ID NO: 10 and can contain fewer than the 1000 nt of SEQ ID NO: 10, due to deletion of nucleotides at the 5′ and/or 3′ ends. For example, the rod-specific promoter component can be the same as nucleotides 106-1000 of SEQ ID NO: 10 or can be the same as nucleotides 1-895 of SEQ ID NO: 10. Preferably, the rod-specific promoter component can comprise or consist of nucleotides 631-895 of SEQ ID NO: 10, which is believed to be the minimal portion of SEQ ID NO:10 required for rod-specific promoter activity.

A rod-specific promoter component can comprise any number of nucleotides from 370 to 1000. For example, the rod-specific promoter component can comprise about 370 nt, about 371 nt, about 372 nt, about 373 nt, about 374 nt, about 375 nt, about 376 nt, about 377 nt, about 378 nt, about 379 nt, about 380 nt, about 381 nt, about 382 nt, about 383 nt, about 384 nt, about 385 nt, about 386 nt, about 387 nt, about 388 nt, about 389 nt, about 390 nt, about 391 nt, about 392 nt, about 393 nt, about 394 nt, about 395 nt, about 396 nt, about 397 nt, about 398 nt, about 399 nt, about 400 nt, about 401 nt, about 402 nt, about 403 nt, about 404 nt, about 405 nt, about 406 nt, about 407 nt, about 408 nt, about 409 nt, about 410 nt, about 411 nt, about 412 nt, about 413 nt, about 414 nt, about 415 nt, about 416 nt, about 417 nt, about 418 nt, about 419 nt, about 420 nt, about 421 nt, about 422 nt, about 423 nt, about 424 nt, about 425 nt, about 426 nt, about 427 nt, about 428 nt, about 429 nt, about 430 nt, about 431 nt, about 432 nt, about 433 nt, about 434 nt, about 435 nt, about 436 nt, about 437 nt, about 438 nt, about 439 nt, about 440 nt, about 441 nt, about 442 nt, about 443 nt, about 444 nt, about 445 nt, about 446 nt, about 447 nt, about 448 nt, about 449 nt, about 450 nt, about 451 nt, about 452 nt, about 453 nt, about 454 nt, about 455 nt, about 456 nt, about 457 nt, about 458 nt, about 459 nt, about 460 nt, about 461 nt, about 462 nt, about 463 nt, about 464 nt, about 465 nt, about 466 nt, about 467 nt, about 468 nt, about 469 nt, about 470 nt, about 471 nt, about 472 nt, about 473 nt, about 474 nt, about 475 nt, about 476 nt, about 477 nt, about 478 nt, about 479 nt, about 480 nt, about 481 nt, about 482 nt, about 483 nt, about 484 nt, about 485 nt, about 486 nt, about 487 nt, about 488 nt, about 489 nt, about 490 nt, about 491 nt, about 492 nt, about 493 nt, about 494 nt, about 495 nt, about 496 nt, about 497 nt, about 498 nt, about 499 nt, about 500 nt, about 501 nt, about 502 nt, about 503 nt, about 504 nt, about 505 nt, about 506 nt, about 507 nt, about 508 nt, about 509 nt, about 510 nt, about 511 nt, about 512 nt, about 513 nt, about 514 nt, about 515 nt, about 516 nt, about 517 nt, about 518 nt, about 519 nt, about 520 nt, about 521 nt, about 522 nt, about 523 nt, about 524 nt, about 525 nt, about 526 nt, about 527 nt, about 528 nt, about 529 nt, about 530 nt, about 531 nt, about 532 nt, about 533 nt, about 534 nt, about 535 nt, about 536 nt, about 537 nt, about 538 nt, about 539 nt, about 540 nt, about 541 nt, about 542 nt, about 543 nt, about 544 nt, about 545 nt, about 546 nt, about 547 nt, about 548 nt, about 549 nt, about 550 nt, about 551 nt, about 552 nt, about 553 nt, about 554 nt, about 555 nt, about 556 nt, about 557 nt, about 558 nt, about 559 nt, about 560 nt, about 561 nt, about 562 nt, about 563 nt, about 564 nt, about 565 nt, about 566 nt, about 567 nt, about 568 nt, about 569 nt, about 570 nt, about 571 nt, about 572 nt, about 573 nt, about 574 nt, about 575 nt, about 576 nt, about 577 nt, about 578 nt, about 579 nt, about 580 nt, about 581 nt, about 582 nt, about 583 nt, about 584 nt, about 585 nt, about 586 nt, about 587 nt, about 588 nt, about 589 nt, about 590 nt, about 591 nt, about 592 nt, about 593 nt, about 594 nt, about 595 nt, about 596 nt, about 597 nt, about 598 nt, about 599 nt, about 600 nt, about 601 nt, about 602 nt, about 603 nt, about 604 nt, about 605 nt, about 606 nt, about 607 nt, about 608 nt, about 609 nt, about 610 nt, about 611 nt, about 612 nt, about 613 nt, about 614 nt, about 615 nt, about 616 nt, about 617 nt, about 618 nt, about 619 nt, about 620 nt, about 621 nt, about 622 nt, about 623 nt, about 624 nt, about 625 nt, about 626 nt, about 627 nt, about 628 nt, about 629 nt, about 630 nt, about 631 nt, about 632 nt, about 633 nt, about 634 nt, about 635 nt, about 636 nt, about 637 nt, about 638 nt, about 639 nt, about 640 nt, about 641 nt, about 642 nt, about 643 nt, about 644 nt, about 645 nt, about 646 nt, about 647 nt, about 648 nt, about 649 nt, about 650 nt, about 651 nt, about 652 nt, about 653 nt, about 654 nt, about 655 nt, about 656 nt, about 657 nt, about 658 nt, about 659 nt, about 660 nt, about 661 nt, about 662 nt, about 663 nt, about 664 nt, about 665 nt, about 666 nt, about 667 nt, about 668 nt, about 669 nt, about 670 nt, about 671 nt, about 672 nt, about 673 nt, about 674 nt, about 675 nt, about 676 nt, about 677 nt, about 678 nt, about 679 nt, about 680 nt, about 681 nt, about 682 nt, about 683 nt, about 684 nt, about 685 nt, about 686 nt, about 687 nt, about 688 nt, about 689 nt, about 690 nt, about 691 nt, about 692 nt, about 693 nt, about 694 nt, about 695 nt, about 696 nt, about 697 nt, about 698 nt, about 699 nt, about 700 nt, about 701 nt, about 702 nt, about 703 nt, about 704 nt, about 705 nt, about 706 nt, about 707 nt, about 708 nt, about 709 nt, about 710 nt, about 711 nt, about 712 nt, about 713 nt, about 714 nt, about 715 nt, about 716 nt, about 717 nt, about 718 nt, about 719 nt, about 720 nt, about 721 nt, about 722 nt, about 723 nt, about 724 nt, about 725 nt, about 726 nt, about 727 nt, about 728 nt, about 729 nt, about 730 nt, about 731 nt, about 732 nt, about 733 nt, about 734 nt, about 735 nt, about 736 nt, about 737 nt, about 738 nt, about 739 nt, about 740 nt, about 741 nt, about 742 nt, about 743 nt, about 744 nt, about 745 nt, about 746 nt, about 747 nt, about 748 nt, about 749 nt, about 750 nt, about 751 nt, about 752 nt, about 753 nt, about 754 nt, about 755 nt, about 756 nt, about 757 nt, about 758 nt, about 759 nt, about 760 nt, about 761 nt, about 762 nt, about 763 nt, about 764 nt, about 765 nt, about 766 nt, about 767 nt, about 768 nt, about 769 nt, about 770 nt, about 771 nt, about 772 nt, about 773 nt, about 774 nt, about 775 nt, about 776 nt, about 777 nt, about 778 nt, about 779 nt, about 780 nt, about 781 nt, about 782 nt, about 783 nt, about 784 nt, about 785 nt, about 786 nt, about 787 nt, about 788 nt, about 789 nt, about 790 nt, about 791 nt, about 792 nt, about 793 nt, about 794 nt, about 795 nt, about 796 nt, about 797 nt, about 798 nt, about 799 nt, about 800 nt, about 801 nt, about 802 nt, about 803 nt, about 804 nt, about 805 nt, about 806 nt, about 807 nt, about 808 nt, about 809 nt, about 810 nt, about 811 nt, about 812 nt, about 813 nt, about 814 nt, about 815 nt, about 816 nt, about 817 nt, about 818 nt, about 819 nt, about 820 nt, about 821 nt, about 822 nt, about 823 nt, about 824 nt, about 825 nt, about 826 nt, about 827 nt, about 828 nt, about 829 nt, about 830 nt, about 831 nt, about 832 nt, about 833 nt, about 834 nt, about 835 nt, about 836 nt, about 837 nt, about 838 nt, about 839 nt, about 840 nt, about 841 nt, about 842 nt, about 843 nt, about 844 nt, about 845 nt, about 846 nt, about 847 nt, about 848 nt, about 849 nt, about 850 nt, about 851 nt, about 852 nt, about 853 nt, about 854 nt, about 855 nt, about 856 nt, about 857 nt, about 858 nt, about 859 nt, about 860 nt, about 861 nt, about 862 nt, about 863 nt, about 864 nt, about 865 nt, about 866 nt, about 867 nt, about 868 nt, about 869 nt, about 870 nt, about 871 nt, about 872 nt, about 873 nt, about 874 nt, about 875 nt, about 876 nt, about 877 nt, about 878 nt, about 879 nt, about 880 nt, about 881 nt, about 882 nt, about 883 nt, about 884 nt, about 885 nt, about 886 nt, about 887 nt, about 888 nt, about 889 nt, about 890 nt, about 891 nt, about 892 nt, about 893 nt, about 894 nt, about 895 nt, about 896 nt, about 897 nt, about 898 nt, about 899 nt, about 900 nt, about 901 nt, about 902 nt, about 903 nt, about 904 nt, about 905 nt, about 906 nt, about 907 nt, about 908 nt, about 909 nt, about 910 nt, about 911 nt, about 912 nt, about 913 nt, about 914 nt, about 915 nt, about 916 nt, about 917 nt, about 918 nt, about 919 nt, about 920 nt, about 921 nt, about 922 nt, about 923 nt, about 924 nt, about 925 nt, about 926 nt, about 927 nt, about 928 nt, about 929 nt, about 930 nt, about 931 nt, about 932 nt, about 933 nt, about 934 nt, about 935 nt, about 936 nt, about 937 nt, about 938 nt, about 939 nt, about 940 nt, about 941 nt, about 942 nt, about 943 nt, about 944 nt, about 945 nt, about 946 nt, about 947 nt, about 948 nt, about 949 nt, about 950 nt, about 951 nt, about 952 nt, about 953 nt, about 954 nt, about 955 nt, about 956 nt, about 957 nt, about 958 nt, about 959 nt, about 960 nt, about 961 nt, about 962 nt, about 963 nt, about 964 nt, about 965 nt, about 966 nt, about 967 nt, about 968 nt, about 969 nt, about 970 nt, about 971 nt, about 972 nt, about 973 nt, about 974 nt, about 975 nt, about 976 nt, about 977 nt, about 978 nt, about 979 nt, about 980 nt, about 981 nt, about 982 nt, about 983 nt, about 984 nt, about 985 nt, about 986 nt, about 987 nt, about 988 nt, about 989 nt, about 990 nt, about 991 nt, about 992 nt, about 993 nt, about 994 nt, about 995 nt, about 996 nt, about 997 nt, about 998 nt, about 999 nt, or about 1000 nt.

Some preferred rod-specific promoter components for use in the promoters of this disclosure have a nucleotide sequence that comprises from about 370 nucleotides to about 1000 nucleotides from the 3′ end of SEQ ID NO: 10.

Some preferred rod-specific promoter components for use in the promoters of this disclosure have a nucleotide sequence that comprises from about 895 nucleotides to about 1000 nucleotides from the 5′ end of SEQ ID NO: 10.

Some preferred rod-specific promoter components for use in the promoters of this disclosure have a nucleotide sequence that comprise or consist of nucleotides 631-895 of SEQ ID NO: 10.

The rod-specific promoter component may comprise a nucleic acid sequence having at least 70% sequence identity to SEQ ID NO: 10 over the length of the rod-specific promoter component. The rod-specific promoter component can have a sequence having at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater sequence identity to SEQ ID NO: 10 over the length of the rod-specific promoter component. Preferably, the rod-specific promoter component can have at least 90% identity to SEQ ID NO: 10 over the length of the rod-specific promoter component.

The rod-specific promoter component may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions, relative to an aligned sequence of the same length from SEQ ID NO: 10.

An exemplary rod-specific promoter component can comprise a sequence of about 895 nt having at least 70% identity to the 3′ end of SEQ ID NO: 10. For example, the rod-specific promoter component can comprise SEQ ID NO: 36. An exemplary rod-specific promoter component can comprise a sequence of about 790 nt having at least 70% identity to the 3′ end of SEQ ID NO: 10. For example, the rod-specific promoter component can comprise SEQ ID NO: 37. An exemplary rod-specific promoter component can comprise a sequence of about 685 nt having at least 70% identity to the 3′ end of SEQ ID NO: 10. For example, the rod-specific promoter component can comprise SEQ ID NO: 38. An exemplary rod-specific promoter component can comprise a sequence of about 580 nt having at least 70% identity to the 3′ end of SEQ ID NO: 10. For example, the rod-specific promoter component can comprise SEQ ID NO: 39. An exemplary rod-specific promoter component can comprise a sequence of about 475 nt having at least 70% identity to the 3′ end of SEQ ID NO: 10. For example, the rod-specific promoter component can comprise SEQ ID NO: 40. An exemplary rod-specific promoter component can comprise a sequence of about 390 nt having at least 70% identity to the 3′ end of SEQ ID NO: 10. For example, the rod-specific promoter component can comprise SEQ ID NO: 41. An exemplary rod-specific promoter component can comprise a sequence of about 895 nt having at least 70% identity to the 5′ end of SEQ ID NO: 10. For example, the rod-specific promoter component can comprise SEQ ID NO: 45.

A rod-specific promoter component can comprise any number of nucleotides from about 260 to about 369 nucleotides of SEQ ID NO:10. For example, the rod-specific promoter component can comprise about 261 nt, 262 nt, 263 nt, 264 nt, 265 nt, 266 nt, 267 nt, 268 nt, 269 nt, 270 nt, 271 nt, 272 nt, 272 nt, 274 nt, 275 nt, 276 nt, 277 nt, 278 nt, 279 nt, 280 nt, 281 nt, 282 nt, 283 nt, 284 nt, 285 nt, 286 nt, 287 nt, 288 nt, 289 nt, 290 nt, 291 nt, 292 nt, 293 nt, 294 nt, 295 nt, 296 nt, 297 nt, 298 nt, 299 nt, 300 nt, 301 nt, 302 nt, 303 nt, 304 nt, 305 nt, 306 nt, 307 nt, 308 nt, 309 nt, 310 nt, 311 nt, 312 nt, 313 nt, 314 nt, 315 nt, 316 nt, 317 nt, 318 nt, 319 nt, 320 nt, 321 nt, 322 nt, 323 nt, 324 nt, 325 nt, 326 nt, 327 nt, 328 nt, 329 nt, 330 nt, 331 nt, 332 nt, 333 nt, 334 nt, 335 nt, 336 nt, 337 nt, 338 nt, 339 nt, 340 nt, 341 nt, 342 nt, 343 nt, 344 nt, 345 nt, 346 nt, 347 nt, 348 nt, 349 nt, 350 nt, 351 nt, 352 nt, 353 nt, 354 nt, 355 nt, 356 nt, 357 nt, 358 nt, 359 nt, 360 nt, 361 nt, 362 nt, 363 nt, 364 nt, 365 nt, 366 nt, 367 nt, 368 nt, or about 369 nt of SEQ ID NO:10. In some preferred aspects, the rod-specific promoter comprises or consist SEQ ID NO: 54 or a sequence that has at least about 70% identity to SEQ ID NO: 54.

In some embodiments, the rod-specific promoter component comprises SEQ ID NO: 36, SEQ ID NO: 37, SEQ ID NO: 38, SEQ ID NO: 39, SEQ ID NO: 40, SEQ ID NO: 41, or SEQ ID NO: 45. In some embodiments, the rod-specific promoter component comprises or consists of nucleotides 631-895 of SEQ ID NO: 10. A particular such promoter, which is also preferred, is referred to as min330 (SEQ ID NO: 54) In some embodiments, in addition to the ProA7-derived component and the rod-specific-promoter derived component, the hybrid promoter can further comprise one or more other rod-specific or cone-specific promoter sequences. In some embodiments, the hybrid promoter can further comprise one or more other cone-specific promoters or nucleotide sequences derived from a cone-specific promoter, such as hG1.7 (SEQ ID NO: 23) or PR1.7 (SEQ ID NO: 22), or variants, fragments, or truncations thereof.

The ProA7-derived component, the rod-specific-promoter-derived component, and any other promoter sequences (if included), can each be included in the hybrid promoter as a single copy or as multiple copies. In some embodiments, the promoter can comprise two or more ProA7-derived components, two or more rod-specific-promoter-derived components, or both. When two or more ProA7-derived components are included in the promoter, the two ProA7-derived components need not be identical, provided they each individually meet the criteria set forth herein regarding ProA7 or variants, fragments, or truncations thereof. Similarly, when two or more rod-specific-promoter-derived components are included in the promoter, the two rod-specific-promoter-derived components need not be identical, provided they each individually meet the criteria set forth herein regarding rod-specific-promoter-derived components.

In the hybrid promoters of this disclosure, the ProA7-derived component(s) and the rod-specific-promoter-derived component(s) can be positioned in any desired order, and with or without other sequences therebetween. As exemplified herein, hybrid promoters that include the same ProA7-derived component and the same rod-specific-promoter-derived component, but in different orders, are effective at driving expression of reporter genes in human cone photoreceptors.

The promoter can comprise from 5′ to 3′ the ProA7-derived component linked to the rod-specific-promoter-derived component with no sequences therebetween. The promoter can comprise from 5′ to 3′ the rod-specific-promoter-derived component linked to the ProA7-derived component with no sequences therebetween. In other examples, the promoter can comprise two ProA7-derived components (component A and component B) and one rod-specific-promoter-derived component, arranged component A/rod-specific-promoter-derived component/component B from 5′ to 3′.

In some embodiments, the promoter can comprise one or more promoter units, wherein each unit comprises a ProA7-derived component and a rod-specific-promoter-derived component in either order. The promoter unit as a separate molecule has cone-specific promoter activity. For example, the promoter can comprise from 1 to about 4 promoter units.

Some particular promoters can include, from 5′ to 3′, a ProA7-derived component comprising SEQ ID NO: 2 and a rod-specific-promoter-derived component comprising SEQ ID NO: 10. A particular such promoter, termed Pro572, has SEQ ID NO: 11.

Some particular promoters can include, from 5′ to 3′, a rod-specific-promoter-derived component comprising SEQ ID NO: 10 and a ProA7-derived component comprising SEQ ID NO: 2. A particular such promoter, termed Pro573, has SEQ ID NO: 13. Some particular promoters can include, from 5′ to 3′, a ProA7-derived component comprising SEQ ID NO: 24 and a rod-specific-promoter-derived component comprising SEQ ID NO: 10. Some particular promoters can include, from 5′ to 3′, a rod-specific-promoter-derived component comprising SEQ ID NO: 10 and a ProA7-derived component comprising SEQ ID NO: 24. Some particular promoters can include, from 5′ to 3′, a ProA7-derived component comprising SEQ ID NO: 25 and a rod-specific-promoter-derived component comprising SEQ ID NO: 10. Some particular promoters can include, from 5′ to 3′, a rod-specific-promoter-derived component comprising SEQ ID NO: 10 and a ProA7-derived component comprising SEQ ID NO: 25.

Some particular promoters can include, from 5′ to 3′, a ProA7-derived component comprising SEQ ID NO: 26 and a rod-specific-promoter-derived component comprising SEQ ID NO: 10. A particular such promoter, which is a preferred promoter, is referred to as Pro572.2 (SEQ ID NO: 12).

In some embodiments, a promoter can include, from 5′ to 3′, a rod-specific-promoter-derived component comprising SEQ ID NO: 10 and a ProA7-derived component comprising SEQ ID NO: 26. A particular such promoter, which is also preferred is referred to as Pro573.2 (SEQ ID NO: 14).

Some particular promoters can include, from 5′ to 3′, a ProA7-derived component comprising SEQ ID NO: 27 and a rod-specific-promoter-derived component comprising SEQ ID NO: 10. Some particular promoters can include, from 5′ to 3′, a rod-specific-promoter-derived component comprising SEQ ID NO: 10 and a ProA7-derived component comprising SEQ ID NO: 27. Some particular promoters can include, from 5′ to 3′, a ProA7-derived component comprising SEQ ID NO: 2 and a rod-specific-promoter-derived component comprising SEQ ID NO: 36. Some particular promoters can include, from 5′ to 3′, a rod-specific-promoter-derived component comprising SEQ ID NO: 36 and a ProA7-derived component comprising SEQ ID NO: 2. Some particular promoters can include, from 5′ to 3′, a ProA7-derived component comprising SEQ ID NO: 2 and a rod-specific-promoter-derived component comprising SEQ ID NO: 37. Some particular promoters can include, from 5′ to 3′, a rod-specific-promoter-derived component comprising SEQ ID NO: 37 and a ProA7-derived component comprising SEQ ID NO: 2. Some particular promoters can include, from 5′ to 3′, a ProA7-derived component comprising SEQ ID NO: 2 and a rod-specific-promoter-derived component comprising SEQ ID NO: 38. Some particular promoters can include, from 5′ to 3′, a rod-specific-promoter-derived component comprising SEQ ID NO: 38 and a ProA7-derived component comprising SEQ ID NO: 2. Some particular promoters can include, from 5′ to 3′, a ProA7-derived component comprising SEQ ID NO: 2 and a rod-specific-promoter-derived component comprising SEQ ID NO: 39. Some particular promoters can include, from 5′ to 3′, a rod-specific-promoter-derived component comprising SEQ ID NO: 39 and a ProA7-derived component comprising SEQ ID NO: 2. Some particular promoters can include, from 5′ to 3′, a ProA7-derived component comprising SEQ ID NO: 2 and a rod-specific-promoter-derived component comprising SEQ ID NO: 40. Some particular promoters can include, from 5′ to 3′, a rod-specific-promoter-derived component comprising SEQ ID NO: 40 and a ProA7-derived component comprising SEQ ID NO: 2. Some particular promoters can include, from 5′ to 3′, a ProA7-derived component comprising SEQ ID NO: 2 and a rod-specific-promoter-derived component comprising SEQ ID NO: 41. Some particular promoters can include, from 5′ to 3′, a rod-specific-promoter-derived component comprising SEQ ID NO: 41 and a ProA7-derived component comprising SEQ ID NO: 2. Some particular promoters can include, from 5′ to 3′, a ProA7-derived component comprising SEQ ID NO: 2 and a rod-specific-promoter-derived component comprising SEQ ID NO: 45. Some particular promoters can include, from 5′ to 3′, a rod-specific-promoter-derived component comprising SEQ ID NO: 45 and a ProA7-derived component comprising SEQ ID NO: 2. Some particular promoters can include, from 5′ to 3′, a ProA7-derived component comprising SEQ ID NO: 24 and a rod-specific-promoter-derived component comprising SEQ ID NO: 36. Some particular promoters can include, from 5′ to 3′, a rod-specific-promoter-derived component comprising SEQ ID NO: 36 and a ProA7-derived component comprising SEQ ID NO: 24. Some particular promoters can include, from 5′ to 3′, a ProA7-derived component comprising SEQ ID NO: 24 and a rod-specific-promoter-derived component comprising SEQ ID NO: 37. Some particular promoters can include, from 5′ to 3′, a rod-specific-promoter-derived component comprising SEQ ID NO: 37 and a ProA7-derived component comprising SEQ ID NO: 24. Some particular promoters can include, from 5′ to 3′, a ProA7-derived component comprising SEQ ID NO: 24 and a rod-specific-promoter-derived component comprising SEQ ID NO: 38. Some particular promoters can include, from 5′ to 3′, a rod-specific-promoter-derived component comprising SEQ ID NO: 38 and a ProA7-derived component comprising SEQ ID NO: 24. Some particular promoters can include, from 5′ to 3′, a ProA7-derived component comprising SEQ ID NO: 24 and a rod-specific-promoter-derived component comprising SEQ ID NO: 39. Some particular promoters can include, from 5′ to 3′, a rod-specific-promoter-derived component comprising SEQ ID NO: 39 and a ProA7-derived component comprising SEQ ID NO: 24. Some particular promoters can include, from 5′ to 3′, a ProA7-derived component comprising SEQ ID NO: 24 and a rod-specific-promoter-derived component comprising SEQ ID NO: 40. Some particular promoters can include, from 5′ to 3′, a rod-specific-promoter-derived component comprising SEQ ID NO: 40 and a ProA7-derived component comprising SEQ ID NO: 24. Some particular promoters can include, from 5′ to 3′, a ProA7-derived component comprising SEQ ID NO: 24 and a rod-specific-promoter-derived component comprising SEQ ID NO: 41. Some particular promoters can include, from 5′ to 3′, a rod-specific-promoter-derived component comprising SEQ ID NO: 41 and a ProA7-derived component comprising SEQ ID NO: 24. Some particular promoters can include, from 5′ to 3′, a ProA7-derived component comprising SEQ ID NO: 24 and a rod-specific-promoter-derived component comprising SEQ ID NO: 45. Some particular promoters can include, from 5′ to 3′, a rod-specific-promoter-derived component comprising SEQ ID NO: 45 and a ProA7-derived component comprising SEQ ID NO: 24. Some particular promoters can include, from 5′ to 3′, a ProA7-derived component comprising SEQ ID NO: 25 and a rod-specific-promoter-derived component comprising SEQ ID NO: 36. Some particular promoters can include, from 5′ to 3′, a rod-specific-promoter-derived component comprising SEQ ID NO: 36 and a ProA7-derived component comprising SEQ ID NO: 25. Some particular promoters can include, from 5′ to 3′, a ProA7-derived component comprising SEQ ID NO: 25 and a rod-specific-promoter-derived component comprising SEQ ID NO: 37. Some particular promoters can include, from 5′ to 3′, a rod-specific-promoter-derived component comprising SEQ ID NO: 37 and a ProA7-derived component comprising SEQ ID NO: 25. Some particular promoters can include, from 5′ to 3′, a ProA7-derived component comprising SEQ ID NO: 25 and a rod-specific-promoter-derived component comprising SEQ ID NO: 38. Some particular promoters can include, from 5′ to 3′, a rod-specific-promoter-derived component comprising SEQ ID NO: 38 and a ProA7-derived component comprising SEQ ID NO: 25. Some particular promoters can include, from 5′ to 3′, a ProA7-derived component comprising SEQ ID NO: 25 and a rod-specific-promoter-derived component comprising SEQ ID NO: 39. Some particular promoters can include, from 5′ to 3′, a rod-specific-promoter-derived component comprising SEQ ID NO: 39 and a ProA7-derived component comprising SEQ ID NO: 25. Some particular promoters can include, from 5′ to 3′, a ProA7-derived component comprising SEQ ID NO: 25 and a rod-specific-promoter-derived component comprising SEQ ID NO: 40. Some particular promoters can include, from 5′ to 3′, a rod-specific-promoter-derived component comprising SEQ ID NO: 40 and a ProA7-derived component comprising SEQ ID NO: 25. Some particular promoters can include, from 5′ to 3′, a ProA7-derived component comprising SEQ ID NO: 25 and a rod-specific-promoter-derived component comprising SEQ ID NO: 41. Some particular promoters can include, from 5′ to 3′, a rod-specific-promoter-derived component comprising SEQ ID NO: 41 and a ProA7-derived component comprising SEQ ID NO: 25. Some particular promoters can include, from 5′ to 3′, a ProA7-derived component comprising SEQ ID NO: 25 and a rod-specific-promoter-derived component comprising SEQ ID NO: 45. Some particular promoters can include, from 5′ to 3′, a rod-specific-promoter-derived component comprising SEQ ID NO: 45 and a ProA7-derived component comprising SEQ ID NO: 25. Some particular promoters can include, from 5′ to 3′, a ProA7-derived component comprising SEQ ID NO: 26 and a rod-specific-promoter-derived component comprising SEQ ID NO: 36. Some particular promoters can include, from 5′ to 3′, a rod-specific-promoter-derived component comprising SEQ ID NO: 36 and a ProA7-derived component comprising SEQ ID NO: 26. Some particular promoters can include, from 5′ to 3′, a ProA7-derived component comprising SEQ ID NO: 26 and a rod-specific-promoter-derived component comprising SEQ ID NO: 37. Some particular promoters can include, from 5′ to 3′, a rod-specific-promoter-derived component comprising SEQ ID NO: 37 and a ProA7-derived component comprising SEQ ID NO: 26. Some particular promoters can include, from 5′ to 3′, a ProA7-derived component comprising SEQ ID NO: 26 and a rod-specific-promoter-derived component comprising SEQ ID NO: 38. Some particular promoters can include, from 5′ to 3′, a rod-specific-promoter-derived component comprising SEQ ID NO: 38 and a ProA7-derived component comprising SEQ ID NO: 26. Some particular promoters can include, from 5′ to 3′, a ProA7-derived component comprising SEQ ID NO: 26 and a rod-specific-promoter-derived component comprising SEQ ID NO: 39. Some particular promoters can include, from 5′ to 3′, a rod-specific-promoter-derived component comprising SEQ ID NO: 39 and a ProA7-derived component comprising SEQ ID NO: 26. Some particular promoters can include, from 5′ to 3′, a ProA7-derived component comprising SEQ ID NO: 26 and a rod-specific-promoter-derived component comprising SEQ ID NO: 40. Some particular promoters can include, from 5′ to 3′, a rod-specific-promoter-derived component comprising SEQ ID NO: 40 and a ProA7-derived component comprising SEQ ID NO: 26. Some particular promoters can include, from 5′ to 3′, a ProA7-derived component comprising SEQ ID NO: 26 and a rod-specific-promoter-derived component comprising SEQ ID NO: 41. Some particular promoters can include, from 5′ to 3′, a rod-specific-promoter-derived component comprising SEQ ID NO: 41 and a ProA7-derived component comprising SEQ ID NO: 26. Some particular promoters can include, from 5′ to 3′, a ProA7-derived component comprising SEQ ID NO: 26 and a rod-specific-promoter-derived component comprising SEQ ID NO: 45. Some particular promoters can include, from 5′ to 3′, a rod-specific-promoter-derived component comprising SEQ ID NO: 45 and a ProA7-derived component comprising SEQ ID NO: 26. Some particular promoters can include, from 5′ to 3′, a ProA7-derived component comprising SEQ ID NO: 27 and a rod-specific-promoter-derived component comprising SEQ ID NO: 36. Some particular promoters can include, from 5′ to 3′, a rod-specific-promoter-derived component comprising SEQ ID NO: 36 and a ProA7-derived component comprising SEQ ID NO: 27. Some particular promoters can include, from 5′ to 3′, a ProA7-derived component comprising SEQ ID NO: 27 and a rod-specific-promoter-derived component comprising SEQ ID NO: 37. Some particular promoters can include, from 5′ to 3′, a rod-specific-promoter-derived component comprising SEQ ID NO: 37 and a ProA7-derived component comprising SEQ ID NO: 27. Some particular promoters can include, from 5′ to 3′, a ProA7-derived component comprising SEQ ID NO: 27 and a rod-specific-promoter-derived component comprising SEQ ID NO: 38. Some particular promoters can include, from 5′ to 3′, a rod-specific-promoter-derived component comprising SEQ ID NO: 38 and a ProA7-derived component comprising SEQ ID NO: 27. Some particular promoters can include, from 5′ to 3′, a ProA7-derived component comprising SEQ ID NO: 27 and a rod-specific-promoter-derived component comprising SEQ ID NO: 39. Some particular promoters can include, from 5′ to 3′, a rod-specific-promoter-derived component comprising SEQ ID NO: 39 and a ProA7-derived component comprising SEQ ID NO: 27. Some particular promoters can include, from 5′ to 3′, a ProA7-derived component comprising SEQ ID NO: 27 and a rod-specific-promoter-derived component comprising SEQ ID NO: 40. Some particular promoters can include, from 5′ to 3′, a rod-specific-promoter-derived component comprising SEQ ID NO: 40 and a ProA7-derived component comprising SEQ ID NO: 27. Some particular promoters can include, from 5′ to 3′, a ProA7-derived component comprising SEQ ID NO: 27 and a rod-specific-promoter-derived component comprising SEQ ID NO: 41. Some particular promoters can include, from 5′ to 3′, a rod-specific-promoter-derived component comprising SEQ ID NO: 41 and a ProA7-derived component comprising SEQ ID NO: 27. Some particular promoters can include, from 5′ to 3′, a ProA7-derived component comprising SEQ ID NO: 27 and a rod-specific-promoter-derived component comprising SEQ ID NO: 45. Some particular promoters can include, from 5′ to 3′, a rod-specific-promoter-derived component comprising SEQ ID NO: 45 and a ProA7-derived component comprising SEQ ID NO: 27

The hybrid promoter can include, from 5′ and 3′, a rod-specific promoter-derived component comprising SEQ ID NO: 54 or a functional variant thereof and a ProSC-derived component comprising SEQ ID 26 or a functional variant thereof. Alternatively, a promoter can include, from 5′ to 3′, a ProSC-derived component comprising SEQ ID NO: 26 or a functional variant thereof and a rod-specific promoter-derived component comprising SEQ ID NO:54 or a functional variant thereof.

In certain preferred embodiments, the hybrid promoter contains about 700 or fewer nucleotides, about 600 or fewer nucleotides, or more preferably, about 500 or fewer nucleotides.

### B. Optogenetic Proteins

As disclosed herein, the nucleic acid comprises a nucleotide sequence encoding a depolarizing optogenetic protein. The depolarizing optogenetic protein can be expressed in a human cone cell transduced with the nucleic acid and localized to the cell membrane of the human cone cell. The depolarizing optogenetic protein can mediate a depolarizing current that can depolarize a human cone cell when exposed to light. Depolarization of the human cone cell can induce light-driven current spikes in RGCs.

The depolarizing optogenetic protein can be activated or excited by any desired type of light, such as green light, red light, blue light, violet light, or yellow light. The light can have a wavelength of between about 400 nm to about 700 nm. For example, the light can have a wavelength of about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600, about 610, about 620, about 630, about 640, about 650, about 660, about 670, about 680, about 690, or about 700 nm.

The human cone cell comprising the depolarizing optogenetic protein described herein can have a resting potential of about −30 to −35 mV. Upon activation (e.g., photostimulation) the depolarizing optogenetic protein can generate a voltage (i.e., an action potential) across the cell membrane of a human cone cell of at least about −40 mV up to about −55 mV or greater. The human cone cell may be depolarized by at least about 1 mV to about 20 mV or more. For example, the human cone cell can be depolarized by at least about 1 mV to about 2 mV, about 1 mV to about 3 mV, about 1 mV to 4 mV, about 1 mV to about 5 mV, about 1 mV to about 6 mV, about 1 mV to about 7 mV, about 1 mV to about 8 mV, about 1 mV to about 9 mV, about 1 mV to about 10 mV, about 1 mV to about 11 mV, about 1 mV to about 12 mV, about 1 mV to about 13 mV, about 1 mV to about 14 mV, about 1 mV to about 19 mV, or about 1 mV to about 20 mV.

Preferably, the depolarizing optogenetic protein can generate a voltage across the cell membrane of a human cone cell that is sufficient to trigger the firing of an action potential that can induce light-driven ganglion cell spiking.

The depolarizing optogenetic protein can mediate a depolarizing current that is about 1 fold, about 2 fold, about 3 fold, about 4 fold, about 5 fold, about 6 fold, about 7 fold, about 8 fold, about 10 fold, about 11 fold, about 12 fold, about 13 fold, about 14 fold, about 15 fold or greater than other light-activated ion gated channel proteins.

The depolarizing optogenetic protein is preferably a light-gated ion channel protein. Suitable light-gated ion channel proteins include, but are not limited to channelrhodopsins (e.g., channelrhodopsin-1 (ChR1), a channelrhodopsin 2 (ChR2)), variants thereof, or combinations of the foregoing. The channelrhodopsin may be a ChrMine polypeptide, a Chrimson polypeptide, a ReaChR polypeptide, a variant thereof, or combinations of the foregoing.

Examples of depolarizing optogenetic proteins that may be suitable for the optogenetic constructs disclosed herein include ChrimsonR, ReaChR, ChrMine, fChrimson, and vfChrimson. A preferred depolarizing optogenetic protein is ReaChR.

The amino acid sequence of ReaChR is provided as SEQ ID NO: 32. Nucleotide sequences encoding ReaChR are provided at SEQ ID NO: 16, SEQ ID NO: 33, and SEQ ID NO: 34. SEQ ID NO: 33 and SEQ ID NO: 34 are examples of nucleic acids that encode ReaChR that are codon optimized for expression in human cells.

Other depolarizing optogenetic proteins that may be adapted for the optogenetic constructs disclosed herein include, but are not limited to, ChR2, ChETA, SFO, VChR1, Chronos, PsChR2, CoChR, CsChR, CheRiff, C1C2, C1V1.

The depolarizing optogenetic protein may be a functional variant of the light-gated ion channel protein. For example, the depolarizing optogenetic protein may be a functional variant of ChrimsonR, ReaChR, ChrMine, fChrimson, or vfChrimson.

The functional variant can differ from the light-gated ion channel protein by one or a few amino acids (including substitutions, deletions, insertions, or any combination thereof), and substantially retain their ability to mediate a depolarizing current that depolarizes a human cone cell when exposed to light. The functional variant can contain at least one or more amino acid substitutions, deletions, or insertions relative to the light-gated ion channel polypeptide. The functional variant can comprise about 1, about 2, about 3, about 4, about 5, about 6, about 7, about 8, about 9, about 10, about 11, about 12, about 13, about 14, about 15, about 16, about 17, about 18, about 19, about 20 or more amino acid alterations compared to the light-gated ion channel protein. The amino acid substitution can be a conservative substitution or a non-conservative substitution, but preferably is a conservative substitution. A “conservative” amino acid substitution, as used herein, generally refers to substitution of one amino acid residue with another amino acid residue from within a recognized group, which can change the structure of the peptide yet biological activity of the peptide is substantially retained. Conservative substitutions of amino acids are known to those skilled in the art. Conservative substitutions of amino acids can include, but are not limited to, substitutions made amongst amino acids within the following groups: (a) M, I, L, V; (b) F, Y, W; (c) K, R, H; (d) A, G; (e) S, T; (f) Q, N; and (g) E, D. For example, a person of ordinary skill in the art would reasonably expect that an isolated replacement of a leucine with an isoleucine or valine, an aspartate with a glutamate, a threonine with a serine, or a similar replacement of an amino acid with a structurally related amino acid will not have a significant effect on the biological activity of the resulting molecule.

The nucleotide sequence encoding the depolarizing optogenetic protein can be modified to optimize codon usage, depending on the organism, cell, or organoid in which it is desired to express the depolarizing optogenetic protein using any suitable methods. Several methods and algorithms for codon optimization are conventional and well-known to those of ordinary skill in the art. Codon optimization of the nucleotide sequence encoding the depolarizing optogenetic protein can increase the rate of translation of the depolarizing optogenetic protein. The depolarizing optogenetic protein can be engineered to include one or more membrane localization sequence(s), such as from other membrane associated proteins (e.g., other optogenetic proteins). Without wishing to be bound by any particular mechanism or theory, it is believed that such engineered depolarizing optogenetic proteins may have enhanced membrane localization. As an exemplary and non-limiting example, a transmembrane domain of ReaChR can be inserted into another depolarizing optogenetic protein, e.g. to replace the transmembrane domain of the other depolarizing optogenetic protein, to provide an additional membrane localization sequence to the other depolarizing optogenetic protein. In another example, the membrane localization sequence of ReaChR (SEQ ID NO:57) can be added to depolarizing optogenetic protein that does not contain one, or can replace the membrane localization sequence of a desired depolarizing optogenetic protein. For example, ChRMine, Chrimson, Jaws, HcKCR1 or eGTACR1 can be engineered to improve membrane localization by replacing the N-terminus with the corresponding membrane localization sequence of ReaChr (SEQ ID NO: 57). In particular, an optogenic protein, such as a depolarizing channel rhodopsin, can be engineered to improve membrane localization by replacing the N-terminus of the channelrhodopsin, e.g. ChR1, with (SEQ ID NO 57). (See, e.g. Lin et al., Biophysical Journal, 96:1803-1814 (2009), Lin et al., Nat. Neurosci. 16(10): 1499-1508 (2013))

### C. Reporter Molecule

The nucleic acid can comprise a nucleotide sequence encoding a reporter molecule, if desired. The optional reporter molecule, when present, can be operably linked to the nucleotide sequence encoding the depolarizing optogenetic protein. The reporter molecule can be detectable in human cone cells. The depolarizing optogenetic protein and the reporter molecule may be components of a fusion protein, with the reporter molecule located C-terminally or N-terminally of the depolarizing optogenetic protein, as desired. The depolarizing optogenetic protein and the reporter molecule can be directly fused to each other, or indirectly fused, for example through an suitable linker sequence. For example, the reporter can be fused to the C- or N-terminus of the depolarizing optogenetic protein directly or indirectly through a suitable linker peptide. Without being bound by theory, the reporter molecule may enhance membrane localization of the optogenetic construct. Suitable reporter molecules include, for example, tdTomato, enhanced yellow fluorescent protein (EYFP), Citrine, green fluorescent protein (GFP), cyan fluorescent protein, red fluorescent protein, or functional variants thereof. A preferred reporter molecule is Citrine.

Without wishing to be bound by any particular mechanism of theory, it is believed that inclusion of a reporter molecule that is operably linked to depolarizing optogenetic protein may enhance membrane localization of the depolarizing optogenetic protein, even though the depolarizing optogenetic proteins are themselves naturally membrane associated.

### D. PRE

As disclosed herein, the nucleic acid comprises a nucleotide sequence encoding a PRE and preferably a WPRE. PREs are nucleic acid sequences that contribute to regulation of expression of a DNS sequence within which the PRE is located. A PRE may include, in some instances, three components (alpha, beta, and gamma). The activity of the PRE may depend on how many of the components are present.

As disclosed and exemplified herein, a WPRE element in combination with the promoter, such as a ProA7 promoter, a variant, fragment, or truncation thereof, or a hybrid promoter comprising a ProA7-derived component and a rod-specific-promoter-derived component, and a depolarizing optogenetic protein can result in high levels of expression of the depolarizing optogenetic protein in human cone cells.

As disclosed above, the inventors surprisingly discovered that the ProA7 promoter in combination with a posttranscriptional regulatory element (PRE), preferably the woodchuck hepatis virus PRE (WPRE), can drive high expression levels of the depolarizing optogenetic protein in human photoreceptor cone cells.

The WPRE can be operably linked to a nucleotide encoding depolarizing optogenetic protein and other expression control elements, e.g. a promoter described herein and a PolyA signal.

Any suitable WPRE may be used, such as naturally occurring WPRE or a WPRE that comprises one or more mutation in the X region. For instance, a suitable WPRE with mutation in the X region is disclosed in U.S. Pat. No. 7,419,829.

A suitable WPRE has been described in US Publication No. US 2021/0032656. Generally, the WPRE can have a nucleotide sequence comprising SEQ ID NO: 3. The WPRE may comprise a nucleic acid sequence having at least 70% sequence identity to SEQ ID NO: 3. The WPRE can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO:3.

The WPRE may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions. The nucleic acid substitution can be a conservative substitution or a non-conservative substitution, but preferably is a conservative substitution.

The WPRE can have a nucleotide sequence comprising SEQ ID NO: 8. The WPRE may comprise a nucleic acid sequence having at least 70% sequence identity to SEQ ID NO: 3. The WPRE can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO:8.

The WPRE may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions. The nucleic acid substitution can be a conservative substitution or a non-conservative substitution, but preferably is a conservative substitution.

Preferably, the WPRE can have a nucleotide sequence comprising SEQ ID NO: 3. The WPRE may comprise a nucleic acid sequence having at least 70% sequence identity to SEQ ID NO: 3. The WPRE can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO:86.

The WPRE may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions. The nucleic acid substitution can be a conservative substitution or a non-conservative substitution, but preferably is a conservative substitution.

### E. Polyadenylation Signal and Inverted Terminal Repeat Sequence

The nucleic acid can also include a nucleotide sequence that encodes a suitable PolyA that is 3′ of the WPRE. Any suitable PolyA signal can be used, such as, an SV40 PolyA signal, rabbit beta-globin PolyA signal, human growth hormone (hGH) PolyA signal, bovine growth hormone PolyA signal, and the like. Human growth hormone (hGH) polyA signal is a preferred PolyA.

The PolyA can be in any suitable orientation, preferably the PolyA is 3′ of the WPRE.

The PolyA can have a nucleotide sequence comprising SEQ ID NO: 9. The PolyA may comprise a nucleic acid sequence having at least 70% sequence identity to SEQ ID NO: 9. The PolyA can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO:9.

The PolyA can have a nucleotide sequence comprising SEQ ID NO: 87. The PolyA may comprise a nucleic acid sequence having at least 70% sequence identity to SEQ ID NO: 87. The PolyA can have a nucleotide sequence having at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or greater sequence identity to SEQ ID NO:87.

The PolyA may comprise about 1, about 2, about 3, about 4, about 5, about 6, about 7, about 8, about 9, about 10, about 11, about 12, about 13, about 14, about 15, about 16, about 17, about 18, about 19, about 20, or more nucleic acid alterations, e.g., substitutions or deletions. The nucleic acid substitution can be a conservative substitution or a non-conservative substitution, but preferably is a conservative substitution.

The nucleic acid can further comprise an AAV inverted terminal repeat sequence (ITR). For example, an AAV ITR 5′ of the promoter and an AAV ITR 3′ of the WPRE or 3′ of the PolyA. Particular examples of AAV ITRs are SEQ ID NO: 15 and SEQ ID NO: 31. ITRs may be independently selected from wild-type ITRs and optionally self-complementary (scAAV) ITRs. Other sequences (e.g., parvovirus terminal repeats) which are functionally equivalent to AAV 5′ ITRs and/or AAV 3′ ITRs can also be used.

### F. Adeno-Associated Viral Vector Capsid

The disclosure also relates to viral vectors that comprise the nucleic acid disclosed herein. The viral vector can be an AAV vector that comprises an AAV capsid. Without being bound by theory, the AAV capsid can improve selective delivery of the nucleic acid to cone cells and may also improve expression of the optogenetic construct (i.e., the nucleic acid).

Many suitable AAV capsids and viral backbones are well-known in the art and multiple AAV capsid serotypes are known and may be suitable for the optogenetic constructs disclosed herein. At least sixteen serotypes of AAV have been described in literature, and are referred to as AAV1, AAV2, AAV3, AAV4, AAV5, AAV6, AAV7, AAV8, AAV9, AAV10, AAV 11, AAV 12, AAV13, AAV 14, AAV15, and AAV16. Many engineered and variant capsids are also well-known in the art.

Exemplary AAV capsids suitable for the optogenetic constructs disclosed herein include, but are not limited to, AAV8-BP2, AAV-PHP.B, AAV-PHP.eB, AAV5, or AAV-NHP26. Preferred AAV capsid proteins are AAV-PHP.eB, AAV8-BP2, or AAV5.

Alternatively to using AAV natural serotypes, artificial AAV serotypes may be used, such as, AAV with a non-naturally occurring capsids. Such an artificial capsid may be generated by any suitable technique, using a selected AAV sequence (e.g., a fragment of a VP1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid or a mutated AAV capsid. A chimeric capsid comprises VP capsid proteins derived from at least two different AAV serotypes or comprises at least one chimeric VP protein combining VP protein regions or domains derived from at least two AAV serotypes.

AAV capsid proteins may also be mutated, in particular to enhance transduction efficiency. Mutated AAV capsids may be obtained from capsid modifications inserted by error prone PCR and/or peptide insertion or by including one or several amino acids substitutions. In particular, mutations may be made in any one or more of tyrosine residues of natural or non-natural capsid proteins (e.g. VP1, VP2, or VP3). Mutated residues may be surface exposed tyrosine residues. Exemplary mutations include, but are not limited to tyrosine-to-phenylalanine substitutions such as Y252F, Y272F, Y444F, Y500F, Y700F, Y704F, Y730F, Y275F, Y281F, Y508F, Y576F, Y612G, Y673F and Y720F.

### G. Combination of Elements

As disclosed herein, the inventors discovered that preferred combinations of expression control elements and specific depolarizing optogenetic proteins and optionally a reporter molecule provide high level and selective expression of the depolarizing optogenetic protein in human cone cells, which is sufficient to restore light sensitivity in human cone cells, particularly human cone cells that are not responsive to light. Preferred expression control elements for the nucleic acids disclosed herein that encode a depolarizing optogenetic protein are a promoter comprising ProA7, or a variant, fragment, or truncation thereof, or a hybrid promoter comprising a ProA7-derived component and a rod-specific-promoter-derived component, and WPRE. The optogenetic construct disclosed herein typically further comprise a suitable polyadenylation signal (PolyA) that is 3′ of the WPRE. The optogenetic construct can further comprise one or more AAV inverted terminal repeat sequence (ITRs). The ITRs can be 5′ of the promoter and/or 3′ of the WPRE or PolyA, when the PolyA is present. Modified ITRs, e.g., self-complementary ITR, or other sequences (e.g., parvovirus terminal repeats) which are functionally equivalent to AAV 5′ ITRs and/or AAV 3′ ITRs can also be used in the optogenetic construct.

The optogenetic construct can include a single copy or multiple copies of the promoter or any element thereof in a hybrid promoter. For examples, the construct can include multiple copies of ProA7, or a variant, fragment, or truncation thereof, multiple copies of ProA330 or a variant, fragment, or truncation thereof, or a hybrid promoter comprising a Pro-A7-derived component and a rod-specific-promoter-derived component (such as a ProA330 derived component), and WPRE. In one embodiment, the construct can include but is not limited to two, three, four, five, six, seven, eight, nine or ten copies of the ProA7 promoter or a variant, fragment, or truncation thereof. In another embodiment the construct can include but is not limited to two, three, four, five, six, seven, eight, nine, or ten copies of the ProA330 promoter or a variant, fragment, or truncation thereof. In some embodiments, the construct can include but is not limited to two, three, four, five, six, seven, eight, nine or ten copies of the hybrid promoter comprising a Pro-A7-derived component and a cone or rod-specific-promoter-derived component. A hybrid promoter comprising a Pro-A7-derived component and a cone or rod-specific-promoter-derived component and optionally any other rod- or cone-specific promoter sequences can each be included as a single copy or as multiple copies. When the promoter comprising ProA7, or a variant, fragment, or truncation thereof, ProA330 or a variant, fragment, or truncation thereof, or a variant, fragment, or truncation thereof, or a hybrid promoter comprising a Pro-A7-derived component and a rod-specific-promoter-derived component are included, the two or more promoter sequences need not be identical. The multiple promoter copies can also be in any orientation. Multiple copies of promoters comprising ProA7, or a variant, fragment, or truncation thereof, Pro330, a variant, fragment, or truncation thereof, or a hybrid promoter comprising a Pro-A7-derived component and a rod-specific-promoter-derived component may be included in any order. The particular combination of the depolarizing optogenetic protein and optional reporter molecule can improve expression and function in the human cone cells, particularly human cone cells that are not responsive to light. For example, the depolarizing optogenetic protein can be ChrimsonR and the reporter molecule can be tdTomato. For example, the depolarizing optogenetic protein can be ChrimsonR and the reporter molecule can be EYFP. For example, the depolarizing optogenetic protein can be ReaChR and the reporter molecule can be Citrine. For example, the depolarizing optogenetic protein can be ChrMine and the reporter molecule can be EYFP. For example, the depolarizing optogenetic protein can be fChrimson and the reporter molecule can be TdTomato. For example, the depolarizing optogenetic protein can be vfChrimson and the reporter molecule can be TdTomato.

A preferred depolarizing optogenetic protein is ReaChr and when present, a preferred reporter molecule is Citrine.

The optogenetic construct can be an AAV vector that comprises the nucleic acid disclosed herein and an AAV capsid.

Exemplary optogenetic constructs (i.e., AAV vectors) comprising a depolarizing optogenetic protein, a reporter molecule which is optional can be omitted, and an AAV capsid protein are described below in Table 1.

TABLE 1
Combinations of depolarizing optogenetic proteins, optional
reporter molecules, and AAV capsid proteins.

| | Reporter Molecule | |
|---|---|---|
| Optogenetic Protein | (optional) | AAV capsid protein |
| ReaChR | Citrine | AAV-PHP.eB |
| ReaChR | Citrine | AAV8-BP2 |
| ReaChR | Citrine | AAV-NHP26 |
| ReaChR | Citrine | AAV5 |
| ReaChR | Citrine | AAV8 |
| ReaChR | Citrine | AAV9 |
| ReaChR | no tag | AAV5 |
| ChrMine | EYFP | AAV-PHP.B |
| ChrMine | EYFP | AAV-PHP.eB |
| ChrMine | EYFP | AAV-BP2 |
| ChrMine | EYFP | AAV-NHP26 |
| ChrimsonR | tdTomato | AAV8-BP2 |
| ChrimsonR | tdTomato | AAV-PHP.B |
| ChrimsonR | tdTomato | AAV-PHP.eB |
| ChrimsonR | tdTomato | AAV-NHP26 |
| vfChrimson | tdTomato | AAV8-BP2 |
| vfChrimson | tdTomato | AAV-PHP.B |
| vfChrimson | tdTomato | AAV-PHP.eB |
| vfChrimson | tdTomato | AAV-NHP26 |
| ChrimsonR | EYFP | AAV8-BP2 |
| ChrimsonR | EYFP | AAV-PHP.B |
| ChrimsonR | EYFP | AAV-PHP.eB |
| ChrimsonR | EYFP | AAV-NHP26 |
| ChrMine | EYFP | AAV8-BP2 |
| ChrMine | EYFP | AAV-PHP |
| ChrMine | EYFP | AAV-PHP |
| ChrMine | EYFP | AAV-NHP26 |
| fChrimson | tdTomato | AAV8-BP2 |
| fChrimson | tdTomato | AAV-PHP.B |
| fChrimson | tdTomato | AAV-PHP.eB |
| fChrimson | tdTomato | AAV-NHP26 |

The optogenetic construct (i.e., an AAV vector) can comprise a nucleic acid that comprises a promoter, ChrimsonR as the depolarizing optogenetic protein, a WPRE, and a PolyA signal. The nucleic acid can further comprise tdTomato as the reporter molecule. The nucleic acid can further comprise an AAV ITR 5′ of the promoter and an AAV ITR that 3′ of the PolyA signal. The optogenetic construct can further comprise an AAV capsid, if desired. The nucleic acid comprising the ChrimsonR depolarizing optogenetic protein may comprise a promoter having a nucleotide sequence having SEQ ID NO: 2. The nucleic acid comprising the ChrimsonR depolarizing optogenetic construct may comprise a WPRE having a nucleotide sequence having SEQ ID NO: 3.

The optogenetic construct (i.e., an AAV vector) can comprise a nucleic acid that comprises a promoter, ChrimsonR as the depolarizing optogenetic protein, a WPRE, and a PolyA signal. The nucleic acid can further comprise tdTomato as the reporter molecule. The nucleic acid can further comprise an AAV ITR 5′ of the promoter and an AAV ITR that 3′ of the PolyA signal. The optogenetic construct can further comprise an AAV capsid, if desired. The nucleic acid comprising the ChrimsonR depolarizing optogenetic protein may comprise a promoter having a nucleotide sequence having SEQ ID NO: 2. The nucleic acid comprising the ChrimsonR depolarizing optogenetic construct may comprise a WPRE having a nucleotide sequence having SEQ ID NO: 86.

The optogenetic construct disclosed herein may comprise a nucleic acid sequence having at least 70% identity to SEQ ID NO: 4 and may comprise a an AAV capsid protein chosen from a AAV-BP2 capsid protein, a AAV-PHP.B capsid protein, a AAV-PHP.eB capsid protein, or a AAV-NH26 capsid protein. For example, the nucleic acid may comprise a nucleotide having at least about 70%, about 75%, about 80%, about 85%, about 90%, about 91%, about 92%, about 93%, about 94%, about 95%, about 96%, about 97%, about 98%, about 99%, or greater sequence identity to SEQ ID NO: 4.

The optogenetic construct (i.e., an AAV) may comprise a nucleic acid that comprises a ProA7 promoter, vfChrimsonR as the depolarizing optogenetic protein, a WPRE, and a PolyA signal. The optogenetic construct may further comprise EYFP as the reporter molecule. The nucleic acid can further comprise an AAV ITR 5′ of the ProA7 promoter and an AAV ITR that 3′ of the PolyA signal. The optogenetic construct can further comprise an AAV capsid, if desired. The optogenetic construct comprising the vfChrimsonR depolarizing optogenetic construct can comprise a ProA7 promoter having a nucleotide sequence having SEQ ID NO: 2. The optogenetic construct comprising the vfChrimsonR depolarizing optogenetic protein can comprise a WPRE having a nucleotide sequence having SEQ ID NO: 3. The optogenetic construct containing vfChrimsonR depolarizing optogenetic protein can comprise a WPRE having a nucleotide sequence having SEQ ID NO: 86.

The optogenetic construct disclosed herein may comprise a nucleic acid sequence having at least 70% identity to SEQ ID NO: 5 and may comprise a an AAV capsid protein. For example, the nucleic acid may comprise a nucleotide having at least about 70%, about 75%, about 80%, about 85%, about 90%, about 91%, about 92%, about 93%, about 94%, about 95%, about 96%, about 97%, about 98%, about 99%, or greater sequence identity to SEQ ID NO: 5.

The optogenetic construct (i.e., an AAV) may comprise a nucleic acid comprising a ProA7 promoter, ChrMine as the depolarizing optogenetic protein, a WPRE, and a PolyA signal. The optogenetic construct may further comprise EYFP as the reporter molecule. The nucleic acid can further comprise an AAV ITR 5′ of the ProA7 promoter and an AAV ITR that 3′ of the PolyA signal. The optogenetic construct may further comprise an AAV capsid protein, if desired. The optogenetic construct comprising the ChrMine depolarizing optogenetic protein can comprise a ProA7 promoter having a nucleotide sequence having SEQ ID NO: 2. The optogenetic construct comprising the ChrMine depolarizing optogenetic construct can comprise a WPRE having a nucleotide sequence having SEQ ID NO: 3. The optogenetic construct comprising the ChrMine depolarizing optogenetic construct can comprise a WPRE having a nucleotide sequence having SEQ ID NO: 86.

The optogenetic construct disclosed herein may comprise a nucleic acid sequence having at least 70% identity to SEQ ID NO: 6 and may comprise a an AAV capsid protein chosen from a AAV8-BP2 capsid protein, a AAV-PHP.B capsid protein, a AAV-PHP.eB capsid protein, or a AAV-NH26 capsid protein. For example, the nucleic acid may comprise a nucleotide having at least about 70%, about 75%, about 80%, about 85%, about 90%, about 91%, about 92%, about 93%, about 94%, about 95%, about 96%, about 97%, about 98%, about 99%, or greater sequence identity to SEQ ID NO: 6.

Particular optogenetic constructs include constructs comprising a nucleic acid that comprises a ProA7 promoter or a variant, fragment, or truncation thereof, or a hybrid promoter comprising a ProA7-derived component and a rod-specific-promoter-derived component, ReaChR as the depolarizing optogenetic protein, EYFP or mCitrine as a reporter molecule, and a WPRE. The optogenetic vector can further comprise an AAV5 capsid, a PHP.eB capsid, an NHP26 capsid (Byrne et al., JCI Insight. 2020; 5(10):e135112. https://doi.org/10.1172/jci.insight.135112), an AAV8 capsid, an AAV8-BP2 capsid, an AAV9 capsid, or a PHP.B capsid. Any of these capsids can be further optimized by sequence variation and screening, if desired.

A preferred optogenetic construct (i.e., an AAV) comprises a nucleic acid that comprises a ProA7 promoter, ReaChR as the depolarizing optogenetic protein, a WPRE, and a hGH PolyA signal. The optogenetic construct may further comprise Citrine as the reporter molecule. The nucleic acid can further comprise an AAV ITR 5′ of the ProA7 promoter and an AAV ITR that 3′ of the PolyA signal. The optogenetic construct can further comprise an AAV capsid protein, if desired. The optogenetic construct comprising the ReaChr depolarizing optogenetic protein can comprise a ProA7 promoter having a nucleotide sequence having SEQ ID NO: 2. The optogenetic construct comprising the ReaChr depolarizing optogenetic construct can comprise a WPRE having a nucleotide sequence having SEQ ID NO: 3. The optogenetic construct comprising the ReaChr depolarizing optogenetic construct can comprise a WPRE having a nucleotide sequence having SEQ ID NO: 86.

The optogenetic construct disclosed herein may comprise a nucleic acid sequence having at least 70% identity to SEQ ID NO: 7. For example, the nucleic acid may comprise a nucleotide having at least about 70%, about 75%, about 80%, about 85%, about 90%, about 91%, about 92%, about 93%, about 94%, about 95%, about 96%, about 97%, about 98%, about 99%, or greater sequence identity to SEQ ID NO: 7. The optogenetic construct comprising a nucleic acid sequence having at least 70% identity to SEQ ID NO: 7 may further comprise an AAV capsid protein chosen from a AAV-PHP.eB capsid protein, AAV8-BP2 capsid protein, or AAV5 capsid protein. The optogenetic construct disclosed herein may comprise a nucleic acid sequence having at least 70% identity to SEQ ID NO: 7 and may comprise an AAV capsid protein chosen from a AAV-PHP.eB capsid protein. The optogenetic construct disclosed herein may comprise a nucleic acid sequence having at least 70% identity to SEQ ID NO: 7 and may comprise an AAV capsid protein chosen from a AAV8-BP2 capsid protein. The optogenetic construct disclosed herein may comprise a nucleic acid sequence having at least 70% identity to SEQ ID NO: 7 and may comprise an AAV capsid protein chosen from a AAV5 capsid protein.

A particular optogenetic construct comprises a nucleic acid as described herein wherein the promoter comprises SEQ ID NO: 2, the depolarizing optogenetic protein is ReaChR, the optional reporter molecule is absent, the PolyA is present and has SEQ ID NO: 9. This nucleic acid may comprise SEQ ID NO: 17. An AAV vector can comprise SEQ ID NO:17 and a suitable capsid, such as AAV5 capsid.

A particular optogenetic construct comprises a nucleic acid as described herein wherein the promoter comprises SEQ ID NO: 14, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 9. This nucleic acid may comprise SEQ ID NO: 18. An AAV vector can comprise SEQ ID NO:18 and a suitable capsid, such as AAV5 capsid.

A particular optogenetic construct comprises a nucleic acid as described herein wherein the promoter comprises SEQ ID NO: 12, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 9. This nucleic acid may comprise SEQ ID NO: 19. An AAV vector can comprise SEQ ID NO:19 and a suitable capsid, such as AAV5 capsid.

A particular optogenetic construct comprises a nucleic acid as described herein wherein the promoter comprises SEQ ID NO: 22, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 9. This nucleic acid may comprise SEQ ID NO: 20. An AAV vector can comprise SEQ ID NO:20 and a suitable capsid, such as AAV5 capsid.

A particular optogenetic construct comprises a nucleic acid as described herein wherein the promoter comprises SEQ ID NO: 23, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 9. This nucleic acid may comprise SEQ ID NO: 21. An AAV vector can comprise SEQ ID NO:21 and a suitable capsid, such as AAV5 capsid.

A particular optogenetic construct comprises a nucleic acid as described herein wherein the promoter comprises SEQ ID NO: 2, the depolarizing optogenetic protein is ReaChR, the optional reporter molecule is absent, the PolyA is present and has SEQ ID NO: 87. This nucleic acid may comprise SEQ ID NO: 17. An AAV vector can comprise SEQ ID NO: 17 and a suitable capsid, such as AAV5 capsid.

A particular optogenetic construct comprises a nucleic acid as described herein wherein the promoter comprises SEQ ID NO: 14, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 87. This nucleic acid may comprise SEQ ID NO: 18. An AAV vector can comprise SEQ ID NO:18 and a suitable capsid, such as AAV5 capsid.

A particular optogenetic construct comprises a nucleic acid as described herein wherein the promoter comprises SEQ ID NO: 12, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 87. This nucleic acid may comprise SEQ ID NO: 19. An AAV vector can comprise SEQ ID NO:19 and a suitable capsid, such as AAV5 capsid.

A particular optogenetic construct comprises a nucleic acid as described herein wherein the promoter comprises SEQ ID NO: 22, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 87. This nucleic acid may comprise SEQ ID NO: 20. An AAV vector can comprise SEQ ID NO:20 and a suitable capsid, such as AAV5 capsid.

A particular optogenetic construct comprises a nucleic acid as described herein wherein the promoter comprises SEQ ID NO: 23, the depolarizing optogenetic protein is ReaChR encoded by SEQ ID NO: 16 or a nucleotide sequence encoding the same polypeptide encoded by SEQ ID NO: 16, the optional reporter molecule is absent, the PolyA is present and has SEQ ID NO: 87. This nucleic acid may comprise SEQ ID NO: 21. An AAV vector can comprise SEQ ID NO:21 and a suitable capsid, such as AAV5 capsid.

A particular optogenetic construct comprises a nucleic acid as described herein wherein the promoter comprises SEQ ID NO: 2, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 10, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 36, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 37, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 38, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 39, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 40, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 41, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 10, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 36, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 37, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 38, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 39, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 40, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 41, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 24 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 10, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 36, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 37, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 38, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 39, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 40, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 41, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 25 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 10, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 36, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 37, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 38, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 39, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 40, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 41, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 10, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 36, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 37, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 38, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 39, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 40, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 41, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO:11 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 12 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 13 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 14 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26 operably linked with SEQ ID NO: 45, a WPRE, the depolarizing optogenetic protein is ReaChR encoded by SEQ ID NO: 16, SEQ ID NO: 33, or SEQ ID NO: 34 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 27, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO:11, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 12, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 13, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 14, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 26, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 9; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid.

A particular optogenetic construct comprises a nucleic acid as described herein wherein the promoter comprises SEQ ID NO: 2, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO: 84.

A particular optogenetic construct comprises a nucleic acid as described herein wherein the promoter comprises SEQ ID NO:11, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO: 84.

A particular optogenetic construct comprises a nucleic acid as described herein wherein the promoter comprises SEQ ID NO: 12, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO: 84.

A particular optogenetic construct comprises a nucleic acid as described herein wherein the promoter comprises SEQ ID NO: 13, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO: 84.

A particular optogenetic construct comprises a nucleic acid as described herein wherein the promoter comprises SEQ ID NO: 14, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO: 84.

A particular optogenetic construct comprises a nucleic acid as described herein wherein the promoter comprises SEQ ID NO: 26, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO:84.

A particular optogenetic construct comprises a nucleic acid as described herein wherein the promoter comprises SEQ ID NO: 85, a WPRE, the depolarizing optogenetic protein is ReaChR encoded by SEQ ID NO: 16, SEQ ID NO: 33, SEQ ID NO: 34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or a nucleotide sequence encoding the same polypeptide encoded by SEQ ID NO: 16 or an optogenetic protein engineered to contain a ReaChR transmembrane domain, the reporter molecule is absent, the PolyA is present and has SEQ ID NO: 87; and an AAV vector comprising an AAV5 capsid, a PHP.eB capsid, an NHP26 capsid, an AAV8 capsid, an AAV8-BP2 capsid, an AAV9 capsid, or a PHP.B capsid, preferably an AAV5 capsid, a PHP.eB capsid, or an NHP26 capsid, more preferably an AAV5 capsid having SEQ ID NO: 84.

A particular optogenetic construct comprises a nucleotide sequence comprising SEQ ID NO: 4. A particular optogenetic construct comprises a nucleotide sequence comprising SEQ ID NO: 5. A particular optogenetic construct comprises a nucleotide sequence comprising SEQ ID NO: 6. A particular optogenetic construct comprises a nucleotide sequence comprising SEQ ID NO: 7. A particular optogenetic construct comprises a nucleotide sequence comprising SEQ ID NO: 17. A particular optogenetic construct comprises a nucleotide sequence comprising SEQ ID NO: 18. A particular optogenetic construct comprises a nucleotide sequence comprising SEQ ID NO: 19. A particular optogenetic construct comprises a nucleotide sequence comprising SEQ ID NO: 20. A particular optogenetic construct comprises a nucleotide sequence comprising SEQ ID NO: 21. A particular optogenetic construct comprises a nucleotide sequence comprising SEQ ID NO: 35. A particular optogenetic construct comprises a nucleotide sequence comprising SEQ ID NO: 55. A particular optogenetic construct comprises a nucleotide sequence comprising SEQ ID NO: 56. A particular optogenetic construct comprises a nucleotide sequence comprising SEQ ID NO: 60. A particular optogenetic construct comprises a nucleotide sequence comprising SEQ ID NO: 67. A particular optogenetic construct comprises a nucleotide sequence comprising SEQ ID NO: 68. A particular optogenetic construct comprises a nucleotide sequence comprising SEQ ID NO: 71. A particular optogenetic construct comprises a nucleotide sequence comprising SEQ ID NO: 72. A particular optogenetic construct comprises a nucleotide sequence comprising SEQ ID NO: 75. A particular optogenetic construct comprises a nucleotide sequence comprising SEQ ID NO: 76. A particular optogenetic construct comprises a nucleotide sequence comprising SEQ ID NO: 77. A particular optogenetic construct comprises a nucleotide sequence comprising SEQ ID NO 80 A particular optogenetic construct comprises a nucleotide sequence comprising SEQ ID NO: 81. A particular optogenetic construct comprises a nucleotide sequence comprising SEQ ID NO: 82. A particular optogenetic construct comprises a nucleotide sequence comprising SEQ ID NO: 83.

Some preferred optogenetic constructs comprise a nucleotide sequence comprising SEQ ID NO: 60, SEQ ID NO: 75, SEQ ID NO:76, SEQ ID NO:77, SEQ ID NO:80, SEQ ID NO:81, SEQ ID NO:82, or SEQ ID NO: 83. A preferred optogenetic construct comprises a nucleotide sequence comprising SEQ ID NO: 60.

### H. Polynucleotides and Vectors

The disclosure further relates to recombinant vectors comprising the nucleic acid disclosed herein or a host cell comprising the vector. The AAV vector can be based on a viral genome with the capsid and other structural proteins removed. The vector provided herein can be suitable for gene therapy, and in particular are suitable for targeting human cone cells. As disclosed herein, the nucleic acid comprises a promoter, a nucleotide sequence encoding a depolarizing optogenetic protein and optionally a reporter molecule, a WPRE and typically a suitable polyA signal. Each of the nucleotide sequences are operable linked.

In addition, the vector may comprise additional elements for the expression of the nucleic acid. For instance, the vector may comprise one or more ITRs, a ribosome binding element, a terminator, an enhancer, a selection marker, an intron, a polyA signal, and/or an origin of replication.

Many different viral and non-viral vectors and methods of their delivery are known to those of skill in the art, such as adenovirus vectors, AAV vectors, retrovirus vectors, lentiviral vectors, herpes virus vectors, liposomes, naked DNA administration and the like. See, e.g., Wright (1997), Br. J. Ophthalmol., 8(1):620-622. Numerous suitable vectors are commercially available. Such vectors typically include polyadenylation signals, etc. in conjunction with multiple cloning sites, as well as additional elements such as origins of replication, selectable marker genes (e.g., LEU2, URA3, TRP 1, HIS3, GFP), centromeric sequences, etc.

The vector suitable for the nucleic acid disclosed herein can be a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, AAV vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, and Rous sarcoma virus vectors.

The vector may be in any form, including, but not limited to, viral particles, such as rAAV particles that include a nucleic acid encoding a depolarizing optogenetic protein as described herein. If desired, the nucleic acids encoding a depolarizing optogenetic protein as described herein can be combined with other suitable nucleic acid delivery agents, for example, complexed with lipids, encapsulated within liposomes, for delivery.

The nucleic acid encoding a depolarizing optogenetic protein disclosed herein may be packaged into a virus capsid to generate a viral particle, preferably an AAV particle.

The virus capsid may be any functional AAV capsid. In one embodiment, the capsid is provided by a single AAV source. Alternatively, the AAV capsid may be derived from more than one source. Any serotype of AAV known in the art, e.g., serotypes AAV1, AAV2, AAV3A, AAV3B, AAV4, AAV5, AAV6, AAV7, AAV8, AAV9, AAV10, AAV11, AAV12, rh10, modified AAV, AAVPhP.B, or yet to be discovered, or a recombinant AAV based thereon, may be used as a source for the AAV capsid.

The viral particle is capable of transducing up to about 10% of primary human cone cells. For instance, the viral particle can be capable of transducing about 10%, about 15%, about 20%, about 25%, about 30%, about 35%, about 40%, about 45%, about 50%, about 55% about 60%, about 65%, about 70%, about 75%, about 80%, about 85%, about 90%, about 91%, about 92%, about 93%, about 94%, about 95%, about 96%, about 97%, about 98%, about 99%, about 100% of the primary human cone cells.

Also provided herein are production methods and cultures for the production of virus particles. The culture can comprise suitable host cells, including, for example, human-derived cell lines such as HeLa, A549, or HEK293 cells, suitable helper virus function, provided by wild-type or mutant adenovirus, e.g. temperature sensitive adenovirus, Herpes virus, or a plasmid construct providing helper functions; AAV rep and cap genes and gene products, the nucleic acid disclosed herein or a vector comprising the nucleic acid, and suitable media and media components to support viral particle production that are well-known in the art.

Suitable host cells can include, but are not limited to, mammalian cells, insect cells, plant cells, microorganisms and yeast. Host cells can also be packaging cells. Exemplary packaging and producer cells are derived from HEK293, A549 or HeLa cells.

The host cell disclosed herein may be transformed or transfected with vector comprising the nucleic acid disclosed herein or viral particle. The host cell may be any animal cell, plant cell, bacterium cell or yeast. The vector disclosed herein may be transferred into host cells using any known technique including viral infection, and may be maintained in the host cell in an ectopic form or may be integrated into the genome.

The nucleic acids described herein, or any component thereof (e.g., promoter, sequence encoding depolarizing optogenetic protein), can be optimized by sequence variation using well-known methods, for example, to achieve desired levels of expression, to reduce immunogenicity, or for other purposes. The optogenetic construct disclosed herein typically further comprise a suitable polyadenylation signal (PolyA) that is 3′ of the WPRE. The optogenetic construct can further comprise one or more AAV inverted terminal repeat sequence (ITRs). Suitable methods for optimizing nucleic acid construct by sequence alternation, including to increase expression, packaging and/or to decrease immunogenicity, for example, are well-known in the art and such modifications of the nucleic acids disclosed herein are considered to be variants of the particular nucleic acids. For example, the nucleic acids described herein, or any component thereof, can be codon optimized, CpG-depleted (See, e.g., U.S. Pat. No. 11,015,210; Y. A. Medvedeva, et al, Bioinformatics—Trends and Methodologies, 449-472 (2011)), modified to remove repeat and hairpin sequences, modified to eliminate alternative reading frames, modified to remove unwanted splice donor and acceptor sites, modified to add stuffer sequence, modified to include a miRNA, siRNA, shRNA, dsRNA, or gRNA sequence (See, e.g., Domenger and Grimm, Human Molecular Genetics, 2019, 28:R1-R12), modified to include an inducible control system (e.g. Tet on/off system) (See, e.g., Gossen et al, Science, 268: 1766-1769 (1995); Harvey et al, Curr. Opin. Chem. Biol., 2:512-518 (1998)), or modified to include an ITR deleted of the terminal resolution site (trs) sequence to generate a scAAV (See, e.g., McCarty et al., Gene Therapy, 2001, 16:1248-54). Methods for optimizing the nucleic acids disclosed herein are conventional and well-known to those of ordinary skill in the art.

### I. Therapeutic Applications

Also provided herein, are methods and uses for the treatment of a disease, disorder, or condition associated with vision loss comprising administering to a subject in need thereof a nucleic acid described herein. The methods disclosed herein can be useful for treating or ameliorating blindness. The methods disclosed herein can be useful for restoring vision. The methods disclosed herein can be useful for restoring sensitivity to light in a human cone photoreceptor cell. The methods disclosed herein can be suitable for treating retinal degeneration.

The methods and compositions disclosed herein can be suitable for treating any disease, disorder, or condition associated vision loss, including, retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, post-retinal detachment, cone dysfunction, a tapetoretinal degeneration, retinal vein occlusion and geographic atrophy. Treatment according to the methods and with the compositions disclosed herein is suitable for a subject that has a disorder impairing vision in which the optic nerve retains at least some function.

While any retinal disease can be suitable for therapy, the inventors have discovery a subset of patients having retinal dystrophies that are particularly suitable for therapy with the optogenetic construct disclosed herein. The inventors have found that patients with inherited retinal dystrophies and low vision are particularly suitable candidates for treatment when having a preserved cone photoreceptor layer in the central retina.

Moreover, the nucleic acid molecules of the invention can be used to manufacture medicaments and/or to treat patients having a disease, disorder, or condition associated with vision loss.

The subject can be a human, dog, cat, horse, or any animal for which a vison restoration is desired.

The nucleic acid disclosed herein can be administered to the subject in an amount sufficient to at least partially restore vision.

The nucleic acid provided herein can be administered to the subject by any suitable route, including but not limited to, intraocular (e.g., subretinal injection, intravitreal injection, suprachoroideal injection), oral, intradermal, intrathecal, intratumoral, intramuscular, intraperitoneal, intravenous, topical, subcutaneous, percutaneous, intranasal and inhalation routes, and via scarification (scratching through the top layers of skin, e.g., using a bifurcated needle). The preferred administration route is by intraocular administration, and more preferred is administration by subretinal injection.

The dosage of the nucleic acid may depend upon the type of composition and upon the subject's age, weight, body surface area, individual condition, the individual pharmacokinetic data, and the mode of administration.

The nucleic acid can be administered to a subject who has or is at risk of developing a condition associated with vision loss. The nucleic acid can be administered before or after the disease becomes symptomatic. For example, before or after partial or complete degeneration of cone cells. The nucleic acid can be administered before or after partial or complete vision loss.

The methods disclosed herein can further comprise administering at least one additional therapeutic agent to the subject. In particular, said therapeutic agent may be a corticosteroid, an antibiotic, an analgesic, an immunosuppressant, or a trophic factor, or any combinations thereof.

### J. Pharmaceutical Compositions

The disclosure also relates to pharmaceutical compositions that comprise the nucleic acid, vectors, and components thereof. The pharmaceutical composition can be administered to a subject for the purposes of restoring light-sensitivity to human cone cells, in particular human cone cells that are not activated with photostimulation so that vision in a subject can be restored. Compositions comprising the nucleic acid are suitable for administration to a subject. The pharmaceutical composition can be supplied as a liquid solution, a suspension, an emulsion, or as solid forms suitable for dissolution or suspension in liquid prior to use.

The pharmaceutical composition may comprise a pharmaceutically acceptable excipient. The term “pharmaceutically acceptable carrier” includes, but is not limited to, any carrier that does not interfere with the effectiveness of the biological activity of the ingredients and that is not toxic to the subject to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Suitable carriers and their formulations are described in Remington: The Science and Practice of Pharmacy, 21st Edition, David B. Troy, ed., Lippincott Williams & Wilkins (2005). Typically, an appropriate amount of a pharmaceutically-acceptable salt is used in the formulation to render the formulation isotonic, although the formulate can be hypertonic or hypotonic if desired. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for direct delivery to the eye with may be administered without undue toxicity. Pharmaceutically acceptable excipients include, but are not limited to, sorbitol, any of the various tween compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Most preferably, the composition is combined with saline, Ringer's balanced salt solution (pH 7.4), and the like.

The pharmaceutical composition may optionally comprise one or more agents that facilitate delivery of the nucleic acid or vectors to a target cell, including but not limited to, transfection reagents or components thereof, such as lipids or polymers.

The pharmaceutical composition disclosed herein can be formulated for administration to the eye, in particular by intraocular injection, e.g., by subretinal and/or intravitreal or suprachoroideal administration. For intravitreal delivery, the pharmaceutical composition disclosed herein can be injected directly into the vitreous. For subretinal delivery, the pharmaceutical composition disclosed herein can be delivered in a localized subretinal bleb between the retinal pigment epithelium (RPE) and the photoreceptor layer in a surgical procedure. This can be accomplished during pars plana vitrectomy (ppV). Subretinal administration can provide the direct access to photoreceptors and the RPE. Suprachoroideal injection can provide access to the photoreceptors from the choroideal layer. Alternatively, the pharmaceutical composition can be delivered into the anterior section of the eye, in particular into the anterior chamber. Subretinal injection is the preferred administration mode.

The amount of pharmaceutical composition to be administered may be determined by standard procedure well known by those of ordinary skill in the art. Physiological data of the patient (e.g. age, size, and weight) and type and severity of the disease being treated have to be taken into account to determine the appropriate dosage.

The pharmaceutical composition may be formulated for administration by injection, e.g., by subretinal or intravitreal injection or suprachoroideal injection. Formulations for injection may be presented in unit dosage form, e.g., in ampoules or in multi-dose containers. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The pharmaceutical composition disclosed herein may also be formulated as a depot preparation or for use in an implanted delivery system. Such long-acting formulations may be administered by implantation, for example, intraocular, or by intraocular injection. The pharmaceutical composition may also be formulated as a depot preparation for use in an implanted drug delivery system or device, particularly for repeated refill of a reservoir in the implanted drug delivery system or device. Thus the pharmaceutical composition may be formulated with suitable polymeric or hydrophobic materials (for example, as an emulsion in an acceptable oil) or ion exchange resins, or as sparingly soluble derivatives, for example, as a sparingly soluble salt.

In an embodiment, the pharmaceutical composition disclosed herein can comprise a vector or viral particle comprising the nucleic acid disclosed herein. Preferably, the vector or viral particle is an AAV vector or particle. The pharmaceutical composition may comprise host cells comprising the nucleic acid disclosed herein or the viral particle comprising the nucleic acid.

Optionally, the pharmaceutical composition comprising host cells may be frozen for storage at any temperature appropriate for storage of the cells. The pharmaceutical composition can comprise viral particles and each unit dosage comprises from 10E+8 to 10E+13 viral particles measured by polymerase chain reaction using a probe specific for the virus genome.

The pharmaceutical composition may further comprise one or several additional active compounds such as corticosteroids, antibiotics, analgesics, immunosuppressants, trophic factors, or any combinations thereof.

### K. Kits

Also disclosed herein are kits comprising the nucleic acid disclosed herein, viral particles comprising the nucleic acid, host cells, or a pharmaceutical composition thereof.

The kit may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), to reconstitute the complex to form a solution for injection purposes.

A kit can further comprise a needle or syringe, preferably packaged in sterile form, for injecting the complex, and/or a packaged alcohol pad. Instructions are optionally included for administration of compositions by a clinician or by the patient.

### L. Definitions

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

As used herein, the term “promoter” refers to any cis-regulatory elements that are generally located upstream (towards the 5′ region) that directs the transcription of a nucleic acid to which it is operable linked.

As used herein, the term “operably linked” in the context of a nucleic acid sequence, refers to the orientation of nucleotide sequences on a single nucleic acid molecule that permits the components (i.e., the nucleic acid sequences) to function in their intended manner. For example, a promoter is operably linked with a nucleic acid sequence encoding an depolarizing optogenetic protein when it is capable of affecting the expression of that optogenetic sequence, i.e., the optogenetic sequence is under the transcriptional control of the promoter.

As used herein, the term “nucleic acid” or “polynucleotide” refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. “Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer.

As used herein, the term “host cell” refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. The progeny of a single cell may not necessarily be completely identical in morphology or in genomic or total DNA complement as the original parent due to natural, accidental, or deliberate mutation.

As used herein, the term “therapeutically effective amount” refers to an amount of a compound described herein (i.e., a nucleic acid) that is sufficient to achieve a desired pharmacological or physiological effect under the conditions of administration. For example, a “therapeutically effective amount” can be an amount that is sufficient to reduce the signs or symptoms of a disease or condition (e.g., visual impairment or blindness). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

As used herein, the terms “treat,” “treatment,” or “treating” and grammatically related terms, refer to an improvement of any sign, symptoms, or consequence of the disease, such as prolonged survival, less morbidity, and/or a lessening of side effects. As is readily appreciated in the art, full eradication of disease is preferred but not a requirement for treatment.

The term “subject” as used herein refers to any animal, such as any mammal, including but not limited to, humans, non-human primates, rodents, and the like. In some embodiments, the mammal is a mouse. In some embodiments, the mammal is a human.

Additional description of the methods and guidance for the practice of the methods are provided herein.

### 4. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### 5. EXAMPLES

### Example 1. A Cone-Specific Promoter (ProA7) Requires a 3′ Regulatory Element for Expression in Human Mature Cones

ChrimsonR is a light activated channel that induces light responses in cells that are not photosensitive, Klapoetke N C et al. (2014), 11(3):338-346. Preliminary studies were conducted on the ChrimsonR-tdTomato (ChrimsonR-tdT) transgene as an optogenetic effector, as it was recently found safe and effective in a clinical trial after intravitreal injection (See Reference 1, Sahel et al.). In this vector the ChrimsonR-tdT sensor is under the control of the ubiquitous CAG promoter allowing for non-specific expression, although mostly ganglion cells (GCs) are targeted after intravitreal injection (See References 1, 2). To achieve cone-specific expression, the cone-specific ProA7 promoter (See Reference 3) (synPVI or Gnat2_500, SEQ ID NO: 2) replaced the CAG promoter in the ChrimsonR-tdT vector.

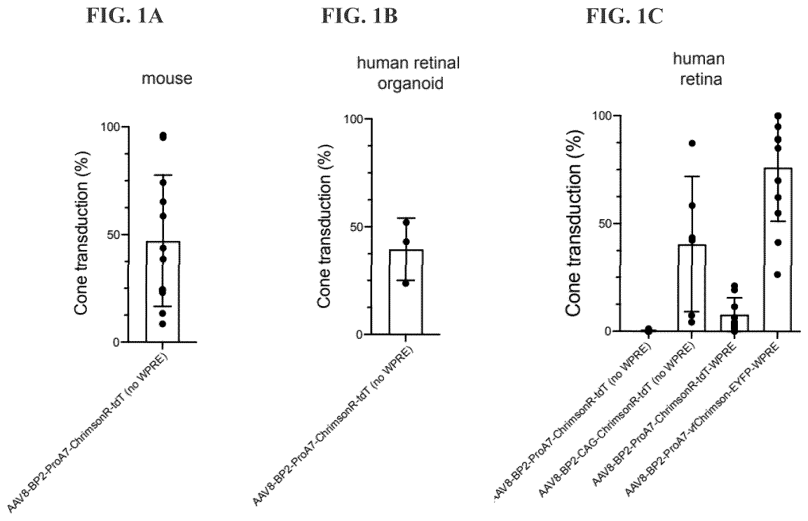

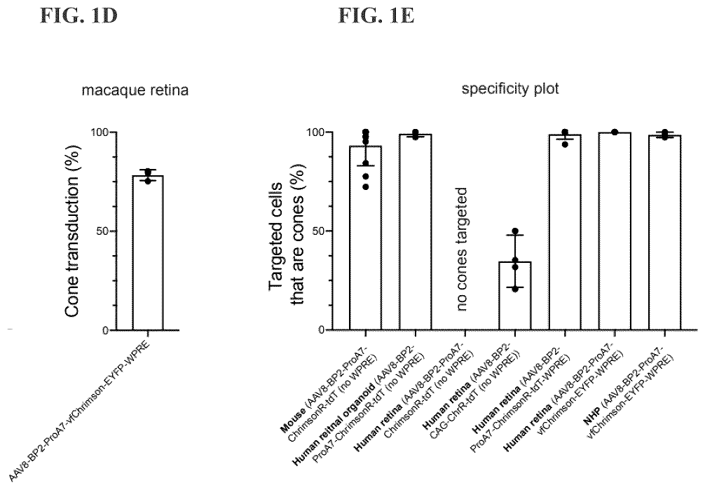

AAV8-BP2-ProA7-ChrimsonR-tdT-hGHpolyA was subretinally injected into wild-type C57BL/6 mice. Efficient expression in mouse cones was noted (FIG. 1A). Next, AAV8-BP2-ProA7-ChrimsonR-tdT-hGHpolyA was tested on three-dimensional 30-week old human retinal organoids. Similarly observed efficient expression was observed (FIG. 1).

The constructs were then placed onto human retinal explants for derived from multi-organ donors for two days followed by a 5 week culture. Surprisingly, no cones were observed to express the ChrimsonR-tdT transgene, which was in striking contrast with the data obtained in mouse and in retinal organoids (FIG. 1C).

One or more of the following factors were hypothesized to be responsible for this finding: (1) ChrimsonR-tdT might not have been expressed in human cones and/or (2) nuclear export of intronless transcripts might have been reduced because the construct lacked 3′ regulatory element.

The AAV8-BP2-CAG-ChrimsonR-tdT-hGHpolyA (no WPRE) vector (SEQ ID NO: 1, See, e.g., WO2017187272A1 and WO 2012/145601 regarding AAV2-7m8 viral vector containing CAG-ChrimsonR-tdTomato) was tested on human retinas, which led to non-specific expression of the transgene in cones and rods (FIG. 1C, 1E). This suggested that ChrimsonR-tdT could be expressed in cones, however a non-specific expression is not desirable for therapy as it would lead to aberrant retinal computation.

Next, the modified woodchuck hepatitis virus post-transcriptional regulatory element (WPRE) was cloned downstream of the ChrimsonR-tdT and new vectors were created (SEQ ID NO: 3 and 4). Weak but specific fluorescence in some cones was observed with AAV8-BP2-ProA7-ChrimsonR-tdT-hGHpolyA (FIG. 1C, FIG. 1D).

Finally, the ChrimsonR-tdT was changed to vfChrimson-EYFP (SEQ ID NO: 5), which led to more efficient and specific expression in human peripheral retinal cones (FIG. 1C and FIG. 1D) as well as in non-human primate (NHP) cones after in vivo subretinal injection (FIG. 1E).

These results altogether suggest that the WPRE element is required for ProA7-mediated expression (but not CAG-mediated expression) and the optogenetic sensor is important to achieve high level expression. The ProA7 linked to the WPRE element leads to efficient and specific expression of optogenetic constructs in human cones.

### Example 2. Systematic Screening of AAV Capsids and Optogenetic Sensors in Human Cones

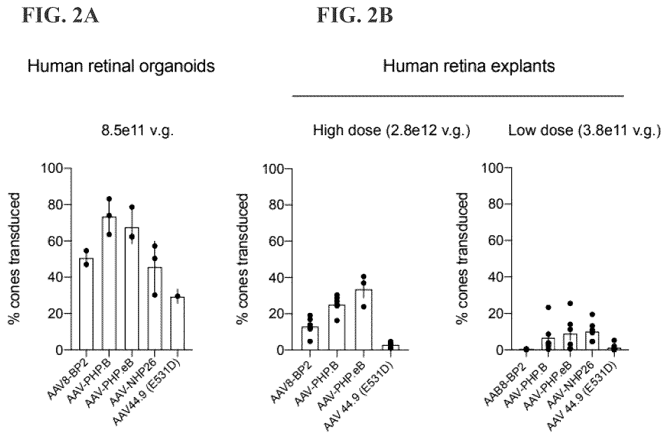

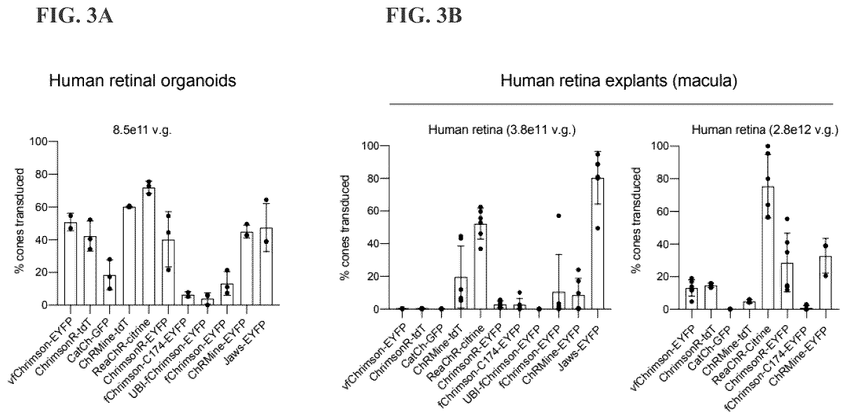

Having identified a combination of vector elements (ProA7+WPRE) that together led to efficient cone-specific expression, the effect of the AAV capsid and the optogenetic sensor on expression was systematically investigate using retinal organoids and human explant retina as model systems (FIG. 2 and FIG. 3). To allow for side-by-side comparison between different vectors, samples were transduced at equal genomic copies of AAV using ddPCR for WPRE as a titering method.

Organoids and macular human retina explants were incubated 5 weeks, and stained with an antibody against the fluorophore and a cone marker (cone-arrestin). The percent cone transduction was determined by manual counting (organoids, 3 investigators) or by an automated script (human retina, positivity cutoff was above 4 S.D. from the mean of the background). In the first screen, the ProA7-vfChrimson-EYFP-WPRE-hGH-polyA vector was packaged in AAV8-BP2, AAV-PHP.B, AAV-PHP.eB, AAV-NHP26 and AAV44.9(E531D) capsid serotypes. All vectors transduced cones in retinal organoids (FIG. 2A).

On human retina, the highest transduction rates were observed with AAV-NHP26, AAV-PHP.B and AAV-PHP.eB. Interestingly, AAV8-BP2 was only effective at a higher dose and led only to a 13.2%±5.0% (mean±s.d.) cone transduction in central (macular) retinal explants (FIG. 2B). AAV44.9(E531D) did not lead to cone transduction.

In the second screen, focusing on comparing optogenetic sensors, several different sensors were packaged in the AAV8-BP2 capsid (FIG. 3): ChrimsonR-tdT, CatCH-GFP, ChrMine-tdT, ReaChR-citrine, ChrimsonR-EYFP, fChrimson-C174-EYFP (contains the CatCH-GFP mutation), ubiquitinated-fChrimson-EYFP, fChrimson-EYFP, ChrMine-EYFP and Jaws-EYFP. All sensors were driven by the ProA7 promoter and were followed by the WPRE element and the hGH-polyA sequence.

Several sensors expressed on organoids (FIG. 3A) and human retinas (FIG. 3B), although expression in general was lower for human retinas. The highest expression was noted with ReaChR-EYFP and Jaws-EYFP but expression was noted with ChrimsonR and ChrMine as well, regardless whether EYFP or tdT tag was used.

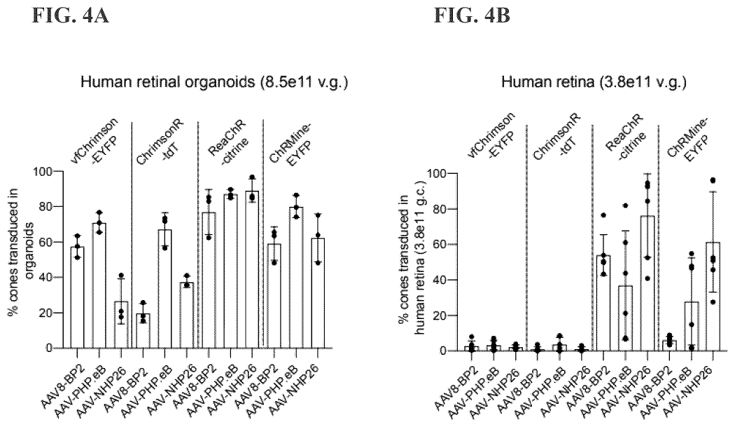

Finally, three capsids (AAV-BP2, AAV-PHP.eB. AAV-NHP26) and four sensors (vfChrimson-EYFP, ChrimsonR-tdT, ReaChR-citrine and ChrMine-EYFP) were combined and a combination screen in retinal organoids and human retinas was conducted (FIG. 4).

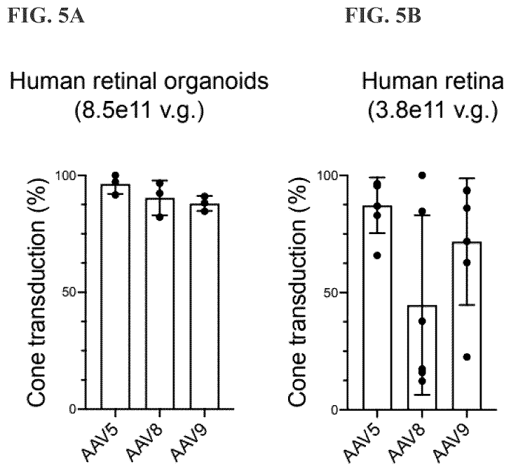

Similarly to previous experiments, the difference between different constructs was higher in human retina. At the lower AAV dose used (3.8e11 v.g.), there was only minimal expression using vfChrimson-EYFP and ChrimsonR-tdT, Robust expression of ReaChR-citrine and ChrMine-EYFP was observed (FIG. 4). Using ReaChR, AAV5, AAV8 and AAV9 were tested, and expression was found for all capsids, with AAV5 being the most effective (FIG. 5). These experiments altogether suggest that the major determinant of effective expression is the vector design and the applied sensor. Different capsids can lead to efficient expression in human cones.

### Example 3. ReaChR-Citrine Restores Light Sensitivity to Human Retinal Explants

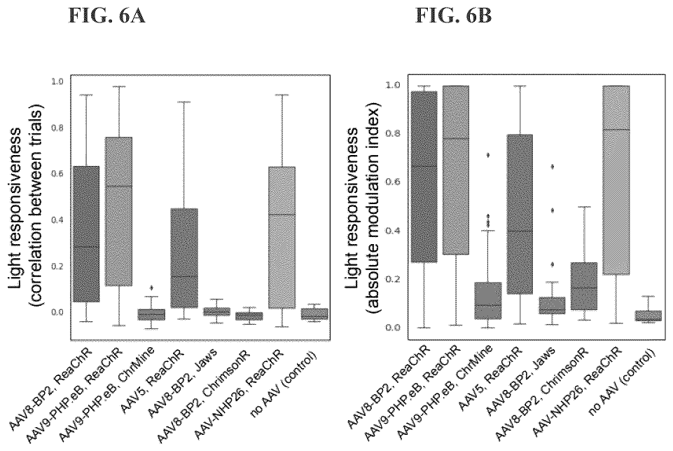

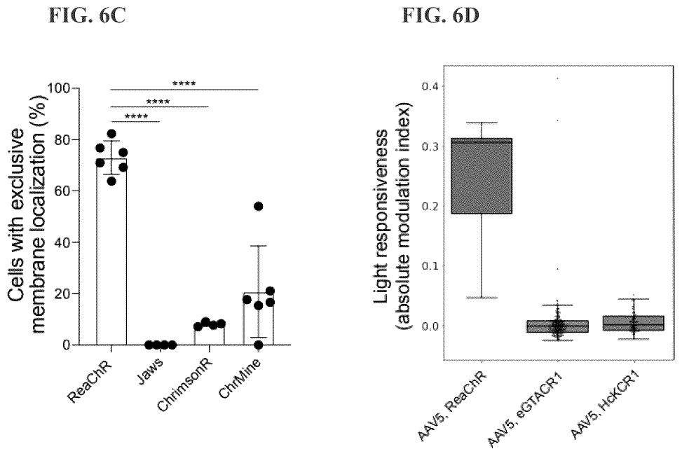

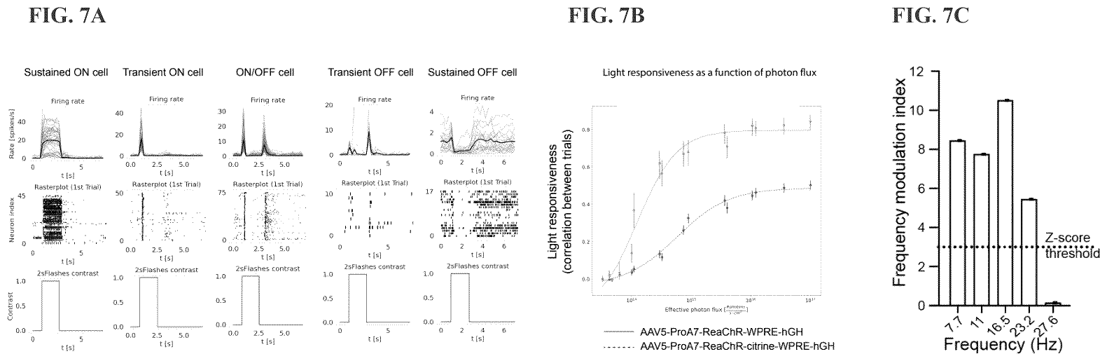

Having identified AAV capsid-optogenetic sensor combinations leading to efficient cone transduction and expression, whether these combinations are also able to drive functional light responses in cultured human retinas was examined (FIG. 6 and FIG. 7). Cultured retinas lose intrinsic photoreceptor light sensitivity within a few hours after explantation. This is due to the loss of the fragile outer segment and the lack of support from the RPE, which is detached for cell culture.

By taking advantage of this feature, whether light sensitivity can be restored in these cultured retinas after application of AAVs was investigated. Indeed, if any light response is observed after AAV transduction in cultured explants, it can only originate from the optogenetic protein tested.

Light responses in AAV-transduced human retinal explants from 4 different donors five to nine weeks after culture were investigated using microelectrode array recordings. Briefly, explants were placed on a 256-microelectrode arrays, ganglion cell layer facing the electrodes, and the activity of several ganglion cells was simultaneously recorded upon light stimulation. Spike sorting was performed after to isolate single cell activity. To screen for light responses, 2 second white light flashes were presented and repeated 10 times, with a 5 s inter-stimulus interval, at intensity of 1.38E+16 photon flux/cm²/s, which was shown previously to reliably activate the optogenetic effectors in NHP explants (Reference 2) and is in the typical range of sensitivity for these microbial opsin (Reference 4).

Non-transduced control retinas never showed light responses (FIGS. 6A-6B). Light responses in retinas treated with AAV vectors carrying the ProA7-ReaChR-citrine-WPRE-hGH transgene were observed. In accordance with findings on expression reported herein, the capsid component was not critical, as light responses were observed for all capsids tested (AAV8-BP2, AAV5, AAV9-PHP.eB or AAV-NHP26, FIGS. 6A-6C). However all the other tested light sensors (ChrMine-EYFP, ChrimsonR-EYFP and Jaws-EYFP) did not lead to light responses (FIGS. 6A-6C). Further testing of additional hyperpolarising optogenetic effectors (HcKCR1 and eGTACR1) also did not lead to light responsiveness (FIG. 6D).

Although all the tested sensors expressed on human retina (FIG. 3 and FIG. 4), ReaChR showed very robust membrane localization (FIG. 6C). This suggests that the superior membrane trafficking of ReaChR is critical to obtain light responses. The importance of membrane localization is not surprising as optogenetic sensors are generally understood to work by moving ions through the cell membrane. ChrMine-EYFP, ChrimsonR-EYFP and Jaws-EYFP are not efficient in membrane localization (FIG. 6C) and this might explain the lack of light responses in human retinas.

### Example 4. Characterization of Light Responses with ReaChR-Citrine

Next, light responses in human retinal explants of a ReaChR-citrine construct (SEQ ID NO: 7) were characterized in more detail (FIGS. 7A-7C). The normal human retina shows five different ganglion cell responses to a light flash (See Reference 5). The first class of cells responds with spiking during the 2 second light flash (sustained ON response). The second class of cells respond with firing only at the beginning of the light flash (transient ON response). The third class responds at the onset and at the offset of the stimulus (ON/OFF response). The fourth class responds at the offset of the stimulus (transient OFF response), while the fifth class decreases its firing over the whole duration of the response (sustained OFF response).

Importantly, ReaChR-citrine transduced human retinas developed the same five different types of responses, suggesting that the construct restored normal human retinal computation (FIG. 7A).

Also, the sensitivity of the response using ReaChR-citrine was characterized. Light responses were observed between ˜1×10¹⁴-1×10¹⁶ photons/cm²/sec, which is in agreement with previous reports obtained in mice using another sensor (See Reference 4).

Finally, the citrine tag was removed and retina cultures were transduced with AAV5-ProA7-ReaChR-WPRE-hGH (SEQ ID NO: 17). Light responses and a higher sensitivity were observed. The sensitivity of the AAV5-ProA7-ReaChR-WPRE-hGH was between ˜5×10¹³-1×10¹⁶ photons/cm²/sec, which is half a log more sensitive than the AAV5-ProA7-ReaChR-citrine WPRE-hGH (SEQ ID NO: 7). (FIG. 7B) FIG. 7C further demonstrates that retinas transduced with the optogenetic vector driven by the cone-specific ProA7 promoter can follow a frequency stimulation up to 23.2 Hz.

These experiments suggest that optogenetic stimulation through cone photoreceptors can restore light sensitivity and physiologic retinal computation in a fixed light intensity range. Furthermore, the removal of the citrine tag does not inhibit light responses but results in a higher sensitivity.

### Example 5. Non-human Primate Studies

A. ReaChR is Expressed in the Fovea after In Vivo Injection into NHPs

Next, AAV5-ProA7-ReaChR-citrine-WPRE-hGH (SEQ ID NO: 7) was subjected to testing in NHPs after in vivo subretinal injection. Three Cynomolgus macaques were injected bilaterally with three different doses of the AAV5-ProA7-ReaChR-citrine-WPRE-hGH (SEQ ID NO: 7) vector (1.5×10¹⁰, 1.5×10¹¹, 3×10¹¹ vector genomes, v.g. per eye). Animals were sacrificed 3 months after injection and the retinas were explanted. Retina pieces were cultured for 1-2 days to abolish endogenous light responses.

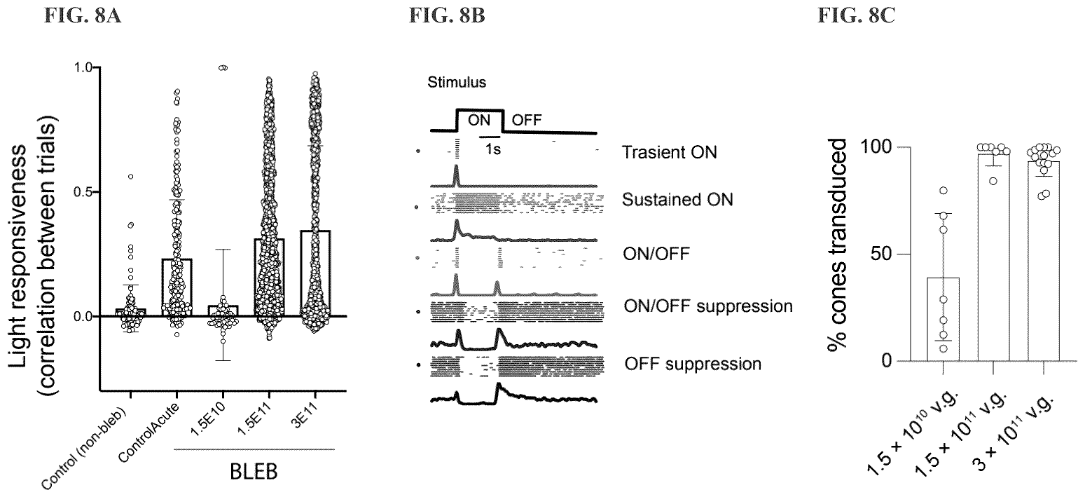

From non-transduced areas (‘non-bleb’), light responsive cells were not observed after this short culture period. However, in the AAV transduced areas (‘bleb’), robust light responses were observed 1.5×10¹¹, 3×10¹¹ v.g. dose, confirming the functionality of the optogenetic effector in vivo (FIG. 8A). In the lowest dose 1.5×10¹⁰ v.g., we did not observe light responses (FIG. 8A). Light responsive cells showed sustained ON, transient ON behavior, ON/OFF, or sustained OFF behavior suggesting restoration of retinal computation (FIG. 8B). After MEA recordings, the tissue pieces were fixed and transduction efficiency was calculated by counter staining cones with cone-arrestin (FIG. 8B). At 1.5×10¹⁰, 1.5×10¹¹, 3×10¹¹ v.g. doses, mean cone transductions of 39.54%, 58.97% and 93.94%, respectively, were observed.

These experiments altogether confirm that the AAV5-ProA7-ReaChR-citrine-WPRE-hGH vector (SEQ ID NO: 7) transduces cone photoreceptors efficiently in the macaque retina after subretinal injection and is functional in vivo.

B. An AAV Vector that Carries an Optogenetic Effector, ReaChR, Under the Control of a Hybrid Promoter is Functional in Non-Human Primates

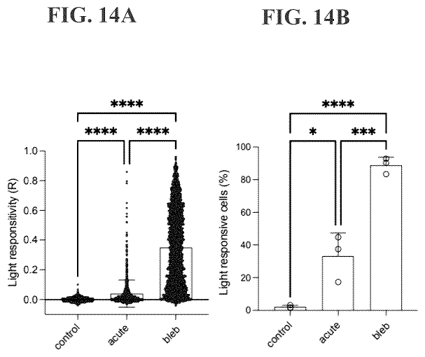

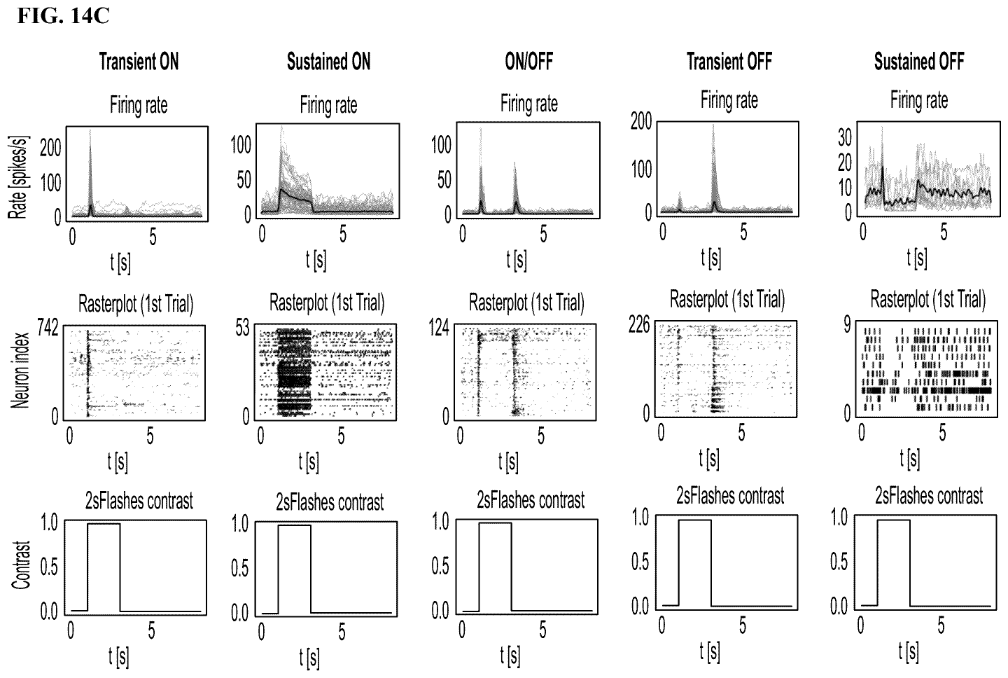

To investigate and compare light responsiveness in healthy primate retina following transduction with the AAV5-Pro573.2-ReaChR-WPRE-hgH poly A vector (SEQ ID NO: 18), cynomolgus macaques were injected via subretinal injection with 1.5E11 vg of the AAV vector, and light responsiveness in healthy non-human primate (NIP) retina was characterized (FIG. 14A-14C).

AAV5-Pro573.2-ReaChR-WPRE-hgH polyA (SEQ ID NO: 18) was injected into 3 Cynomolgus macaques at 1.5e11 v.g. dose (n=6). The control vector, AAV5-ProA7-ReaChR-citrine-WRPE-hGH (SEQ ID NO: 7), was injected into two eyes nasally in two separate macaques. Animals were followed up using optical coherence tomography (OCT) to assess retinal morphology post-injection. After 3-4 months, animals were euthanized and the eye was examined for gross pathology. Next, gene expression and light responsiveness studies were performed.

The retina tissues from the injected area (‘bleb’) and non-injected area (‘non-bleb’) were placed in culture for 1-2 days to diminish endogenous light responses and measure ontogenetically driven light responses using multi-electrode array (MEA) recordings. ‘Acute’ refers to freshly dissected retina, where light responses can be measured in a healthy, non-treated region of the retina. Both the ‘control’ and ‘bleb’ samples were cultured to eliminate normal light responses. The ‘control’ is from a non-treated area of the retina. The AAV transduced area (‘bleb’) showed robust light responsiveness (FIGS. 14A and 14B) that was enhanced compared to the freshly dissected retina (‘acute’) where normal light responses can be measured. No light responsive cells were observed in the control sample.

AAV-Pro573.2-ReaChr-WPRE-hgH poly A transduced NHP retinas showed the full diversity of light responses for the five different type of responses indicating light responsiveness and retinal computation similar to normal (FIG. 14C).

These experiments demonstrate that optogenetic stimulation through NHP cone photoreceptors can result in light sensitivity and physiologic retinal computation in a fixed light intensity range. Moreover, removal of the citrine tag does not inhibit light responses as light responses have been observed with the AAV-Pro573.2-ReaChr-WPRE-hgH poly A construct, which lacks citrine. Importantly, these results are observed in a preclinical animal model that is closest to humans and inferred to be the best model for translational studies of human therapeutics. Furthermore, the effectiveness of vector administration—using the same method that would be used for administration of the AAV vector to human patients in clinical trials—was employed in these NHP experiments and shown to be successful.

### Example 6. Efficiency of AAV5-ProA7-ReaChR-Citrine-WPRE-hGH in 4 Different Human Donors

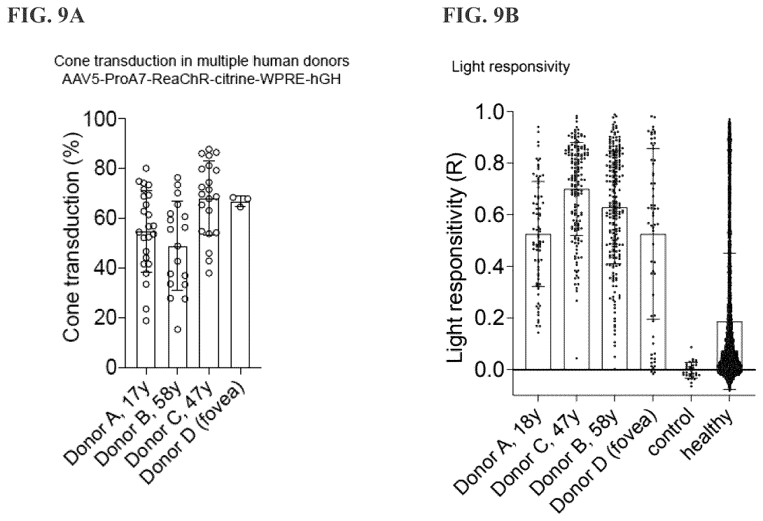

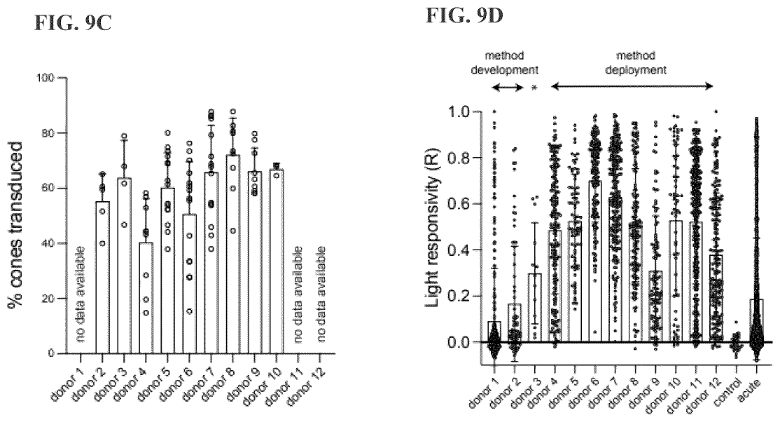

Next, whether the AAV5-ProA7-ReaChR-citrine-WPRE-hGH vector (SEQ ID NO: 7) transduces cone photoreceptors was analyzed in 4 additional independent human donors. Human retinas were cultured for 4-6 weeks before analysis. Robust light responses were observed in all cases (FIG. 9B). Efficient cone transduction was detected in all 4 donors (FIG. 9A, average efficiency was found to be between 49.06-68.03%). In donor 4, the vectors were tested on the fovea. 66.92% of cones were transduced with the AAV5-ProA7-ReaChR-citrine-WPRE-hGH (SEQ ID NO: 7) vector.

Human explants from 12 human donors ages 18 to 80 years old (8 males and 4 females), were also tested and the results are compared in FIG. 9C and FIG. 9D. FIG. 9C shows efficient cone transduction (average efficiency between 40-80%) and FIG. 9D demonstrates robust light responses in AAV5-ProA7-ReaChr-citrine-WPRE-hGH polyA vector transduced donor retina samples. Donor A from FIGS. 9A and 9B corresponds with donor 5 in FIGS. 9C and 9D. Donor B from FIGS. 9A and 9B corresponds with donor 7 in FIGS. 9C and 9D. Donor C from FIGS. 9A and 9B corresponds with donor 6 in the FIGS. 9C and 9D.

These results suggest that the desirable features of high transduction efficiency and effective light responsiveness of the AAV5-ProA7-ReaChR-citrine-WPRE-hGH polyA optogenetic vector are reproducible in transduced human retina explants from individuals with different ages and gender.

### Example 7. Hybrid Promoters Containing a 185 Base Pair Sequence of ProA7 is Also Functional

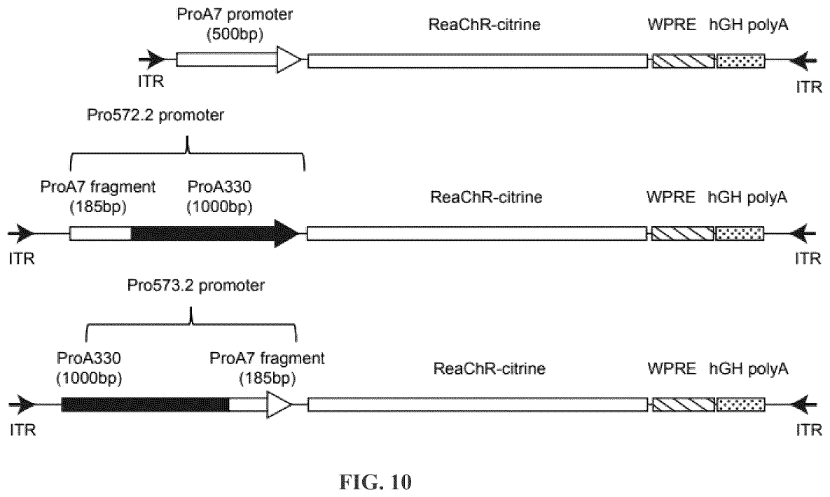

Experiments described herein confirmed the functionality of the AAV5-ProA7-ReaChR-citrine-WPRE-hGH vector (SEQ ID NO: 7, FIG. 10) in retinal organoids, human retinal explants and in vivo in the macaque retina. The functionality of hybrid promoters containing a 185 base pair region of the ProA7 promoter and a rod-specific promoter having SEQ ID NO: 10 was further analyzed (FIG. 10). These promoters were named ProX572.2 (SEQ ID NO: 12) and ProX573.2 (SEQ ID NO:14), depending on the order of the 185 base pair ProA7 fragment and the rod-specific promoter (FIG. 10). ProX572.2 is also known as Pro572.2; ProX573.2 is also known as Pro573.2.

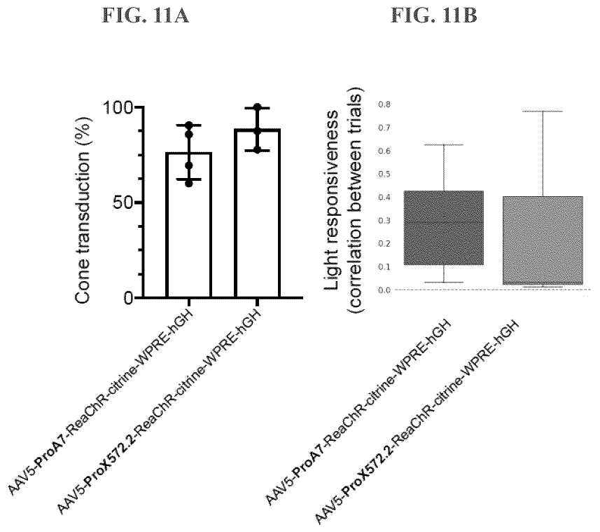

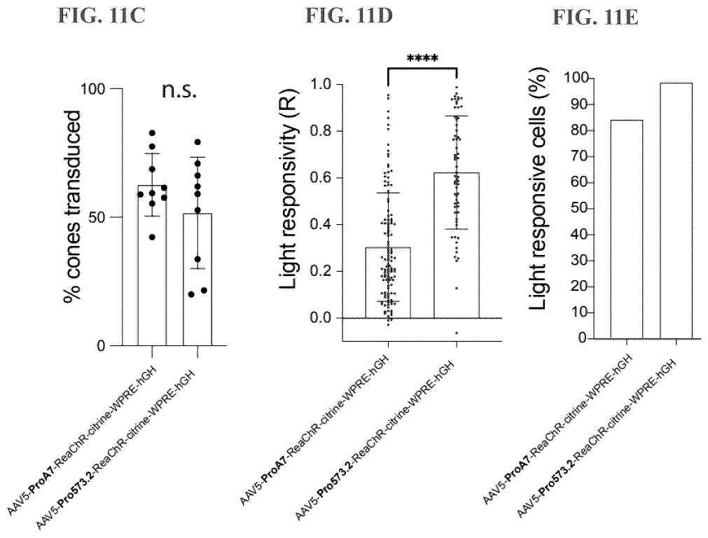

Expression and function were tested using AAV5-ProX572.2-ReaChR-citrine-WPRE hGH polyA on human retinal explants. Expression levels were similar between the AAV5-ProA7-ReaChR-citrine-WPRE-hGH polyA (SEQ ID NO: 7) and AAV5-ProX572.2-ReaChR-citrine-WPRE hGH polyA vectors (SEQ ID NO: 55) (FIG. 11A) Similar light responsiveness was observed in the optogenetic vector driven by the ProX572.2 hybrid promoter compared to the ProA7 vector (FIG. 11B). This data suggests that a 185 base pair fragment of the ProA7 promoter 5′ to the ProA330 promoter is sufficient to drive functional expression of optogenetic sensors in cone photoreceptors.

The functionality of the AAV5-Pro573.2-ReaChr-citrine-WPRE-hGH vector (SEQ ID NO: 56) was also assessed in human retinal explants and compared to the AAV5-ProA7-ReaChR-citrine-WPRE-hGH vector control (FIG. 11C-FIG. 11E). The Pro573.2 promoter differed from the Pro572.2 promoter in the orientation of the ProA7 fragment: The 185 bp fragment of the 3′ end of ProA7 was located 3′ to the ProA330 promoter. Both the ProA7 and Pro573.2 optogenetic vectors expressed in cone photoreceptors at comparable levels (FIG. 11E). Strikingly, the Pro573.2 optogenetic vector showed much stronger light responses than the vector driven by the cone-specific promoter, ProA7 (FIG. 11C and FIG. 11D). These results demonstrate that the hybrid promoter, Pro573.2, containing a fragment of the cone-specific promoter (ProA7) sequence 3′ of the Pro330 promoter and the 1000 bp of the Pro330 rod-specific promoter sequence, possesses superior functionality compared to the cone-specific promoter (ProA7) alone.

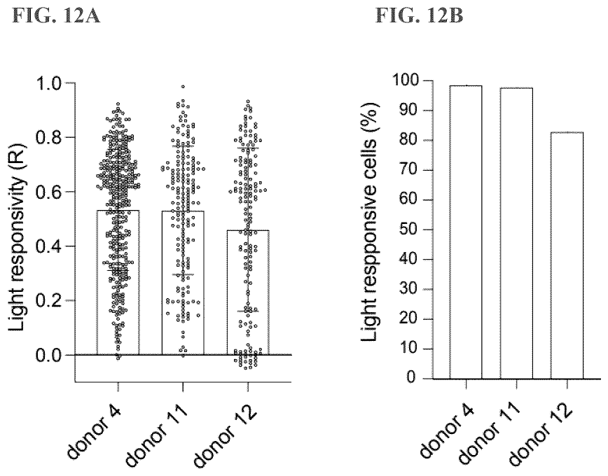

Expression and function were also tested using the optogenetic vector, AAV5-Pro573.2-ReaChR-WPRE-hGH, with the citrine tag removed, on human retinal explants. Robust light responses was observed following transduction of human retinal explants from 3 donors (FIG. 12A and FIG. 12B).

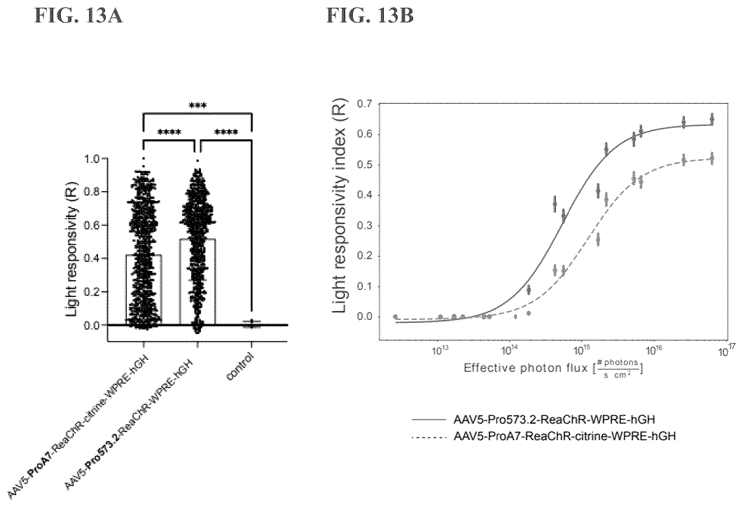

Comparison experiments in human retinal explants between the AAV5-ProA7-ReaChr-citrine-WPRE-hGH vector (SEQ ID NO: 7) and AAV5-Pro573.2-ReaChR-WPRE-hGH vector (SEQ ID NO: 18) resulted in the Pro573.2 promoter driving a stronger light response in a higher percentage of cells (FIG. 13A) and a more sensitive light response compared to the ProA7 promoter containing vector (FIG. 13B). This data suggests that a 185 base pair fragment of the ProA7 promoter is sufficient to drive functional expression of optogenetic sensors in cone photoreceptors in any orientation within the construct, and that removal of the citrine tag does not affect expression or functionality of the optogenetic protein.

### Example 8. Restoration of Light Responsiveness in a Mouse Model of Ocular Disease that Receives an AAV Vector Carrying an Optogenetic Effector, ReaChR, Under the Control of a Hybrid Promoter

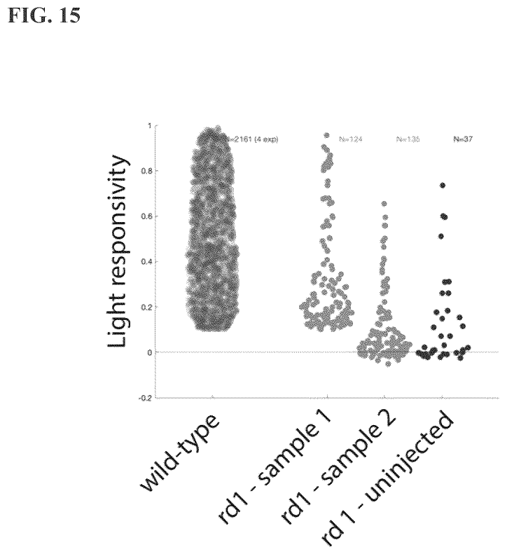

To determine if ReaChR can restore light responses in the blind retina, we injected rd1 mice, a well-established model for retinal degeneration. Animals are blind already at postnatal day 28, but retain non-functional dormant cones (Reference 4). Animals were injected with AAV5-ProA7-ReaChR-Citrine-WPRE-hGH polyA construct at post-natal day 77 and sacrificed at postnatal day 105. Retinal light responses were measured using the multi-electrode array measurement. We have detected light responsive ganglion cells in the treated retinas (FIG. 15). This indicates that light responsiveness can be elicited with a depolarizing optogenetic channel even in the blind state.

### Example 9. Multiple Copies of Promoters Enhance Gene Expression in Cone and Rod Photoreceptors

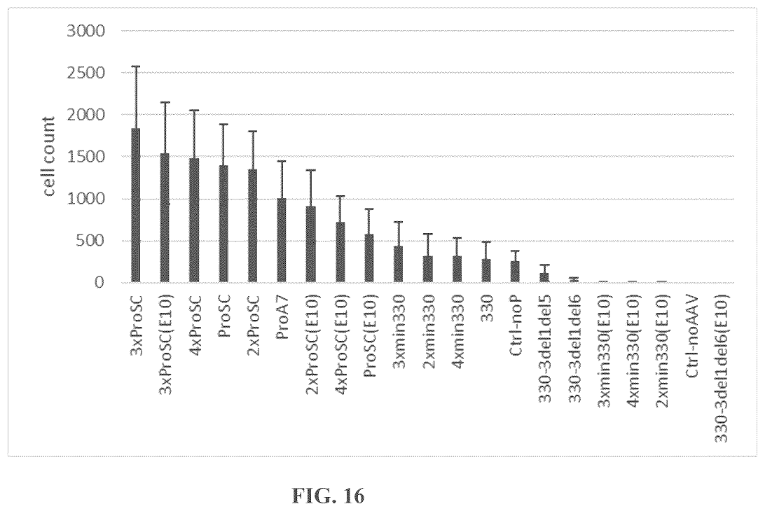

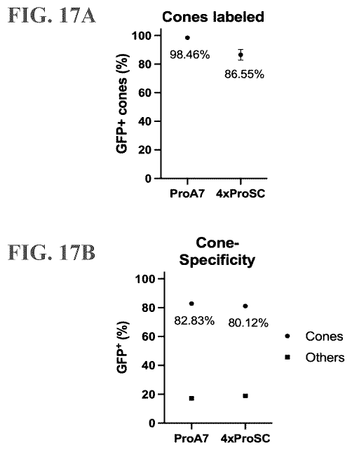

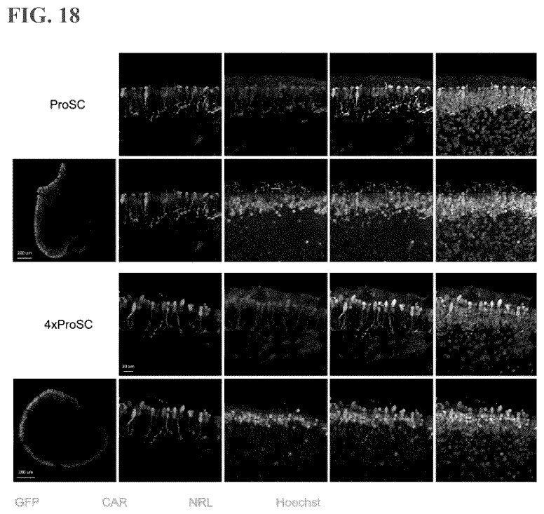

To determine the functionality of including multiple copies of rod and cone-specific promoters on gene expression, AAV vectors containing promoter replicates (2×, 3×, 4×) of the ProSC and min330 promoters driving eGFP expression were tested in organoid cultures and compared to vectors containing a single copy of the ProSC or min330 promoter, as well as to vectors containing the full-length ProA7 and ProA330 promoters. Analysis of live GFP intensity was quantified in the retinal organoids and enhanced GFP expression was observed following transduction with the AAV vectors containing the multimerized promoter constructs (FIG. 16). Variant 4xProSC remains specific for cone photoreceptors in human retina (FIGS. 17A and 17B) and in human retinal organoids (FIG. 18). These results suggest that multiple copies of the cone and rod specific promoters, as single elements or in the context of a hybrid promoter, can enhance gene expression following AAV transduction of human retina or human retinal organoids.

### 9.1 Methods

### 9.1.1 Animal and Human Retinas

### Mice

Animals were used in accordance with standard ethical guidelines as stated in the European Communities Guidelines on the Care and Use of Laboratory Animals. C57BL/6J wild-type mice (6-8 weeks old) were obtained from the Charles River Laboratories. Mice were housed at the animal facility of the Institute of Molecular and Clinical Ophthalmology Basel (IOB) and were maintained in a pathogen-free environment with ad libitum access to food and drinking water. All animal experiments and procedures were approved by the Veterinary Department of the Canton of Basel-Stadt (permit number: 31896/3048).

### NHPs

Healthy cynomolgus monkeys (Macaca fascicularis, aged 5-19 years, weight 5.6-10.8 kg) were housed at the Simian Laboratory Europe Facility. Animals were maintained and monitored in accordance with the guidelines of European Directive 2010/63/EU and handled in strict accordance with good animal practice as defined by the French National Charter on the Ethics of Animal Experimentation.

### Human Retinas

Human retina samples were obtained from multi-organ donations, as described previously (See References 3, 5). All tissue samples were obtained in accordance with the tenets of the Declaration of Helsinki, and experimental protocols were approved by ethics committees (permit numbers: Hungary: ETT TUKEB 34851-212018/EKU, Switzerland: EKNZ 19 Dec. 2014 and (EKNZ 2021-01773)).

### Human Retina Explants.

Briefly, after enucleation, the vitreous was removed and the retina was separated from the underlying RPE/choroid. We then cultured 5×5 mm tissue pieces on polycarbonate membrane inserts (VWR, Dietikon, Switzerland, catalogue number: 734-2720) with photoreceptor side down. Culture media was then added and retinal explants were cultured as described previously (Reference 3). Human retina explants were transduced with 3.8×10¹¹ v.g. (standard dose) or 2.8×10¹² v.g. (high dose). The AAV was pipetted on top of the retina and plates were incubated for 2 days. Culture media was then changed every 48 hours for 5 weeks and tissues were processed for MEA-recordings and/or histology.

### Retinal Organoids

Retinal organoids were derived from the 01F49i-N-B7 (short name: F49B7) iPSC line (Reference 5). This is a female line derived from an anonymized donor. Retinal organoids were transduced using 8.5×10¹¹ v.g. of different AAV constructs.

### 9.2 DNA Constructs and Cloning

The pAAV-CAG-ChrimsonR-tdTomato-hGH-polyA (See References 1, 2) is based on a construct reported by PCT Publication WO2017187272A1. This construct was used in a recent clinical trial targeting ganglion cells in the retina (See Reference 1). To obtain the pAAV-ProA7-ChrimsonR-tdTomato-hGH-polyA, the ProA7 promoter (See Reference 3) (FIG. 10) (500 bp long, Gnat2 upstream sequence) was cloned into this plasmid using PCR and Gibson assembly. To obtain the pAAV-ProA7-ChrimsonR-tdTomato-WPRE-hGH-polyA plasmid, the WPRE element was PCR amplified from the pAAV-synPVI-vfChrimson-EYFP-WPRE and Gibson ligated into the pAAV-ProA7-ChrimsonR-tdTomato-hGH-polyA plasmid.

Further optogenetic effectors were synthesized by GenScript, based on published sequences, as follows: ReaChR-Citrine (See Reference 6), CatCh-EGFP (See Reference 7), vf-Chrimson-EYFP (See Reference 8), fChrimson-EYFP (See Reference 8), Jaws-EGFP (See Reference 9), ChRMine-EYFP (See Reference 10), and ChRMine-tdTomato (See Reference 10). All effectors were placed into the pAAV-synPVI-vfChrimson-EYFP-WPRE backbone. We created the UBI-f-Chrimson-EYFP by synthesizing the ubiquitin tag and placing it in frame of the fChrimson (GenScript). The L132C position in f-Chrimson-EYFP was mutagenized to obtain a Chrimson with the CatCh mutation (GenScript). AAV capsids (AAV5, AAV8, AAV9, AAV-PHP.B (See Reference 11), AAV-PHP.eB (See Reference 12, AAV8-BP2 (See Reference 13), AAV44.9 (See Reference 14), AAV44.9E531D (See Reference 14), AAV-NHP26 (See Reference 15)) were obtained from Addgene or synthesized with GenScript.

### 9.3 AAV Vector Production

AAV production for mouse retina, organoid and human retina experiments were performed using standard adenovirus helper method using 15 cm plates, as described previously (See Reference 3). The pHGT1-Adeno1 helper plasmid harboring the adenoviral genes was kindly provided by C. Cepko. Vectors were purified on an iodixanol gradient and were resuspended in PBS containing 0.001% Pluronic. For NHP, 10-layer cell stacks were used for AAV production.

AAVs were titered with dual-color qPCR (Life Technologies) and ddPCR (BioRad) for the ITR element as well as for the WPRE element (where applicable). PCR primers and probes were as follows: ITR forward: 5′-GGAACCCCTAGTGATGGAGTT-3′ (SEQ ID NO: 90), ITR reverse: 5′-CGGCCTCAGTGAGCGA-3′ (SEQ ID NO: 91), ITR probe: 5′-CACTCCCTCTCTGCGCGCTCG-3′ (FAM) (SEQ ID NO: 92), WPRE forward: 5′-GGCTGTTGGGCACTGACAA-3′ (SEQ ID NO; 93), WPRE reverse: 5′-CCAAGGAAAGGACGATGATTTC-3′ (SEQ ID NO: 94), WPRE probe: 5′-TCCGTGGTGTTGTCG-3′ (VIC) (SEQ ID NO: 95).

To allow for comparison across different AAV vectors, the AAVs were titer matched based on the ddPCR for WPRE value. To allow for precise comparison, the following method was applied: after AAV purification, the AAV was aliquoted to 40 μL aliquots. The vials were then frozen at −80° C. At least 2 weeks later, one aliquot was thawed and titration was performed from 5 μL. The leftover AAV was applied on organoids the same day. The other 40 μL aliquots were used in human retina experiments using the titers obtained from the respective aliquot used for organoids and titering.

### 9.4 Mouse In Vivo Injections

Subretinal injections were performed on wild-type mice (6-8 weeks old) anesthetized with 2.5% isoflurane. After making a small incision on the sclera with a sharp 30-G needle, the vector solution was injected through this incision into the subretinal space using a blunt 5-μl Hamilton syringe mounted on a micromanipulator. The injected genomic copies were 1.7×10¹⁰ vector genomes, using ddPCR as titration method. After 5 weeks, animals were sacrificed and the eyecups were removed.

### 9.5 NHP In Vivo Injections

NHP injection, materials used for the injection: De Juan/Awh Subretinal Injection Cannula (25/41G), Bausch and Lomb, 12.03.25, 20916 Hamilton® GASTIGHT® syringe, PTFE luer lock 1750TLL, 0.5 mL, Sigma, 20916, BD 1 mL Syringe, Luer-Lok Tip, BD via WVR 309628, Masterflex Transfer Tubing, Microbore PTFE, 0.012″ ID×0.030″ OD; 100 ft/roll, Cole-Palmer 06417-11.

NHP Injection procedure and perioperative medication: Injection of AAV-ProA7-ReaChR-citrine-WPRE-hGH polyA: Pre-injection fundus photos and OCT scans of the macula and the optic nerve head were obtained to exclude ocular pathologies. Three days before injection, animals started to receive 0.75 mg/kg intramuscular dexamethasone for one week. The day before injection, animals started to receive 15 mg/kg intramuscular amoxicillin followed by two more doses 48 hours apart. Animals were fasted before the day of operation (access to water was maintained). On the day of the surgery, animals were anesthetized using ketamine (10 mg/kg, intramuscular) and transported to the operating room. The pupils were dilated using 0.5% tropicamide and 10% phenylephrine. Animals then received propofol (5-10 mg/kg) followed by intubation. The anesthesia was maintained by isoflurane (1-2.5%) and animals were monitored during the procedure. After disinfection of the ocular surface, two ports were made on the limbus using 25G trocars: one for the endoillumination port and one for the 41G subretinal microinjection cannula, respectively. After gently touching the retina with the subretinal cannula first a pre-bleb was performed with BSS, followed by administration of 70 μL of test compound solution using manual injection. Shortly after injection we performed OCT imaging to visualize the subretinal blebs. Animals received subconjunctival antibiotic immediately after the procedure and tobramycin ointment for 7 days following the procedure.

Injection of AAV-Pro573.2-ReaChR-WPRE-hGH polyA: We switched from a manual injection to a foot-pedal controlled system through the Constellation vitrectomy platform (Alcon). We performed core vitrectomy in all eyes. Injection was done using the viscous fluid microdose system of the Constellation device. Injection pressure was controlled by setting the maximum pressure on the Constellation device. Briefly, after disinfection of the ocular surface, three ports were made on the limbus using 25G trocars: one for the endoillumination port, one for infusion line to keep the intraocular pressure (IOP) constant and one for manipulative tools. First, a core vitrectomy was performed. Next, we performed a pre-bleb injection using BSS. For injection the IOP was kept at 5 mmHg and injection pressure was set to a maximum 16 psi (in one case, we had to increase to 20 psi). After successful pre-bleb injection, we decreased injection pressure to a maximum 8 psi, and the subretinal injection of the virus solution was performed into the pre-bleb. Shortly after injection we performed OCT imaging to visualize the subretinal blebs. Animals received subconjunctival antibiotic immediately after the procedure and tobramycin ointment for 7 days following the procedure.

NHP Enucleation and sample processing: Animals were enucleated under terminal, deep anesthesia with circulation still in place. The eye was immediately placed in Ames supplemented with 50 μg/mL Kanamycin, which was oxygenated with a mixture of 95% oxygen, 5% carbon dioxide for 30 minutes. First, the anterior segment was removed with a scalpel and scissors, then the vitreous was removed. The eyecup was transferred from the surgical center to the laboratory and then processed. The blebbed retina was dissected with 3-5 mm biopsy punches dependent on the original size of the bleb, so that the majority of the tissue was derived from the blebbed retina. A corresponding non-bleb macular control region was taken with a 4 mm biopsy punch. Some of the non-bleb retina areas were subjected to MEA recordings to detect acute responses. The bleb area was cut to 4 quadrants and retinas were cultured similarly to human retinal explants1. 1-2 days later these retina pieces (along with control, non-bleb retinal pieces) were subjected to MEA recordings. After MEA recordings, 3 of 4 pieces of each condition were fixed for histology and 1 of 4 pieces was processed for gDNA/RNA isolation.

### 9.6 Human Retina Explant Culture

Human retina cultures were maintained at 37° C. in 5% CO2 in DMEM/F-12 nutrient medium (Thermo Fisher Scientific), supplemented with 0.1% BSA, 10 μM O-acetyl-L-carnitine hydrochloride, 1 mM fumaric acid, 0.5 mM galactose, 1 mM glucose, 0.5 mM glycine, 10 mM HEPES, 0.05 mM mannose, 13 mM sodium bicarbonate, 3 mM taurine, 0.1 mM putrescine dihydrochloride, 0.35 μM retinol, 0.3 μM retinyl acetate, 0.2 μM (±)-α-tocopherol, 0.5 mM ascorbic acid, 0.05 μM sodium selenite, 0.02 μM hydrocortisone, 0.02 μM progesterone, 1 μM insulin and 0.003 μM 3,3′,5′-triiodo-L-thyronine (Sigma-Aldrich). For AAV transduction experiments, 2.8×10¹² (high dose) or 3.8×10¹¹ (low dose) vector genomes were used per human retina explant (based on ddPCR for WPRE or ChrimsonR in the case of the ChrimsonR construct, where no WPRE element was present). The virus was diluted in 20 μL complete media and was applied on the top of retinal pieces (from the ‘vitreal’ side). 48 hours later, the media was changed. Human retinal explants were incubated for another 5 weeks before processing them for analysis.

### 9.7 Organoid Culture

iPSCs were maintained in mTeSR1 medium (STEMCELL Technologies) and cultured at 37° C. and 5% C02 in a humidified incubator. Mycoplasma testing was regularly performed using the MycoAlert PLUS Mycoplasma Detection Kit (Lonza). Retinal organoids were derived using the agarose microwell array seeding and scraping method (AMASS), as described previously (See, Reference 2).

From day 42, embryoid bodies were cultured in 3:1 medium supplemented with 10% heat-inactivated FBS (Millipore) and 100 μM taurine (Sigma) with media changes every other day. From week 10, the culture medium was supplemented with 1 μM retinoic acid (Sigma). From week 14, the B27 supplement in 3:1 media was replaced by N2 supplement (GIBCO, #17502-048) and retinoic acid was reduced to 0.5 μM. Retinal organoids were cultured up to at least 28 weeks before performing experiments.

For organoid AAV transduction, 8.5×10¹¹ (regular dose) vector genomes were used per organoid (based on ddPCR for WPRE or ChrimsonR in the case of the ChrimsonR construct, where no WPRE element was present). The virus was diluted in 30 μL complete media, which was applied then on organoids in 96 well plates. After 4 hours, 70 μL of complete media was pipetted on top. The next day, another 100 μL volume was applied without changing the media. The following day (48 hours after AAV application), the media was completely changed. Retinal organoids were cultured for at least 4 weeks before processing. Media was changed every 2-3 days.

### 9.8 Light Stimulation for MEA Recordings

NHP and human retina multi-electrode array (MEA) recordings: MEA recordings in human retinal explants were performed using a USB-MEA256- or the MEA2100 system (MultiChannel Systems, Reutlingen Germany). Human retinal recordings were performed between 5 and 8 weeks of culture. NHP retinal recordings were performed 1-2 days after explantation. Human retinal explants were carefully removed from culture dish by flushing some oxygenated Ames medium, and were transferred to a petri dish filled with Ames with the help of a Moria spoon. Vitreous was then detached with help of sharp forceps (Fine Science Tools, USA) under a stereomicroscope (Olympus, Germany). The tissue was placed on a MEA (256MEA100/30iR-ITO-pr, MultiChannel Systems, Germany), ganglion cell layer facing the electrodes, and pressed down to the electrodes with a slice holder (Science Products, Germany). The MEA was next placed inside the MEA system. Retinas were continuously perfused with Ame's medium (US Biological Life Sciences, USA) bubbled with 95% 02 and 5% C02 (carbogen) at a rate of 1-3 ml/minute. Temperature of the media was kept constant at 37° C. using a inline solution heather and its temperature controller (Harvard System, USA). Tissue and its fluorophore-fluorescence were visualized with a IX73 inverted microscope (Olympus, Germany) at 4× and 20× objective, using the appropriate filter.

Light stimulation for MEA recordings: To active the optogenetic effector, white light stimuli were generated using a DLP projector (K10, Acer, USA (projector #1), EH500 DLP Projector, Optoma, Taiwan (projector #2) or NEC, Japan (projector #3)) with optics of the microscope modified to project sharply onto the retinal surface. Projector and stimulus timing were controlled and recorded using a custom Python software developed by Zoltan Raics. Light intensity was varied using neutral density (ND) filters (Thorlabs, New Jersey, USA) ranging from Optical Density 4 (0.01% light transmission) up to optical density 1 (10% light transmission) in a logarithmic scale. Output intensities for the different light stimuli are described in Table 1. We first applied a preliminary protocol of 2 seconds full field light flashes at 100% contrast repeated 10 times with 5 s interstimulus interval (ISI) using OD2 filter to detect light-responsive regions. This configuration give rise to a total photon flux of 1.12E16 to 1.34E16, depending on the projector. Then, we applied a pre-defined set of different light stimuli: (1) full field white light flashes of 2 seconds duration followed by 10 s ISI. Flashes were repeated 5 times with contrasts varying between 12.5%, 25%, 50% and 100%, presented in this order. Responses to this set of stimuli were recorded across four different ND filters (OD4, OD3, OD2 and OD1) to vary light intensity in order to determine the threshold of response. (2) full field flicker stimulus using OD2 filter frequencies presented were 0, 7.7 Hz, 11, 16, 23.2, 27.6, and 33.1 Hz. Responses to this stimulus protocol were used to determine the maximal frequency retinal ganglion cells would follow. Light intensities of the different stimuli were expressed in photon flux. Total photon flux was calculated from the irradiance of the white light stimulus, measured with power sensor (S170C, Thorlabs, USA) connected to a power meter (PM100D, Thorlabs, USA) and from its spectrum (Ocean Optics, USA) using the energy of the photon formula. Original irradiance values were corrected for the wavelength responsivity curve of the power sensor. Effective photon flux was calculated from total photon flux corrected by the responsivity wavelength curve of the optogenetic effector ReaChR.

TABLE 2
Irradiances (mW/cm²) for ND filters and stimulus contrast used

| | 100% | 50% | | |
|---|---|---|---|---|
| ND-Filter | Contrast | Contrast | 25% Contrast | 12.5% Contrast |
| ND1 | 661.8 | 110 | 25.2 | 6.9 |
| ND2 | 56.6 | 10.1 | 2.2 | 0.6 |
| ND3 | 6.3 | 1.1 | 0.3 | 0.07 |
| ND4 | 0.6 | 0.1 | 0.03 | 0.01 |

### 9.10. Data Analysis for MEA Recordings

### Analysis of 2 s Flashes

### Measure for Light Responsivity

To quantify if there were light responsive cells after cone activation using different AAV constructs, a measure for light responsiveness was developed. The approach, as described in reference 5, was followed by computing the correlation between trials. For each unit, a peristimulus time histogram (PSTH) of the spike trains was computed for each trial with a bin size of 250 ms. The correlation coefficient was then computed between the individual trials and their significance (p-value).

If a cell showed significant positive correlation (r>0, p<0.01) the cell was labeled light responsive. The higher the correlation between trials, the more reliable the cell response to light stimulation, which also gives a measure of how well a cell responds towards the stimulus. This measure was used to compare different constructs (and control) for treatment to isolate which AAV vector is the most effective.

### Quantifying the Percentage of Light Responsive Cells

Of interest was the percentage of light responsive cells among all cells that show activity, for different viral constructs, to find suitable candidates for optogenetic treatment. To quantify the ratio of light responsive vs. non-light responsive cells, the above defined measure was used. For each construct, the number of light responsive cells was counted and divided by the total number of cells for that construct.

### Investigation of RGC Types

The primate retina comprises 4 major cell types that make up the vast majority of cells: ON-Midget, OFF-Midget, ON-Parasol, OFF-Parasol. These cell types can be functionally classified by their responses towards light flashes.

To investigate whether optogenetic activation of the cones engages these functional cell types, cell responses to the 2 s flashes stimuli were grouped. The trial average PSTH was computed as a feature vector for each cell. To identify functional cell types, unsupervised machine learning methods were used. First, dimensionality of the feature vector was reduced by using a non-linear dimensionality reduction technique (UMAP). The reduced feature vector was used to visualize the population of cells as well as an input for clustering the cells using Hierarchical Clustering. The firing rates and spike trains for each cluster were visualized individually to analyze and quantify functional cell types in the data set. This procedure was applied to the other light stimuli but mainly focused on the 2 s-Flash stimulus for cell type classification.

### Analysis of Contrast Flashes

### Analysis of Effective Photon Flux on Light Responsiveness

To investigate and compare the light responsiveness of healthy primate retina to different light levels as quantified by effective photon flux, responses to the contrast flashes stimulus were analyzed. The combination of 4 different contrast steps as well as 4 different ND-filters led to a total of 16 different levels of effective photon flux, which were measured in each setup. For each level of effective photon flux, the trial-to-trial correlation was computed, similar to that described in the analysis of the 2 s flashes stimulus. The trial-to-trial correlation was plotted as a function of the effective photon flux.

### Analysis of Responses to Chirpsweep

### Flicker Frequency Analysis

To investigate whether RGC responses after optogenetic cone activation are able to follow a range of flicker frequencies, frequency analysis of their responses towards the Chirpsweep stimulus was conducted. Analysis focused on the full-contrast frequency chirp where the frequency is modulated from 2 Hz to 8 Hz. Spike trains were cut respectively around that frequency range and a PSTH was computed, this time with a bin size of the inverse of the projector frequency (60 Hz) to ensure capture of high frequency modulation of the responses. Short-time Fourier transform (STFT) was used to visualize the power of the PSTH resolved in time as well as frequency. This applied for single cells as well as for populations.

Since single cell responses can be noisy, the population analysis was focused on. To that extent, the responses were clustered (See Analysis of 2 s Flashes stimulus, Investigation of RGC types) into 5 clusters. Then the PSTH was averaged across the population to reduce noise. The STFT for each individual cluster was plotted to investigate the frequency response for different cell types.

### 9.11 Histology, Imaging and Image Analysis

### Human Retina Histology

Human retinas were fixed for 30 min in 4% (wt/vol) paraformaldehyde in PBS and washed with PBS for 24 hours at 4° C. After cryoprotection with 30% (wt/vol) sucrose, retinas were subjected to three cycles of freeze-thaw cycles. Sucrose was washed out by soaking the retinas in PBS for 15 minutes in 24-well plates. Blocking was performed using 200 μL blocking buffer (10% normal donkey serum (NDS, Chemicon), 1% (wt/vol) BSA, 0.5% (vol/vol) Triton X-100 and 0.01% (wt/vol) sodium azide (Sigma-Aldrich) in PBS) for 1 hour at room temperature. Antibodies were diluted in staining buffer (3% (vol/vol) NDS, 1% (wt/vol) BSA, 0.01% (wt/vol) sodium azide and 0.5% Triton X-100 in PBS).

Primary antibodies were incubated with the tissue in 24 well plates for 3-5 days. The following primary antibodies were used: anti-arrestin 3, goat polyclonal (NovusBio, NBP1-37003, 1:200), anti-arrestin 3, 7G6 antibody (See, Reference 16), mouse monoclonal (a gift from W. Baehr. 1:400), anti-GFP, rabbit polyclonal IgG fraction, (Thermo, A11122, 1:200), anti-RFP, rabbit polyclonal (Rockland 600-401-379, used to detect tdTomato, 1:200). Retinas were washed 3 times with PBS.

Secondary antibody staining was performed for one hour. The following secondary antibodies were applied in the study: anti-goat Alexa Fluor 647, donkey IgG (H+L) (Thermo, A-21447, 1:200), anti-mouse Alex Fluor 647, donkey IgG (H+L) (Thermo, A31571, 1:200), anti-rabbit Alexa-Fluor 488, donkey IgG (H+L) (Thermo, A21206, 1:200) and anti-rabbit Alexa Fluor 568, IgG (H+L) (Thermo, A10042, 1:200).

For nuclear staining, Hoechst dye was used in 1:1,000 dilution, stained together with the secondary antibodies. Retinas were washed again 3 times with PBS. Retinas were mounted on SuperFrost slides (Fischer Scientific) using Prolong Gold antifade mountant (Thermo).

### Organoid Histology

Organoids were fixed for 4 hours at 4° C. in 4% PFA in PBS. After fixation, samples were washed 3 times 30 minutes with PBS and cryopreserved in 30% sucrose in PBS overnight at 4° C. Organoids were then embedded in 7.5% gelatin and 10% sucrose. Cryosections (20-40 m) were generated using a cryostat (MICROM International) on organoids. Sections were mounted onto Superfrost Plus slides (Thermo), dried for 4 to 16 hours at room temperature and stored at −80° C. until use.

Antibody staining was performed similarly as described above for human retinas. Briefly, for immunostainings of cryosections, slides were first dried for 30 minutes at room temperature and then rehydrated for 5-10 minutes in PBS. After blocking in blocking buffer (see above), primary antibodies were diluted in staining buffer and incubated with the tissue overnight. Washing was done using 3×15 minutes in PBS with 0.1% TWEEN 20 (Sigma). Organoids were mounted on SuperFrost slides (Fischer Scientific) using Prolong Gold antifade mountant (Thermo).

### Mouse Retina Histology

Mouse eyecups were explanted after euthanasia and fixed for 24 hours in 4% (vol/vol) PFA in PBS. Retinas were embedded in sucrose and then were subjected to 3 cycles of freeze-thaw. After blocking (as described above), retinas were stained with the following primary antibodies: anti-mouse cone arrestin, rabbit polyclonal (Merck Millipore: AB15282) and anti-RFP, rat monoclonal (AB_2336064, Chromotek). For secondary antibodies, we applied anti-rabbit Alexa-Fluor 488, donkey IgG (H+L) (Thermo, A21206, 1:200) and anti-rat Cy3.

### NHP Retina Histology

NHP eyes were removed in deep terminal anesthesia. After removal of eyes, the vitreous was removed and the retinas were immersed into 4% (vol/vol) PFA in PBS. Retinas were processed similarly to human retinas (described above).

### Imaging

Imaging was performed using a Axio Imager M2 upright microscope, Yokogawa CSU W1 dual camera T2 spinning disk confocal scanning unit, Visitron VS-Homogenizer or an Olympus IXplore Spin confocal spinning disc microscope system. Three images were acquired at randomly selected locations using the 40× objective.

### Image Analysis

Image analysis was performed with Imaris. Briefly, cones were segmented based on cone arrestin fluorescence. Background fluorescent intensity was measured in five randomly regions. Positivity for a fluorophore was determined using a 5× mean+standard deviation of the background cutoff.

### Multiple Copies of Promoters Enhance Gene Expression in Cone and Rod Photoreceptors Vector Constructs and Production

Promoter oligomers were designed using Geneious Prime and ordered for gene synthesis. For cone specific promoters, 1×, 2×, 3×, and 4× ProSC promoter replicates were cloned into the pAAV-SynP330-EGFP-WPRE plasmid before the optimized Kozak sequence (GCCACC) and the translation start codon of eGFP coding sequence, followed by a woodchuck hepatitis virus posttranscriptional regulatory element (WPRE). For rod-specific promoters, 1×, 2×, 3×, and 4xmin330 were cloned into the pAAV-SynP330-EGFP-WPRE plasmid before the optimized Kozak sequence (GCCACC) and the translation start codon of eGFP coding sequence, followed by a woodchuck hepatitis virus posttranscriptional regulatory element (WPRE). Two deletion mutants of min330, 330-3del1del5 and 330-3del1del6, were also cloned into the pAAV-SynP330-EGFP-WPRE plasmid before the optimized Kozak sequence (GCCACC) and the translation start codon of eGFP coding sequence, followed by a woodchuck hepatitis virus posttranscriptional regulatory element (WPRE).

AAVs of serotype 5 and PHP.eB were made as described by Grieger et al. (2006). Genome copy (GC) number titration was performed using real-time PCR (Applied Biosystems, TaqMan reagents).

Retinal organoids or Human retina explant cultures were transduced in triplicate at week 28 with a total of 1E10 and 1E11 v.g. per well of AAV containing cell lysate mixed carrying the replicate promoter variants (serotypes, AAV5 and AAVPhP.eB) inducing the expression of enhanced GFP.

### Mouse Injections

rd1 animals were used in this study, which are blind beyond postnatal day 28. Subretinal injections were performed under 2.5% isoflurane anaesthesia. A small incision was performed in the sclera near the lens using a sharp 30-G needle. A blunt 5 um Hamilton syringe was then inserted into the incision site and 2 μL virus solution was injected into the subretinal space. We injected AAV-ProA7-ReaChR-citrine-EYFP-WPRE. Light responses were analyzed using MEA on freshly explanted retinas, as described above.

### REFERENCES

1. Sahel, J.-A. et al. Partial recovery of visual function in a blind patient after optogenetic therapy. Nat Med 27, 1223-1229 (2021).
2. Gauvain, G. et al. Optogenetic therapy: high spatiotemporal resolution and pattern discrimination compatible with vision restoration in non-human primates. Commun Biol 4, 125 (2021).
3. Juttner, J. et al. Targeting neuronal and glial cell types with synthetic promoter AAVs in mice, non-human primates and humans. Nat Neurosci 22, 1345-1356 (2019).
4. Busskamp, V. et al. Genetic reactivation of cone photoreceptors restores visual responses in retinitis pigmentosa. (2010). doi:10.1126/science.1190897.
5. Cowan, C. S. et al. Cell Types of the Human Retina and Its Organoids at Single-Cell Resolution. Cell 182, 1623-1640.e34 (2020).
6. Lin, J. Y., Knutsen, P. M., Muller, A., Kleinfeld, D. & Tsien, R. Y. ReaChR: a red-shifted variant of channelrhodopsin enables deep transcranial optogenetic excitation. Nature Neuroscience 16, 1499-1508 (2013).
7. Kleinlogel, S. et al. Ultra light-sensitive and fast neuronal activation with the Ca2+-permeable channelrhodopsin CatCh. Nat Neurosci 14, 513-518 (2011).
8. Mager, T. et al. High frequency neural spiking and auditory signaling by ultrafast red-shifted optogenetics. Nature Communications 9, 1750 (2018).
9. Chuong, A. S. et al. Noninvasive optical inhibition with a red-shifted microbial rhodopsin. Nat Neurosci 17, 1123-1129 (2014).
10. Marshel, J. H. et al. Cortical layer-specific critical dynamics triggering perception. Science 365, (2019).
11. Deverman, B. E. et al. Cre-dependent selection yields AAV variants for widespread gene transfer to the adult brain. Nature Biotechnology (2016) doi:10.1038/nbt.3440.
12. Chan, K. Y. et al. Engineered AAVs for efficient noninvasive gene delivery to the central and peripheral nervous systems. Nature Neuroscience (2017) doi:10.1038/nn.4593.
13. Cronin, T. et al. Efficient transduction and optogenetic stimulation of retinal bipolar cells by a synthetic adeno-associated virus capsid and promoter. EMBO Molecular Medicine (2014) doi:10.15252/emmm.201404077.
14. Boye, S. L. et al. Novel AAV44.9-Based Vectors Display Exceptional Characteristics for Retinal Gene Therapy. Mol. Ther. 28, 1464-1478 (2020).
15. Byrne, L. C. et al. In vivo directed evolution of AAV in the primate retina. http://biorxiv.org/lookup/doi/10.1101/847459 (2019) doi:10.1101/847459.

### 6. NUMBERED EMBODIMENTS

1. A nucleic acid, comprising:
(i) a ProA7 promoter;
(ii) a nucleotide sequence encoding a depolarizing optogenetic protein and optionally a reporter molecule; and
(iii) a Woodchuck Hepatitis Virus Posttranscriptional Regulatory element (WPRE);
wherein the ProA7 promoter, the nucleotide sequence encoding a depolarizing optogenetic protein and optionally a reporter molecule and the WPRE are operably linked;
with the proviso that the depolarizing optogenetic protein is not CatCh.

2. The nucleic acid of numbered embodiment 2, further comprising a nucleotide sequence encoding a polyadenylation signal (PolyA) that is 3′ of the nucleotide sequence encoding the WPRE; wherein the nucleotide sequence encoding the PolyA and the nucleotide sequence encoding the WPRE are operably linked.

3. The nucleic acid of numbered embodiment 1 or 2, further comprising a nucleotide sequence encoding an AAV inverted terminal repeat sequence (ITR).

4. The nucleic acid of numbered embodiment 3, comprising a first AAV ITR that is 5′ of the ProA7 promoter and a second AAV ITR that is 3′ of the WPRE and preferably 3′ of the PolyA signal.

5. The nucleic acid of any one of numbered embodiments 1-4, wherein the depolarizing optogenetic protein is a light responsive polypeptide.

6. The nucleic acid of numbered embodiment 5, wherein the light responsive polypeptide is a light-gated ion channel polypeptide.

7. The nucleic acid of numbered embodiment 6, wherein the light-gated ion channel polypeptide is a channelrhodopsin or a functional variant thereof.

8. The nucleic acid of numbered embodiment 7, wherein the light-gated ion channel polypeptides is a channelrhodopsin.

9. The nucleic acid of numbered embodiment 8, wherein the channelrhodopsin is a channelrhodopsin-1 (ChR1), a channelrhodopsin 2 (ChR2), a functional variant thereof.

10. The nucleic acid of numbered embodiment 8, wherein the channelrhodopsin is a ChrMine polypeptide, a Chrimson polypeptide, a ReaChR polypeptide, a functional variant thereof.

11. The nucleic acid of numbered embodiment 7, wherein the ChrMine polypeptide is a Chrimson polypeptide.

12. The nucleic acid of numbered embodiment 11, wherein ChrMine polypeptide is a ReaChR polypeptide.

13. The nucleic acid of numbered embodiment 10, wherein the channelrhodopsin is ChrimsonR, ReaChR, ChrMine, fChrimson, or vfChrimson.

14. The nucleic acid of any one of the preceding numbered embodiments, wherein the nucleic acid encodes a reporter molecule selected from tdTomato, enhanced yellow fluorescent protein (EYFP), Citrine, green fluorescent protein (GFP), and variants thereof.

15. The nucleic acid of any one of numbered embodiments 1 and 13, wherein the nucleic acid does not encode the optional a reporter molecule.

16. The nucleic acid of any one of the numbered embodiments 1-13, wherein the nucleic acid encodes the depolarizing optogenetic protein and the reporter molecule.

17. The nucleic acid of numbered embodiment 16, wherein the depolarizing optogenetic protein is ChrimsonR and the reporter molecule is tdTomato; wherein the depolarizing optogenetic protein is ChrimsonR and the reporter molecule is EYFP; wherein the depolarizing optogenetic protein is ReaChR and the reporter molecule is Citrine; wherein the depolarizing optogenetic protein is ChrMine and the reporter molecule is EYFP; wherein the depolarizing optogenetic protein is fChrimson and the reporter molecule is TdTomato; or wherein the depolarizing optogenetic protein is vfChrimson and the reporter molecule is TdTomato.

18. The nucleic acid of numbered embodiment 16 or 17, wherein the nucleic acid encodes a fusion protein that comprises a depolarizing optogenetic protein that is fused to the reporter molecule.

19. The nucleic acid of any one of numbered embodiments 1-18, wherein the nucleic acid comprises a nucleotide sequence having at least 70% identity to SEQ ID NO: 4, SEQ ID NO: 5, SEQ ID NO: 6, or SEQ ID NO: 7.

20. The nucleic acid of any one of numbered embodiments 1-18, wherein the ProA7 promoter comprises a nucleic acid sequence having at least 70% identity to SEQ ID NO: 2.

21. The nucleic acid of any one of numbered embodiments 1-18, wherein the WPRE comprises a nucleotide sequence having at least 70% identity to SEQ ID NO: 3.

22. A nucleic acid, comprising:
(i) a ProA7 promoter;
(ii) a nucleotide sequence encoding a ReaChR protein and optionally a reporter molecule; and
(iii) a Woodchuck Hepatitis Virus Posttranscriptional Regulatory element (WPRE);
wherein the ProA7 promoter, the nucleotide sequence encoding a depolarizing optogenetic protein and optionally a reporter molecule and the WPRE are operably linked.

23. The nucleic acid of numbered embodiment 23, further comprising a nucleotide sequence encoding a polyadenylation signal (PolyA) that is 3′ of the nucleotide sequence encoding the WPRE; wherein the nucleotide sequence encoding the PolyA and the nucleotide sequence encoding the WPRE are operably linked.

24. The nucleic acid of numbered embodiment 22 or 23, further comprising a nucleotide sequence encoding an AAV inverted terminal repeat sequence (ITR).

25. The nucleic acid of numbered embodiment 24, further comprising a first AAV ITR that is 5′ of the ProA7 promoter and a second AAV ITR that is 3′ of the PolyA signal.

26. The nucleic acid of any one of numbered embodiments 22-25, wherein the nucleic acid does not encode the optional reporter molecule.

27. The nucleic acid of any one of numbered embodiments 22-26, where the nucleic acid encodes the ReaChR protein and the reporter molecule.

28. The nucleic acid of numbered embodiment 27, wherein the nucleic acid encodes a fusion protein that comprises a ReaChR Protein that is fused to a reporter molecule.

29. The nucleic acid of any one of numbered embodiments 22-28, wherein the reporter molecule is tdTomato, enhanced yellow fluorescent protein (EYFP), Citrine, green fluorescent protein (GFP), or variants thereof.

30. The nucleic acid of numbered embodiment 22, wherein the nucleic acid comprises a nucleic acid sequence having at least 70% identity to SEQ ID NO: 7.

31. The nucleic acid of any one of the preceding numbered embodiments, further comprising an intron.

32. A viral particle comprising the nucleic acid of any one of the preceding numbered embodiments.

33. The viral particle of numbered embodiment 32, wherein the viral particle is an AAV particle.

34. The viral particle of numbered embodiment 32 or 33, wherein the viral particle is capable of transducing about 10% of degenerated human cone cells.

35. A host cell comprising the nucleic acid of any one of numbered embodiments 1-31 or the viral particle of any one of numbered embodiments 32-33.

36. An AAV vector, comprising: (i) a nucleic acid of any one of numbered embodiments 1-21; and (ii) an AAV capsid.

37. The AAV vector of numbered embodiment 36, wherein the AAV capsid is an AAV8-BP2 capsid, an AAV-PHP.B capsid, an AAV-PHP.eB capsid, an AAV5 capsid, an AAV-NHP26 capsid, or an AAV-NHP26 capsid.

38. The AAV vector of numbered embodiment 36, wherein
a) when the depolarizing optogenetic protein is ReaChR, the optional reporter molecule is Citrine, and the AAV capsid is an AAV-PHP.eB, an AAV8-BP2, an AAV-NHP26 or an AAV5;
b) when the depolarizing optogenetic protein is ChrMine, the optional reporter molecule is EYFP, and the AAV capsid is an AAV-PHP.B, an AAV-PHP.eB, AAV-BP2, or an AAV-NHP26;
c) when the depolarizing optogenetic protein is ChrimsonR, the optional reporter molecule is tdTomato, and the AAV capsid is an AAV8-BP2, an AAV-PHP.B, an AAV-PHP.eB, or an AAV-NHP26;
d) when the depolarizing optogenetic protein is vfChrimson, the optional reporter molecule is tdTomato, and the AAV capsid is an AAV8-BP2, an AAV-PHP.B, an AAV-PHP.eB, an AAV-NHP26;
e) when the depolarizing optogenetic protein is ChrimsonR, the optional reporter molecule is EYFP, and the AAV capsid is an AAV8-BP2, an AAV-PHP.B, an AAV-PHP.eB, or an AAV-NHP26; or
f) when the depolarizing optogenetic protein is fChrimson, the optional reporter molecule is tdTomato, and the AAV capsid is an AAV8-BP2, an AAV-PHP.B, an AAV-PHP.eB, or an AAV-NHP26.

39. The AAV vector of numbered embodiment 38, wherein the nucleic acid does not encode the optional reporter molecule.

40. The AAV vector of numbered embodiment 36, wherein the nucleic acid comprises a nucleotide sequence with at least 70% identity to SEQ ID NO: 6; and wherein the AAV capsid is chosen from a AAV-PHP.eB capsid, a AAV-NHP26 capsid, a AAV-NHP26, or a AAV-PHP.B capsid.

41. An AAV vector, comprising:
(i) a nucleic acid of any one of numbered embodiments 22-31; and
(ii) an AAV capsid.

42. The AAV vector of numbered embodiment 41, wherein the AAV capsid is AAV-PHP.eB capsid, AAV-NHP26, a AAV5 capsid, or a AAV-PHP.BP2 capsid.

43. The AAV vector of numbered embodiment 41, wherein the optional reporter molecule is Citrine, and the AAV capsid is AAV-PHP.eB.

44. The AAV vector of numbered embodiment 41, wherein the optional reporter molecule is Citrine, and the AAV capsid is AAV8-BP2.

45. The AAV vector of numbered embodiment 41, wherein the optional reporter molecule is Citrine, and the AAV capsid is AAV5.

46. The AAV vector of any one of numbered embodiments 41-45, wherein the nucleic acid comprises a nucleotide sequence with at least 70% identity to SEQ ID NO: 7.

42. The AAV vector of any one of numbered embodiments 36-46, wherein the AAV vector is capable of transducing about 10% of human photoreceptor cone cells.

43. A host cell comprising the AAV viral vector of any one of numbered embodiments 26-42.

44. A pharmaceutical composition comprising:
(i) the nucleic acid of any one of numbered embodiments 1-31, the viral particle of any one of numbered embodiments 32-34, the AAV vector of any one of numbered embodiments 36-42, or the host cell of numbered embodiment 35 or 43; and
(ii) a pharmaceutically acceptable excipient.

45. A method of delivering a depolarizing optogenetic protein to a human cone cell in a subject in need thereof, the method comprising administering to the subject the nucleic acid of any one of numbered embodiments 1-31, the viral particle of any one of numbered embodiments 32-34, the AAV vector of any one of numbered embodiments 36-42, the host cell of numbered embodiment 35 or 43 or the pharmaceutical composition of numbered embodiment 44.

45. A method for treating a retinal disease, the method comprising administering to a subject in need thereof an effective amount of the nucleic acid of any one of numbered embodiments 1-31, the viral particle of any one of numbered embodiments 32-34, the AAV vector of any one of numbered embodiments 36-42, the host cell of numbered embodiment 35 or 43 or the pharmaceutical composition of numbered embodiment 44.

46. A method for restoring vision, the method comprising administering to a subject in need thereof an effective amount of the nucleic acid of any one of numbered embodiments 1-31, the viral particle of any one of numbered embodiments 32-34, the AAV vector of any one of numbered embodiments 36-42, the host cell of numbered embodiment 35 or 43 or the pharmaceutical composition of numbered embodiment 44.

47. A method for restoring sensitivity to light in a human cone photoreceptor cell, the method comprising administering the nucleic acid of any one of numbered embodiments 1-31, the viral particle of any one of numbered embodiments 32-34, the AAV vector of any one of numbered embodiments 36-42, the host cell of numbered embodiment 35 or 43 or the pharmaceutical composition of numbered embodiment 44.

48. A method for treating retinal degeneration in a subject, the method comprising administering to a subject in need thereof an effective amount of the nucleic acid of any one of numbered embodiments 1-31, the viral particle of any one of numbered embodiments 32-34, the AAV vector of any one of numbered embodiments 36-42, the host cell of numbered embodiment 35 or 43 or the pharmaceutical composition of numbered embodiment 44.

49. The method of any one of numbered embodiments 44-48, wherein the subject has or is at risk of developing retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, post-retinal detachment, cone dysfunction, a tapetoretinal degeneration, retinal vein occlusion, geographic atrophy, or a disorder impairing vision in which the optic nerve retains at least some function.

50. The method of any one of numbered embodiments 44-48, wherein the nucleic acid, the viral particle, the AAV vector, the host cell or the pharmaceutical composition is administered by subretinal injection.

51. The method of any one of numbered embodiments 45-50, wherein the nucleic acid, the viral particle, the AAV vector, the host cell or the pharmaceutical composition is administered before or after initiation of photoreceptor loss.

52. A nucleic acid of any one of numbered embodiments 1-31, viral particle of any one of numbered embodiments 32-34, AAV vector of any one of numbered embodiments 36-42, host cell of numbered embodiment 35 or 43 or pharmaceutical composition of numbered embodiment 44 for use in treating a retinal disease, restoring vision, restoring sensitivity to light in a human cone photoreceptor cell, or treating retinal degeneration.

53. The method of any one of numbered embodiments 45-51 or the use of numbered embodiment 52, the depolarizing optogenetic protein is expressed in a cell membrane of a human cone cell.

54. The method of any one of numbered embodiments 45-51 or the use of numbered embodiment 52, wherein the depolarizing optogenetic protein is capable of mediating a depolarizing current that depolarizes a human cone cell when exposed to light.

55. The method or use of numbered embodiment 54, wherein depolarization of the human cone cell induces light-driven ganglion cell spiking that is comparable to light-driven ganglion cell spiking in a functional human cone cell.

56. The method or use of numbered embodiment 55, wherein the light-driven ganglion cell spiking is assessed using a multi-electrode array.

57. The method of any one of numbered embodiments 44-48 or the use of numbered embodiment 52, wherein the nucleic acid is capable of restoring light sensitivity when introduced into a human cone cell, wherein restoration of light sensitivity occurs when (i) the depolarizing optogenetic protein is expressed in a cell membrane of the human cone cell, (ii) the depolarizing optogenetic protein is capable of mediating a depolarizing current that depolarizes a human cone cell when exposed to light, and (iii) depolarization of the human cone cell induces light-driven ganglion cell spiking.

58. An isolated nucleic acid, comprising:
(i) a promoter comprising:
   (a) a first nucleotide sequence selected from the group consisting of a nucleotide sequence of at least 150 nucleotides which has at least 70% identity to a sequence of equal length from SEQ ID NO: 2, a nucleotide sequence which has at least 70% identity to SEQ ID NO: 22, a nucleotide sequence which has at least 70% identity to SEQ ID NO: 23, and combinations thereof, and a second nucleotide sequence of at least about 370 nucleotides having at least 70% identity to a sequence of equal length from the sequence of SEQ ID NO: 10; or
   (b) a cone-specific promoter of at least about 150 nucleotides and no more than 499 nucleotides which has at least 70% identity to a sequence of equal length from the sequence of SEQ ID NO: 2;
(ii) a nucleotide sequence encoding a depolarizing optogenetic protein and optionally a reporter molecule; and
(iii) a Woodchuck Hepatitis Virus Posttranscriptional Regulatory element (WPRE); wherein the promoter, the nucleotide sequence encoding a depolarizing optogenetic protein and optionally a reporter molecule and the WPRE are operably linked.

59. The isolated nucleic acid of numbered embodiment 58, further comprising a nucleotide sequence encoding a polyadenylation signal (PolyA) that is 3′ of the nucleotide sequence encoding the WPRE; wherein the nucleotide sequence encoding the PolyA and the nucleotide sequence encoding the WPRE are operably linked.

60. The isolated nucleic acid of numbered embodiment 58 or 59, further comprising a nucleotide sequence encoding an AAV inverted terminal repeat sequence (ITR).

61. The isolated nucleic acid of numbered embodiment 60, comprising a first AAV ITR that is 5′ of the promoter and a second AAV ITR that is 3′ of the WPRE and preferably 3′ of the PolyA signal.

62. The isolated nucleic acid of any one of numbered embodiments 58-61, wherein the depolarizing optogenetic protein is a light responsive polypeptide.

63. The isolated nucleic acid of numbered embodiment 62, wherein the light responsive polypeptide is a light-gated ion channel polypeptide.

64. The isolated nucleic acid of numbered embodiment 63, wherein the light-gated ion channel polypeptide is a channelrhodopsin or a functional variant thereof.

65. The isolated nucleic acid of numbered embodiment 64, wherein the light-gated ion channel polypeptide is a channelrhodopsin.

66. The isolated nucleic acid of numbered embodiment 65, wherein the channelrhodopsin is a ReaChR polypeptide or a functional variant thereof.

67. The isolated nucleic acid of any one of numbered embodiments 58-66, wherein the isolated nucleic acid does not encode the optional reporter molecule.

68. The isolated nucleic acid of any one of numbered embodiments 58-67, wherein the WPRE comprises a nucleotide sequence having at least 70% identity to SEQ ID NO: 3.

69. The isolated nucleic acid of any one of numbered embodiments 58-68, wherein the promoter comprises:
two or more first nucleotide sequences;
two or more second nucleotide sequences;
two or more cone-specific promoters; or
two or more thereof.

70. The nucleic acid of any one of the preceding numbered embodiments, further comprising an intron.

71. The nucleic acid of any one of the preceding numbered embodiments, wherein the promoter comprises one or more sequences selected from the group consisting of SEQ ID NO: 2, SEQ ID NO: 10; SEQ ID NO: 11, SEQ ID NO: 12, SEQ ID NO: 13, SEQ ID NO: 14, SEQ ID NO: 22, SEQ ID NO: 23; SEQ ID NO: 24, SEQ ID NO: 25, SEQ ID NO: 26, SEQ ID NO: 27, SEQ ID NO: 30; SEQ ID NO: 36; SEQ ID NO: 37; SEQ ID NO: 38; SEQ ID NO: 39; SEQ ID NO: 40; SEQ ID NO: 41; and SEQ ID NO: 45.

72. A viral particle comprising the nucleic acid of any one of the numbered embodiments.

73. The viral particle of numbered embodiment 72, wherein the viral particle is an AAV particle.

74. The viral particle of numbered embodiment 72 or 73, wherein the viral particle is capable of transducing about 10% of degenerated human cone cells.

75. A host cell comprising the nucleic acid of any one of numbered embodiments 58-71 or the viral particle of any one of numbered embodiments 72-74.

76. An AAV vector, comprising:
(i) a nucleic acid of any one of numbered embodiments 58-71; and
(ii) an AAV capsid.

77. The AAV vector of numbered embodiment 76, wherein the AAV capsid is an AAV5 capsid, a PHP.eB capsid, an NHP26 capsid, an AAV8 capsid, an AAV8-BP2 capsid, an AAV9 capsid, or a PHP.B capsid.

78. The AAV vector of numbered embodiment 77, wherein
a) the promoter comprises SEQ ID NO: 2, the depolarizing optogenetic protein is ReaChR, the optional reporter molecule is absent, the PolyA is present and has SEQ ID NO: 9, and the AAV capsid is an AAV5;
b) the promoter comprises SEQ ID NO: 14, the depolarizing optogenetic protein is ReaChR, the optional reporter molecule is absent, the PolyA is present and has SEQ ID NO: 9, and the AAV capsid is an AAV5; or
c) the promoter comprises SEQ ID NO: 12, the depolarizing optogenetic protein is ReaChR, the optional reporter molecule is absent, the PolyA is present and has SEQ ID NO: 9, and the AAV capsid is an AAV5.

79. A host cell comprising the AAV vector of any one of numbered embodiments 76-78.

80. A pharmaceutical composition comprising:
(i) the nucleic acid of any one of numbered embodiments 58-71, the viral particle of any one of numbered embodiments 72-74, the AAV vector of any one of numbered embodiments 76-78, or the host cell of numbered embodiment 75 or 79; and
(ii) a pharmaceutically acceptable excipient.

81. A method of delivering a depolarizing optogenetic protein to a human cone cell in a subject in need thereof, the method comprising administering to the subject the nucleic acid of any one of numbered embodiments 58-71, the viral particle of any one of numbered embodiments 72-74, the AAV vector of any one of numbered embodiments 76-78, or the host cell of numbered embodiment 75 or 79, or the pharmaceutical composition of numbered embodiment 80.

82. A method for treating a retinal disease, the method comprising administering to a subject in need thereof an effective amount of the nucleic acid of any one of numbered embodiments 58-71, the viral particle of any one of numbered embodiments 72-74, the AAV vector of any one of numbered embodiments 76-78, or the host cell of numbered embodiment 75 or 79, or the pharmaceutical composition of numbered embodiment 80.

83. A method for restoring vision, the method comprising administering to a subject in need thereof an effective amount of the nucleic acid of any one of numbered embodiments 58-71, the viral particle of any one of numbered embodiments 72-74, the AAV vector of any one of numbered embodiments 76-78, or the host cell of numbered embodiment 75 or 78, or the pharmaceutical composition of numbered embodiment 80.

84. A method for restoring sensitivity to light in a human cone photoreceptor cell, the method comprising administering to the human cone photoreceptor cell in need thereof the nucleic acid of any one of numbered embodiments 58-71, the viral particle of any one of numbered embodiments 72-74, the AAV vector of any one of numbered embodiments 76-78, or the host cell of numbered embodiment 75 or 78, or the pharmaceutical composition of numbered embodiment 80.

85. A method for treating retinal degeneration in a subject, the method comprising administering to a subject in need thereof an effective amount of the nucleic acid of any one of numbered embodiments 58-71, the viral particle of any one of numbered embodiments 72-74, the AAV vector of any one of numbered embodiments 76-78, or the host cell of numbered embodiment 75 or 78, or the pharmaceutical composition of numbered embodiment 80.

86. The method of any one of numbered embodiments 81-85, wherein the subject has or is at risk of developing retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, or a tapetoretinal degeneration retinal vein occlusion.

87. The method of any one of numbered embodiments 81-86, wherein the nucleic acid, the viral particle, the AAV vector, the host cell or the pharmaceutical composition is administered by subretinal injection.

88. The method of any one of numbered embodiments 81-87, wherein the nucleic acid, the viral particle, the AAV vector, the host cell or the pharmaceutical composition is administered before or after initiation of photoreceptor loss.

89. The nucleic acid of any one of numbered embodiments 58-71, the viral particle of any one of numbered embodiments 72-74, the AAV vector of any one of numbered embodiments 76-78, or the host cell of numbered embodiment 75 or 78, or the pharmaceutical composition of numbered embodiment 80 for use in treating a retinal disease, restoring vision, restoring sensitivity to light in a human cone photoreceptor cell, or treating retinal degeneration.

90. The method or use of any one of numbered embodiments 81-89, wherein the depolarizing optogenetic protein is expressed in a cell membrane of a human cone cell.

91. The method or use of any one of numbered embodiments 81-90 wherein the depolarizing optogenetic protein is capable of mediating a depolarizing current that depolarizes a human cone cell when exposed to light.

92. The method or use of numbered embodiment 91, wherein depolarization of the human cone cell induces light-driven ganglion cell spiking that is comparable to light-driven ganglion cell spiking in a functional human cone cell.

93. The method or use of numbered embodiment 92, wherein the light-driven ganglion cell spiking is assessed using a multi-electrode array.

94. The method or use of any one of numbered embodiments 81-93, wherein the nucleic acid is capable of restoring light sensitivity when introduced into a human cone cell, wherein restoration of light sensitivity occurs when (i) the depolarizing optogenetic protein is expressed in a cell membrane of the human cone cell, (ii) the depolarizing optogenetic protein is capable of mediating a depolarizing current that depolarizes a human cone cell when exposed to light, and (iii) depolarization of the human cone cell induces light-driven ganglion cell spiking.

### 7. SEQUENCE DISCLOSURE

| SEQ ID NO: | Description | Sequence |
|---|---|---|
| 1 | 479_pssAAV-CAG- | GTCCTGCAGGCAGCTGCGCGCTCGCTCGCTCACTGAGGCCGCCCGGGCAAAG |
| | ChrimsonR-tdTomato | CCCGGGCGTCGGGCGACCTTTGGTCGCCCGGCCTCAGTGAGCGAGCGAGCGC |
| | WO2017187272A1 | GCAGAGAGGGAGTGGCCAACTCCATCACTAGGGGTTCCTGCGGCCGCACGCG |
| | | TCGTGGTACCTCTGGTCGTTACATAACTTACGGTAAATGGCCCGCCTGGCTG |
| | | ACCGCCCAACGACCCCCGCCCATTGACGTCAATAATGACGTATGTTCCCATA |
| | | GTAACGCCAATAGGGACTTTCCATTGACGTCAATGGGTGGAGTATTTACGGT |
| | | AAACTGCCCACTTGGCAGTACATCAAGTGTATCATATGCCAAGTACGCCCCC |
| | | TATTGACGTCAATGACGGTAAATGGCCCGCCTGGCATTATGCCCAGTACATG |
| | | ACCTTATGGGACTTTCCTACTTGGCAGTACATCTACTCGAGGCCACGTTCTG |
| | | CTTCACTCTCCCCATCTCCCCCCCCTCCCCACCCCCAATTTTGTATTTATTT |
| | | ATTTTTTAATTATTTTGTGCAGCGATGGGGGGGGGGGGGGGGGGGGCGCGCG |
| | | CCAGGCGGGGCGGGGCGGGGCGAGGGGCGGGGCGAGGCGGAGAGGTGCGGCG |
| | | GCAGCCAATCAGAGCGGCGCGCTCCGAAAGTTTCCTTTTATGGCGAGGCGGC |
| | | GGCGGCGGCGGCCCTATAAAAAGCGAAGCGCGCGGCGGGCGGGAGCGGGATC |
| | | AGCCACCGCGGTGGCGGCCTAGAGTCGACGAGGAACTGAAAAACCAGAAAGT |
| | | TAACTGGTAAGTTTAGTCTTTTTGTCTTTTATTTCAGGTCCCGGATCCGGTG |
| | | GTGGTGCAAATCAAAGAACTGCTCCTCAGTGGATGTTGCCTTTACTTCTAGG |
| | | CCTGTACGGAAGTGTTACTTCTGCTCTAAAAGCTGCGGAATTGTACCCGCGG |
| | | CCGATCCACCGGTCGCATCGATATTCTGAGCCGCCACCATGGCTGAGCTGAT |
| | | CAGCAGCGCCACCAGATCTCTGTTTGCCGCCGGAGGCATCAACCCTTGGCCT |
| | | AACCCCTACCACCACGAGGACATGGGCTGTGGAGGAATGACACCTACAGGCG |
| | | AGTGCTTCAGCACCGAGTGGTGGTGTGACCCTTCTTACGGACTGAGCGACGC |
| | | CGGATACGGATATTGCTTCGTGGAGGCCACAGGCGGCTACCTGGTCGTGGGA |
| | | GTGGAGAAGAAGCAGGCTTGGCTGCACAGCAGAGGCACACCAGGAGAAAAGA |
| | | TCGGCGCCCAGGTCTGCCAGTGGATTGCTTTCAGCATCGCCATCGCCCTGCT |
| | | GACATTCTACGGCTTCAGCGCCTGGAAGGCCACTTGCGGTTGGGAGGAGGTC |
| | | TACGTCTGTTGCGTCGAGGTGCTGTTCGTGACCCTGGAGATCTTCAAGGAGT |
| | | TCAGCAGCCCCGCCACAGTGTACCTGTCTACCGGCAACCACGCCTATTGCCT |
| | | GCGCTACTTCGAGTGGCTGCTGTCTTGCCCCGTGATCCTGATCAGACTGAGC |
| | | AACCTGAGCGGCCTGAAGAACGACTACAGCAAGCGGACCATGGGCCTGATCG |
| | | TGTCTTGCGTGGGAATGATCGTGTTCGGCATGGCCGCAGGACTGGCTACCGA |
| | | TTGGCTCAAGTGGCTGCTGTATATCGTGTCTTGCATCTACGGCGGCTACATG |
| | | TACTTCCAGGCCGCCAAGTGCTACGTGGAAGCCAACCACAGCGTGCCTAAAG |
| | | GCCATTGCCGCATGGTCGTGAAGCTGATGGCCTACGCTTACTTCGCCTCTTG |
| | | GGGCAGCTACCCAATCCTCTGGGCAGTGGGACCAGAAGGACTGCTGAAGCTG |
| | | AGCCCTTACGCCAACAGCATCGGCCACAGCATCTGCGACATCATCGCCAAGG |
| | | AGTTTTGGACCTTCCTGGCCCACCACCTGAGGATCAAGATCCACGAGCACAT |
| | | CCTGATCCACGGCGACATCCGGAAGACCACCAAGATGGAGATCGGAGGCGAG |
| | | GAGGTGGAAGTGGAAGAGTTCGTGGAGGAGGAGGACGAGGACACAGTGGCGG |
| | | CACCGGTAGTAGCAGTGAGTAAGGGCGAGGAAGTGATCAAAGAGTTCATGCG |
| | | GTTTAAGGTGAGAATGGAAGGAAGCATGAACGGCCACGAGTTCGAAATTGAG |
| | | GGAGAAGGAGAGGGACGGCCCTACGAGGGCACCCAGACAGCCAAGCTGAAAG |
| | | TGACAAAGGGCGGGCCTCTGCCATTCGCTTGGGACATCCTGAGCCCACAGTT |
| | | TATGTACGGCTCCAAGGCCTATGTGAAACATCCAGCTGACATTCCCGATTAT |
| | | AAGAAACTGAGCTTCCCCGAGGGGTTTAAGTGGGAAAGAGTGATGAACTTCG |
| | | AGGACGGAGGCCTGGTGACTGTGACCCAGGACAGCTCCCTGCAGGATGGGAC |
| | | CCTGATCTACAAGGTGAAAATGAGAGGGACAAATTTTCCCCCTGATGGACCT |
| | | GTGATGCAGAAGAAAACTATGGGATGGGAGGCCTCCACCGAAAGGCTGTATC |
| | | CACGCGACGGGGTGCTGAAAGGAGAAATCCACCAGGCTCTGAAGCTGAAAGA |
| | | TGGGGGACATTACCTGGTGGAGTTCAAGACAATCTACATGGCCAAGAAACCT |
| | | GTGCAGCTGCCAGGCTACTATTACGTGGACACAAAACTGGATATCACTTCAC |
| | | ACAACGAGGACTACACTATTGTGGAGCAGTATGAACGGAGCGAGGGGAGACA |
| | | CCATCTGTTCCTGGGCCATGGGACTGGAAGTACCGGCTCAGGGTCTAGTGGA |
| | | ACCGCCTCAAGCGAGGATAACAATATGGCTGTGATCAAAGAGTTCATGAGGT |
| | | TTAAGGTGCGCATGGAGGGCAGCATGAATGGGCACGAATTTGAGATTGAAGG |
| | | AGAGGGCGAAGGGAGGCCTTACGAGGGCACACAGACTGCCAAGCTGAAAGTG |
| | | ACCAAGGGAGGACCACTGCCTTTCGCTTGGGATATCCTGTCTCCTCAGTTTA |
| | | TGTACGGAAGTAAGGCCTATGTCAAGCATCCCGCTGACATTCCTGATTACAA |
| | | GAAACTGTCTTTCCCAGAGGGCTTTAAGTGGGAGAGAGTGATGAATTTTGAA |
| | | GATGGAGGCCTGGTGACCGTGACACAGGACTCCTCTCTGCAGGATGGCACTC |
| | | TGATCTACAAAGTCAAAATGCGCGGCACCAATTTTCCACCCGATGGGCCCGT |
| | | GATGCAGAAGAAAACAATGGGGTGGGAGGCCAGCACTGAACGGCTGTATCCT |
| | | AGAGACGGAGTGCTGAAGGGCGAAATCCACCAGGCCCTGAAGCTGAAAGACG |
| | | GCGGCCACTACCTGGTGGAGTTCAAAACCATCTACATGGCCAAGAAACCAGT |
| | | GCAGCTGCCCGGCTATTACTATGTGGACACCAAGCTGGATATCACATCCCAC |
| | | AATGAAGACTACACCATTGTGGAACAGTATGAGAGGTCTGAAGGACGCCACC |
| | | ATCTGTTTCTGTACGGCATGGATGAGCTGTATAAGTAAAGAAGCTTGCCTCG |
| | | AGCAGCGCTGCTCGAGAGATCTACGGGTGGCATCCCTGTGACCCCTCCCCAG |
| | | TGCCTCTCCTGGCCCTGGAAGTTGCCACTCCAGTGCCCACCAGCCTTGTCCT |
| | | AATAAAATTAAGTTGCATCATTTTGTCTGACTAGGTGTCCTTCTATAATATT |
| | | ATGGGGTGGAGGGGGGTGGTATGGAGCAAGGGGCAAGTTGGGAAGACAACCT |
| | | GTAGGGCCTGCGGGGTCTATTGGGAACCAAGCTGGAGTGCAGTGGCACAATC |
| | | TTGGCTCACTGCAATCTCCGCCTCCTGGGTTCAAGCGATTCTCCTGCCTCAG |
| | | CCTCCCGAGTTGTTGGGATTCCAGGCATGCATGACCAGGCTCAGCTAATTTT |
| | | TGTTTTTTTGGTAGAGACGGGGTTTCACCATATTGGCCAGGCTGGTCTCCAA |
| | | CTCCTAATCTCAGGTGATCTACCCACCTTGGCCTCCCAAATTGCTGGGATTA |
| | | CAGGCGTGAACCACTGCTCCCTTCCCTGTCCTTCTGATTTTGTAGGTAACCA |
| | | CGTGCGGACCGAGCGGCCGCAGGAACCCCTAGTGATGGAGTTGGCCACTCCC |
| | | TCTCTGCGCGCTCGCTCGCTCACTGAGGCCGGGCGACCAAAGGTCGCCCGAC |
| | | GCCCGGGCTTTGCCCGGGCGGCCTCAGTGAGCGAGCGAGCGCGCAGCTGCCT |
| | | GCAGGG |
| 2 | ProA7 promoter | CATCCTGAGAGATGAGCCAGGACAAAGAACCAGTAATAGCTCCTGGAGCAGC |
| | (Gnat2_500), | ACATCTGTTTTGCCAGGATTATCCCTTGGATCTCTTAAAACCGAGACCTTGT |
| | WO2017046084A1 | AATCTGAAGACTCAACTTGGGCTGTACCCTTAACCTTCAGCTCTATGATGCA |
| | | AGTGAGTCCACAGGACCGGAGGCTTTGAGATGAGCTTTTCAGAAGGGAGGAG |
| | | TTGGCCGCTTGCTCCCAGAGCTCCAGCACCTGCATTCTTCTGGCTATGTCAG |
| | | AAGCCAGATCATTTCCCTCGTTAAAAACAAAAACAAAAAAACAAACAAACAA |
| | | AATGTTAGTCTTTGCCCTTTATCTGCCTGGCAAAGCTTTTAATTGGCTTGAT |
| | | CTGTCATTCCGCTAGACATAAAGGGGACAATCCCCGGATTAGGAAGGAGCTC |
| | | TCCAGCTCGGGTAAGGAGTCTCAAGGCAAGGTAGGCAAGCACCACCGGTCCG |
| | | CACTCTCGCCCAGCTTTTACGGGAAGAAGAGA |
| 3 | WPRE element | GCTTATCGATAATCAACCTCTGGATTACAAAATTTGTGAAAGATTGACTGGT |
| | | ATTCTTAACTATGTTGCTCCTTTTACGCTATGTGGATACGCTGCTTTAATGC |
| | | CTTTGTATCATGCTATTGCTTCCCGTATGGCTTTCATTTTCTCCTCCTTGTA |
| | | TAAATCCTGGTTGCTGTCTCTTTATGAGGAGTTGTGGCCCGTTGTCAGGCAA |
| | | CGTGGCGTGGTGTGCACTGTGTTTGCTGACGCAACCCCCACTGGTTGGGGCA |
| | | TTGCCACCACCTGTCAGCTCCTTTCCGGGACTTTCGCTTTCCCCCTCCCTAT |
| | | TGCCACGGCGGAACTCATCGCCGCCTGCCTTGCCCGCTGCTGGACAGGGGCT |
| | | CGGCTGTTGGGCACTGACAATTCCGTGGTGTTGTCGGGGAAATCATCGTCCT |
| | | TTCCTTGGCTGCTCGCCTATGTTGCCACCTGGATTCTGCGCGGGACGTCCTT |
| | | CTGCTACGTCCCTTCGGCCCTCAATCCAGCGGACCTTCCTTCCCGCGGCCTG |
| | | CTGCCGGCTCTGCGGCCTCTTCCGCGTCTTCGCCTTCGCCCTCAGACGAGTC |
| | | GGATCTCCCTTTGGGCCGCCTCCCCGCATCGATACCG |
| 4 | ProA7-ChrimsonR- | GCTGCGCGCTCGCTCGCTCACTGAGGCCGCCCGGGCAAAGCCCGGGCGTCGG |
| | tdTomato-WPRE | GCGACCTTTGGTCGCCCGGCCTCAGTGAGCGAGCGAGCGCGCAGAGAGGGAG |
| | vector | TGGCCAACTCCATCACTAGGGGTTCCTGCGGCCGCACGCGTCATCCTGAGAG |
| | | ATGAGCCAGGACAAAGAACCAGTAATAGCTCCTGGAGCAGCACATCTGTTTT |
| | | GCCAGGATTATCCCTTGGATCTCTTAAAACCGAGACCTTGTAATCTGAAGAC |
| | | TCAACTTGGGCTGTACCCTTAACCTTCAGCTCTATGATGCAAGTGAGTCCAC |
| | | AGGACCGGAGGCTTTGAGATGAGCTTTTCAGAAGGGAGGAGTTGGCCGCTTG |
| | | CTCCCAGAGCTCCAGCACCTGCATTCTTCTGGCTATGTCAGAAGCCAGATCA |
| | | TTTCCCTCGTTAAAAACAAAAACAAAAAAACAAACAAACAAAATGTTAGTCT |
| | | TTGCCCTTTATCTGCCTGGCAAAGCTTTTAATTGGCTTGATCTGTCATTCCG |
| | | CTAGACATAAAGGGGACAATCCCCGGATTAGGAAGGAGCTCTCCAGCTCGGG |
| | | TAAGGAGTCTCAAGGCAAGGTAGGCAAGCACCACCGGTCCGCACTCTCGCCC |
| | | AGCTTTTACGGGAAGAAGAGAGGATCCGCCACCATGGCTGAGCTGATCAGCA |
| | | GCGCCACCAGATCTCTGTTTGCCGCCGGAGGCATCAACCCTTGGCCTAACCC |
| | | CTACCACCACGAGGACATGGGCTGTGGAGGAATGACACCTACAGGCGAGTGC |
| | | TTCAGCACCGAGTGGTGGTGTGACCCTTCTTACGGACTGAGCGACGCCGGAT |
| | | ACGGATATTGCTTCGTGGAGGCCACAGGCGGCTACCTGGTCGTGGGAGTGGA |
| | | GAAGAAGCAGGCTTGGCTGCACAGCAGAGGCACACCAGGAGAAAAGATCGGC |
| | | GCCCAGGTCTGCCAGTGGATTGCTTTCAGCATCGCCATCGCCCTGCTGACAT |
| | | TCTACGGCTTCAGCGCCTGGAAGGCCACTTGCGGTTGGGAGGAGGTCTACGT |
| | | CTGTTGCGTCGAGGTGCTGTTCGTGACCCTGGAGATCTTCAAGGAGTTCAGC |
| | | AGCCCCGCCACAGTGTACCTGTCTACCGGCAACCACGCCTATTGCCTGCGCT |
| | | ACTTCGAGTGGCTGCTGTCTTGCCCCGTGATCCTGATCAGACTGAGCAACCT |
| | | GAGCGGCCTGAAGAACGACTACAGCAAGCGGACCATGGGCCTGATCGTGTCT |
| | | TGCGTGGGAATGATCGTGTTCGGCATGGCCGCAGGACTGGCTACCGATTGGC |
| | | TCAAGTGGCTGCTGTATATCGTGTCTTGCATCTACGGCGGCTACATGTACTT |
| | | CCAGGCCGCCAAGTGCTACGTGGAAGCCAACCACAGCGTGCCTAAAGGCCAT |
| | | TGCCGCATGGTCGTGAAGCTGATGGCCTACGCTTACTTCGCCTCTTGGGGCA |
| | | GCTACCCAATCCTCTGGGCAGTGGGACCAGAAGGACTGCTGAAGCTGAGCCC |
| | | TTACGCCAACAGCATCGGCCACAGCATCTGCGACATCATCGCCAAGGAGTTT |
| | | TGGACCTTCCTGGCCCACCACCTGAGGATCAAGATCCACGAGCACATCCTGA |
| | | TCCACGGCGACATCCGGAAGACCACCAAGATGGAGATCGGAGGCGAGGAGGT |
| | | GGAAGTGGAAGAGTTCGTGGAGGAGGAGGACGAGGACACAGTGGCGGCACCG |
| | | GTAGTAGCAGTGAGTAAGGGCGAGGAAGTGATCAAAGAGTTCATGCGGTTTA |
| | | AGGTGAGAATGGAAGGAAGCATGAACGGCCACGAGTTCGAAATTGAGGGAGA |
| | | AGGAGAGGGACGGCCCTACGAGGGCACCCAGACAGCCAAGCTGAAAGTGACA |
| | | AAGGGCGGGCCTCTGCCATTCGCTTGGGACATCCTGAGCCCACAGTTTATGT |
| | | ACGGCTCCAAGGCCTATGTGAAACATCCAGCTGACATTCCCGATTATAAGAA |
| | | ACTGAGCTTCCCCGAGGGGTTTAAGTGGGAAAGAGTGATGAACTTCGAGGAC |
| | | GGAGGCCTGGTGACTGTGACCCAGGACAGCTCCCTGCAGGATGGGACCCTGA |
| | | TCTACAAGGTGAAAATGAGAGGGACAAATTTTCCCCCTGATGGACCTGTGAT |
| | | GCAGAAGAAAACTATGGGATGGGAGGCCTCCACCGAAAGGCTGTATCCACGC |
| | | GACGGGGTGCTGAAAGGAGAAATCCACCAGGCTCTGAAGCTGAAAGATGGGG |
| | | GACATTACCTGGTGGAGTTCAAGACAATCTACATGGCCAAGAAACCTGTGCA |
| | | GCTGCCAGGCTACTATTACGTGGACACAAAACTGGATATCACTTCACACAAC |
| | | GAGGACTACACTATTGTGGAGCAGTATGAACGGAGCGAGGGGAGACACCATC |
| | | TGTTCCTGGGCCATGGGACTGGAAGTACCGGCTCAGGGTCTAGTGGAACCGC |
| | | CTCAAGCGAGGATAACAATATGGCTGTGATCAAAGAGTTCATGAGGTTTAAG |
| | | GTGCGCATGGAGGGCAGCATGAATGGGCACGAATTTGAGATTGAAGGAGAGG |
| | | GCGAAGGGAGGCCTTACGAGGGCACACAGACTGCCAAGCTGAAAGTGACCAA |
| | | GGGAGGACCACTGCCTTTCGCTTGGGATATCCTGTCTCCTCAGTTTATGTAC |
| | | GGAAGTAAGGCCTATGTCAAGCATCCCGCTGACATTCCTGATTACAAGAAAC |
| | | TGTCTTTCCCAGAGGGCTTTAAGTGGGAGAGAGTGATGAATTTTGAAGATGG |
| | | AGGCCTGGTGACCGTGACACAGGACTCCTCTCTGCAGGATGGCACTCTGATC |
| | | TACAAAGTCAAAATGCGCGGCACCAATTTTCCACCCGATGGGCCCGTGATGC |
| | | AGAAGAAAACAATGGGGTGGGAGGCCAGCACTGAACGGCTGTATCCTAGAGA |
| | | CGGAGTGCTGAAGGGCGAAATCCACCAGGCCCTGAAGCTGAAAGACGGCGGC |
| | | CACTACCTGGTGGAGTTCAAAACCATCTACATGGCCAAGAAACCAGTGCAGC |
| | | TGCCCGGCTATTACTATGTGGACACCAAGCTGGATATCACATCCCACAATGA |
| | | AGACTACACCATTGTGGAACAGTATGAGAGGTCTGAAGGACGCCACCATCTG |
| | | TTTCTGTACGGCATGGATGAGCTGTATAAGTAAAGAAGCTTGCCTCGAGCAG |
| | | CGCTTATCGATAATCAACCTCTGGATTACAAAATTTGTGAAAGATTGACTGG |
| | | TATTCTTAACTATGTTGCTCCTTTTACGCTATGTGGATACGCTGCTTTAATG |
| | | CCTTTGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGTCAGGCA |
| | | ACGTGGCGTGGTGTGCACTGTGTTTGCTGACGCAACCCCCACTGGTTGGGGC |
| | | ATTGCCACCACCTGTCAGCTCCTTTCCGGGACTTTCGCTTTCCCCCTCCCTA |
| | | TTGCCACGGCGGAACTCATCGCCGCCTGCCTTGCCCGCTGCTGGACAGGGGC |
| | | TCGGCTGTTGGGCACTGACAATTCCGTGGTGTTGTCGGGGAAATCATCGTCC |
| | | TTTCCTTGGCTGCTCGCCTATGTTGCCACCTGGATTCTGCGCGGGACGTCCT |
| | | TCTGCTACGTCCCTTCGGCCCTCAATCCAGCGGACCTTCCTTCCCGCGGCCT |
| | | GCTGCCGGCTCTGCGGCCTCTTCCGCGTCTTCGCCTTCGCCCTCAGACGAGT |
| | | CGGATCTCCCTTTGGGCCGCCTCCCCGCATCGATACCGGCTGCTCGAGAGAT |
| | | CTACGGGTGGCATCCCTGTGACCCCTCCCCAGTGCCTCTCCTGGCCCTGGAA |
| | | GTTGCCACTCCAGTGCCCACCAGCCTTGTCCTAATAAAATTAAGTTGCATCA |
| | | TTTTGTCTGACTAGGTGTCCTTCTATAATATTATGGGGTGGAGGGGGGTGGT |
| | | ATGGAGCAAGGGGCAAGTTGGGAAGACAACCTGTAGGGCCTGCGGGGTCTAT |
| | | TGGGAACCAAGCTGGAGTGCAGTGGCACAATCTTGGCTCACTGCAATCTCCG |
| | | CCTCCTGGGTTCAAGCGATTCTCCTGCCTCAGCCTCCCGAGTTGTTGGGATT |
| | | CCAGGCATGCATGACCAGGCTCAGCTAATTTTTGTTTTTTTGGTAGAGACGG |
| | | GGTTTCACCATATTGGCCAGGCTGGTCTCCAACTCCTAATCTCAGGTGATCT |
| | | ACCCACCTTGGCCTCCCAAATTGCTGGGATTACAGGCGTGAACCACTGCTCC |
| | | CTTCCCTGTCCTTCTGATTTTGTAGGTAACCACGTGCGGACCGAGCGGCCGC |
| | | AGGAACCCCTAGTGATGGAGTTGGCCACTCCCTCTCTGCGCGCTCGCTCGCT |
| | | CACTGAGGCCGGGCGACCAAAGGTCGCCCGACGCCCGGGCTTTGCCCGGGCG |
| | | GCCTCAGTGAGCGAGCGAGCGCGCAGCTGCCTGCAGG |
| 5 | ProA7-vfChrimsonR- | TCCTGCAGGCAGCTGCGCGCTCGCTCGCTCACTGAGGCCGCCCGGGCGTCGG |
| | EYFP-WPRE vector | GCGACCTTTGGTCGCCCGGCCTCAGTGAGCGAGCGAGCGCGCAGAGAGGGAG |
| | | TGGCCAACTCCATCACTAGGGGTTCCTGCGGCCGCACGCGTCATCCTGAGAG |
| | | ATGAGCCAGGACAAAGAACCAGTAATAGCTCCTGGAGCAGCACATCTGTTTT |
| | | GCCAGGATTATCCCTTGGATCTCTTAAAACCGAGACCTTGTAATCTGAAGAC |
| | | TCAACTTGGGCTGTACCCTTAACCTTCAGCTCTATGATGCAAGTGAGTCCAC |
| | | AGGACCGGAGGCTTTGAGATGAGCTTTTCAGAAGGGAGGAGTTGGCCGCTTG |
| | | CTCCCAGAGCTCCAGCACCTGCATTCTTCTGGCTATGTCAGAAGCCAGATCA |
| | | TTTCCCTCGTTAAAAACAAAAACAAAAAAACAAACAAACAAAATGTTAGTCT |
| | | TTGCCCTTTATCTGCCTGGCAAAGCTTTTAATTGGCTTGATCTGTCATTCCG |
| | | CTAGACATAAAGGGGACAATCCCCGGATTAGGAAGGAGCTCTCCAGCTCGGG |
| | | TAAGGAGTCTCAAGGCAAGGTAGGCAAGCACCACCGGTCCGCACTCTCGCCC |
| | | AGCTTTTACGGGAAGAAGAGAGGATCCGCCACCATGGCTGAGCTGATCAGCA |
| | | GCGCCACCAGATCTCTGTTTGCCGCCGGAGGCATCAACCCTTGGCCTAACCC |
| | | CTACCACCACGAGGACATGGGCTGTGGAGGAATGACACCTACAGGCGAGTGC |
| | | TTCAGCACCGAGTGGTGGTGTGACCCTTCTTACGGACTGAGCGACGCCGGAT |
| | | ACGGATATTGCTTCGTGGAGGCCACAGGCGGCTACCTGGTCGTGGGAGTGGA |
| | | GAAGAAGCAGGCTTGGCTGCACAGCAGAGGCACACCAGGAGAAAAGATCGGC |
| | | GCCCAGGTCTGCCAGTGGATTGCTTTCAGCATCGCCATCGCCCTGCTGACAT |
| | | TCTACGGCTTCAGCGCCTGGAAGGCCACTTGCGGTTGGGAGGAGGTCTACGT |
| | | CTGTTGCGTCGAGGTGCTGTTCGTGACCCTGGAGATCTTCAAGGAGTTCAGC |
| | | AGCCCCGCCACAGTGTACCTGTCTACCGGCAACCACGCCTATTGCCTGCGCT |
| | | ACTTCGAGTGGCTGCTGTCTTGCCCCGTGATCCTGATCAGACTGAGCAACCT |
| | | GAGCGGCCTGAAGAACGACTACAGCAAGCGGACCATGGGCCTGATCGTGTCT |
| | | TGCGTGGGAATGATCGTGTTCGGCATGGCCGCAGGACTGGCTACCGATTGGC |
| | | TCAAGTGGCTGCTGTATATCGTGTCTTGCATCTACGGCGGCTACATGTACTT |
| | | CCAGGCCGCCAAGTGCTACGTGGAAGCCAACCACAGCGTGCCTAAAGGCCAT |
| | | TGCCGCATGGTCGTGAAGCTGATGGCCTACGCTTTCTTCGCCTCTTGGGGCA |
| | | TGTACCCAATCCTCTGGGCAGTGGGACCAGAAGGACTGCTGAAGCTGAGCCC |
| | | TTACGCCAACAGCATCGGCCACAGCATCTGCGACATCATCGCCAAGGAGTTT |
| | | TGGACCTTCCTGGCCCACCACCTGAGGATCAAGATCCACGAGCACATCCTGA |
| | | TCCACGGCGACATCCGGAAGACCACCAAGATGGAGATCGGAGGCGAGGAGGT |
| | | GGAAGTGGAAGAGTTCGTGGAGGAGGAGGACGAGGACACAGTGACCGCGGCC |
| | | GCCACCATGGTGAGCAAGGGCGAGGAGCTGTTCACCGGGGTGGTGCCCATCC |
| | | TGGTCGAGCTGGACGGCGACGTAAACGGCCACAAGTTCAGCGTGTCCGGCGA |
| | | GGGCGAGGGCGATGCCACCTACGGCAAGCTGACCCTGAAGTTCATCTGCACC |
| | | ACCGGCAAGCTGCCCGTGCCCTGGCCCACCCTCGTGACCACCTTCGGCTACG |
| | | GCCTGCAGTGCTTCGCCCGCTACCCCGACCACATGAAGCAGCACGACTTCTT |
| | | CAAGTCCGCCATGCCCGAAGGCTACGTCCAGGAGCGCACCATCTTCTTCAAG |
| | | GACGACGGCAACTACAAGACCCGCGCCGAGGTGAAGTTCGAGGGCGACACCC |
| | | TGGTGAACCGCATCGAGCTGAAGGGCATCGACTTCAAGGAGGACGGCAACAT |
| | | CCTGGGGCACAAGCTGGAGTACAACTACAACAGCCACAACGTCTATATCATG |
| | | GCCGACAAGCAGAAGAACGGCATCAAGGTGAACTTCAAGATCCGCCACAACA |
| | | TCGAGGACGGCAGCGTGCAGCTCGCCGACCACTACCAGCAGAACACCCCCAT |
| | | CGGCGACGGCCCCGTGCTGCTGCCCGACAACCACTACCTGAGCTACCAGTCC |
| | | GCCCTGAGCAAAGACCCCAACGAGAAGCGCGATCACATGGTCCTGCTGGAGT |
| | | TCGTGACCGCCGCCGGGATCACTCTCGGCATGGACGAGCTGTACAAGTAAAG |
| | | CGGCCGCCGGCAAGTAAGCTTATCGATAATCAACCTCTGGATTACAAAATTT |
| | | GTGAAAGATTGACTGGTATTCTTAACTATGTTGCTCCTTTTACGCTATGTGG |
| | | ATACGCTGCTTTAATGCCTTTGTATCATGCTATTGCTTCCCGTATGGCTTTC |
| | | ATTTTCTCCTCCTTGTATAAATCCTGGTTGCTGTCTCTTTATGAGGAGTTGT |
| | | GGCCCGTTGTCAGGCAACGTGGCGTGGTGTGCACTGTGTTTGCTGACGCAAC |
| | | CCCCACTGGTTGGGGCATTGCCACCACCTGTCAGCTCCTTTCCGGGACTTTC |
| | | GCTTTCCCCCTCCCTATTGCCACGGCGGAACTCATCGCCGCCTGCCTTGCCC |
| | | GCTGCTGGACAGGGGCTCGGCTGTTGGGCACTGACAATTCCGTGGTGTTGTC |
| | | GGGGAAATCATCGTCCTTTCCTTGGCTGCTCGCCTATGTTGCCACCTGGATT |
| | | CTGCGCGGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCTTCGCCT |
| | | TCGCCCTCAGACGAGTCGGATCTCCCTTTGGGCCGCCTCCCCGCATCGATAC |
| | | CGAGCGCTGCTCGAGAGATCTACGGGTGGCATCCCTGTGACCCCTCCCCAGT |
| | | GCCTCTCCTGGCCCTGGAAGTTGCCACTCCAGTGCCCACCAGCCTTGTCCTA |
| | | ATAAAATTAAGTTGCATCATTTTGTCTGACTAGGTGTCCTTCTATAATATTA |
| | | TGGGGTGGAGGGGGGTGGTATGGAGCAAGGGGCAAGTTGGGAAGACAACCTG |
| | | TAGGGCCTGCGGGGTCTATTGGGAACCAAGCTGGAGTGCAGTGGCACAATCT |
| | | TGGCTCACTGCAATCTCCGCCTCCTGGGTTCAAGCGATTCTCCTGCCTCAGC |
| | | CTCCCGAGTTGTTGGGATTCCAGGCATGCATGACCAGGCTCAGCTAATTTTT |
| | | GTTTTTTTGGTAGAGACGGGGTTTCACCATATTGGCCAGGCTGGTCTCCAAC |
| | | TCCTAATCTCAGGTGATCTACCCACCTTGGCCTCCCAAATTGCTGGGATTAC |
| | | AGGCGTGAACCACTGCTCCCTTCCCTGTCCTTCTGATTTTGTAGGTAACCAC |
| | | GTGCGGACCGAGCGGCCGCAGGAACCCCTAGTGATGGAGTTGGCCACTCCCT |
| | | CTCTGCGCGCTCGCTCGCTCACTGAGGCCGGGCGACCAAAGGTCGCCCGACG |
| | | CCCGGGCTTTGCCCGGGCGGCCTCAGTGAGCGAGCGAGCGCGCAGCTGCCTG |
| | | C |
| 6 | ProA7-ChrMine- | GCAGCTGCGCGCTCGCTCGCTCACTGAGGCCGCCCGGGCGTCGGGCGACCTT |
| | EYFP-WPRE | TGGTCGCCCGGCCTCAGTGAGCGAGCGAGCGCGCAGAGAGGGAGTGGCCAAC |
| | | TCCATCACTAGGGGTTCCTGCGGCCGCACGCGTCATCCTGAGAGATGAGCCA |
| | | GGACAAAGAACCAGTAATAGCTCCTGGAGCAGCACATCTGTTTTGCCAGGAT |
| | | TATCCCTTGGATCTCTTAAAACCGAGACCTTGTAATCTGAAGACTCAACTTG |
| | | GGCTGTACCCTTAACCTTCAGCTCTATGATGCAAGTGAGTCCACAGGACCGG |
| | | AGGCTTTGAGATGAGCTTTTCAGAAGGGAGGAGTTGGCCGCTTGCTCCCAGA |
| | | GCTCCAGCACCTGCATTCTTCTGGCTATGTCAGAAGCCAGATCATTTCCCTC |
| | | GTTAAAAACAAAAACAAAAAAACAAACAAACAAAATGTTAGTCTTTGCCCTT |
| | | TATCTGCCTGGCAAAGCTTTTAATTGGCTTGATCTGTCATTCCGCTAGACAT |
| | | AAAGGGGACAATCCCCGGATTAGGAAGGAGCTCTCCAGCTCGGGTAAGGAGT |
| | | CTCAAGGCAAGGTAGGCAAGCACCACCGGTCCGCACTCTCGCCCAGCTTTTA |
| | | CGGGAAGAAGAGAGGATCCGCCACCATGGCCCACGCCCCCGGCACCGACCAG |
| | | ATGTTCTACGTGGGCACCATGGACGGCTGGTACCTGGACACCAAGCTGAACA |
| | | GCGTGGCCATCGGCGCCCACTGGAGCTGCTTCATCGTGCTGACCATCACCAC |
| | | CTTCTACCTGGGCTACGAGAGCTGGACCAGCAGGGGCCCCAGCAAGAGGACC |
| | | AGCTTCTACGCCGGCTACCAGGAGGAGCAGAACCTGGCCCTGTTCGTGAACT |
| | | TCTTCGCCATGCTGAGCTACTTCGGCAAGATCGTGGCCGACACCCTGGGCCA |
| | | CAACTTCGGCGACGTGGGCCCCTTCATCATCGGCTTCGGCAACTACAGGTAC |
| | | GCCGACTACATGCTGACCTGCCCCATGCTGGTGTACGACCTGCTGTACCAGC |
| | | TGAGGGCCCCCTACAGGGTGAGCTGCAGCGCCATCATCTTCGCCATCCTGAT |
| | | GAGCGGCGTGCTGGCCGAGTTCTACGCCGAGGGCGACCCCAGGCTGAGGAAC |
| | | GGCGCCTACGCCTGGTACGGCTTCGGCTGCTTCTGGTTCATCTTCGCCTACA |
| | | GCATCGTGATGAGCATCGTGGCCAAGCAGTACAGCAGGCTGGCCCAGCTGGC |
| | | CCAGGACACCGGCGCCGAGCACAGCCTGCACGTGCTGAAGTTCGCCGTGTTC |
| | | ACCTTCAGCATGCTGTGGATTCTGTTCCCCCTGGTGTGGGCCATCTGCCCCA |
| | | GGGGCTTCGGCTGGATCGACGACAACTGGACCGAGGTGGCCCACTGCGTGTG |
| | | CGACATCGTGGCCAAGAGCTGCTACGGCTTCGCCCTGGCCAGGTTCAGGAAG |
| | | ACCTACGACGAGGAGCTGTTCAGGCTGCTGGAGCAGCTGGGCCACGACGAGG |
| | | ACGAGTTCCAGAAGCTGGAGCTGGACATGAGGCTGAGCAGCAACGGCGAGAG |
| | | GCTGAGGAGGCTGAGCGCGGCACCGGTAGTAGCAATGGTGAGCAAGGGCGAG |
| | | GAGCTGTTCACCGGGGTGGTGCCCATCCTGGTCGAGCTGGACGGCGACGTAA |
| | | ACGGCCACAAGTTCAGCGTGTCCGGCGAGGGCGAGGGCGATGCCACCTACGG |
| | | CAAGCTGACCCTGAAGTTCATCTGCACCACCGGCAAGCTGCCCGTGCCCTGG |
| | | CCCACCCTCGTGACCACCTTCGGCTACGGCCTGCAGTGCTTCGCCCGCTACC |
| | | CCGACCACATGAAGCAGCACGACTTCTTCAAGTCCGCCATGCCCGAAGGCTA |
| | | CGTCCAGGAGCGCACCATCTTCTTCAAGGACGACGGCAACTACAAGACCCGC |
| | | GCCGAGGTGAAGTTCGAGGGCGACACCCTGGTGAACCGCATCGAGCTGAAGG |
| | | GCATCGACTTCAAGGAGGACGGCAACATCCTGGGGCACAAGCTGGAGTACAA |
| | | CTACAACAGCCACAACGTCTATATCATGGCCGACAAGCAGAAGAACGGCATC |
| | | AAGGTGAACTTCAAGATCCGCCACAACATCGAGGACGGCAGCGTGCAGCTCG |
| | | CCGACCACTACCAGCAGAACACCCCCATCGGCGACGGCCCCGTGCTGCTGCC |
| | | CGACAACCACTACCTGAGCTACCAGTCCGCCCTGAGCAAAGACCCCAACGAG |
| | | AAGCGCGATCACATGGTCCTGCTGGAGTTCGTGACCGCCGCCGGGATCACTC |
| | | TCGGCATGGACGAGCTGTACAAGTAAAGCGGCCGCCGGCAAGTAAGCTTATC |
| | | GATAATCAACCTCTGGATTACAAAATTTGTGAAAGATTGACTGGTATTCTTA |
| | | ACTATGTTGCTCCTTTTACGCTATGTGGATACGCTGCTTTAATGCCTTTGTA |
| | | TCATGCTATTGCTTCCCGTATGGCTTTCATTTTCTCCTCCTTGTATAAATCC |
| | | TGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCAC |
| | | CACCTGTCAGCTCCTTTCCGGGACTTTCGCTTTCCCCCTCCCTATTGCCACG |
| | | GCGGAACTCATCGCCGCCTGCCTTGCCCGCTGCTGGACAGGGGCTCGGCTGT |
| | | TGGGCACTGACAATTCCGTGGTGTTGTCGGGGAAATCATCGTCCTTTCCTTG |
| | | GCTGCTCGCCTATGTTGCCACCTGGATTCTGCGCGGGACGTCCTTCTGCTAC |
| | | GTCCCTTCGGCCCTCAATCCAGCGGACCTTCCTTCCCGCGGCCTGCTGCCGG |
| | | CTCTGCGGCCTCTTCCGCGTCTTCGCCTTCGCCCTCAGACGAGTCGGATCTC |
| | | CCTTTGGGCCGCCTCCCCGCATCGATACCGAGCGCTGCTCGAGAGATCTACG |
| | | GGTGGCATCCCTGTGACCCCTCCCCAGTGCCTCTCCTGGCCCTGGAAGTTGC |
| | | CACTCCAGTGCCCACCAGCCTTGTCCTAATAAAATTAAGTTGCATCATTTTG |
| | | TCTGACTAGGTGTCCTTCTATAATATTATGGGGTGGAGGGGGGTGGTATGGA |
| | | GCAAGGGGCAAGTTGGGAAGACAACCTGTAGGGCCTGCGGGGTCTATTGGGA |
| | | ACCAAGCTGGAGTGCAGTGGCACAATCTTGGCTCACTGCAATCTCCGCCTCC |
| | | TGGGTTCAAGCGATTCTCCTGCCTCAGCCTCCCGAGTTGTTGGGATTCCAGG |
| | | CATGCATGACCAGGCTCAGCTAATTTTTGTTTTTTTGGTAGAGACGGGGTTT |
| | | CACCATATTGGCCAGGCTGGTCTCCAACTCCTAATCTCAGGTGATCTACCCA |
| | | CCTTGGCCTCCCAAATTGCTGGGATTACAGGCGTGAACCACTGCTCCCTTCC |
| | | CTGTCCTTCTGATTTTGTAGGTAACCACGTGCGGACCGAGCGGCCGCAGGAA |
| | | CCCCTAGTGATGGAGTTGGCCACTCCCTCTCTGCGCGCTCGCTCGCTCACTG |
| | | AGGCCGGGCGACCAAAGGTCGCCCGACGCCCGGGCTTTGCCCGGGCGGCCTC |
| | | AGTGAGCGAGCGAGCGCGCAGCTGCCTGCAGGGGCGCC |
| 7 | ProA7-ReaChR- | GTCCTGCAGGCAGCTGCGCGCTCGCTCGCTCACTGAGGCCGCCCGGGCGTCG |
| | Ctirine-WPRE | GGCGACCTTTGGTCGCCCGGCCTCAGTGAGCGAGCGAGCGCGCAGAGAGGGA |
| | | GTGGCCAACTCCATCACTAGGGGTTCCTGCGGCCGCACGCGTCATCCTGAGA |
| | | GATGAGCCAGGACAAAGAACCAGTAATAGCTCCTGGAGCAGCACATCTGTTT |
| | | TGCCAGGATTATCCCTTGGATCTCTTAAAACCGAGACCTTGTAATCTGAAGA |
| | | CTCAACTTGGGCTGTACCCTTAACCTTCAGCTCTATGATGCAAGTGAGTCCA |
| | | CAGGACCGGAGGCTTTGAGATGAGCTTTTCAGAAGGGAGGAGTTGGCCGCTT |
| | | GCTCCCAGAGCTCCAGCACCTGCATTCTTCTGGCTATGTCAGAAGCCAGATC |
| | | ATTTCCCTCGTTAAAAACAAAAACAAAAAAACAAACAAACAAAATGTTAGTC |
| | | TTTGCCCTTTATCTGCCTGGCAAAGCTTTTAATTGGCTTGATCTGTCATTCC |
| | | GCTAGACATAAAGGGGACAATCCCCGGATTAGGAAGGAGCTCTCCAGCTCGG |
| | | GTAAGGAGTCTCAAGGCAAGGTAGGCAAGCACCACCGGTCCGCACTCTCGCC |
| | | CAGCTTTTACGGGAAGAAGAGAGGATCCGCCACCATGGTGAGCAGAAGACCC |
| | | TGGCTGCTGGCCCTGGCCCTGGCCGTGGCCCTGGCCGCCGGCAGCGCCGGCG |
| | | CCAGCACCGGCAGCGACGCCACCGTGCCCGTGGCCACCCAGGACGGCCCCGA |
| | | CTACGTGTTCCACAGAGCCCACGAGAGAATGCTGTTCCAGACCAGCTACACC |
| | | CTGGAGAACAACGGCAGCGTGATCTGCATCCCCAACAACGGCCAGTGCTTCT |
| | | GCCTGGCCTGGCTGAAGAGTAACGGCACCAACGCCGAGAAGCTGGCCGCCAA |
| | | CATCCTGCAGTGGGTGGTGTTTGCGCTGAGCGTGGCGTGCCTGGGCTGGTAT |
| | | GCGTATCAGGCGTGGCGCGCGACCTGCGGCTGGGAAGAAGTGTATGTGGCGC |
| | | TGATTGAAATGATGAAAAGCATTATTGAAGCGTTTCATGAATTTGATAGCCC |
| | | GGCGACCCTGTGGCTGAGCAGCGGCAACGGCGTGGTGTGGATGCGCTATGGC |
| | | GAATGGCTGCTGACCTGCCCCGTGATTCTGATTCATCTGAGCAACCTGACCG |
| | | GCCTGAAAGATGATTATAGCAAACGCACCATGGGCCTGCTGGTGAGCGACGT |
| | | GGGCTGCATTGTGTGGGGCGCGACCAGCGCGATGTGCACCGGCTGGACCAAA |
| | | ATTCTGTTTTTTCTGATTAGCCTGAGCTATGGCATGTATACCTATTTTCATG |
| | | CGGCCAAAGTGTATATTGAAGCGTTTCATACCGTGCCGAAAGGCCTGTGCAG |
| | | ACAGCTGGTGAGAGCCATGGCCTGGCTGTTCTTCGTGAGCTGGGGCATGTTC |
| | | CCCGTGCTGTTCCTGCTGGGCCCCGAGGGCTTCGGCCATATTAGCCCGTATG |
| | | GCAGCGCGATTGGCCATAGCATTCTGGATCTGATTGCGAAGAACATGTGGGG |
| | | CGTGCTGGGCAACTATCTGCGCGTGAAAATTCATGAACATATTCTGCTGTAT |
| | | GGCGATATTCGCAAAAAACAGAAAATTACCATTGCGGGCCAGGAAATGGAAG |
| | | TGGAAACCCTGGTGGCGGAAGAAGAAGATAAGTACGAGAGCAGCCTCGAGAT |
| | | GGTGAGCAAGGGCGAGGAGCTGTTCACCGGGGTGGTGCCCATCCTGGTCGAG |
| | | CTGGACGGCGACGTAAACGGCCACAAGTTCAGCGTGTCCGGCGAGGGCGAGG |
| | | GCGATGCCACCTACGGCAAGCTGACCCTGAAGTTCATCTGCACCACCGGCAA |
| | | GCTGCCCGTGCCCTGGCCCACCCTCGTGACCACCTTCGGCTACGGCCTGATG |
| | | TGCTTCGCCCGCTACCCCGACCACATGAAGCAGCACGACTTCTTCAAGTCCG |
| | | CCATGCCCGAAGGCTACGTCCAGGAGCGCACCATCTTCTTCAAGGACGACGG |
| | | CAACTACAAGACCCGCGCCGAGGTGAAGTTCGAGGGCGACACCCTGGTGAAC |
| | | CGCATCGAGCTGAAGGGCATCGACTTCAAGGAGGACGGCAACATCCTGGGGC |
| | | ACAAGCTGGAGTACAACTACAACAGCCACAACGTCTATATCATGGCCGACAA |
| | | GCAGAAGAACGGCATCAAGGTGAACTTCAAGATCCGCCACAACATCGAGGAC |
| | | GGCAGCGTGCAGCTCGCCGACCACTACCAGCAGAACACCCCCATCGGCGACG |
| | | GCCCCGTGCTGCTGCCCGACAACCACTACCTGAGCTACCAGTCCGCCCTGAG |
| | | CAAAGACCCCAACGAGAAGCGCGATCACATGGTCCTGCTGGAGTTCGTGACC |
| | | GCCGCCGGGATCACTCTCGGCATGGACGAGCTGTACAAGTAAAGCGGCCGCC |
| | | GGCAAGTAAGCTTATCGATAATCAACCTCTGGATTACAAAATTTGTGAAAGA |
| | | TTGACTGGTATTCTTAACTATGTTGCTCCTTTTACGCTATGTGGATACGCTG |
| | | CTTTAATGCCTTTGTATCATGCTATTGCTTCCCGTATGGCTTTCATTTTCTC |
| | | CTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTG |
| | | GTTGGGGCATTGCCACCACCTGTCAGCTCCTTTCCGGGACTTTCGCTTTCCC |
| | | CCTCCCTATTGCCACGGCGGAACTCATCGCCGCCTGCCTTGCCCGCTGCTGG |
| | | ACAGGGGCTCGGCTGTTGGGCACTGACAATTCCGTGGTGTTGTCGGGGAAAT |
| | | CATCGTCCTTTCCTTGGCTGCTCGCCTATGTTGCCACCTGGATTCTGCGCGG |
| | | GACGTCCTTCTGCTACGTCCCTTCGGCCCTCAATCCAGCGGACCTTCCTTCC |
| | | CGCGGCCTGCTGCCGGCTCTGCGGCCTCTTCCGCGTCTTCGCCTTCGCCCTC |
| | | AGACGAGTCGGATCTCCCTTTGGGCCGCCTCCCCGCATCGATACCGAGCGCT |
| | | GCTCGAGAGATCTACGGGTGGCATCCCTGTGACCCCTCCCCAGTGCCTCTCC |
| | | TGGCCCTGGAAGTTGCCACTCCAGTGCCCACCAGCCTTGTCCTAATAAAATT |
| | | AAGTTGCATCATTTTGTCTGACTAGGTGTCCTTCTATAATATTATGGGGTGG |
| | | AGGGGGGTGGTATGGAGCAAGGGGCAAGTTGGGAAGACAACCTGTAGGGCCT |
| | | GCGGGGTCTATTGGGAACCAAGCTGGAGTGCAGTGGCACAATCTTGGCTCAC |
| | | TGCAATCTCCGCCTCCTGGGTTCAAGCGATTCTCCTGCCTCAGCCTCCCGAG |
| | | TTGTTGGGATTCCAGGCATGCATGACCAGGCTCAGCTAATTTTTGTTTTTTT |
| | | GGTAGAGACGGGGTTTCACCATATTGGCCAGGCTGGTCTCCAACTCCTAATC |
| | | TCAGGTGATCTACCCACCTTGGCCTCCCAAATTGCTGGGATTACAGGCGTGA |
| | | ACCACTGCTCCCTTCCCTGTCCTTCTGATTTTGTAGGTAACCACGTGCGGAC |
| | | CGAGCGGCCGCAGGAACCCCTAGTGATGGAGTTGGCCACTCCCTCTCTGCGC |
| | | GCTCGCTCGCTCACTGAGGCCGGGCGACCAAAGGTCGCCCGACGCCCGGGCT |
| | | TTGCCCGGGCGGCCTCAGTGAGCGAGCGAGCGCGCAGCTGCCTGCAGGG |
| 8 | Woodchuck hepatitis | AATCAACCTCTGGATTACAAAAATTTGTGAAAGATTGACTGGTATTCTTAAC |
| | virus with X mutation | TATGTTGCTCCTTTTACGCTATGTGGATACGCTGCTTTAATGCCTTTGTATC |
| | | ATGCTATTGCTTCCCGTATGGCTTTCATTTTCTCCTCCTTGTATAAATCCTG |
| | | GTTGCTGTCTCTTTATGAGGAGTTGTGGCCCGTTGTCAGGCAACGTGGCGTG |
| | | GTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGC |
| | | GGAACTCATCGCCGCCTGCCTTGCCCGCTGCTGGACAGGGGCTCGGCTGTTG |
| | | GGCACTGACAATTCCGTGGTGTTGTCGGGGAAGGTCTGCTGAGACTCGGGGC |
| | | TGCTCGCCTGTGTTGCCACCTGGATTCTGCGCGGGACGTCCTTCTGCTACGT |
| | | CCCTTCGGCCCTCAATCCAGCGGACCTTCCTTCCCGCGGCCTGCTGCCGGCT |
| | | CTGCGGCCTCTTCCGCGTCTTCGCCTTCGCCCTCAGACGAGTCGGATCTCCC |
| | | TTTGGGCCGCCTCCCCGCCTG |
| 9 | Human growth | ACGGGTGGCATCCCTGTGACCCCTCCCCAGTGCCTCTCCTGGCCCTGGAAGT |
| | hormone poly A | TGCCACTCCAGTGCCCACCAGCCTTGTCCTAATAAAATTAAGTTGCATCATT |
| | | TTGTCTGACTAGGTGTCCTTCTATAATATTATGGGGTGGAGGGGGGTGGTAT |
| | | GGAGCAAGGGGCAAGTTGGGAAGACAACCTGTAGGGCCTGCGGGGTCTATTG |
| | | GGAACCAAGCTGGAGTGCAGTGGCACAATCTTGGCTCACTGCAATCTCCGCC |
| | | TCCTGGGTTCAAGCGATTCTCCTGCCTCAGCCTCCCGAGTTGTTGGGATTCC |
| | | AGGCATGCATGACCAGGCTCAGCTAATTTTTGTTTTTTTGGTAGAGACGGGG |
| | | TTTCACCATATTGGCCAGGCTGGTCTCCAACTCCTAATCTCAGGTGATCTAC |
| | | CCACCTTGGCCTCCCAAATTGCTGGGATTACAGGCGTGAACCACTGCTCCCT |
| | | TCCCTGTCCTT |
| 10 | ProA330 (rod-specific | AACCCAAGAAATTACAGGCTGAAACCAGAAAAGAACACATTAAAGC |
| | promoter) | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCAGTCTTCTGTGTCAGATAT |
| | | TTAAGAAAACTAACAGAGGTCAGAGAAGACACACCTACAGCAAGTA |
| | | GACTGTCCCTGTGCTGCCTTTTTGCAACCCCTGCTTTGGCAGTGCT |
| | | CAAGCCCACCTCCTGCTCTGTGCAGACATCTCTTCTTTGCTCTTAC |
| | | TAGACCAAGGTGAAAGAAAACTCTCACCTTCTCCCATCTGGCCCCA |
| | | CAGCATCTGGAACACACTGATCCTCATAATCCTTGTTCTTGAGAAA |
| | | TATTAATGACTTAATCTCCCAAGCTTGCTCCCTCTCCTGTGCAGGC |
| | | CATCTCAGTATGTTTTGCAGACAAGACCCAGAGAAGTCCAGACTGG |
| | | ACTTGTTGCAGACTGCAAAACTGCCATTGGAAGGCCTCCGTCCCAG |
| | | TCCTTCTACAGAGTAGCCAGTGGGATTCCCAGCC |
| 11 | ProX572 (also referred | CATCCTGAGAGATGAGCCAGGACAAAGAACCAGTAATAGCTCCTGG |
| | to as Pro572) | AGCAGCACATCTGTTTTGCCAGGATTATCCCTTGGATCTCTTAAAA |
| | | CCGAGACCTTGTAATCTGAAGACTCAACTTGGGCTGTACCCTTAAC |
| | | CTTCAGCTCTATGATGCAAGTGAGTCCACAGGACCGGAGGCTTTGA |
| | | GATGAGCTTTTCAGAAGGGAGGAGTTGGCCGCTTGCTCCCAGAGCT |
| | | CCAGCACCTGCATTCTTCTGGCTATGTCAGAAGCCAGATCATTTCC |
| | | CTCGTTAAAAACAAAAACAAAAAAACAAACAAACAAAATGTTAGTC |
| | | TTTGCCCTTTATCTGCCTGGCAAAGCTTTTAATTGGCTTGATCTGT |
| | | CATTCCGCTAGACATAAAGGGGACAATCCCCGGATTAGGAAGGAGC |
| | | TCTCCAGCTCGGGTAAGGAGTCTCAAGGCAAGGTAGGCAAGCACCA |
| | | CCGGTCCGCACTCTCGCCCAGCTTTTACGGGAAGAAGAGAAACCCA |
| | | AGAAATTACAGGCTGAAACCAGAAAAGAACACATTAAAGCACCAAG |
| | | AGAAAGTTGGAGTGGGTTGAAGGGAAACAGATTTTTAAAGTTAAGG |
| | | CTCTGTGAAATGGGTAGAATTAACTACAGGTTAAAAATAAAATGTT |
| | | AACTAAAGGTTGCCTCTGAGTAACAGGATTATGGGTGATTTTAATT |
| | | GTCTTCTTTGTGTATGTTCAACAGTGACTATAATATGTATTACTTT |
| | | TGGAATAAAGGAAAACCTGAAAGGTGTGTTGTTTTATAAGGGCCCT |
| | | TAGGTTGCCAAAATTAGAGTCATTGAAATCTAAAGCTGATAAAAAC |
| | | TTTAGTGCAAAGATTGTGACATGGGAGACTACACATACCAGATCCA |
| | | TAATGTACATGAGGACAGTAGGCCGAGGGGCCCTGCACATTGAAAG |
| | | CCCACATGGGAGAAGCCCTTGGGAAGGGGAGTGGAAGGATGAGGCA |
| | | AGGGGCCGGGGGGATGCAGAGGCTGGCAGGCAGTCATTTCTCAGCT |
| | | TCAGCCATTCCCGCCATGGGGGAATGTGGACAGAGAAGCCAAACAA |
| | | ATCTCCTAAACAGTAAATGTCAGTCTTCTGTGTCAGATATTTAAGA |
| | | AAACTAACAGAGGTCAGAGAAGACACACCTACAGCAAGTAGACTGT |
| | | CCCTGTGCTGCCTTTTTGCAACCCCTGCTTTGGCAGTGCTCAAGCC |
| | | CACCTCCTGCTCTGTGCAGACATCTCTTCTTTGCTCTTACTAGACC |
| | | AAGGTGAAAGAAAACTCTCACCTTCTCCCATCTGGCCCCACAGCAT |
| | | CTGGAACACACTGATCCTCATAATCCTTGTTCTTGAGAAATATTAA |
| | | TGACTTAATCTCCCAAGCTTGCTCCCTCTCCTGTGCAGGCCATCTC |
| | | AGTATGTTTTGCAGACAAGACCCAGAGAAGTCCAGACTGGACTTGT |
| | | TGCAGACTGCAAAACTGCCATTGGAAGGCCTCCGTCCCAGTCCTTC |
| | | TACAGAGTAGCCAGTGGGATTCCCAGCC |
| 12 | ProX572.2 (also | GTTAGTCTTTGCCCTTTATCTGCCTGGCAAAGCTTTTAATTGGCTT |
| | referred to as | GATCTGTCATTCCGCTAGACATAAAGGGGACAATCCCCGGATTAGG |
| | Pro572.2) | AAGGAGCTCTCCAGCTCGGGTAAGGAGTCTCAAGGCAAGGTAGGCA |
| | | AGCACCACCGGTCCGCACTCTCGCCCAGCTTTTACGGGAAGAAGAG |
| | | AAACCCAAGAAATTACAGGCTGAAACCAGAAAAGAACACATTAAAG |
| | | CACCAAGAGAAAGTTGGAGTGGGTTGAAGGGAAACAGATTTTTAAA |
| | | GTTAAGGCTCTGTGAAATGGGTAGAATTAACTACAGGTTAAAAATA |
| | | AAATGTTAACTAAAGGTTGCCTCTGAGTAACAGGATTATGGGTGAT |
| | | TTTAATTGTCTTCTTTGTGTATGTTCAACAGTGACTATAATATGTA |
| | | TTACTTTTGGAATAAAGGAAAACCTGAAAGGTGTGTTGTTTTATAA |
| | | GGGCCCTTAGGTTGCCAAAATTAGAGTCATTGAAATCTAAAGCTGA |
| | | TAAAAACTTTAGTGCAAAGATTGTGACATGGGAGACTACACATACC |
| | | AGATCCATAATGTACATGAGGACAGTAGGCCGAGGGGCCCTGCACA |
| | | TTGAAAGCCCACATGGGAGAAGCCCTTGGGAAGGGGAGTGGAAGGA |
| | | TGAGGCAAGGGGCCGGGGGGATGCAGAGGCTGGCAGGCAGTCATTT |
| | | CTCAGCTTCAGCCATTCCCGCCATGGGGGAATGTGGACAGAGAAGC |
| | | CAAACAAATCTCCTAAACAGTAAATGTCAGTCTTCTGTGTCAGATA |
| | | TTTAAGAAAACTAACAGAGGTCAGAGAAGACACACCTACAGCAAGT |
| | | AGACTGTCCCTGTGCTGCCTTTTTGCAACCCCTGCTTTGGCAGTGC |
| | | TCAAGCCCACCTCCTGCTCTGTGCAGACATCTCTTCTTTGCTCTTA |
| | | CTAGACCAAGGTGAAAGAAAACTCTCACCTTCTCCCATCTGGCCCC |
| | | ACAGCATCTGGAACACACTGATCCTCATAATCCTTGTTCTTGAGAA |
| | | ATATTAATGACTTAATCTCCCAAGCTTGCTCCCTCTCCTGTGCAGG |
| | | CCATCTCAGTATGTTTTGCAGACAAGACCCAGAGAAGTCCAGACTG |
| | | GACTTGTTGCAGACTGCAAAACTGCCATTGGAAGGCCTCCGTCCCA |
| | | GTCCTTCTACAGAGTAGCCAGTGGGATTCCCAGCC |
| 13 | ProX573 (also referred | AACCCAAGAAATTACAGGCTGAAACCAGAAAAGAACACATTAAAGC |
| | to as Pro573) | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCAGTCTTCTGTGTCAGATAT |
| | | TTAAGAAAACTAACAGAGGTCAGAGAAGACACACCTACAGCAAGTA |
| | | GACTGTCCCTGTGCTGCCTTTTTGCAACCCCTGCTTTGGCAGTGCT |
| | | CAAGCCCACCTCCTGCTCTGTGCAGACATCTCTTCTTTGCTCTTAC |
| | | TAGACCAAGGTGAAAGAAAACTCTCACCTTCTCCCATCTGGCCCCA |
| | | CAGCATCTGGAACACACTGATCCTCATAATCCTTGTTCTTGAGAAA |
| | | TATTAATGACTTAATCTCCCAAGCTTGCTCCCTCTCCTGTGCAGGC |
| | | CATCTCAGTATGTTTTGCAGACAAGACCCAGAGAAGTCCAGACTGG |
| | | ACTTGTTGCAGACTGCAAAACTGCCATTGGAAGGCCTCCGTCCCAG |
| | | TCCTTCTACAGAGTAGCCAGTGGGATTCCCAGCCCATCCTGAGAGA |
| | | TGAGCCAGGACAAAGAACCAGTAATAGCTCCTGGAGCAGCACATCT |
| | | GTTTTGCCAGGATTATCCCTTGGATCTCTTAAAACCGAGACCTTGT |
| | | AATCTGAAGACTCAACTTGGGCTGTACCCTTAACCTTCAGCTCTAT |
| | | GATGCAAGTGAGTCCACAGGACCGGAGGCTTTGAGATGAGCTTTTC |
| | | AGAAGGGAGGAGTTGGCCGCTTGCTCCCAGAGCTCCAGCACCTGCA |
| | | TTCTTCTGGCTATGTCAGAAGCCAGATCATTTCCCTCGTTAAAAAC |
| | | AAAAACAAAAAAACAAACAAACAAAATGTTAGTCTTTGCCCTTTAT |
| | | CTGCCTGGCAAAGCTTTTAATTGGCTTGATCTGTCATTCCGCTAGA |
| | | CATAAAGGGGACAATCCCCGGATTAGGAAGGAGCTCTCCAGCTCGG |
| | | GTAAGGAGTCTCAAGGCAAGGTAGGCAAGCACCACCGGTCCGCACT |
| | | CTCGCCCAGCTTTTACGGGAAGAAGAGA |
| 14 | ProX573.2 (also | AACCCAAGAAATTACAGGCTGAAACCAGAAAAGAACACATTAAAGC |
| | referred to as | ACCAAGAGAAAGTTGGAGTGGGTTGAAGGGAAACAGATTTTTAAAG |
| | Pro573.2) | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCAGTCTTCTGTGTCAGATAT |
| | | TTAAGAAAACTAACAGAGGTCAGAGAAGACACACCTACAGCAAGTA |
| | | GACTGTCCCTGTGCTGCCTTTTTGCAACCCCTGCTTTGGCAGTGCT |
| | | CAAGCCCACCTCCTGCTCTGTGCAGACATCTCTTCTTTGCTCTTAC |
| | | TAGACCAAGGTGAAAGAAAACTCTCACCTTCTCCCATCTGGCCCCA |
| | | CAGCATCTGGAACACACTGATCCTCATAATCCTTGTTCTTGAGAAA |
| | | TATTAATGACTTAATCTCCCAAGCTTGCTCCCTCTCCTGTGCAGGC |
| | | CATCTCAGTATGTTTTGCAGACAAGACCCAGAGAAGTCCAGACTGG |
| | | ACTTGTTGCAGACTGCAAAACTGCCATTGGAAGGCCTCCGTCCCAG |
| | | TCCTTCTACAGAGTAGCCAGTGGGATTCCCAGCCGTTAGTCTTTGC |
| | | CCTTTATCTGCCTGGCAAAGCTTTTAATTGGCTTGATCTGTCATTC |
| | | CGCTAGACATAAAGGGGACAATCCCCGGATTAGGAAGGAGCTCTCC |
| | | AGCTCGGGTAAGGAGTCTCAAGGCAAGGTAGGCAAGCACCACCGGT |
| | | CCGCACTCTCGCCCAGCTTTTACGGGAAGAAGAGA |
| 15 | AAV L-ITR | CCTGCAGGCAGCTGCGCGCTCGCTCGCTCACTGAGGCCGCCCGGGC |
| | | GTCGGGCGACCTTTGGTCGCCCGGCCTCAGTGAGCGAGCGAGCGCG |
| | | CAGAGAGGGAGTGGCCAACTCCATCACTAGGGGTTCCT |
| 16 | ReaChR | ATGGTGAGCAGAAGACCCTGGCTGCTGGCCCTGGCCCTGGCCGTGG |
| | | CCCTGGCCGCCGGCAGCGCCGGCGCCAGCACCGGCAGCGACGCCAC |
| | | CGTGCCCGTGGCCACCCAGGACGGCCCCGACTACGTGTTCCACAGA |
| | | GCCCACGAGAGAATGCTGTTCCAGACCAGCTACACCCTGGAGAACA |
| | | ACGGCAGCGTGATCTGCATCCCCAACAACGGCCAGTGCTTCTGCCT |
| | | GGCCTGGCTGAAGAGTAACGGCACCAACGCCGAGAAGCTGGCCGCC |
| | | AACATCCTGCAGTGGGTGGTGTTTGCGCTGAGCGTGGCGTGCCTGG |
| | | GCTGGTATGCGTATCAGGCGTGGCGCGCGACCTGCGGCTGGGAAGA |
| | | AGTGTATGTGGCGCTGATTGAAATGATGAAAAGCATTATTGAAGCG |
| | | TTTCATGAATTTGATAGCCCGGCGACCCTGTGGCTGAGCAGCGGCA |
| | | ACGGCGTGGTGTGGATGCGCTATGGCGAATGGCTGCTGACCTGCCC |
| | | CGTGATTCTGATTCATCTGAGCAACCTGACCGGCCTGAAAGATGAT |
| | | TATAGCAAACGCACCATGGGCCTGCTGGTGAGCGACGTGGGCTGCA |
| | | TTGTGTGGGGCGCGACCAGCGCGATGTGCACCGGCTGGACCAAAAT |
| | | TCTGTTTTTTCTGATTAGCCTGAGCTATGGCATGTATACCTATTTT |
| | | CATGCGGCCAAAGTGTATATTGAAGCGTTTCATACCGTGCCGAAAG |
| | | GCCTGTGCAGACAGCTGGTGAGAGCCATGGCCTGGCTGTTCTTCGT |
| | | GAGCTGGGGCATGTTCCCCGTGCTGTTCCTGCTGGGCCCCGAGGGC |
| | | TTCGGCCATATTAGCCCGTATGGCAGCGCGATTGGCCATAGCATTC |
| | | TGGATCTGATTGCGAAGAACATGTGGGGCGTGCTGGGCAACTATCT |
| | | GCGCGTGAAAATTCATGAACATATTCTGCTGTATGGCGATATTCGC |
| | | AAAAAACAGAAAATTACCATTGCGGGCCAGGAAATGGAAGTGGAAA |
| | | CCCTGGTGGCGGAAGAAGAAGATAAGTACGAGAGCAGCCTCGAG |
| 17 | AAV5-ProA7- | CCTGCAGGCAGCTGCGCGCTCGCTCGCTCACTGAGGCCGCCCGGGC |
| | ReaChR-WPRE-hGH | GTCGGGCGACCTTTGGTCGCCCGGCCTCAGTGAGCGAGCGAGCGCG |
| | | CAGAGAGGGAGTGGCCAACTCCATCACTAGGGGTTCCTGCGGCCGC |
| | | ACGCGTCATCCTGAGAGATGAGCCAGGACAAAGAACCAGTAATAGC |
| | | TCCTGGAGCAGCACATCTGTTTTGCCAGGATTATCCCTTGGATCTC |
| | | TTAAAACCGAGACCTTGTAATCTGAAGACTCAACTTGGGCTGTACC |
| | | CTTAACCTTCAGCTCTATGATGCAAGTGAGTCCACAGGACCGGAGG |
| | | CTTTGAGATGAGCTTTTCAGAAGGGAGGAGTTGGCCGCTTGCTCCC |
| | | AGAGCTCCAGCACCTGCATTCTTCTGGCTATGTCAGAAGCCAGATC |
| | | ATTTCCCTCGTTAAAAACAAAAACAAAAAAACAAACAAACAAAATG |
| | | TTAGTCTTTGCCCTTTATCTGCCTGGCAAAGCTTTTAATTGGCTTG |
| | | ATCTGTCATTCCGCTAGACATAAAGGGGACAATCCCCGGATTAGGA |
| | | AGGAGCTCTCCAGCTCGGGTAAGGAGTCTCAAGGCAAGGTAGGCAA |
| | | GCACCACCGGTCCGCACTCTCGCCCAGCTTTTACGGGAAGAAGAGA |
| | | GGATCCGCCACCATGGTGAGCAGAAGACCCTGGCTGCTGGCCCTGG |
| | | CCCTGGCCGTGGCCCTGGCCGCCGGCAGCGCCGGCGCCAGCACCGG |
| | | CAGCGACGCCACCGTGCCCGTGGCCACCCAGGACGGCCCCGACTAC |
| | | GTGTTCCACAGAGCCCACGAGAGAATGCTGTTCCAGACCAGCTACA |
| | | CCCTGGAGAACAACGGCAGCGTGATCTGCATCCCCAACAACGGCCA |
| | | GTGCTTCTGCCTGGCCTGGCTGAAGAGTAACGGCACCAACGCCGAG |
| | | AAGCTGGCCGCCAACATCCTGCAGTGGGTGGTGTTTGCGCTGAGCG |
| | | TGGCGTGCCTGGGCTGGTATGCGTATCAGGCGTGGCGCGCGACCTG |
| | | CGGCTGGGAAGAAGTGTATGTGGCGCTGATTGAAATGATGAAAAGC |
| | | ATTATTGAAGCGTTTCATGAATTTGATAGCCCGGCGACCCTGTGGC |
| | | TGAGCAGCGGCAACGGCGTGGTGTGGATGCGCTATGGCGAATGGCT |
| | | GCTGACCTGCCCCGTGATTCTGATTCATCTGAGCAACCTGACCGGC |
| | | CTGAAAGATGATTATAGCAAACGCACCATGGGCCTGCTGGTGAGCG |
| | | ACGTGGGCTGCATTGTGTGGGGCGCGACCAGCGCGATGTGCACCGG |
| | | CTGGACCAAAATTCTGTTTTTTCTGATTAGCCTGAGCTATGGCATG |
| | | TATACCTATTTTCATGCGGCCAAAGTGTATATTGAAGCGTTTCATA |
| | | CCGTGCCGAAAGGCCTGTGCAGACAGCTGGTGAGAGCCATGGCCTG |
| | | GCTGTTCTTCGTGAGCTGGGGCATGTTCCCCGTGCTGTTCCTGCTG |
| | | GGCCCCGAGGGCTTCGGCCATATTAGCCCGTATGGCAGCGCGATTG |
| | | GCCATAGCATTCTGGATCTGATTGCGAAGAACATGTGGGGCGTGCT |
| | | GGGCAACTATCTGCGCGTGAAAATTCATGAACATATTCTGCTGTAT |
| | | GGCGATATTCGCAAAAAACAGAAAATTACCATTGCGGGCCAGGAAA |
| | | TGGAAGTGGAAACCCTGGTGGCGGAAGAAGAAGATAAGTACGAGAG |
| | | CAGCCTCGAGTAAAGCGGCCGCCGGCAAGTAAGCTTATCGATAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCT |
| | | TTGTATCATGCTATTGCTTCCCGTATGGCTTTCATTTTCTCCTCCT |
| | | TGTATAAATCCTGGTTGCTGTCTCTTTATGAGGAGTTGTGGCCCGT |
| | | TGTCAGGCAACGTGGCGTGGTGTGCACTGTGTTTGCTGACGCAACC |
| | | CCCACTGGTTGGGGCATTGCCACCACCTGTCAGCTCCTTTCCGGGA |
| | | CTTTCGCTTTCCCCCTCCCTATTGCCACGGCGGAACTCATCGCCGC |
| | | CTGCCTTGCCCGCTGCTGGACAGGGGCTCGGCTGTTGGGCACTGAC |
| | | AATTCCGTGGTGTTGTCGGGGAAATCATCGTCCTTTCCTTGGCTGC |
| | | TCGCCTATGTTGCCACCTGGATTCTGCGCGGGACGTCCTTCTGCTA |
| | | CGTCCCTTCGGCCCTCAATCCAGCGGACCTTCCTTCCCGCGGCCTG |
| | | CTGCCGGCTCTGCGGCCTCTTCCGCGTCTTCGCCTTCGCCCTCAGA |
| | | CGAGTCGGATCTCCCTTTGGGCCGCCTCCCCGCATCGATACCGAGC |
| | | GCTGCTCGAGAGATCTACGGGTGGCATCCCTGTGACCCCTCCCCAG |
| | | TGCCTCTCCTGGCCCTGGAAGTTGCCACTCCAGTGCCCACCAGCCT |
| | | TGTCCTAATAAAATTAAGTTGCATCATTTTGTCTGACTAGGTGTCC |
| | | TTCTATAATATTATGGGGTGGAGGGGGGTGGTATGGAGCAAGGGGC |
| | | AAGTTGGGAAGACAACCTGTAGGGCCTGCGGGGTCTATTGGGAACC |
| | | AAGCTGGAGTGCAGTGGCACAATCTTGGCTCACTGCAATCTCCGCC |
| | | TCCTGGGTTCAAGCGATTCTCCTGCCTCAGCCTCCCGAGTTGTTGG |
| | | GATTCCAGGCATGCATGACCAGGCTCAGCTAATTTTTGTTTTTTTG |
| | | GTAGAGACGGGGTTTCACCATATTGGCCAGGCTGGTCTCCAACTCC |
| | | TAATCTCAGGTGATCTACCCACCTTGGCCTCCCAAATTGCTGGGAT |
| | | TACAGGCGTGAACCACTGCTCCCTTCCCTGTCCTTCTGATTTTGTA |
| | | GGTAACCACGTGCGGACCGAGCGGCCGCAGGAACCCCTAGTGATGG |
| | | AGTTGGCCACTCCCTCTCTGCGCGCTCGCTCGCTCACTGAGGCCGG |
| | | GCGACCAAAGGTCGCCCGACGCCCGGGCTTTGCCCGGGCGGCCTCA |
| | | GTGAGCGAGCGAGCGCGCAGCTGCCTGCAGG |
| 18 | AAV5-Pro573.2- | CCTGCAGGCAGCTGCGCGCTCGCTCGCTCACTGAGGCCGCCCGGGC |
| | ReaChR-WPRE-hGH | GTCGGGCGACCTTTGGTCGCCCGGCCTCAGTGAGCGAGCGAGCGCG |
| | | CAGAGAGGGAGTGGCCAACTCCATCACTAGGGGTTCCTGCGGCCGC |
| | | ACGCGTAACCCAAGAAATTACAGGCTGAAACCAGAAAAGAACACAT |
| | | TAAAGCACCAAGAGAAAGTTGGAGTGGGTTGAAGGGAAACAGATTT |
| | | TTAAAGTTAAGGCTCTGTGAAATGGGTAGAATTAACTACAGGTTAA |
| | | AAATAAAATGTTAACTAAAGGTTGCCTCTGAGTAACAGGATTATGG |
| | | GTGATTTTAATTGTCTTCTTTGTGTATGTTCAACAGTGACTATAAT |
| | | ATGTATTACTTTTGGAATAAAGGAAAACCTGAAAGGTGTGTTGTTT |
| | | TATAAGGGCCCTTAGGTTGCCAAAATTAGAGTCATTGAAATCTAAA |
| | | GCTGATAAAAACTTTAGTGCAAAGATTGTGACATGGGAGACTACAC |
| | | ATACCAGATCCATAATGTACATGAGGACAGTAGGCCGAGGGGCCCT |
| | | GCACATTGAAAGCCCACATGGGAGAAGCCCTTGGGAAGGGGAGTGG |
| | | AAGGATGAGGCAAGGGGCCGGGGGGATGCAGAGGCTGGCAGGCAGT |
| | | CATTTCTCAGCTTCAGCCATTCCCGCCATGGGGGAATGTGGACAGA |
| | | GAAGCCAAACAAATCTCCTAAACAGTAAATGTCAGTCTTCTGTGTC |
| | | AGATATTTAAGAAAACTAACAGAGGTCAGAGAAGACACACCTACAG |
| | | CAAGTAGACTGTCCCTGTGCTGCCTTTTTGCAACCCCTGCTTTGGC |
| | | AGTGCTCAAGCCCACCTCCTGCTCTGTGCAGACATCTCTTCTTTGC |
| | | TCTTACTAGACCAAGGTGAAAGAAAACTCTCACCTTCTCCCATCTG |
| | | GCCCCACAGCATCTGGAACACACTGATCCTCATAATCCTTGTTCTT |
| | | GAGAAATATTAATGACTTAATCTCCCAAGCTTGCTCCCTCTCCTGT |
| | | GCAGGCCATCTCAGTATGTTTTGCAGACAAGACCCAGAGAAGTCCA |
| | | GACTGGACTTGTTGCAGACTGCAAAACTGCCATTGGAAGGCCTCCG |
| | | TCCCAGTCCTTCTACAGAGTAGCCAGTGGGATTCCCAGCCGTTAGT |
| | | CTTTGCCCTTTATCTGCCTGGCAAAGCTTTTAATTGGCTTGATCTG |
| | | TCATTCCGCTAGACATAAAGGGGACAATCCCCGGATTAGGAAGGAG |
| | | CTCTCCAGCTCGGGTAAGGAGTCTCAAGGCAAGGTAGGCAAGCACC |
| | | ACCGGTCCGCACTCTCGCCCAGCTTTTACGGGAAGAAGAGAGGATC |
| | | CGCCACCATGGTGAGCAGAAGACCCTGGCTGCTGGCCCTGGCCCTG |
| | | GCCGTGGCCCTGGCCGCCGGCAGCGCCGGCGCCAGCACCGGCAGCG |
| | | ACGCCACCGTGCCCGTGGCCACCCAGGACGGCCCCGACTACGTGTT |
| | | CCACAGAGCCCACGAGAGAATGCTGTTCCAGACCAGCTACACCCTG |
| | | GAGAACAACGGCAGCGTGATCTGCATCCCCAACAACGGCCAGTGCT |
| | | TCTGCCTGGCCTGGCTGAAGAGTAACGGCACCAACGCCGAGAAGCT |
| | | GGCCGCCAACATCCTGCAGTGGGTGGTGTTTGCGCTGAGCGTGGCG |
| | | TGCCTGGGCTGGTATGCGTATCAGGCGTGGCGCGCGACCTGCGGCT |
| | | GGGAAGAAGTGTATGTGGCGCTGATTGAAATGATGAAAAGCATTAT |
| | | TGAAGCGTTTCATGAATTTGATAGCCCGGCGACCCTGTGGCTGAGC |
| | | AGCGGCAACGGCGTGGTGTGGATGCGCTATGGCGAATGGCTGCTGA |
| | | CCTGCCCCGTGATTCTGATTCATCTGAGCAACCTGACCGGCCTGAA |
| | | AGATGATTATAGCAAACGCACCATGGGCCTGCTGGTGAGCGACGTG |
| | | GGCTGCATTGTGTGGGGCGCGACCAGCGCGATGTGCACCGGCTGGA |
| | | CCAAAATTCTGTTTTTTCTGATTAGCCTGAGCTATGGCATGTATAC |
| | | CTATTTTCATGCGGCCAAAGTGTATATTGAAGCGTTTCATACCGTG |
| | | CCGAAAGGCCTGTGCAGACAGCTGGTGAGAGCCATGGCCTGGCTGT |
| | | TCTTCGTGAGCTGGGGCATGTTCCCCGTGCTGTTCCTGCTGGGCCC |
| | | CGAGGGCTTCGGCCATATTAGCCCGTATGGCAGCGCGATTGGCCAT |
| | | AGCATTCTGGATCTGATTGCGAAGAACATGTGGGGCGTGCTGGGCA |
| | | ACTATCTGCGCGTGAAAATTCATGAACATATTCTGCTGTATGGCGA |
| | | TATTCGCAAAAAACAGAAAATTACCATTGCGGGCCAGGAAATGGAA |
| | | GTGGAAACCCTGGTGGCGGAAGAAGAAGATAAGTACGAGAGCAGCC |
| | | TCGAGTAAAGCGGCCGCCGGCAAGTAAGCTTATCGATAATCAACCT |
| | | CTGGATTACAAAATTTGTGAAAGATTGACTGGTATTCTTAACTATG |
| | | TTGCTCCTTTTACGCTATGTGGATACGCTGCTTTAATGCCTTTGTA |
| | | TCATGCTATTGCTTCCCGTATGGCTTTCATTTTCTCCTCCTTGTAT |
| | | AAATCCTGGTTGCTGTCTCTTTATGAGGAGTTGTGGCCCGTTGTCA |
| | | GGCAACGTGGCGTGGTGTGCACTGTGTTTGCTGACGCAACCCCCAC |
| | | TGGTTGGGGCATTGCCACCACCTGTCAGCTCCTTTCCGGGACTTTC |
| | | GCTTTCCCCCTCCCTATTGCCACGGCGGAACTCATCGCCGCCTGCC |
| | | TTGCCCGCTGCTGGACAGGGGCTCGGCTGTTGGGCACTGACAATTC |
| | | CGTGGTGTTGTCGGGGAAATCATCGTCCTTTCCTTGGCTGCTCGCC |
| | | TATGTTGCCACCTGGATTCTGCGCGGGACGTCCTTCTGCTACGTCC |
| | | CTTCGGCCCTCAATCCAGCGGACCTTCCTTCCCGCGGCCTGCTGCC |
| | | GGCTCTGCGGCCTCTTCCGCGTCTTCGCCTTCGCCCTCAGACGAGT |
| | | CGGATCTCCCTTTGGGCCGCCTCCCCGCATCGATACCGAGCGCTGC |
| | | TCGAGAGATCTACGGGTGGCATCCCTGTGACCCCTCCCCAGTGCCT |
| | | CTCCTGGCCCTGGAAGTTGCCACTCCAGTGCCCACCAGCCTTGTCC |
| | | TAATAAAATTAAGTTGCATCATTTTGTCTGACTAGGTGTCCTTCTA |
| | | TAATATTATGGGGTGGAGGGGGGTGGTATGGAGCAAGGGGCAAGTT |
| | | GGGAAGACAACCTGTAGGGCCTGCGGGGTCTATTGGGAACCAAGCT |
| | | GGAGTGCAGTGGCACAATCTTGGCTCACTGCAATCTCCGCCTCCTG |
| | | GGTTCAAGCGATTCTCCTGCCTCAGCCTCCCGAGTTGTTGGGATTC |
| | | CAGGCATGCATGACCAGGCTCAGCTAATTTTTGTTTTTTTGGTAGA |
| | | GACGGGGTTTCACCATATTGGCCAGGCTGGTCTCCAACTCCTAATC |
| | | TCAGGTGATCTACCCACCTTGGCCTCCCAAATTGCTGGGATTACAG |
| | | GCGTGAACCACTGCTCCCTTCCCTGTCCTTCTGATTTTGTAGGTAA |
| | | CCACGTGCGGACCGAGCGGCCGCAGGAACCCCTAGTGATGGAGTTG |
| | | GCCACTCCCTCTCTGCGCGCTCGCTCGCTCACTGAGGCCGGGCGAC |
| | | CAAAGGTCGCCCGACGCCCGGGCTTTGCCCGGGCGGCCTCAGTGAG |
| | | CGAGCGAGCGCGCAGCTGCCTGCAGG |
| 19 | AAV5-Pro572.2- | CCTGCAGGCAGCTGCGCGCTCGCTCGCTCACTGAGGCCGCCCGGGC |
| | ReaChR-WPRE-hGH | GTCGGGCGACCTTTGGTCGCCCGGCCTCAGTGAGCGAGCGAGCGCG |
| | | CAGAGAGGGAGTGGCCAACTCCATCACTAGGGGTTCCTGCGGCCGC |
| | | ACGCGTGTTAGTCTTTGCCCTTTATCTGCCTGGCAAAGCTTTTAAT |
| | | TGGCTTGATCTGTCATTCCGCTAGACATAAAGGGGACAATCCCCGG |
| | | ATTAGGAAGGAGCTCTCCAGCTCGGGTAAGGAGTCTCAAGGCAAGG |
| | | TAGGCAAGCACCACCGGTCCGCACTCTCGCCCAGCTTTTACGGGAA |
| | | GAAGAGAAACCCAAGAAATTACAGGCTGAAACCAGAAAAGAACACA |
| | | TTAAAGCACCAAGAGAAAGTTGGAGTGGGTTGAAGGGAAACAGATT |
| | | TTTAAAGTTAAGGCTCTGTGAAATGGGTAGAATTAACTACAGGTTA |
| | | AAAATAAAATGTTAACTAAAGGTTGCCTCTGAGTAACAGGATTATG |
| | | GGTGATTTTAATTGTCTTCTTTGTGTATGTTCAACAGTGACTATAA |
| | | TATGTATTACTTTTGGAATAAAGGAAAACCTGAAAGGTGTGTTGTT |
| | | TTATAAGGGCCCTTAGGTTGCCAAAATTAGAGTCATTGAAATCTAA |
| | | AGCTGATAAAAACTTTAGTGCAAAGATTGTGACATGGGAGACTACA |
| | | CATACCAGATCCATAATGTACATGAGGACAGTAGGCCGAGGGGCCC |
| | | TGCACATTGAAAGCCCACATGGGAGAAGCCCTTGGGAAGGGGAGTG |
| | | GAAGGATGAGGCAAGGGGCCGGGGGGATGCAGAGGCTGGCAGGCAG |
| | | TCATTTCTCAGCTTCAGCCATTCCCGCCATGGGGGAATGTGGACAG |
| | | AGAAGCCAAACAAATCTCCTAAACAGTAAATGTCAGTCTTCTGTGT |
| | | CAGATATTTAAGAAAACTAACAGAGGTCAGAGAAGACACACCTACA |
| | | GCAAGTAGACTGTCCCTGTGCTGCCTTTTTGCAACCCCTGCTTTGG |
| | | CAGTGCTCAAGCCCACCTCCTGCTCTGTGCAGACATCTCTTCTTTG |
| | | CTCTTACTAGACCAAGGTGAAAGAAAACTCTCACCTTCTCCCATCT |
| | | GGCCCCACAGCATCTGGAACACACTGATCCTCATAATCCTTGTTCT |
| | | TGAGAAATATTAATGACTTAATCTCCCAAGCTTGCTCCCTCTCCTG |
| | | TGCAGGCCATCTCAGTATGTTTTGCAGACAAGACCCAGAGAAGTCC |
| | | AGACTGGACTTGTTGCAGACTGCAAAACTGCCATTGGAAGGCCTCC |
| | | GTCCCAGTCCTTCTACAGAGTAGCCAGTGGGATTCCCAGCCGGATC |
| | | CGCCACCATGGTGAGCAGAAGACCCTGGCTGCTGGCCCTGGCCCTG |
| | | GCCGTGGCCCTGGCCGCCGGCAGCGCCGGCGCCAGCACCGGCAGCG |
| | | ACGCCACCGTGCCCGTGGCCACCCAGGACGGCCCCGACTACGTGTT |
| | | CCACAGAGCCCACGAGAGAATGCTGTTCCAGACCAGCTACACCCTG |
| | | GAGAACAACGGCAGCGTGATCTGCATCCCCAACAACGGCCAGTGCT |
| | | TCTGCCTGGCCTGGCTGAAGAGTAACGGCACCAACGCCGAGAAGCT |
| | | GGCCGCCAACATCCTGCAGTGGGTGGTGTTTGCGCTGAGCGTGGCG |
| | | TGCCTGGGCTGGTATGCGTATCAGGCGTGGCGCGCGACCTGCGGCT |
| | | GGGAAGAAGTGTATGTGGCGCTGATTGAAATGATGAAAAGCATTAT |
| | | TGAAGCGTTTCATGAATTTGATAGCCCGGCGACCCTGTGGCTGAGC |
| | | AGCGGCAACGGCGTGGTGTGGATGCGCTATGGCGAATGGCTGCTGA |
| | | CCTGCCCCGTGATTCTGATTCATCTGAGCAACCTGACCGGCCTGAA |
| | | AGATGATTATAGCAAACGCACCATGGGCCTGCTGGTGAGCGACGTG |
| | | GGCTGCATTGTGTGGGGCGCGACCAGCGCGATGTGCACCGGCTGGA |
| | | CCAAAATTCTGTTTTTTCTGATTAGCCTGAGCTATGGCATGTATAC |
| | | CTATTTTCATGCGGCCAAAGTGTATATTGAAGCGTTTCATACCGTG |
| | | CCGAAAGGCCTGTGCAGACAGCTGGTGAGAGCCATGGCCTGGCTGT |
| | | TCTTCGTGAGCTGGGGCATGTTCCCCGTGCTGTTCCTGCTGGGCCC |
| | | CGAGGGCTTCGGCCATATTAGCCCGTATGGCAGCGCGATTGGCCAT |
| | | AGCATTCTGGATCTGATTGCGAAGAACATGTGGGGCGTGCTGGGCA |
| | | ACTATCTGCGCGTGAAAATTCATGAACATATTCTGCTGTATGGCGA |
| | | TATTCGCAAAAAACAGAAAATTACCATTGCGGGCCAGGAAATGGAA |
| | | GTGGAAACCCTGGTGGCGGAAGAAGAAGATAAGTACGAGAGCAGCC |
| | | TCGAGTAAAGCGGCCGCCGGCAAGTAAGCTTATCGATAATCAACCT |
| | | CTGGATTACAAAATTTGTGAAAGATTGACTGGTATTCTTAACTATG |
| | | TTGCTCCTTTTACGCTATGTGGATACGCTGCTTTAATGCCTTTGTA |
| | | TCATGCTATTGCTTCCCGTATGGCTTTCATTTTCTCCTCCTTGTAT |
| | | AAATCCTGGTTGCTGTCTCTTTATGAGGAGTTGTGGCCCGTTGTCA |
| | | GGCAACGTGGCGTGGTGTGCACTGTGTTTGCTGACGCAACCCCCAC |
| | | TGGTTGGGGCATTGCCACCACCTGTCAGCTCCTTTCCGGGACTTTC |
| | | GCTTTCCCCCTCCCTATTGCCACGGCGGAACTCATCGCCGCCTGCC |
| | | TTGCCCGCTGCTGGACAGGGGCTCGGCTGTTGGGCACTGACAATTC |
| | | CGTGGTGTTGTCGGGGAAATCATCGTCCTTTCCTTGGCTGCTCGCC |
| | | TATGTTGCCACCTGGATTCTGCGCGGGACGTCCTTCTGCTACGTCC |
| | | CTTCGGCCCTCAATCCAGCGGACCTTCCTTCCCGCGGCCTGCTGCC |
| | | GGCTCTGCGGCCTCTTCCGCGTCTTCGCCTTCGCCCTCAGACGAGT |
| | | CGGATCTCCCTTTGGGCCGCCTCCCCGCATCGATACCGAGCGCTGC |
| | | TCGAGAGATCTACGGGTGGCATCCCTGTGACCCCTCCCCAGTGCCT |
| | | CTCCTGGCCCTGGAAGTTGCCACTCCAGTGCCCACCAGCCTTGTCC |
| | | TAATAAAATTAAGTTGCATCATTTTGTCTGACTAGGTGTCCTTCTA |
| | | TAATATTATGGGGTGGAGGGGGGTGGTATGGAGCAAGGGGCAAGTT |
| | | GGGAAGACAACCTGTAGGGCCTGCGGGGTCTATTGGGAACCAAGCT |
| | | GGAGTGCAGTGGCACAATCTTGGCTCACTGCAATCTCCGCCTCCTG |
| | | GGTTCAAGCGATTCTCCTGCCTCAGCCTCCCGAGTTGTTGGGATTC |
| | | CAGGCATGCATGACCAGGCTCAGCTAATTTTTGTTTTTTTGGTAGA |
| | | GACGGGGTTTCACCATATTGGCCAGGCTGGTCTCCAACTCCTAATC |
| | | TCAGGTGATCTACCCACCTTGGCCTCCCAAATTGCTGGGATTACAG |
| | | GCGTGAACCACTGCTCCCTTCCCTGTCCTTCTGATTTTGTAGGTAA |
| | | CCACGTGCGGACCGAGCGGCCGCAGGAACCCCTAGTGATGGAGTTG |
| | | GCCACTCCCTCTCTGCGCGCTCGCTCGCTCACTGAGGCCGGGCGAC |
| | | CAAAGGTCGCCCGACGCCCGGGCTTTGCCCGGGCGGCCTCAGTGAG |
| | | CGAGCGAGCGCGCAGCTGCCTGCAGG |
| 20 | AAV5-PR1.7- | CCTGCAGGCAGCTGCGCGCTCGCTCGCTCACTGAGGCCGCCCGGGC |
| | ReaChR-WPRE-hGH | GTCGGGCGACCTTTGGTCGCCCGGCCTCAGTGAGCGAGCGAGCGCG |
| | | CAGAGAGGGAGTGGCCAACTCCATCACTAGGGGTTCCTGCGGCCGC |
| | | AGGAGGCTGAGGGGTGGGGAAAGGGCATGGGTGTTTCATGAGGACA |
| | | GAGCTTCCGTTTCATGCAATGAAAAGAGTTTGGAGACGGATGGTGG |
| | | TGACTGGACTATACACTTACACACGGTAGCGATGGTACACTTTGTA |
| | | TTATGTATATTTTACCACGATCTTTTTAAAGTGTCAAAGGCAAATG |
| | | GCCAAATGGTTCCTTGTCCTATAGCTGTAGCAGCCATCGGCTGTTA |
| | | GTGACAAAGCCCCTGAGTCAAGATGACAGCAGCCCCCATAACTCCT |
| | | AATCGGCTCTCCCGCGTGGAGTCATTTAGGAGTAGTCGCATTAGAG |
| | | ACAAGTCCAACATCTAATCTTCCACCCTGGCCAGGGCCCCAGCTGG |
| | | CAGCGAGGGTGGGAGACTCCGGGCAGAGCAGAGGGCGCTGACATTG |
| | | GGGCCCGGCCTGGCTTGGGTCCCTCTGGCCTTTCCCCAGGGGCCCT |
| | | CTTTCCTTGGGGCTTTCTTGGGCCGCCACTGCTCCCGCTCCTCTCC |
| | | CCCCATCCCACCCCCTCACCCCCTCGTTCTTCATATCCTTCTCTAG |
| | | TGCTCCCTCCACTTTCATCCACCCTTCTGCAAGAGTGTGGGACCAC |
| | | AAATGAGTTTTCACCTGGCCTGGGGACACACGTGCCCCCACAGGTG |
| | | CTGAGTGACTTTCTAGGACAGTAATCTGCTTTAGGCTAAAATGGGA |
| | | CTTGATCTTCTGTTAGCCCTAATCATCAATTAGCAGAGCCGGTGAA |
| | | GGTGCAGAACCTACCGCCTTTCCAGGCCTCCTCCCACCTCTGCCAC |
| | | CTCCACTCTCCTTCCTGGGATGTGGGGGCTGGCACACGTGTGGCCC |
| | | AGGGCATTGGTGGGATTGCACTGAGCTGGGTCATTAGCGTAATCCT |
| | | GGACAAGGGCAGACAGGGCGAGCGGAGGGCCAGCTCCGGGGCTCAG |
| | | GCAAGGCTGGGGGCTTCCCCCAGACACCCCACTCCTCCTCTGCTGG |
| | | ACCCCCACTTCATAGGGCACTTCGTGTTCTCAAAGGGCTTCCAAAT |
| | | AGCATGGTGGCCTTGGATGCCCAGGGAAGCCTCAGAGTTGCTTATC |
| | | TCCCTCTAGACAGAAGGGGAATCTCGGTCAAGAGGGAGAGGTCGCC |
| | | CTGTTCAAGGCCACCCAGCCAGCTCATGGCGGTAATGGGACAAGGC |
| | | TGGCCAGCCATCCCACCCTCAGAAGGGACCCGGTGGGGCAGGTGAT |
| | | CTCAGAGGAGGCTCACTTCTGGGTCTCACATTCTTGGATCCGGTTC |
| | | CAGGCCTCGGCCCTAAATAGTCTCCCTGGGCTTTCAAGAGAACCAC |
| | | ATGAGAAAGGAGGATTCGGGCTCTGAGCAGTTTCACCACCCACCCC |
| | | CCAGTCTGCAAATCCTGACCCGTGGGTCCACCTGCCCCAAAGGCGG |
| | | ACGCAGGACAGTAGAAGGGAACAGAGAACACATAAACACAGAGAGG |
| | | GCCACAGCGGCTCCCACAGTCACCGCCACCTTCCTGGCGGGGATGG |
| | | GTGGGGCGTCTGAGTTTGGTTCCCAGCAAATCCCTCTGAGCCGCCC |
| | | TTGCGGGCTCGCCTCAGGAGCAGGGGAGCAAGAGGTGGGAGGAGGA |
| | | GGTCTAAGTCCCAGGCCCAATTAAGAGATCAGGTAGTGTAGGGTTT |
| | | GGGAGCTTTTAAGGTGAAGAGGCCCGGGCTGATCCCACAGGCCAGT |
| | | ATAAAGCGCCGTGACCCTCAGGTGATGCGCCAGGGCCGGCTGCCGT |
| | | CGGGGACAGGGCTTTCCATAGCCCGCCACCATGGTGAGCAGAAGAC |
| | | CCTGGCTGCTGGCCCTGGCCCTGGCCGTGGCCCTGGCCGCCGGCAG |
| | | CGCCGGCGCCAGCACCGGCAGCGACGCCACCGTGCCCGTGGCCACC |
| | | CAGGACGGCCCCGACTACGTGTTCCACAGAGCCCACGAGAGAATGC |
| | | TGTTCCAGACCAGCTACACCCTGGAGAACAACGGCAGCGTGATCTG |
| | | CATCCCCAACAACGGCCAGTGCTTCTGCCTGGCCTGGCTGAAGAGT |
| | | AACGGCACCAACGCCGAGAAGCTGGCCGCCAACATCCTGCAGTGGG |
| | | TGGTGTTTGCGCTGAGCGTGGCGTGCCTGGGCTGGTATGCGTATCA |
| | | GGCGTGGCGCGCGACCTGCGGCTGGGAAGAAGTGTATGTGGCGCTG |
| | | ATTGAAATGATGAAAAGCATTATTGAAGCGTTTCATGAATTTGATA |
| | | GCCCGGCGACCCTGTGGCTGAGCAGCGGCAACGGCGTGGTGTGGAT |
| | | GCGCTATGGCGAATGGCTGCTGACCTGCCCCGTGATTCTGATTCAT |
| | | CTGAGCAACCTGACCGGCCTGAAAGATGATTATAGCAAACGCACCA |
| | | TGGGCCTGCTGGTGAGCGACGTGGGCTGCATTGTGTGGGGCGCGAC |
| | | CAGCGCGATGTGCACCGGCTGGACCAAAATTCTGTTTTTTCTGATT |
| | | AGCCTGAGCTATGGCATGTATACCTATTTTCATGCGGCCAAAGTGT |
| | | ATATTGAAGCGTTTCATACCGTGCCGAAAGGCCTGTGCAGACAGCT |
| | | GGTGAGAGCCATGGCCTGGCTGTTCTTCGTGAGCTGGGGCATGTTC |
| | | CCCGTGCTGTTCCTGCTGGGCCCCGAGGGCTTCGGCCATATTAGCC |
| | | CGTATGGCAGCGCGATTGGCCATAGCATTCTGGATCTGATTGCGAA |
| | | GAACATGTGGGGCGTGCTGGGCAACTATCTGCGCGTGAAAATTCAT |
| | | GAACATATTCTGCTGTATGGCGATATTCGCAAAAAACAGAAAATTA |
| | | CCATTGCGGGCCAGGAAATGGAAGTGGAAACCCTGGTGGCGGAAGA |
| | | AGAAGATAAGTACGAGAGCAGCCTCGAGTAAAGCGGCCGCCGGCAA |
| | | GTAAGCTTATCGATAATCAACCTCTGGATTACAAAATTTGTGAAAG |
| | | ATTGACTGGTATTCTTAACTATGTTGCTCCTTTTACGCTATGTGGA |
| | | TACGCTGCTTTAATGCCTTTGTATCATGCTATTGCTTCCCGTATGG |
| | | CTTTCATTTTCTCCTCCTTGTATAAATCCTGGTTGCTGTCTCTTTA |
| | | TGAGGAGTTGTGGCCCGTTGTCAGGCAACGTGGCGTGGTGTGCACT |
| | | GTGTTTGCTGACGCAACCCCCACTGGTTGGGGCATTGCCACCACCT |
| | | GTCAGCTCCTTTCCGGGACTTTCGCTTTCCCCCTCCCTATTGCCAC |
| | | GGCGGAACTCATCGCCGCCTGCCTTGCCCGCTGCTGGACAGGGGCT |
| | | CGGCTGTTGGGCACTGACAATTCCGTGGTGTTGTCGGGGAAATCAT |
| | | CGTCCTTTCCTTGGCTGCTCGCCTATGTTGCCACCTGGATTCTGCG |
| | | CGGGACGTCCTTCTGCTACGTCCCTTCGGCCCTCAATCCAGCGGAC |
| | | CTTCCTTCCCGCGGCCTGCTGCCGGCTCTGCGGCCTCTTCCGCGTC |
| | | TTCGCCTTCGCCCTCAGACGAGTCGGATCTCCCTTTGGGCCGCCTC |
| | | CCCGCATCGATACCGAGCGCTGCTCGAGAGATCTACGGGTGGCATC |
| | | CCTGTGACCCCTCCCCAGTGCCTCTCCTGGCCCTGGAAGTTGCCAC |
| | | TCCAGTGCCCACCAGCCTTGTCCTAATAAAATTAAGTTGCATCATT |
| | | TTGTCTGACTAGGTGTCCTTCTATAATATTATGGGGTGGAGGGGGG |
| | | TGGTATGGAGCAAGGGGCAAGTTGGGAAGACAACCTGTAGGGCCTG |
| | | CGGGGTCTATTGGGAACCAAGCTGGAGTGCAGTGGCACAATCTTGG |
| | | CTCACTGCAATCTCCGCCTCCTGGGTTCAAGCGATTCTCCTGCCTC |
| | | AGCCTCCCGAGTTGTTGGGATTCCAGGCATGCATGACCAGGCTCAG |
| | | CTAATTTTTGTTTTTTTGGTAGAGACGGGGTTTCACCATATTGGCC |
| | | AGGCTGGTCTCCAACTCCTAATCTCAGGTGATCTACCCACCTTGGC |
| | | CTCCCAAATTGCTGGGATTACAGGCGTGAACCACTGCTCCCTTCCC |
| | | TGTCCTTCTGATTTTGTAGGTAACCACGTGCGGACCGAGCGGCCGC |
| | | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAGCTGCCTGC |
| | | AGG |
| 21 | AAV5-hG1.7- | CCTGCAGGCAGCTGCGCGCTCGCTCGCTCACTGAGGCCGCCCGGGC |
| | ReaChR-WPRE-hGH | GTCGGGCGACCTTTGGTCGCCCGGCCTCAGTGAGCGAGCGAGCGCG |
| | | CAGAGAGGGAGTGGCCAACTCCATCACTAGGGGTTCCTGCGGCCGC |
| | | ATAGGAATAGAAGGGTGGGTGCAGGAGGCTGAGGGGTGGGGAAAGG |
| | | GCATGGGTGTTTCATGAGGACAGAGCTTCCGTTTCATGCAATGAAA |
| | | AGAGTTTGGAGACGGATGGTGGTGACTGGACTATACACTTACACAC |
| | | GGTAGCGATGGTACACTTTGTATTATGTATATTTTACCACGATCTT |
| | | TTTAAAGTGTCAAAGGCAAATGGCCAAATGGTTCCTTGTCCTATAG |
| | | CTGTAGCAGCCATCGGCTGTTAGTGACAAAGCCCCTGAGTCAAGAT |
| | | GACAGCAGCCCCCATAACTCCTAATCGGCTCTCCCGCGTGGAGTCA |
| | | TTTAGGAGTAGTCGCATTAGAGACAAGTCCAACATCTAATCTTCCA |
| | | CCCTGGCCAGGGCCCCAGCTGGCAGCGAGGGTGGGAGACTCCGGGC |
| | | AGAGCAGAGGGCGCTGACATTGGGGCCCGGCCTGGCTTGGGTCCCT |
| | | CTGGCCTTTCCCCAGGGGCCCTCTTTCCTTGGGGCTTTCTTGGGCC |
| | | GCCACTGCTCCCGCTCCTCTCCCCCCATCCCACCCCCTCACCCCCT |
| | | CGTTCTTCATATCCTTCTCTAGTGCTCCCTCCACTTTCATCCACCC |
| | | TTCTGCAAGAGTGTGGGACCACAAATGAGTTTTCACCTGGCCTGGG |
| | | GACACACGTGCCCCCACAGGTGCTGAGTGACTTTCTAGGACAGTAA |
| | | TCTGCTTTAGGCTAAAATGGGACTTGATCTTCTGTTAGCCCTAATC |
| | | ATCAATTAGCAGAGCCGGTGAAGGTGCAGAACCTACCGCCTTTCCA |
| | | GGCCTCCTCCCACCTCTGCCACCTCCACTCTCCTTCCTGGGATGTG |
| | | GGGGCTGGCACACGTGTGGCCCAGGGCATTGGTGGGATTGCACTGA |
| | | GCTGGGTCATTAGCGTAATCCTGGACAAGGGCAGACAGGGCGAGCG |
| | | GAGGGCCAGCTCCGGGGCTCAGGCAAGGCTGGGGGCTTCCCCCAGA |
| | | CACCCCACTCCTCCTCTGCTGGACCCCCACTTCATAGGGCACTTCG |
| | | TGTTCTCAAAGGGCTTCCAAATAGCATGGTGGCCTTGGATGCCCAG |
| | | GGAAGCCTCAGAGTTGCTTATCTCCCTCTAGACAGAAGGGGAATCT |
| | | CGGTCAAGAGGGAGAGGTCGCCCTGTTCAAGGCCACCCAGCCAGCT |
| | | CATGGCGGTAATGGGACAAGGCTGGCCAGCCATCCCACCCTCAGAA |
| | | GGGACCCGGTGGGGCAGGTGATCTCAGAGGAGGCTCACTTCTGGGT |
| | | CTCACATTCTTGACAGGTATTTGCCACTAAGCCCAGCTAATTGTTT |
| | | TTTATTTAGTAGAAACGGGGTTTCACCATGTTAGTCAGGCTGGTCG |
| | | GGAACTCCTGACCTCAGGAGATCTACCCGCCTTGGCCTCCCAAAGT |
| | | GCTGGGATTACAGGCGTGTGCCACTGTGCCCAGCCACTTTTTTTTA |
| | | GACAGAGTCTTGGTCTGTTGCCCAGGCTAGAGTTCAGTGGCGCCAT |
| | | CTCAGCTCACTGCAACCTCCGCCTCCCAGATTCAAGCGATTCTCCT |
| | | GCCTCGACCTCCCAGTAGCTGGGATTACAGGTTTCCAGCAAATCCC |
| | | TCTGAGCCGCCCCCGGGGGCTCGCCTCAGGAGCAAGGAAGCAAGGG |
| | | GTGGGAGGAGGAGGTCTAAGTCCCAGGCCCAATTAAGAGATCAGAT |
| | | GGTGTAGGATTTGGGAGCTTTTAAGGTGAAGAGGCCCGGGCTGATC |
| | | CCACTGGCCGGTATAAAGCACCGTGACCCTCAGGTGACGCACCATC |
| | | TAGAGCTGCCGTCGGGGACAGGGCTTTCCATAGCCCGCCACCATGG |
| | | TGAGCAGAAGACCCTGGCTGCTGGCCCTGGCCCTGGCCGTGGCCCT |
| | | GGCCGCCGGCAGCGCCGGCGCCAGCACCGGCAGCGACGCCACCGTG |
| | | CCCGTGGCCACCCAGGACGGCCCCGACTACGTGTTCCACAGAGCCC |
| | | ACGAGAGAATGCTGTTCCAGACCAGCTACACCCTGGAGAACAACGG |
| | | CAGCGTGATCTGCATCCCCAACAACGGCCAGTGCTTCTGCCTGGCC |
| | | TGGCTGAAGAGTAACGGCACCAACGCCGAGAAGCTGGCCGCCAACA |
| | | TCCTGCAGTGGGTGGTGTTTGCGCTGAGCGTGGCGTGCCTGGGCTG |
| | | GTATGCGTATCAGGCGTGGCGCGCGACCTGCGGCTGGGAAGAAGTG |
| | | TATGTGGCGCTGATTGAAATGATGAAAAGCATTATTGAAGCGTTTC |
| | | ATGAATTTGATAGCCCGGCGACCCTGTGGCTGAGCAGCGGCAACGG |
| | | CGTGGTGTGGATGCGCTATGGCGAATGGCTGCTGACCTGCCCCGTG |
| | | ATTCTGATTCATCTGAGCAACCTGACCGGCCTGAAAGATGATTATA |
| | | GCAAACGCACCATGGGCCTGCTGGTGAGCGACGTGGGCTGCATTGT |
| | | GTGGGGCGCGACCAGCGCGATGTGCACCGGCTGGACCAAAATTCTG |
| | | TTTTTTCTGATTAGCCTGAGCTATGGCATGTATACCTATTTTCATG |
| | | CGGCCAAAGTGTATATTGAAGCGTTTCATACCGTGCCGAAAGGCCT |
| | | GTGCAGACAGCTGGTGAGAGCCATGGCCTGGCTGTTCTTCGTGAGC |
| | | TGGGGCATGTTCCCCGTGCTGTTCCTGCTGGGCCCCGAGGGCTTCG |
| | | GCCATATTAGCCCGTATGGCAGCGCGATTGGCCATAGCATTCTGGA |
| | | TCTGATTGCGAAGAACATGTGGGGCGTGCTGGGCAACTATCTGCGC |
| | | GTGAAAATTCATGAACATATTCTGCTGTATGGCGATATTCGCAAAA |
| | | AACAGAAAATTACCATTGCGGGCCAGGAAATGGAAGTGGAAACCCT |
| | | GGTGGCGGAAGAAGAAGATAAGTACGAGAGCAGCCTCGAGTAAAGC |
| | | GGCCGCCGGCAAGTAAGCTTATCGATAATCAACCTCTGGATTACAA |
| | | AATTTGTGAAAGATTGACTGGTATTCTTAACTATGTTGCTCCTTTT |
| | | ACGCTATGTGGATACGCTGCTTTAATGCCTTTGTATCATGCTATTG |
| | | CTTCCCGTATGGCTTTCATTTTCTCCTCCTTGTATAAATCCTGGTT |
| | | GCTGTCTCTTTATGAGGAGTTGTGGCCCGTTGTCAGGCAACGTGGC |
| | | GTGGTGTGCACTGTGTTTGCTGACGCAACCCCCACTGGTTGGGGCA |
| | | TTGCCACCACCTGTCAGCTCCTTTCCGGGACTTTCGCTTTCCCCCT |
| | | CCCTATTGCCACGGCGGAACTCATCGCCGCCTGCCTTGCCCGCTGC |
| | | TGGACAGGGGCTCGGCTGTTGGGCACTGACAATTCCGTGGTGTTGT |
| | | CGGGGAAATCATCGTCCTTTCCTTGGCTGCTCGCCTATGTTGCCAC |
| | | CTGGATTCTGCGCGGGACGTCCTTCTGCTACGTCCCTTCGGCCCTC |
| | | AATCCAGCGGACCTTCCTTCCCGCGGCCTGCTGCCGGCTCTGCGGC |
| | | CTCTTCCGCGTCTTCGCCTTCGCCCTCAGACGAGTCGGATCTCCCT |
| | | TTGGGCCGCCTCCCCGCATCGATACCGAGCGCTGCTCGAGAGATCT |
| | | ACGGGTGGCATCCCTGTGACCCCTCCCCAGTGCCTCTCCTGGCCCT |
| | | GGAAGTTGCCACTCCAGTGCCCACCAGCCTTGTCCTAATAAAATTA |
| | | AGTTGCATCATTTTGTCTGACTAGGTGTCCTTCTATAATATTATGG |
| | | GGTGGAGGGGGGTGGTATGGAGCAAGGGGCAAGTTGGGAAGACAAC |
| | | CTGTAGGGCCTGCGGGGTCTATTGGGAACCAAGCTGGAGTGCAGTG |
| | | GCACAATCTTGGCTCACTGCAATCTCCGCCTCCTGGGTTCAAGCGA |
| | | TTCTCCTGCCTCAGCCTCCCGAGTTGTTGGGATTCCAGGCATGCAT |
| | | GACCAGGCTCAGCTAATTTTTGTTTTTTTGGTAGAGACGGGGTTTC |
| | | ACCATATTGGCCAGGCTGGTCTCCAACTCCTAATCTCAGGTGATCT |
| | | ACCCACCTTGGCCTCCCAAATTGCTGGGATTACAGGCGTGAACCAC |
| | | TGCTCCCTTCCCTGTCCTTCTGATTTTGTAGGTAACCACGTGCGGA |
| | | CCGAGCGGCCGCAGGAACCCCTAGTGATGGAGTTGGCCACTCCCTC |
| | | TCTGCGCGCTCGCTCGCTCACTGAGGCCGGGCGACCAAAGGTCGCC |
| | | CGACGCCCGGGCTTTGCCCGGGCGGCCTCAGTGAGCGAGCGAGCGC |
| | | GCAGCTGCCTGCAGG |
| 22 | PR1.7 | GGAGGCTGAGGGGTGGGGAAAGGGCATGGGTGTTTCATGAGGACAG |
| | | AGCTTCCGTTTCATGCAATGAAAAGAGTTTGGAGACGGATGGTGGT |
| | | GACTGGACTATACACTTACACACGGTAGCGATGGTACACTTTGTAT |
| | | TATGTATATTTTACCACGATCTTTTTAAAGTGTCAAAGGCAAATGG |
| | | CCAAATGGTTCCTTGTCCTATAGCTGTAGCAGCCATCGGCTGTTAG |
| | | TGACAAAGCCCCTGAGTCAAGATGACAGCAGCCCCCATAACTCCTA |
| | | ATCGGCTCTCCCGCGTGGAGTCATTTAGGAGTAGTCGCATTAGAGA |
| | | CAAGTCCAACATCTAATCTTCCACCCTGGCCAGGGCCCCAGCTGGC |
| | | AGCGAGGGTGGGAGACTCCGGGCAGAGCAGAGGGCGCTGACATTGG |
| | | GGCCCGGCCTGGCTTGGGTCCCTCTGGCCTTTCCCCAGGGGCCCTC |
| | | TTTCCTTGGGGCTTTCTTGGGCCGCCACTGCTCCCGCTCCTCTCCC |
| | | CCCATCCCACCCCCTCACCCCCTCGTTCTTCATATCCTTCTCTAGT |
| | | GCTCCCTCCACTTTCATCCACCCTTCTGCAAGAGTGTGGGACCACA |
| | | AATGAGTTTTCACCTGGCCTGGGGACACACGTGCCCCCACAGGTGC |
| | | TGAGTGACTTTCTAGGACAGTAATCTGCTTTAGGCTAAAATGGGAC |
| | | TTGATCTTCTGTTAGCCCTAATCATCAATTAGCAGAGCCGGTGAAG |
| | | GTGCAGAACCTACCGCCTTTCCAGGCCTCCTCCCACCTCTGCCACC |
| | | TCCACTCTCCTTCCTGGGATGTGGGGGCTGGCACACGTGTGGCCCA |
| | | GGGCATTGGTGGGATTGCACTGAGCTGGGTCATTAGCGTAATCCTG |
| | | GACAAGGGCAGACAGGGCGAGCGGAGGGCCAGCTCCGGGGCTCAGG |
| | | CAAGGCTGGGGGCTTCCCCCAGACACCCCACTCCTCCTCTGCTGGA |
| | | CCCCCACTTCATAGGGCACTTCGTGTTCTCAAAGGGCTTCCAAATA |
| | | GCATGGTGGCCTTGGATGCCCAGGGAAGCCTCAGAGTTGCTTATCT |
| | | CCCTCTAGACAGAAGGGGAATCTCGGTCAAGAGGGAGAGGTCGCCC |
| | | TGTTCAAGGCCACCCAGCCAGCTCATGGCGGTAATGGGACAAGGCT |
| | | GGCCAGCCATCCCACCCTCAGAAGGGACCCGGTGGGGCAGGTGATC |
| | | TCAGAGGAGGCTCACTTCTGGGTCTCACATTCTTGGATCCGGTTCC |
| | | AGGCCTCGGCCCTAAATAGTCTCCCTGGGCTTTCAAGAGAACCACA |
| | | TGAGAAAGGAGGATTCGGGCTCTGAGCAGTTTCACCACCCACCCCC |
| | | CAGTCTGCAAATCCTGACCCGTGGGTCCACCTGCCCCAAAGGCGGA |
| | | CGCAGGACAGTAGAAGGGAACAGAGAACACATAAACACAGAGAGGG |
| | | CCACAGCGGCTCCCACAGTCACCGCCACCTTCCTGGCGGGGATGGG |
| | | TGGGGCGTCTGAGTTTGGTTCCCAGCAAATCCCTCTGAGCCGCCCT |
| | | TGCGGGCTCGCCTCAGGAGCAGGGGAGCAAGAGGTGGGAGGAGGAG |
| | | GTCTAAGTCCCAGGCCCAATTAAGAGATCAGGTAGTGTAGGGTTTG |
| | | GGAGCTTTTAAGGTGAAGAGGCCCGGGCTGATCCCACAGGCCAGTA |
| | | TAAAGCGCCGTGACCCTCAGGTGATGCGCCAGGGCCGGCTGCCGTC |
| | | GGGGACAGGGCTTTCCATAGCC |
| 23 | hG1.7 | TAGGAATAGAAGGGTGGGTGCAGGAGGCTGAGGGGTGGGGAAAGGG |
| | | CATGGGTGTTTCATGAGGACAGAGCTTCCGTTTCATGCAATGAAAA |
| | | GAGTTTGGAGACGGATGGTGGTGACTGGACTATACACTTACACACG |
| | | GTAGCGATGGTACACTTTGTATTATGTATATTTTACCACGATCTTT |
| | | TTAAAGTGTCAAAGGCAAATGGCCAAATGGTTCCTTGTCCTATAGC |
| | | TGTAGCAGCCATCGGCTGTTAGTGACAAAGCCCCTGAGTCAAGATG |
| | | ACAGCAGCCCCCATAACTCCTAATCGGCTCTCCCGCGTGGAGTCAT |
| | | TTAGGAGTAGTCGCATTAGAGACAAGTCCAACATCTAATCTTCCAC |
| | | CCTGGCCAGGGCCCCAGCTGGCAGCGAGGGTGGGAGACTCCGGGCA |
| | | GAGCAGAGGGCGCTGACATTGGGGCCCGGCCTGGCTTGGGTCCCTC |
| | | TGGCCTTTCCCCAGGGGCCCTCTTTCCTTGGGGCTTTCTTGGGCCG |
| | | CCACTGCTCCCGCTCCTCTCCCCCCATCCCACCCCCTCACCCCCTC |
| | | GTTCTTCATATCCTTCTCTAGTGCTCCCTCCACTTTCATCCACCCT |
| | | TCTGCAAGAGTGTGGGACCACAAATGAGTTTTCACCTGGCCTGGGG |
| | | ACACACGTGCCCCCACAGGTGCTGAGTGACTTTCTAGGACAGTAAT |
| | | CTGCTTTAGGCTAAAATGGGACTTGATCTTCTGTTAGCCCTAATCA |
| | | TCAATTAGCAGAGCCGGTGAAGGTGCAGAACCTACCGCCTTTCCAG |
| | | GCCTCCTCCCACCTCTGCCACCTCCACTCTCCTTCCTGGGATGTGG |
| | | GGGCTGGCACACGTGTGGCCCAGGGCATTGGTGGGATTGCACTGAG |
| | | CTGGGTCATTAGCGTAATCCTGGACAAGGGCAGACAGGGCGAGCGG |
| | | AGGGCCAGCTCCGGGGCTCAGGCAAGGCTGGGGGCTTCCCCCAGAC |
| | | ACCCCACTCCTCCTCTGCTGGACCCCCACTTCATAGGGCACTTCGT |
| | | GTTCTCAAAGGGCTTCCAAATAGCATGGTGGCCTTGGATGCCCAGG |
| | | GAAGCCTCAGAGTTGCTTATCTCCCTCTAGACAGAAGGGGAATCTC |
| | | GGTCAAGAGGGAGAGGTCGCCCTGTTCAAGGCCACCCAGCCAGCTC |
| | | ATGGCGGTAATGGGACAAGGCTGGCCAGCCATCCCACCCTCAGAAG |
| | | GGACCCGGTGGGGCAGGTGATCTCAGAGGAGGCTCACTTCTGGGTC |
| | | TCACATTCTTGACAGGTATTTGCCACTAAGCCCAGCTAATTGTTTT |
| | | TTATTTAGTAGAAACGGGGTTTCACCATGTTAGTCAGGCTGGTCGG |
| | | GAACTCCTGACCTCAGGAGATCTACCCGCCTTGGCCTCCCAAAGTG |
| | | CTGGGATTACAGGCGTGTGCCACTGTGCCCAGCCACTTTTTTTTAG |
| | | ACAGAGTCTTGGTCTGTTGCCCAGGCTAGAGTTCAGTGGCGCCATC |
| | | TCAGCTCACTGCAACCTCCGCCTCCCAGATTCAAGCGATTCTCCTG |
| | | CCTCGACCTCCCAGTAGCTGGGATTACAGGTTTCCAGCAAATCCCT |
| | | CTGAGCCGCCCCCGGGGGCTCGCCTCAGGAGCAAGGAAGCAAGGGG |
| | | TGGGAGGAGGAGGTCTAAGTCCCAGGCCCAATTAAGAGATCAGATG |
| | | GTGTAGGATTTGGGAGCTTTTAAGGTGAAGAGGCCCGGGCTGATCC |
| | | CACTGGCCGGTATAAAGCACCGTGACCCTCAGGTGACGCACCATCT |
| | | AGAGCTGCCGTCGGGGACAGGGCTTTCCATAGCC |
| 24 | ProA7 5′1 | ATCTGAAGACTCAACTTGGGCTGTACCCTTAACCTTCAGCTCTATG |
| | | ATGCAAGTGAGTCCACAGGACCGGAGGCTTTGAGATGAGCTTTTCA |
| | | GAAGGGAGGAGTTGGCCGCTTGCTCCCAGAGCTCCAGCACCTGCAT |
| | | TCTTCTGGCTATGTCAGAAGCCAGATCATTTCCCTCGTTAAAAACA |
| | | AAAACAAAAAAACAAACAAACAAAATGTTAGTCTTTGCCCTTTATC |
| | | TGCCTGGCAAAGCTTTTAATTGGCTTGATCTGTCATTCCGCTAGAC |
| | | ATAAAGGGGACAATCCCCGGATTAGGAAGGAGCTCTCCAGCTCGGG |
| | | TAAGGAGTCTCAAGGCAAGGTAGGCAAGCACCACCGGTCCGCACTC |
| | | TCGCCCAGCTTTTACGGGAAGAAGAGA |
| 25 | ProA7 5′2 | GGCCGCTTGCTCCCAGAGCTCCAGCACCTGCATTCTTCTGGCTATG |
| | | TCAGAAGCCAGATCATTTCCCTCGTTAAAAACAAAAACAAAAAAAC |
| | | AAACAAACAAAATGTTAGTCTTTGCCCTTTATCTGCCTGGCAAAGC |
| | | TTTTAATTGGCTTGATCTGTCATTCCGCTAGACATAAAGGGGACAA |
| | | TCCCCGGATTAGGAAGGAGCTCTCCAGCTCGGGTAAGGAGTCTCAA |
| | | GGCAAGGTAGGCAAGCACCACCGGTCCGCACTCTCGCCCAGCTTTT |
| | | ACGGGAAGAAGAGA |
| 26 | ProA7 5′3 | GTTAGTCTTTGCCCTTTATCTGCCTGGCAAAGCTTTTAATTGGCTT |
| | | GATCTGTCATTCCGCTAGACATAAAGGGGACAATCCCCGGATTAGG |
| | | AAGGAGCTCTCCAGCTCGGGTAAGGAGTCTCAAGGCAAGGTAGGCA |
| | | AGCACCACCGGTCCGCACTCTCGCCCAGCTTTTACGGGAAGAAGAG |
| | | A |
| 27 | ProA7 5′4 | TTAATTGGCTTGATCTGTCATTCCGCTAGACATAAAGGGGACAATC |
| | | CCCGGATTAGGAAGGAGCTCTCCAGCTCGGGTAAGGAGTCTCAAGG |
| | | CAAGGTAGGCAAGCACCACCGGTCCGCACTCTCGCCCAGCTTTTAC |
| | | GGGAAGAAGAGA |
| 28 | ProA7 5′5 | AGGGGACAATCCCCGGATTAGGAAGGAGCTCTCCAGCTCGGGTAAG |
| | | GAGTCTCAAGGCAAGGTAGGCAAGCACCACCGGTCCGCACTCTCGC |
| | | CCAGCTTTTACGGGAAGAAGAGA |
| 29 | ProA7 5′6 | GCTCGGGTAAGGAGTCTCAAGGCAAGGTAGGCAAGCACCACCGGTC |
| | | CGCACTCTCGCCCAGCTTTTACGGGAAGAAGAGA |
| 30 | ProA7 3′1 | CATCCTGAGAGATGAGCCAGGACAAAGAACCAGTAATAGCTCCTGG |
| | | AGCAGCACATCTGTTTTGCCAGGATTATCCCTTGGATCTCTTAAAA |
| | | CCGAGACCTTGTAATCTGAAGACTCAACTTGGGCTGTACCCTTAAC |
| | | CTTCAGCTCTATGATGCAAGTGAGTCCACAGGACCGGAGGCTTTGA |
| | | GATGAGCTTTTCAGAAGGGAGGAGTTGGCCGCTTGCTCCCAGAGCT |
| | | CCAGCACCTGCATTCTTCTGGCTATGTCAGAAGCCAGATCATTTCC |
| | | CTCGTTAAAAACAAAAACAAAAAAACAAACAAACAAAATGTTAGTC |
| | | TTTGCCCTTTATCTGCCTGGCAAAGCTTTTAATTGGCTTGATCTGT |
| | | CATTCCGCTAGACATAAAGGGGACAAT |
| 31 | AAV R-ITR | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAGCTGCCTGC |
| | | AGG |
| 32 | ReaChR | MVSRRPWLLALALAVALAAGSAGASTGSDATVPVATQDGPDYVFHR |
| | | AHERMLFQTSYTLENNGSVICIPNNGQCFCLAWLKSNGTNAEKLAA |
| | | NILQWVVFALSVACLGWYAYQAWRATCGWEEVYVALIEMMKSIIEA |
| | | FHEFDSPATLWLSSGNGVVWMRYGEWLLTCPVILIHLSNLTGLKDD |
| | | YSKRTMGLLVSDVGCIVWGATSAMCTGWTKILFFLISLSYGMYTYF |
| | | HAAKVYIEAFHTVPKGLCRQLVRAMAWLFFVSWGMFPVLFLLGPEG |
| | | FGHISPYGSAIGHSILDLIAKNMWGVLGNYLRVKIHEHILLYGDIR |
| | | KKQKITIAGQEMEVETLVAEEEDKYESSLE |
| 33 | ReaChR, codon | ATGGTGTCTAGAAGACCTTGGCTGCTGGCTCTGGCTCTGGCAGTGG |
| | optimized | CCCTGGCTGCAGGTTCTGCCGGCGCCAGCACCGGCAGCGACGCCAC |
| | | AGTGCCTGTGGCCACACAGGACGGCCCTGATTACGTGTTCCACAGA |
| | | GCCCACGAGAGAATGCTGTTTCAGACCTCTTATACCCTGGAAAACA |
| | | ATGGCAGCGTGATCTGCATCCCCAACAACGGCCAATGTTTCTGCCT |
| | | GGCCTGGCTCAAGAGCAACGGCACCAACGCCGAGAAGCTGGCCGCC |
| | | AACATCCTGCAGTGGGTCGTGTTTGCCCTGAGCGTGGCTTGTCTGG |
| | | GCTGGTACGCCTACCAGGCCTGGCGGGCCACATGCGGCTGGGAGGA |
| | | AGTGTACGTCGCCCTGATCGAGATGATGAAAAGCATCATCGAAGCC |
| | | TTCCACGAGTTCGACTCTCCAGCCACCCTGTGGCTGAGCTCTGGCA |
| | | ACGGCGTGGTGTGGATGCGCTACGGCGAGTGGCTGCTCACCTGCCC |
| | | TGTGATCCTGATCCACCTGTCCAACCTGACCGGCCTGAAGGACGAC |
| | | TATAGCAAGCGGACCATGGGCCTGCTGGTGTCCGATGTGGGCTGTA |
| | | TCGTGTGGGGCGCTACCAGCGCCATGTGCACCGGATGGACCAAGAT |
| | | TCTGTTTTTCCTGATTAGCCTGAGCTACGGCATGTACACCTACTTC |
| | | CACGCCGCTAAAGTGTACATCGAGGCCTTCCATACAGTGCCTAAGG |
| | | GCCTGTGCAGACAGCTGGTGCGGGCCATGGCCTGGCTGTTCTTCGT |
| | | GAGCTGGGGAATGTTCCCCGTGCTGTTCCTGCTTGGACCTGAGGGC |
| | | TTCGGCCACATCAGCCCCTACGGGTCAGCCATCGGCCACAGCATCC |
| | | TGGACCTGATCGCCAAGAACATGTGGGGCGTGCTGGGAAATTACCT |
| | | GAGAGTGAAGATCCACGAACACATCCTGCTGTACGGCGACATCAGA |
| | | AAGAAGCAGAAAATAACAATCGCCGGCCAGGAGATGGAAGTCGAGA |
| | | CACTGGTTGCTGAAGAGGAAGATAAGTACGAGAGCAGCCTGGAA |
| 34 | ReaChR, codon | ATGGTGTCAAGACGACCCTGGCTTCTGGCACTCGCGCTTGCAGTCG |
| | optimized | CTCTTGCTGCCGGGTCTGCAGGCGCCAGCACAGGTAGTGATGCCAC |
| | | CGTTCCCGTGGCAACGCAGGATGGCCCGGATTACGTGTTTCATAGA |
| | | GCACACGAAAGAATGCTTTTCCAGACGAGCTACACTCTGGAAAACA |
| | | ATGGTAGCGTTATTTGTATACCCAACAACGGACAGTGCTTTTGCTT |
| | | GGCTTGGCTTAAATCTAATGGGACTAACGCTGAGAAGCTCGCAGCA |
| | | AACATACTCCAATGGGTGGTGTTTGCTTTGTCCGTTGCGTGTCTTG |
| | | GTTGGTACGCTTATCAAGCCTGGCGAGCGACATGCGGATGGGAAGA |
| | | GGTTTATGTTGCCCTCATAGAGATGATGAAGTCAATAATCGAGGCT |
| | | TTTCATGAGTTCGATAGTCCCGCGACTCTGTGGTTGAGTAGTGGTA |
| | | ATGGTGTTGTGTGGATGCGGTATGGGGAGTGGCTCCTTACATGCCC |
| | | AGTTATTTTGATTCACCTGTCAAATTTGACTGGTCTTAAAGACGAC |
| | | TACAGCAAACGCACGATGGGTTTGTTGGTCAGTGATGTCGGTTGCA |
| | | TCGTTTGGGGGGCTACGTCCGCTATGTGTACCGGGTGGACGAAAAT |
| | | TCTGTTCTTTCTGATTAGCTTGTCTTACGGGATGTATACATATTTC |
| | | CACGCCGCTAAGGTCTACATCGAAGCCTTTCATACTGTGCCGAAGG |
| | | GATTGTGTCGCCAATTGGTCCGCGCGATGGCGTGGCTGTTTTTTGT |
| | | CAGCTGGGGAATGTTTCCTGTTTTGTTCTTGTTGGGTCCTGAAGGA |
| | | TTTGGGCATATTTCCCCTTATGGTTCAGCCATTGGACACTCTATTC |
| | | TGGATCTGATTGCAAAAAATATGTGGGGCGTGCTGGGAAACTACTT |
| | | GAGGGTGAAAATCCACGAGCACATACTGCTCTATGGTGACATCCGA |
| | | AAGAAGCAAAAAATTACTATAGCTGGGCAGGAGATGGAGGTGGAGA |
| | | CCTTGGTTGCAGAGGAGGAGGACAAGTACGAATCTTCCCTGGAG |
| 35 | AAV-ProA7-ReaChR- | CCTGCAGGCAGCTGCGCGCTCGCTCGCTCACTGAGGCCGCCCGGGC |
| | citrine-WPRE-hGH | GTCGGGCGACCTTTGGTCGCCCGGCCTCAGTGAGCGAGCGAGCGCG |
| | poly A | CAGAGAGGGAGTGGCCAACTCCATCACTAGGGGTTCCTGCGGCCGC |
| | | ACGCGTCATCCTGAGAGATGAGCCAGGACAAAGAACCAGTAATAGC |
| | | TCCTGGAGCAGCACATCTGTTTTGCCAGGATTATCCCTTGGATCTC |
| | | TTAAAACCGAGACCTTGTAATCTGAAGACTCAACTTGGGCTGTACC |
| | | CTTAACCTTCAGCTCTATGATGCAAGTGAGTCCACAGGACCGGAGG |
| | | CTTTGAGATGAGCTTTTCAGAAGGGAGGAGTTGGCCGCTTGCTCCC |
| | | AGAGCTCCAGCACCTGCATTCTTCTGGCTATGTCAGAAGCCAGATC |
| | | ATTTCCCTCGTTAAAAACAAAAACAAAAAAACAAACAAACAAAATG |
| | | TTAGTCTTTGCCCTTTATCTGCCTGGCAAAGCTTTTAATTGGCTTG |
| | | ATCTGTCATTCCGCTAGACATAAAGGGGACAATCCCCGGATTAGGA |
| | | AGGAGCTCTCCAGCTCGGGTAAGGAGTCTCAAGGCAAGGTAGGCAA |
| | | GCACCACCGGTCCGCACTCTCGCCCAGCTTTTACGGGAAGAAGAGA |
| | | GGATCCGCCACCATGGTGAGCAGAAGACCCTGGCTGCTGGCCCTGG |
| | | CCCTGGCCGTGGCCCTGGCCGCCGGCAGCGCCGGCGCCAGCACCGG |
| | | CAGCGACGCCACCGTGCCCGTGGCCACCCAGGACGGCCCCGACTAC |
| | | GTGTTCCACAGAGCCCACGAGAGAATGCTGTTCCAGACCAGCTACA |
| | | CCCTGGAGAACAACGGCAGCGTGATCTGCATCCCCAACAACGGCCA |
| | | GTGCTTCTGCCTGGCCTGGCTGAAGAGTAACGGCACCAACGCCGAG |
| | | AAGCTGGCCGCCAACATCCTGCAGTGGGTGGTGTTTGCGCTGAGCG |
| | | TGGCGTGCCTGGGCTGGTATGCGTATCAGGCGTGGCGCGCGACCTG |
| | | CGGCTGGGAAGAAGTGTATGTGGCGCTGATTGAAATGATGAAAAGC |
| | | ATTATTGAAGCGTTTCATGAATTTGATAGCCCGGCGACCCTGTGGC |
| | | TGAGCAGCGGCAACGGCGTGGTGTGGATGCGCTATGGCGAATGGCT |
| | | GCTGACCTGCCCCGTGATTCTGATTCATCTGAGCAACCTGACCGGC |
| | | CTGAAAGATGATTATAGCAAACGCACCATGGGCCTGCTGGTGAGCG |
| | | ACGTGGGCTGCATTGTGTGGGGCGCGACCAGCGCGATGTGCACCGG |
| | | CTGGACCAAAATTCTGTTTTTTCTGATTAGCCTGAGCTATGGCATG |
| | | TATACCTATTTTCATGCGGCCAAAGTGTATATTGAAGCGTTTCATA |
| | | CCGTGCCGAAAGGCCTGTGCAGACAGCTGGTGAGAGCCATGGCCTG |
| | | GCTGTTCTTCGTGAGCTGGGGCATGTTCCCCGTGCTGTTCCTGCTG |
| | | GGCCCCGAGGGCTTCGGCCATATTAGCCCGTATGGCAGCGCGATTG |
| | | GCCATAGCATTCTGGATCTGATTGCGAAGAACATGTGGGGCGTGCT |
| | | GGGCAACTATCTGCGCGTGAAAATTCATGAACATATTCTGCTGTAT |
| | | GGCGATATTCGCAAAAAACAGAAAATTACCATTGCGGGCCAGGAAA |
| | | TGGAAGTGGAAACCCTGGTGGCGGAAGAAGAAGATAAGTACGAGAG |
| | | CAGCCTCGAGATGGTGAGCAAGGGCGAGGAGCTGTTCACCGGGGTG |
| | | GTGCCCATCCTGGTCGAGCTGGACGGCGACGTAAACGGCCACAAGT |
| | | TCAGCGTGTCCGGCGAGGGCGAGGGCGATGCCACCTACGGCAAGCT |
| | | GACCCTGAAGTTCATCTGCACCACCGGCAAGCTGCCCGTGCCCTGG |
| | | CCCACCCTCGTGACCACCTTCGGCTACGGCCTGATGTGCTTCGCCC |
| | | GCTACCCCGACCACATGAAGCAGCACGACTTCTTCAAGTCCGCCAT |
| | | GCCCGAAGGCTACGTCCAGGAGCGCACCATCTTCTTCAAGGACGAC |
| | | GGCAACTACAAGACCCGCGCCGAGGTGAAGTTCGAGGGCGACACCC |
| | | TGGTGAACCGCATCGAGCTGAAGGGCATCGACTTCAAGGAGGACGG |
| | | CAACATCCTGGGGCACAAGCTGGAGTACAACTACAACAGCCACAAC |
| | | GTCTATATCATGGCCGACAAGCAGAAGAACGGCATCAAGGTGAACT |
| | | TCAAGATCCGCCACAACATCGAGGACGGCAGCGTGCAGCTCGCCGA |
| | | CCACTACCAGCAGAACACCCCCATCGGCGACGGCCCCGTGCTGCTG |
| | | CCCGACAACCACTACCTGAGCTACCAGTCCGCCCTGAGCAAAGACC |
| | | CCAACGAGAAGCGCGATCACATGGTCCTGCTGGAGTTCGTGACCGC |
| | | CGCCGGGATCACTCTCGGCATGGACGAGCTGTACAAGTAAAGCGGC |
| | | CGCCGGCAAGTAAGCTTATCGATAATCAACCTCTGGATTACAAAAT |
| | | TTGTGAAAGATTGACTGGTATTCTTAACTATGTTGCTCCTTTTACG |
| | | CTATGTGGATACGCTGCTTTAATGCCTTTGTATCATGCTATTGCTT |
| | | CCCGTATGGCTTTCATTTTCTCCTCCTTGTATAAATCCTGGTTGCT |
| | | GTCTCTTTATGAGGAGTTGTGGCCCGTTGTCAGGCAACGTGGCGTG |
| | | GTGTGCACTGTGTTTGCTGACGCAACCCCCACTGGTTGGGGCATTG |
| | | CCACCACCTGTCAGCTCCTTTCCGGGACTTTCGCTTTCCCCCTCCC |
| | | TATTGCCACGGCGGAACTCATCGCCGCCTGCCTTGCCCGCTGCTGG |
| | | ACAGGGGCTCGGCTGTTGGGCACTGACAATTCCGTGGTGTTGTCGG |
| | | GGAAATCATCGTCCTTTCCTTGGCTGCTCGCCTATGTTGCCACCTG |
| | | GATTCTGCGCGGGACGTCCTTCTGCTACGTCCCTTCGGCCCTCAAT |
| | | CCAGCGGACCTTCCTTCCCGCGGCCTGCTGCCGGCTCTGCGGCCTC |
| | | TTCCGCGTCTTCGCCTTCGCCCTCAGACGAGTCGGATCTCCCTTTG |
| | | GGCCGCCTCCCCGCATCGATACCGAGCGCTGCTCGAGAGATCTACG |
| | | GGTGGCATCCCTGTGACCCCTCCCCAGTGCCTCTCCTGGCCCTGGA |
| | | AGTTGCCACTCCAGTGCCCACCAGCCTTGTCCTAATAAAATTAAGT |
| | | TGCATCATTTTGTCTGACTAGGTGTCCTTCTATAATATTATGGGGT |
| | | GGAGGGGGGTGGTATGGAGCAAGGGGCAAGTTGGGAAGACAACCTG |
| | | TAGGGCCTGCGGGGTCTATTGGGAACCAAGCTGGAGTGCAGTGGCA |
| | | CAATCTTGGCTCACTGCAATCTCCGCCTCCTGGGTTCAAGCGATTC |
| | | TCCTGCCTCAGCCTCCCGAGTTGTTGGGATTCCAGGCATGCATGAC |
| | | CAGGCTCAGCTAATTTTTGTTTTTTTGGTAGAGACGGGGTTTCACC |
| | | ATATTGGCCAGGCTGGTCTCCAACTCCTAATCTCAGGTGATCTACC |
| | | CACCTTGGCCTCCCAAATTGCTGGGATTACAGGCGTGAACCACTGC |
| | | TCCCTTCCCTGTCCTTCTGATTTTGTAGGTAACCACGTGCGGACCG |
| | | AGCGGCCGCAGGAACCCCTAGTGATGGAGTTGGCCACTCCCTCTCT |
| | | GCGCGCTCGCTCGCTCACTGAGGCCGGGCGACCAAAGGTCGCCCGA |
| | | CGCCCGGGCTTTGCCCGGGCGGCCTCAGTGAGCGAGCGAGCGCGCA |
| | | GCTGCCTGCAGG |
| 36 | ProA330 5′1 | AAATGGGTAGAATTAACTACAGGTTAAAAATAAAATGTTAACTAAA |
| | | GGTTGCCTCTGAGTAACAGGATTATGGGTGATTTTAATTGTCTTCT |
| | | TTGTGTATGTTCAACAGTGACTATAATATGTATTACTTTTGGAATA |
| | | AAGGAAAACCTGAAAGGTGTGTTGTTTTATAAGGGCCCTTAGGTTG |
| | | CCAAAATTAGAGTCATTGAAATCTAAAGCTGATAAAAACTTTAGTG |
| | | CAAAGATTGTGACATGGGAGACTACACATACCAGATCCATAATGTA |
| | | CATGAGGACAGTAGGCCGAGGGGCCCTGCACATTGAAAGCCCACAT |
| | | GGGAGAAGCCCTTGGGAAGGGGAGTGGAAGGATGAGGCAAGGGGCC |
| | | GGGGGGATGCAGAGGCTGGCAGGCAGTCATTTCTCAGCTTCAGCCA |
| | | TTCCCGCCATGGGGGAATGTGGACAGAGAAGCCAAACAAATCTCCT |
| | | AAACAGTAAATGTCAGTCTTCTGTGTCAGATATTTAAGAAAACTAA |
| | | CAGAGGTCAGAGAAGACACACCTACAGCAAGTAGACTGTCCCTGTG |
| | | CTGCCTTTTTGCAACCCCTGCTTTGGCAGTGCTCAAGCCCACCTCC |
| | | TGCTCTGTGCAGACATCTCTTCTTTGCTCTTACTAGACCAAGGTGA |
| | | AAGAAAACTCTCACCTTCTCCCATCTGGCCCCACAGCATCTGGAAC |
| | | ACACTGATCCTCATAATCCTTGTTCTTGAGAAATATTAATGACTTA |
| | | ATCTCCCAAGCTTGCTCCCTCTCCTGTGCAGGCCATCTCAGTATGT |
| | | TTTGCAGACAAGACCCAGAGAAGTCCAGACTGGACTTGTTGCAGAC |
| | | TGCAAAACTGCCATTGGAAGGCCTCCGTCCCAGTCCTTCTACAGAG |
| | | TAGCCAGTGGGATTCCCAGCC |
| 37 | ProA330 5′2 | ACAGTGACTATAATATGTATTACTTTTGGAATAAAGGAAAACCTGA |
| | | AAGGTGTGTTGTTTTATAAGGGCCCTTAGGTTGCCAAAATTAGAGT |
| | | CATTGAAATCTAAAGCTGATAAAAACTTTAGTGCAAAGATTGTGAC |
| | | ATGGGAGACTACACATACCAGATCCATAATGTACATGAGGACAGTA |
| | | GGCCGAGGGGCCCTGCACATTGAAAGCCCACATGGGAGAAGCCCTT |
| | | GGGAAGGGGAGTGGAAGGATGAGGCAAGGGGCCGGGGGGATGCAGA |
| | | GGCTGGCAGGCAGTCATTTCTCAGCTTCAGCCATTCCCGCCATGGG |
| | | GGAATGTGGACAGAGAAGCCAAACAAATCTCCTAAACAGTAAATGT |
| | | CAGTCTTCTGTGTCAGATATTTAAGAAAACTAACAGAGGTCAGAGA |
| | | AGACACACCTACAGCAAGTAGACTGTCCCTGTGCTGCCTTTTTGCA |
| | | ACCCCTGCTTTGGCAGTGCTCAAGCCCACCTCCTGCTCTGTGCAGA |
| | | CATCTCTTCTTTGCTCTTACTAGACCAAGGTGAAAGAAAACTCTCA |
| | | CCTTCTCCCATCTGGCCCCACAGCATCTGGAACACACTGATCCTCA |
| | | TAATCCTTGTTCTTGAGAAATATTAATGACTTAATCTCCCAAGCTT |
| | | GCTCCCTCTCCTGTGCAGGCCATCTCAGTATGTTTTGCAGACAAGA |
| | | CCCAGAGAAGTCCAGACTGGACTTGTTGCAGACTGCAAAACTGCCA |
| | | TTGGAAGGCCTCCGTCCCAGTCCTTCTACAGAGTAGCCAGTGGGAT |
| | | TCCCAGCC |
| 38 | ProA330 5′3 | AGCTGATAAAAACTTTAGTGCAAAGATTGTGACATGGGAGACTACA |
| | | CATACCAGATCCATAATGTACATGAGGACAGTAGGCCGAGGGGCCC |
| | | TGCACATTGAAAGCCCACATGGGAGAAGCCCTTGGGAAGGGGAGTG |
| | | GAAGGATGAGGCAAGGGGCCGGGGGGATGCAGAGGCTGGCAGGCAG |
| | | TCATTTCTCAGCTTCAGCCATTCCCGCCATGGGGGAATGTGGACAG |
| | | AGAAGCCAAACAAATCTCCTAAACAGTAAATGTCAGTCTTCTGTGT |
| | | CAGATATTTAAGAAAACTAACAGAGGTCAGAGAAGACACACCTACA |
| | | GCAAGTAGACTGTCCCTGTGCTGCCTTTTTGCAACCCCTGCTTTGG |
| | | CAGTGCTCAAGCCCACCTCCTGCTCTGTGCAGACATCTCTTCTTTG |
| | | CTCTTACTAGACCAAGGTGAAAGAAAACTCTCACCTTCTCCCATCT |
| | | GGCCCCACAGCATCTGGAACACACTGATCCTCATAATCCTTGTTCT |
| | | TGAGAAATATTAATGACTTAATCTCCCAAGCTTGCTCCCTCTCCTG |
| | | TGCAGGCCATCTCAGTATGTTTTGCAGACAAGACCCAGAGAAGTCC |
| | | AGACTGGACTTGTTGCAGACTGCAAAACTGCCATTGGAAGGCCTCC |
| | | GTCCCAGTCCTTCTACAGAGTAGCCAGTGGGATTCCCAGCC |
| 39 | ProA330 5′4 | CCCACATGGGAGAAGCCCTTGGGAAGGGGAGTGGAAGGATGAGGCA |
| | | AGGGGCCGGGGGGATGCAGAGGCTGGCAGGCAGTCATTTCTCAGCT |
| | | TCAGCCATTCCCGCCATGGGGGAATGTGGACAGAGAAGCCAAACAA |
| | | ATCTCCTAAACAGTAAATGTCAGTCTTCTGTGTCAGATATTTAAGA |
| | | AAACTAACAGAGGTCAGAGAAGACACACCTACAGCAAGTAGACTGT |
| | | CCCTGTGCTGCCTTTTTGCAACCCCTGCTTTGGCAGTGCTCAAGCC |
| | | CACCTCCTGCTCTGTGCAGACATCTCTTCTTTGCTCTTACTAGACC |
| | | AAGGTGAAAGAAAACTCTCACCTTCTCCCATCTGGCCCCACAGCAT |
| | | CTGGAACACACTGATCCTCATAATCCTTGTTCTTGAGAAATATTAA |
| | | TGACTTAATCTCCCAAGCTTGCTCCCTCTCCTGTGCAGGCCATCTC |
| | | AGTATGTTTTGCAGACAAGACCCAGAGAAGTCCAGACTGGACTTGT |
| | | TGCAGACTGCAAAACTGCCATTGGAAGGCCTCCGTCCCAGTCCTTC |
| | | TACAGAGTAGCCAGTGGGATTCCCAGCC |
| 40 | ProA330 5′5 | CCATGGGGGAATGTGGACAGAGAAGCCAAACAAATCTCCTAAACAG |
| | | TAAATGTCAGTCTTCTGTGTCAGATATTTAAGAAAACTAACAGAGG |
| | | TCAGAGAAGACACACCTACAGCAAGTAGACTGTCCCTGTGCTGCCT |
| | | TTTTGCAACCCCTGCTTTGGCAGTGCTCAAGCCCACCTCCTGCTCT |
| | | GTGCAGACATCTCTTCTTTGCTCTTACTAGACCAAGGTGAAAGAAA |
| | | ACTCTCACCTTCTCCCATCTGGCCCCACAGCATCTGGAACACACTG |
| | | ATCCTCATAATCCTTGTTCTTGAGAAATATTAATGACTTAATCTCC |
| | | CAAGCTTGCTCCCTCTCCTGTGCAGGCCATCTCAGTATGTTTTGCA |
| | | GACAAGACCCAGAGAAGTCCAGACTGGACTTGTTGCAGACTGCAAA |
| | | ACTGCCATTGGAAGGCCTCCGTCCCAGTCCTTCTACAGAGTAGCCA |
| | | GTGGGATTCCCAGCC |
| 41 | ProA330 5′6 | ACCTACAGCAAGTAGACTGTCCCTGTGCTGCCTTTTTGCAACCCCT |
| | | GCTTTGGCAGTGCTCAAGCCCACCTCCTGCTCTGTGCAGACATCTC |
| | | TTCTTTGCTCTTACTAGACCAAGGTGAAAGAAAACTCTCACCTTCT |
| | | CCCATCTGGCCCCACAGCATCTGGAACACACTGATCCTCATAATCC |
| | | TTGTTCTTGAGAAATATTAATGACTTAATCTCCCAAGCTTGCTCCC |
| | | TCTCCTGTGCAGGCCATCTCAGTATGTTTTGCAGACAAGACCCAGA |
| | | GAAGTCCAGACTGGACTTGTTGCAGACTGCAAAACTGCCATTGGAA |
| | | GGCCTCCGTCCCAGTCCTTCTACAGAGTAGCCAGTGGGATTCCCAG |
| | | CC |
| 42 | ProA330 5′7 | CTAGACCAAGGTGAAAGAAAACTCTCACCTTCTCCCATCTGGCCCC |
| | | ACAGCATCTGGAACACACTGATCCTCATAATCCTTGTTCTTGAGAA |
| | | ATATTAATGACTTAATCTCCCAAGCTTGCTCCCTCTCCTGTGCAGG |
| | | CCATCTCAGTATGTTTTGCAGACAAGACCCAGAGAAGTCCAGACTG |
| | | GACTTGTTGCAGACTGCAAAACTGCCATTGGAAGGCCTCCGTCCCA |
| | | GTCCTTCTACAGAGTAGCCAGTGGGATTCCCAGCC |
| 43 | ProA330 5′8 | AATCTCCCAAGCTTGCTCCCTCTCCTGTGCAGGCCATCTCAGTATG |
| | | TTTTGCAGACAAGACCCAGAGAAGTCCAGACTGGACTTGTTGCAGA |
| | | CTGCAAAACTGCCATTGGAAGGCCTCCGTCCCAGTCCTTCTACAGA |
| | | GTAGCCAGTGGGATTCCCAGCC |
| 44 | ProA330 5′9 | ATTGGAAGGCCTCCGTCCCAGTCCTTCTACAGAGTAGCCAGTGGGA |
| | | TTCCCAGCC |
| 45 | ProA330 3′1 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCAGTCTTCTGTGTCAGATAT |
| | | TTAAGAAAACTAACAGAGGTCAGAGAAGACACACCTACAGCAAGTA |
| | | GACTGTCCCTGTGCTGCCTTTTTGCAACCCCTGCTTTGGCAGTGCT |
| | | CAAGCCCACCTCCTGCTCTGTGCAGACATCTCTTCTTTGCTCTTAC |
| | | TAGACCAAGGTGAAAGAAAACTCTCACCTTCTCCCATCTGGCCCCA |
| | | CAGCATCTGGAACACACTGATCCTCATAATCCTTGTTCTTGAGAAA |
| | | TATTAATGACTTAATCTCCCAAGCTTGCTCCCTCTCCTGTGCAGGC |
| | | CATCTCAGTATGTTTTGCAGA |
| 46 | ProA330 3′2 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCAGTCTTCTGTGTCAGATAT |
| | | TTAAGAAAACTAACAGAGGTCAGAGAAGACACACCTACAGCAAGTA |
| | | GACTGTCCCTGTGCTGCCTTTTTGCAACCCCTGCTTTGGCAGTGCT |
| | | CAAGCCCACCTCCTGCTCTGTGCAGACATCTCTTCTTTGCTCTTAC |
| | | TAGACCAAGGTGAAAGAAAACTCTCACCTTCTCCCATCTGGCCCCA |
| | | CAGCATCT |
| 47 | ProA330 3′3 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCAGTCTTCTGTGTCAGATAT |
| | | TTAAGAAAACTAACAGAGGTCAGAGAAGACACACCTACAGCAAGTA |
| | | GACTGTCCCTGTGCTGCCTTTTTGCAACCCCTGCTTTGGCA |
| 48 | ProA330 3′4 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGGGGGGATGCAGAGGCTGGCAGGCAGTCATTTC |
| | | TCAGCTTCAGCCATTCCCGCCATGGGGGAATGTGGACAGAGAAGCC |
| | | AAACAAATCTCCTAAACAGTAAATGTCA |
| 49 | ProA330 3′5 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GATCCATAATGTACATGAGGACAGTAGGCCGAGGGGCCCTGCACAT |
| | | TGAAAGCCCACATGGGAGAAGCCCTTGGGAAGGGGAGTGGAAGGAT |
| | | GAGGCAAGGGGCCGG |
| 50 | ProA330 3′6 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGTTGTTTTATAAG |
| | | GGCCCTTAGGTTGCCAAAATTAGAGTCATTGAAATCTAAAGCTGAT |
| | | AAAAACTTTAGTGCAAAGATTGTGACATGGGAGACTACACATACCA |
| | | GA |
| 51 | ProA330 3′7 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTCTGAGTAACAGGATTATGGGTGATT |
| | | TTAATTGTCTTCTTTGTGTATGTTCAACAGTGACTATAATATGTAT |
| | | TACTTTTGGAATAAAGGAAAACCTGAAAGGTGTGT |
| 52 | ProA330 3′8 | AACCCAAGAAATTACAGGCTGAAACCAGAAAAGAACACATTAAAGC |
| | | ACCAAGAGAAAGTTGGAGTGGGTTGAAGGGAAACAGATTTTTAAAG |
| | | TTAAGGCTCTGTGAAATGGGTAGAATTAACTACAGGTTAAAAATAA |
| | | AATGTTAACTAAAGGTTGCCTC |
| 53 | ProA330 3′9 | AACCCAAGAAATTACAGGCTGAAACCAGAAAAGAACACATTAAAGC |
| | | ACCAAGAGA |
| 54 | min330 | ACCTACAGCAAGTAGACTGTCCCTGTGCTGCCTTTTTGCAACCCCT |
| | | GCTTTGGCAGTGCTCAAGCCCACCTCCTGCTCTGTGCAGACATCTC |
| | | TTCTTTGCTCTTACTAGACCAAGGTGAAAGAAAACTCTCACCTTCT |
| | | CCCATCTGGCCCCACAGCATCTGGAACACACTGATCCTCATAATCC |
| | | TTGTTCTTGAGAAATATTAATGACTTAATCTCCCAAGCTTGCTCCC |
| | | TCTCCTGTGCAGGCCATCTCAGTATGTTTTGCAGA |
| 55 | AAV5-Pro572.2- | CCTGCAGGCAGCTGCGCGCTCGCTCGCTCACTGAGGCCGCCCGGGC |
| | ReaChR-Citrine- | GTCGGGCGACCTTTGGTCGCCCGGCCTCAGTGAGCGAGCGAGCGCG |
| | WPRE-hGH ITR to | CAGAGAGGGAGTGGCCAACTCCATCACTAGGGGTTCCTGCGGCCGC |
| | ITR | ACGCGTGTTAGTCTTTGCCCTTTATCTGCCTGGCAAAGCTTTTAAT |
| | | TGGCTTGATCTGTCATTCCGCTAGACATAAAGGGGACAATCCCCGG |
| | | ATTAGGAAGGAGCTCTCCAGCTCGGGTAAGGAGTCTCAAGGCAAGG |
| | | TAGGCAAGCACCACCGGTCCGCACTCTCGCCCAGCTTTTACGGGAA |
| | | GAAGAGAAACCCAAGAAATTACAGGCTGAAACCAGAAAAGAACACA |
| | | TTAAAGCACCAAGAGAAAGTTGGAGTGGGTTGAAGGGAAACAGATT |
| | | TTTAAAGTTAAGGCTCTGTGAAATGGGTAGAATTAACTACAGGTTA |
| | | AAAATAAAATGTTAACTAAAGGTTGCCTCTGAGTAACAGGATTATG |
| | | GGTGATTTTAATTGTCTTCTTTGTGTATGTTCAACAGTGACTATAA |
| | | TATGTATTACTTTTGGAATAAAGGAAAACCTGAAAGGTGTGTTGTT |
| | | TTATAAGGGCCCTTAGGTTGCCAAAATTAGAGTCATTGAAATCTAA |
| | | AGCTGATAAAAACTTTAGTGCAAAGATTGTGACATGGGAGACTACA |
| | | CATACCAGATCCATAATGTACATGAGGACAGTAGGCCGAGGGGCCC |
| | | TGCACATTGAAAGCCCACATGGGAGAAGCCCTTGGGAAGGGGAGTG |
| | | GAAGGATGAGGCAAGGGGCCGGGGGGATGCAGAGGCTGGCAGGCAG |
| | | TCATTTCTCAGCTTCAGCCATTCCCGCCATGGGGGAATGTGGACAG |
| | | AGAAGCCAAACAAATCTCCTAAACAGTAAATGTCAGTCTTCTGTGT |
| | | CAGATATTTAAGAAAACTAACAGAGGTCAGAGAAGACACACCTACA |
| | | GCAAGTAGACTGTCCCTGTGCTGCCTTTTTGCAACCCCTGCTTTGG |
| | | CAGTGCTCAAGCCCACCTCCTGCTCTGTGCAGACATCTCTTCTTTG |
| | | CTCTTACTAGACCAAGGTGAAAGAAAACTCTCACCTTCTCCCATCT |
| | | GGCCCCACAGCATCTGGAACACACTGATCCTCATAATCCTTGTTCT |
| | | TGAGAAATATTAATGACTTAATCTCCCAAGCTTGCTCCCTCTCCTG |
| | | TGCAGGCCATCTCAGTATGTTTTGCAGACAAGACCCAGAGAAGTCC |
| | | AGACTGGACTTGTTGCAGACTGCAAAACTGCCATTGGAAGGCCTCC |
| | | GTCCCAGTCCTTCTACAGAGTAGCCAGTGGGATTCCCAGCCGGATC |
| | | CGCCACCATGGTGAGCAGAAGACCCTGGCTGCTGGCCCTGGCCCTG |
| | | GCCGTGGCCCTGGCCGCCGGCAGCGCCGGCGCCAGCACCGGCAGCG |
| | | ACGCCACCGTGCCCGTGGCCACCCAGGACGGCCCCGACTACGTGTT |
| | | CCACAGAGCCCACGAGAGAATGCTGTTCCAGACCAGCTACACCCTG |
| | | GAGAACAACGGCAGCGTGATCTGCATCCCCAACAACGGCCAGTGCT |
| | | TCTGCCTGGCCTGGCTGAAGAGTAACGGCACCAACGCCGAGAAGCT |
| | | GGCCGCCAACATCCTGCAGTGGGTGGTGTTTGCGCTGAGCGTGGCG |
| | | TGCCTGGGCTGGTATGCGTATCAGGCGTGGCGCGCGACCTGCGGCT |
| | | GGGAAGAAGTGTATGTGGCGCTGATTGAAATGATGAAAAGCATTAT |
| | | TGAAGCGTTTCATGAATTTGATAGCCCGGCGACCCTGTGGCTGAGC |
| | | AGCGGCAACGGCGTGGTGTGGATGCGCTATGGCGAATGGCTGCTGA |
| | | CCTGCCCCGTGATTCTGATTCATCTGAGCAACCTGACCGGCCTGAA |
| | | AGATGATTATAGCAAACGCACCATGGGCCTGCTGGTGAGCGACGTG |
| | | GGCTGCATTGTGTGGGGCGCGACCAGCGCGATGTGCACCGGCTGGA |
| | | CCAAAATTCTGTTTTTTCTGATTAGCCTGAGCTATGGCATGTATAC |
| | | CTATTTTCATGCGGCCAAAGTGTATATTGAAGCGTTTCATACCGTG |
| | | CCGAAAGGCCTGTGCAGACAGCTGGTGAGAGCCATGGCCTGGCTGT |
| | | TCTTCGTGAGCTGGGGCATGTTCCCCGTGCTGTTCCTGCTGGGCCC |
| | | CGAGGGCTTCGGCCATATTAGCCCGTATGGCAGCGCGATTGGCCAT |
| | | AGCATTCTGGATCTGATTGCGAAGAACATGTGGGGCGTGCTGGGCA |
| | | ACTATCTGCGCGTGAAAATTCATGAACATATTCTGCTGTATGGCGA |
| | | TATTCGCAAAAAACAGAAAATTACCATTGCGGGCCAGGAAATGGAA |
| | | GTGGAAACCCTGGTGGCGGAAGAAGAAGATAAGTACGAGAGCAGCC |
| | | TCGAGATGGTGAGCAAGGGCGAGGAGCTGTTCACCGGGGTGGTGCC |
| | | CATCCTGGTCGAGCTGGACGGCGACGTAAACGGCCACAAGTTCAGC |
| | | GTGTCCGGCGAGGGCGAGGGCGATGCCACCTACGGCAAGCTGACCC |
| | | TGAAGTTCATCTGCACCACCGGCAAGCTGCCCGTGCCCTGGCCCAC |
| | | CCTCGTGACCACCTTCGGCTACGGCCTGATGTGCTTCGCCCGCTAC |
| | | CCCGACCACATGAAGCAGCACGACTTCTTCAAGTCCGCCATGCCCG |
| | | AAGGCTACGTCCAGGAGCGCACCATCTTCTTCAAGGACGACGGCAA |
| | | CTACAAGACCCGCGCCGAGGTGAAGTTCGAGGGCGACACCCTGGTG |
| | | AACCGCATCGAGCTGAAGGGCATCGACTTCAAGGAGGACGGCAACA |
| | | TCCTGGGGCACAAGCTGGAGTACAACTACAACAGCCACAACGTCTA |
| | | TATCATGGCCGACAAGCAGAAGAACGGCATCAAGGTGAACTTCAAG |
| | | ATCCGCCACAACATCGAGGACGGCAGCGTGCAGCTCGCCGACCACT |
| | | ACCAGCAGAACACCCCCATCGGCGACGGCCCCGTGCTGCTGCCCGA |
| | | CAACCACTACCTGAGCTACCAGTCCGCCCTGAGCAAAGACCCCAAC |
| | | GAGAAGCGCGATCACATGGTCCTGCTGGAGTTCGTGACCGCCGCCG |
| | | GGATCACTCTCGGCATGGACGAGCTGTACAAGTAAAGCGGCCGCCG |
| | | GCAAGTAAGCTTATCGATAATCAACCTCTGGATTACAAAATTTGTG |
| | | AAAGATTGACTGGTATTCTTAACTATGTTGCTCCTTTTACGCTATG |
| | | TGGATACGCTGCTTTAATGCCTTTGTATCATGCTATTGCTTCCCGT |
| | | ATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTG |
| | | CACTGTGTTTGCTGACGCAACCCCCACTGGTTGGGGCATTGCCACC |
| | | ACCTGTCAGCTCCTTTCCGGGACTTTCGCTTTCCCCCTCCCTATTG |
| | | CCACGGCGGAACTCATCGCCGCCTGCCTTGCCCGCTGCTGGACAGG |
| | | GGCTCGGCTGTTGGGCACTGACAATTCCGTGGTGTTGTCGGGGAAA |
| | | TCATCGTCCTTTCCTTGGCTGCTCGCCTATGTTGCCACCTGGATTC |
| | | TGCGCGGGACGTCCTTCTGCTACGTCCCTTCGGCCCTCAATCCAGC |
| | | GGACCTTCCTTCCCGCGGCCTGCTGCCGGCTCTGCGGCCTCTTCCG |
| | | CGTCTTCGCCTTCGCCCTCAGACGAGTCGGATCTCCCTTTGGGCCG |
| | | CCTCCCCGCATCGATACCGAGCGCTGCTCGAGAGATCTACGGGTGG |
| | | CATCCCTGTGACCCCTCCCCAGTGCCTCTCCTGGCCCTGGAAGTTG |
| | | CCACTCCAGTGCCCACCAGCCTTGTCCTAATAAAATTAAGTTGCAT |
| | | CATTTTGTCTGACTAGGTGTCCTTCTATAATATTATGGGGTGGAGG |
| | | GGGGTGGTATGGAGCAAGGGGCAAGTTGGGAAGACAACCTGTAGGG |
| | | CCTGCGGGGTCTATTGGGAACCAAGCTGGAGTGCAGTGGCACAATC |
| | | TTGGCTCACTGCAATCTCCGCCTCCTGGGTTCAAGCGATTCTCCTG |
| | | CCTCAGCCTCCCGAGTTGTTGGGATTCCAGGCATGCATGACCAGGC |
| | | TCAGCTAATTTTTGTTTTTTTGGTAGAGACGGGGTTTCACCATATT |
| | | GGCCAGGCTGGTCTCCAACTCCTAATCTCAGGTGATCTACCCACCT |
| | | TGGCCTCCCAAATTGCTGGGATTACAGGCGTGAACCACTGCTCCCT |
| | | TCCCTGTCCTTCTGATTTTGTAGGTAACCACGTGCGGACCGAGCGG |
| | | CCGCAGGAACCCCTAGTGATGGAGTTGGCCACTCCCTCTCTGCGCG |
| | | CTCGCTCGCTCACTGAGGCCGGGCGACCAAAGGTCGCCCGACGCCC |
| | | GGGCTTTGCCCGGGCGGCCTCAGTGAGCGAGCGAGCGCGCAGCTGC |
| | | CTGCAGG |
| 56 | AAV5-Pro573.2- | CCTGCAGGCAGCTGCGCGCTCGCTCGCTCACTGAGGCCGCCCGGGC |
| | ReaChR-Citrine- | GTCGGGCGACCTTTGGTCGCCCGGCCTCAGTGAGCGAGCGAGCGCG |
| | WPRE-hGH ITR to | CAGAGAGGGAGTGGCCAACTCCATCACTAGGGGTTCCTGCGGCCGC |
| | ITR | ACGCGTAACCCAAGAAATTACAGGCTGAAACCAGAAAAGAACACAT |
| | | TAAAGCACCAAGAGAAAGTTGGAGTGGGTTGAAGGGAAACAGATTT |
| | | TTAAAGTTAAGGCTCTGTGAAATGGGTAGAATTAACTACAGGTTAA |
| | | AAATAAAATGTTAACTAAAGGTTGCCTCTGAGTAACAGGATTATGG |
| | | GTGATTTTAATTGTCTTCTTTGTGTATGTTCAACAGTGACTATAAT |
| | | ATGTATTACTTTTGGAATAAAGGAAAACCTGAAAGGTGTGTTGTTT |
| | | TATAAGGGCCCTTAGGTTGCCAAAATTAGAGTCATTGAAATCTAAA |
| | | GCTGATAAAAACTTTAGTGCAAAGATTGTGACATGGGAGACTACAC |
| | | ATACCAGATCCATAATGTACATGAGGACAGTAGGCCGAGGGGCCCT |
| | | GCACATTGAAAGCCCACATGGGAGAAGCCCTTGGGAAGGGGAGTGG |
| | | AAGGATGAGGCAAGGGGCCGGGGGGATGCAGAGGCTGGCAGGCAGT |
| | | CATTTCTCAGCTTCAGCCATTCCCGCCATGGGGGAATGTGGACAGA |
| | | GAAGCCAAACAAATCTCCTAAACAGTAAATGTCAGTCTTCTGTGTC |
| | | AGATATTTAAGAAAACTAACAGAGGTCAGAGAAGACACACCTACAG |
| | | CAAGTAGACTGTCCCTGTGCTGCCTTTTTGCAACCCCTGCTTTGGC |
| | | AGTGCTCAAGCCCACCTCCTGCTCTGTGCAGACATCTCTTCTTTGC |
| | | TCTTACTAGACCAAGGTGAAAGAAAACTCTCACCTTCTCCCATCTG |
| | | GCCCCACAGCATCTGGAACACACTGATCCTCATAATCCTTGTTCTT |
| | | GAGAAATATTAATGACTTAATCTCCCAAGCTTGCTCCCTCTCCTGT |
| | | GCAGGCCATCTCAGTATGTTTTGCAGACAAGACCCAGAGAAGTCCA |
| | | GACTGGACTTGTTGCAGACTGCAAAACTGCCATTGGAAGGCCTCCG |
| | | TCCCAGTCCTTCTACAGAGTAGCCAGTGGGATTCCCAGCCGTTAGT |
| | | CTTTGCCCTTTATCTGCCTGGCAAAGCTTTTAATTGGCTTGATCTG |
| | | TCATTCCGCTAGACATAAAGGGGACAATCCCCGGATTAGGAAGGAG |
| | | CTCTCCAGCTCGGGTAAGGAGTCTCAAGGCAAGGTAGGCAAGCACC |
| | | ACCGGTCCGCACTCTCGCCCAGCTTTTACGGGAAGAAGAGAGGATC |
| | | CGCCACCATGGTGAGCAGAAGACCCTGGCTGCTGGCCCTGGCCCTG |
| | | GCCGTGGCCCTGGCCGCCGGCAGCGCCGGCGCCAGCACCGGCAGCG |
| | | ACGCCACCGTGCCCGTGGCCACCCAGGACGGCCCCGACTACGTGTT |
| | | CCACAGAGCCCACGAGAGAATGCTGTTCCAGACCAGCTACACCCTG |
| | | GAGAACAACGGCAGCGTGATCTGCATCCCCAACAACGGCCAGTGCT |
| | | TCTGCCTGGCCTGGCTGAAGAGTAACGGCACCAACGCCGAGAAGCT |
| | | GGCCGCCAACATCCTGCAGTGGGTGGTGTTTGCGCTGAGCGTGGCG |
| | | TGCCTGGGCTGGTATGCGTATCAGGCGTGGCGCGCGACCTGCGGCT |
| | | GGGAAGAAGTGTATGTGGCGCTGATTGAAATGATGAAAAGCATTAT |
| | | TGAAGCGTTTCATGAATTTGATAGCCCGGCGACCCTGTGGCTGAGC |
| | | AGCGGCAACGGCGTGGTGTGGATGCGCTATGGCGAATGGCTGCTGA |
| | | CCTGCCCCGTGATTCTGATTCATCTGAGCAACCTGACCGGCCTGAA |
| | | AGATGATTATAGCAAACGCACCATGGGCCTGCTGGTGAGCGACGTG |
| | | GGCTGCATTGTGTGGGGCGCGACCAGCGCGATGTGCACCGGCTGGA |
| | | CCAAAATTCTGTTTTTTCTGATTAGCCTGAGCTATGGCATGTATAC |
| | | CTATTTTCATGCGGCCAAAGTGTATATTGAAGCGTTTCATACCGTG |
| | | CCGAAAGGCCTGTGCAGACAGCTGGTGAGAGCCATGGCCTGGCTGT |
| | | TCTTCGTGAGCTGGGGCATGTTCCCCGTGCTGTTCCTGCTGGGCCC |
| | | CGAGGGCTTCGGCCATATTAGCCCGTATGGCAGCGCGATTGGCCAT |
| | | AGCATTCTGGATCTGATTGCGAAGAACATGTGGGGCGTGCTGGGCA |
| | | ACTATCTGCGCGTGAAAATTCATGAACATATTCTGCTGTATGGCGA |
| | | TATTCGCAAAAAACAGAAAATTACCATTGCGGGCCAGGAAATGGAA |
| | | GTGGAAACCCTGGTGGCGGAAGAAGAAGATAAGTACGAGAGCAGCC |
| | | TCGAGATGGTGAGCAAGGGCGAGGAGCTGTTCACCGGGGTGGTGCC |
| | | CATCCTGGTCGAGCTGGACGGCGACGTAAACGGCCACAAGTTCAGC |
| | | GTGTCCGGCGAGGGCGAGGGCGATGCCACCTACGGCAAGCTGACCC |
| | | TGAAGTTCATCTGCACCACCGGCAAGCTGCCCGTGCCCTGGCCCAC |
| | | CCTCGTGACCACCTTCGGCTACGGCCTGATGTGCTTCGCCCGCTAC |
| | | CCCGACCACATGAAGCAGCACGACTTCTTCAAGTCCGCCATGCCCG |
| | | AAGGCTACGTCCAGGAGCGCACCATCTTCTTCAAGGACGACGGCAA |
| | | CTACAAGACCCGCGCCGAGGTGAAGTTCGAGGGCGACACCCTGGTG |
| | | AACCGCATCGAGCTGAAGGGCATCGACTTCAAGGAGGACGGCAACA |
| | | TCCTGGGGCACAAGCTGGAGTACAACTACAACAGCCACAACGTCTA |
| | | TATCATGGCCGACAAGCAGAAGAACGGCATCAAGGTGAACTTCAAG |
| | | ATCCGCCACAACATCGAGGACGGCAGCGTGCAGCTCGCCGACCACT |
| | | ACCAGCAGAACACCCCCATCGGCGACGGCCCCGTGCTGCTGCCCGA |
| | | CAACCACTACCTGAGCTACCAGTCCGCCCTGAGCAAAGACCCCAAC |
| | | GAGAAGCGCGATCACATGGTCCTGCTGGAGTTCGTGACCGCCGCCG |
| | | GGATCACTCTCGGCATGGACGAGCTGTACAAGTAAAGCGGCCGCCG |
| | | GCAAGTAAGCTTATCGATAATCAACCTCTGGATTACAAAATTTGTG |
| | | AAAGATTGACTGGTATTCTTAACTATGTTGCTCCTTTTACGCTATG |
| | | TGGATACGCTGCTTTAATGCCTTTGTATCATGCTATTGCTTCCCGT |
| | | ATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTG |
| | | CACTGTGTTTGCTGACGCAACCCCCACTGGTTGGGGCATTGCCACC |
| | | ACCTGTCAGCTCCTTTCCGGGACTTTCGCTTTCCCCCTCCCTATTG |
| | | CCACGGCGGAACTCATCGCCGCCTGCCTTGCCCGCTGCTGGACAGG |
| | | GGCTCGGCTGTTGGGCACTGACAATTCCGTGGTGTTGTCGGGGAAA |
| | | TCATCGTCCTTTCCTTGGCTGCTCGCCTATGTTGCCACCTGGATTC |
| | | TGCGCGGGACGTCCTTCTGCTACGTCCCTTCGGCCCTCAATCCAGC |
| | | GGACCTTCCTTCCCGCGGCCTGCTGCCGGCTCTGCGGCCTCTTCCG |
| | | CGTCTTCGCCTTCGCCCTCAGACGAGTCGGATCTCCCTTTGGGCCG |
| | | CCTCCCCGCATCGATACCGAGCGCTGCTCGAGAGATCTACGGGTGG |
| | | CATCCCTGTGACCCCTCCCCAGTGCCTCTCCTGGCCCTGGAAGTTG |
| | | CCACTCCAGTGCCCACCAGCCTTGTCCTAATAAAATTAAGTTGCAT |
| | | CATTTTGTCTGACTAGGTGTCCTTCTATAATATTATGGGGTGGAGG |
| | | GGGGTGGTATGGAGCAAGGGGCAAGTTGGGAAGACAACCTGTAGGG |
| | | CCTGCGGGGTCTATTGGGAACCAAGCTGGAGTGCAGTGGCACAATC |
| | | TTGGCTCACTGCAATCTCCGCCTCCTGGGTTCAAGCGATTCTCCTG |
| | | CCTCAGCCTCCCGAGTTGTTGGGATTCCAGGCATGCATGACCAGGC |
| | | TCAGCTAATTTTTGTTTTTTTGGTAGAGACGGGGTTTCACCATATT |
| | | GGCCAGGCTGGTCTCCAACTCCTAATCTCAGGTGATCTACCCACCT |
| | | TGGCCTCCCAAATTGCTGGGATTACAGGCGTGAACCACTGCTCCCT |
| | | TCCCTGTCCTTCTGATTTTGTAGGTAACCACGTGCGGACCGAGCGG |
| | | CCGCAGGAACCCCTAGTGATGGAGTTGGCCACTCCCTCTCTGCGCG |
| | | CTCGCTCGCTCACTGAGGCCGGGCGACCAAAGGTCGCCCGACGCCC |
| | | GGGCTTTGCCCGGGCGGCCTCAGTGAGCGAGCGAGCGCGCAGCTGC |
| | | CTGCAGG |
| 57 | ReaChR, Membrane | MVSRRPWLLALALAVALAAGSAGASTGSDATVPVATQDGPDYVFHR |
| | Localization Sequence | AHERMLFQTSYTLENNGSVICIPNNGQCFCLAWLKSNGTNAEKLAA |
| | (ChIEF) | NIL |
| 58 | ReaChr, Codon- | ATGGTGTCTAGAAGACCTTGGCTGCTGGCCCTGGCTCTGGCTGTGG |
| | optimized version 1, | CCCTTGCTGCTGGATCTGCTGGAGCCAGCACAGGCTCAGATGCCAC |
| | CpG-depleted, | AGTGCCAGTGGCCACACAGGACGGCCCAGATTATGTGTTTCACAGA |
| | alternative reading | GCCCATGAGAGAATGCTGTTCCAAACAAGCTATACACTGGAAAACA |
| | frame eliminated | ACGGATCTGTGATCTGCATCCCCAACAACGGCCAGTGCTTCTGCCT |
| | sequence | GGCTTGGCTTAAGAGTAATGGCACCAATGCAGAGAAGCTGGCTGCC |
| | | AACATCCTGCAGTGGGTTGTGTTTGCCCTGTCTGTTGCTTGCCTGG |
| | | GCTGGTATGCCTACCAAGCCTGGAGGGCTACCTGTGGGTGGGAAGA |
| | | GGTGTATGTGGCCCTGATTGAGATGATGAAAAGCATCATTGAGGCC |
| | | TTCCATGAGTTTGACAGCCCTGCCACCCTGTGGCTGTCCTCTGGGA |
| | | ATGGAGTGGTGTGGATGAGGTATGGAGAGTGGCTCCTGACCTGTCC |
| | | TGTGATCCTGATCCACCTGTCTAATCTGACAGGACTGAAAGACGAC |
| | | TACAGCAAAAGGACTATGGGCCTGCTGGTCTCTGATGTGGGATGTA |
| | | TTGTGTGGGGAGCCACCTCTGCCATGTGCACAGGCTGGACCAAGAT |
| | | CCTGTTTTTCCTGATTTCCCTGAGCTATGGAATGTACACCTACTTC |
| | | CATGCTGCAAAAGTGTACATTGAGGCTTTCCACACAGTGCCTAAGG |
| | | GTCTGTGTAGACAGCTGGTGAGGGCTATGGCCTGGCTTTTCTTTGT |
| | | GTCCTGGGGCATGTTCCCTGTGCTGTTTCTGCTGGGCCCTGAGGGC |
| | | TTTGGCCACATCTCTCCTTATGGCTCTGCCATTGGCCACAGCATCC |
| | | TGGACCTGATAGCCAAAAACATGTGGGGAGTGCTGGGAAATTACCT |
| | | GAGAGTGAAGATCCATGAGCACATTCTGCTGTATGGAGACATCAGA |
| | | AAGAAACAGAAGATCACCATTGCAGGCCAGGAAATGGAAGTTGAGA |
| | | CACTGGTGGCTGAAGAAGAAGATAAGTATGAGAGCAGCCTGGAA |
| 59 | ReaChR, Codon- | ATGGTGAGCAGAAGACCCTGGCTTTTGGCCTTGGCCCTGGCTGTGG |
| | optimized version | CCCTTGCTGCTGGATCTGCTGGGGCATCTACAGGCTCTGATGCTAC |
| | 2, CpG-depleted, | AGTTCCAGTTGCAACCCAGGACGGCCCTGACTATGTGTTCCACAGA |
| | alternative reading | GCCCATGAGAGAATGCTGTTCCAGACCAGCTACACCCTGGAGAACA |
| | frame eliminated | ACGGCTCTGTGATCTGCATCCCCAACAACGGCCAGTGCTTCTGCCT |
| | sequence | TGCATGGCTGAAGAGTAATGGCACCAATGCTGAGAAGCTGGCTGCC |
| | | AACATCCTGCAGTGGGTGGTGTTTGCACTGTCTGTGGCATGCCTGG |
| | | GCTGGTATGCATATCAGGCTTGGAGGGCAACCTGTGGGTGGGAAGA |
| | | AGTGTATGTGGCACTGATTGAAATGATGAAAAGCATTATTGAAGCC |
| | | TTCCATGAATTTGATAGCCCAGCCACCCTGTGGCTGAGCTCTGGCA |
| | | ACGGAGTGGTGTGGATGAGGTATGGAGAGTGGCTGCTGACCTGCCC |
| | | TGTGATTCTGATTCATCTGAGCAACCTGACAGGCCTGAAAGATGAT |
| | | TATAGCAAAAGGACTATGGGCCTGCTGGTGTCTGATGTGGGCTGCA |
| | | TTGTGTGGGGAGCAACCTCTGCAATGTGCACAGGCTGGACCAAAAT |
| | | TCTGTTCTTCCTGATTAGCCTGTCTTATGGCATGTATACCTATTTT |
| | | CATGCAGCCAAAGTGTATATTGAAGCATTTCATACAGTGCCAAAAG |
| | | GCCTGTGCAGACAGCTGGTGAGAGCTATGGCCTGGCTGTTCTTTGT |
| | | GAGCTGGGGAATGTTCCCTGTGCTGTTCCTGCTGGGCCCTGAGGGC |
| | | TTTGGCCATATTAGCCCTTATGGCTCTGCAATTGGCCATAGCATTC |
| | | TGGATCTGATTGCAAAGAATATGTGGGGAGTGCTGGGCAACTATCT |
| | | GAGGGTGAAGATTCATGAACACATTCTGCTGTATGGAGATATTAGG |
| | | AAAAAACAGAAAATTACCATTGCAGGCCAGGAGATGGAAGTGGAAA |
| | | CCCTGGTGGCAGAAGAAGAAGATAAGTATGAGAGCAGCCTGGAG |
| 60 | NFS-GX-009, AAV- | TTGGCCACTCCCTCTCTGCGCGCTCGCTCGCTCACTGAGGCCGCCC |
| | ProX573.2-(T528)- | GGGCAAAGCCCGGGCGTCGGGCGACCTTTGGTCGCCCGGCCTCAGT |
| | ReaChr, Codon- | GAGCGAGCGAGCGCGCAGAGAGGGAGTGGCCAACTCCATCACTAGG |
| | optimized version 1, | GGTTCCTTTGTCGACTCAACCCAAGAAATTACAGGCTGAAACCAGA |
| | CpG-depleted, | AAAGAACACATTAAAGCACCAAGAGAAAGTTGGAGTGGGTTGAAGG |
| | alternative reading | GAAACAGATTTTTAAAGTTAAGGCTCTGTGAAATGGGTAGAATTAA |
| | frame eliminated | CTACAGGTTAAAAATAAAATGTTAACTAAAGGTTGCCTCTGAGTAA |
| | sequence-WPRE-hGH | CAGGATTATGGGTGATTTTAATTGTCTTCTTTGTGTATGTTCAACA |
| | poly A | GTGACTATAATATGTATTACTTTTGGAATAAAGGAAAACCTGAAAG |
| | | GTGTGTTGTTTTATAAGGGCCCTTAGGTTGCCAAAATTAGAGTCAT |
| | | TGAAATCTAAAGCTGATAAAAACTTTAGTGCAAAGATTGTGACATG |
| | | GGAGACTACACATACCAGATCCATAATGTACATGAGGACAGTAGGC |
| | | CGAGGGGCCCTGCACATTGAAAGCCCACATGGGAGAAGCCCTTGGG |
| | | AAGGGGAGTGGAAGGATGAGGCAAGGGGCCGGGGGGATGCAGAGGC |
| | | TGGCAGGCAGTCATTTCTCAGCTTCAGCCATTCCCGCCTTGGGGGA |
| | | ATGTGGACAGAGAAGCCAAACAAATCTCCTAAACAGTAAATGTCAG |
| | | TCTTCTGTGTCAGATATTTAAGAAAACTAACAGAGGTCAGAGAAGA |
| | | CACACCTACAGCAAGTAGACTGTCCCTGTGCTGCCTTTTTGCAACC |
| | | CCTGCTTTGGCAGTGCTCAAGCCCACCTCCTGCTCTGTGCAGACAT |
| | | CTCTTCTTTGCTCTTACTAGACCAAGGTGAAAGAAAACTCTCACCT |
| | | TCTCCCATCTGGCCCCACAGCATCTGGAACACACTGATCCTCATAA |
| | | TCCTTGTTCTTGAGAAATATTAATGACTTAATCTCCCAAGCTTGCT |
| | | CCCTCTCCTGTGCAGGCCATCTCAGTATGTTTTGCAGACAAGACCC |
| | | AGAGAAGTCCAGACTGGACTTGTTGCAGACTGCAAAACTGCCATTG |
| | | GAAGGCCTCCGTCCCAGTCCTTCTACAGAGTAGCCAGTGGGATTCC |
| | | CAGCCGTTAGTCTTTGCCCTTTATCTGCCTGGCAAAGCTTTTAATT |
| | | GGCTTGATCTGTCATTCCGCTAGACATAAAGGGGACAATCCCCGGA |
| | | TTAGGAAGGAGCTCTCCAGCTCGGGTAAGGAGTCTCAAGGCAAGGT |
| | | AGGCAAGCACCACCGGTCCGCACTCTCGCCCAGCTTTTACGGGAAG |
| | | AAGAGACCGCCACCATGGTGTCTAGAAGACCTTGGCTGCTGGCCCT |
| | | GGCTCTGGCTGTGGCCCTTGCTGCTGGATCTGCTGGAGCCAGCACA |
| | | GGCTCAGATGCCACAGTGCCAGTGGCCACACAGGACGGCCCAGATT |
| | | ATGTGTTTCACAGAGCCCATGAGAGAATGCTGTTCCAAACAAGCTA |
| | | TACACTGGAAAACAACGGATCTGTGATCTGCATCCCCAACAACGGC |
| | | CAGTGCTTCTGCCTGGCTTGGCTTAAGAGTAATGGCACCAATGCAG |
| | | AGAAGCTGGCTGCCAACATCCTGCAGTGGGTTGTGTTTGCCCTGTC |
| | | TGTTGCTTGCCTGGGCTGGTATGCCTACCAAGCCTGGAGGGCTACC |
| | | TGTGGGTGGGAAGAGGTGTATGTGGCCCTGATTGAGATGATGAAAA |
| | | GCATCATTGAGGCCTTCCATGAGTTTGACAGCCCTGCCACCCTGTG |
| | | GCTGTCCTCTGGGAATGGAGTGGTGTGGATGAGGTATGGAGAGTGG |
| | | CTCCTGACCTGTCCTGTGATCCTGATCCACCTGTCTAATCTGACAG |
| | | GACTGAAAGACGACTACAGCAAAAGGACTATGGGCCTGCTGGTCTC |
| | | TGATGTGGGATGTATTGTGTGGGGAGCCACCTCTGCCATGTGCACA |
| | | GGCTGGACCAAGATCCTGTTTTTCCTGATTTCCCTGAGCTATGGAA |
| | | TGTACACCTACTTCCATGCTGCAAAAGTGTACATTGAGGCTTTCCA |
| | | CACAGTGCCTAAGGGTCTGTGTAGACAGCTGGTGAGGGCTATGGCC |
| | | TGGCTTTTCTTTGTGTCCTGGGGCATGTTCCCTGTGCTGTTTCTGC |
| | | TGGGCCCTGAGGGCTTTGGCCACATCTCTCCTTATGGCTCTGCCAT |
| | | TGGCCACAGCATCCTGGACCTGATAGCCAAAAACATGTGGGGAGTG |
| | | CTGGGAAATTACCTGAGAGTGAAGATCCATGAGCACATTCTGCTGT |
| | | ATGGAGACATCAGAAAGAAACAGAAGATCACCATTGCAGGCCAGGA |
| | | AATGGAAGTTGAGACACTGGTGGCTGAAGAAGAAGATAAGTATGAG |
| | | AGCAGCCTGGAATAATCAACCTCTGGATTACAAAATTTGTGAAAGA |
| | | TTGACTGGTATTCTTAACTATGTTGCTCCTTTTACGCTATGTGGAT |
| | | ACGCTGCTTTAATGCCTTTGTATCATGCTATTGCTTCCCGTATGGC |
| | | TTTCATTTTCTCCTCCTTGTATAAATCCTGGTTGCTGTCTCTTTAT |
| | | GAGGAGTTGTGGCCCGTTGTCAGGCAACGTGGCGTGGTGTGCACTG |
| | | TGTTTGCTGACGCAACCCCCACTGGTTGGGGCATTGCCACCACCTG |
| | | TCAGCTCCTTTCCGGGACTTTCGCTTTCCCCCTCCCTATTGCCACG |
| | | GCGGAACTCATCGCCGCCTGCCTTGCCCGCTGCTGGACAGGGGCTC |
| | | GGCTGTTGGGCACTGACAATTCCGTGGTGTTGTCGGGGAAATCATC |
| | | GTCCTTTCCTTGGCTGCTCGCCTATGTTGCCACCTGGATTCTGCGC |
| | | GGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCT |
| | | TCGCCTTCGCCCTCAGACGAGTCGGATCTCCCTTTGGGCCGCCTCC |
| | | CCGCGGGTGGCATCCCTGTGACCCCTCCCCAGTGCCTCTCCTGGCC |
| | | CTGGAAGTTGCCACTCCAGTGCCCACCAGCCTTGTCCTAATAAAAT |
| | | TAAGTTGCATCATTTTGTCTGACTAGGTGTCCTTCTATAATATTAT |
| | | GGGGTGGAGGGGGGTGGTATGGAGCAAGGGGCAAGTTGGGAAGACA |
| | | ACCTGTAGGGCCTGCGGGGTCTATTGGGAACCAAGCTGGAGTGCAG |
| | | TGGCACAATCTTGGCTCACTGCAATCTCCGCCTCCTGGGTTCAAGC |
| | | GATTCTCCTGCCTCAGCCTCCCGAGTTGTTGGGATTCCAGGCATGC |
| | | ATGACCAGGCTCAGCTAATTTTTGTTTTTTTGGTAGAGACGGGGTT |
| | | TCACCATATTGGCCAGGCTGGTCTCCAACTCCTAATCTCAGGTGAT |
| | | CTACCCACCTTGGCCTCCCAAATTGCTGGGATTACAGGCGTGAACC |
| | | ACTGCTCCCTTCCCTGTCCTTCTGATTTTGAGTCCTAGGAGGAACC |
| | | CCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCC |
| | | GGGCGGCCTCAGTGAGCGAGCGAGCGCGCAGAGAGGGAGTGGCCAA |
| 61 | ReaChR-non-codon | ATGGTGAGCAGAAGACCCTGGCTGCTGGCCCTGGCCCTGGCCGTGG |
| | optimized | CCCTGGCCGCCGGCAGCGCCGGCGCCAGCACCGGCAGCGACGCCAC |
| | | CGTGCCCGTGGCCACCCAGGACGGCCCCGACTACGTGTTCCACAGA |
| | | GCCCACGAGAGAATGCTGTTCCAGACCAGCTACACCCTGGAGAACA |
| | | ACGGCAGCGTGATCTGCATCCCCAACAACGGCCAGTGCTTCTGCCT |
| | | GGCCTGGCTGAAGAGTAACGGCACCAACGCCGAGAAGCTGGCCGCC |
| | | AACATCCTGCAGTGGGTGGTGTTTGCGCTGAGCGTGGCGTGCCTGG |
| | | GCTGGTATGCGTATCAGGCGTGGCGCGCGACCTGCGGCTGGGAAGA |
| | | AGTGTATGTGGCGCTGATTGAAATGATGAAAAGCATTATTGAAGCG |
| | | TTTCATGAATTTGATAGCCCGGCGACCCTGTGGCTGAGCAGCGGCA |
| | | ACGGCGTGGTGTGGATGCGCTATGGCGAATGGCTGCTGACCTGCCC |
| | | CGTGATTCTGATTCATCTGAGCAACCTGACCGGCCTGAAAGATGAT |
| | | TATAGCAAACGCACCATGGGCCTGCTGGTGAGCGACGTGGGCTGCA |
| | | TTGTGTGGGGCGCGACCAGCGCGATGTGCACCGGCTGGACCAAAAT |
| | | TCTGTTTTTTCTGATTAGCCTGAGCTATGGCATGTATACCTATTTT |
| | | CATGCGGCCAAAGTGTATATTGAAGCGTTTCATACCGTGCCGAAAG |
| | | GCCTGTGCAGACAGCTGGTGAGAGCCATGGCCTGGCTGTTCTTCGT |
| | | GAGCTGGGGCATGTTCCCCGTGCTGTTCCTGCTGGGCCCCGAGGGC |
| | | TTCGGCCATATTAGCCCGTATGGCAGCGCGATTGGCCATAGCATTC |
| | | TGGATCTGATTGCGAAGAACATGTGGGGCGTGCTGGGCAACTATCT |
| | | GCGCGTGAAAATTCATGAACATATTCTGCTGTATGGCGATATTCGC |
| | | AAAAAACAGAAAATTACCATTGCGGGCCAGGAAATGGAAGTGGAAA |
| | | CCCTGGTGGCGGAAGAAGAAGATAAGTACGAGAGCAGCCTCGAG |
| 62 | coSensorE_NFS-GX- | ATGGTGTCTAGAAGACCTTGGCTGCTGGCCCTGGCTCTGGCCGTGG |
| | 003 | CCCTTGCCGCTGGAAGCGCTGGCGCCAGCACCGGCTCCGACGCCAC |
| | | AGTGCCAGTGGCCACACAGGACGGCCCTGATTACGTGTTTCACAGA |
| | | GCCCACGAGAGAATGCTGTTCCAAACAAGCTATACACTGGAAAACA |
| | | ACGGCAGCGTGATCTGCATCCCCAACAACGGCCAGTGCTTCTGCCT |
| | | GGCCTGGCTGAAGAGCAACGGCACCAACGCCGAGAAGCTGGCTGCC |
| | | AACATCCTGCAGTGGGTCGTGTTCGCCCTGTCTGTTGCTTGCCTGG |
| | | GCTGGTACGCCTACCAGGCCTGGCGCGCTACCTGCGGCTGGGAAGA |
| | | GGTGTATGTGGCCCTGATCGAGATGATGAAAAGCATCATCGAGGCC |
| | | TTCCACGAGTTCGACAGCCCTGCCACCCTGTGGCTGTCCAGCGGCA |
| | | ACGGCGTGGTGTGGATGCGGTACGGAGAATGGCTCCTGACCTGTCC |
| | | TGTGATCCTGATCCACCTGTCTAATCTGACAGGACTGAAGGACGAC |
| | | TACAGCAAGCGGACCATGGGCCTGCTGGTCTCTGATGTGGGATGTA |
| | | TCGTGTGGGGAGCCACCAGCGCCATGTGCACCGGCTGGACCAAGAT |
| | | CCTGTTTTTCCTGATTTCCCTGAGCTACGGCATGTACACCTACTTC |
| | | CATGCCGCAAAAGTGTACATCGAGGCTTTCCACACCGTGCCTAAGG |
| | | GTCTGTGTAGACAGCTGGTGCGGGCCATGGCCTGGCTGTTCTTCGT |
| | | GTCCTGGGGCATGTTCCCCGTGCTGTTTCTGCTGGGCCCCGAGGGC |
| | | TTCGGCCACATCTCTCCTTACGGCAGCGCCATCGGCCACAGCATCC |
| | | TGGACCTGATCGCCAAGAACATGTGGGGCGTGCTGGGAAATTACCT |
| | | GAGAGTGAAGATCCACGAGCACATTCTGCTGTACGGCGACATCAGA |
| | | AAGAAACAGAAGATCACCATCGCCGGCCAGGAGATGGAAGTCGAGA |
| | | CACTGGTGGCCGAAGAAGAAGATAAGTACGAGAGCAGCCTGGAA |
| 63 | CGd_SensorE_NFS- | ATGGTGAGCAGAAGACCCTGGCTGCTGGCCCTGGCCCTGGCTGTGG |
| | GX-003 | CCCTGGCTGCTGGCTCTGCTGGAGCCAGCACAGGCTCTGATGCCAC |
| | | AGTGCCTGTGGCCACCCAGGATGGCCCTGACTATGTGTTCCACAGA |
| | | GCCCATGAGAGAATGCTGTTCCAGACCAGCTACACCCTGGAGAACA |
| | | ATGGCTCTGTGATCTGCATCCCCAACAATGGCCAGTGCTTCTGCCT |
| | | GGCCTGGCTGAAGAGTAATGGCACCAATGCTGAGAAGCTGGCTGCC |
| | | AACATCCTGCAGTGGGTGGTGTTTGCACTGTCTGTGGCATGCCTGG |
| | | GCTGGTATGCATATCAGGCATGGAGGGCAACCTGTGGCTGGGAAGA |
| | | AGTGTATGTGGCACTGATTGAAATGATGAAAAGCATTATTGAAGCA |
| | | TTTCATGAATTTGATAGCCCAGCCACCCTGTGGCTGAGCTCTGGCA |
| | | ATGGAGTGGTGTGGATGAGGTATGGAGAATGGCTGCTGACCTGCCC |
| | | TGTGATTCTGATTCATCTGAGCAACCTGACAGGCCTGAAAGATGAT |
| | | TATAGCAAAAGGACCATGGGCCTGCTGGTGTCTGATGTGGGCTGCA |
| | | TTGTGTGGGGAGCAACCTCTGCAATGTGCACAGGCTGGACCAAAAT |
| | | TCTGTTTTTTCTGATTAGCCTGAGCTATGGCATGTATACCTATTTT |
| | | CATGCAGCCAAAGTGTATATTGAAGCATTTCATACAGTGCCAAAAG |
| | | GCCTGTGCAGACAGCTGGTGAGAGCCATGGCCTGGCTGTTCTTTGT |
| | | GAGCTGGGGCATGTTCCCTGTGCTGTTCCTGCTGGGCCCTGAGGGC |
| | | TTTGGCCATATTAGCCCTTATGGCTCTGCAATTGGCCATAGCATTC |
| | | TGGATCTGATTGCAAAGAACATGTGGGGAGTGCTGGGCAACTATCT |
| | | GAGGGTGAAAATTCATGAACATATTCTGCTGTATGGAGATATTAGG |
| | | AAAAAACAGAAAATTACCATTGCAGGCCAGGAAATGGAAGTGGAAA |
| | | CCCTGGTGGCAGAAGAAGAAGATAAGTATGAGAGCAGCCTGGAG |
| 64 | CGd_coSensorE_NFS- | ATGGTGTCTAGAAGACCTTGGCTGCTGGCCCTGGCTCTGGCTGTGG |
| | GX-003 | CCCTTGCTGCTGGATCTGCTGGAGCCAGCACAGGCTCAGATGCCAC |
| | | AGTGCCAGTGGCCACACAGGATGGCCCTGATTATGTGTTTCACAGA |
| | | GCCCATGAGAGAATGCTGTTCCAAACAAGCTATACACTGGAAAACA |
| | | ATGGATCTGTGATCTGCATCCCCAACAATGGCCAGTGCTTCTGCCT |
| | | GGCCTGGCTGAAGAGCAATGGCACCAATGCAGAGAAGCTGGCTGCC |
| | | AACATCCTGCAGTGGGTTGTGTTTGCCCTGTCTGTTGCTTGCCTGG |
| | | GCTGGTATGCCTACCAGGCCTGGAGGGCTACCTGTGGCTGGGAAGA |
| | | GGTGTATGTGGCCCTGATTGAGATGATGAAAAGCATCATTGAGGCC |
| | | TTCCATGAGTTTGACAGCCCTGCCACCCTGTGGCTGTCCTCTGGCA |
| | | ATGGAGTGGTGTGGATGAGGTATGGAGAATGGCTCCTGACCTGTCC |
| | | TGTGATCCTGATCCACCTGTCTAATCTGACAGGACTGAAGGATGAC |
| | | TACAGCAAAAGGACCATGGGCCTGCTGGTCTCTGATGTGGGATGTA |
| | | TTGTGTGGGGAGCCACCTCTGCCATGTGCACAGGCTGGACCAAGAT |
| | | CCTGTTTTTCCTGATTTCCCTGAGCTATGGCATGTACACCTACTTC |
| | | CATGCTGCAAAAGTGTACATTGAGGCTTTCCACACAGTGCCTAAGG |
| | | GTCTGTGTAGACAGCTGGTGAGGGCCATGGCCTGGCTGTTCTTTGT |
| | | GTCCTGGGGCATGTTCCCTGTGCTGTTTCTGCTGGGCCCTGAGGGC |
| | | TTTGGCCACATCTCTCCTTATGGCTCTGCCATTGGCCACAGCATCC |
| | | TGGACCTGATAGCCAAGAACATGTGGGGAGTGCTGGGAAATTACCT |
| | | GAGAGTGAAGATCCATGAGCACATTCTGCTGTATGGAGACATCAGA |
| | | AAGAAACAGAAGATCACCATTGCAGGCCAGGAGATGGAAGTTGAGA |
| | | CACTGGTGGCTGAAGAAGAAGATAAGTATGAGAGCAGCCTGGAA |
| 65 | RHd_CpG_SensorE_ | ATGGTGAGCAGAAGACCCTGGCTTTTGGCCTTGGCCCTGGCTGTGG |
| | NFS-GX-003 | CCCTTGCTGCTGGATCTGCTGGGGCATCTACAGGCTCTGATGCTAC |
| | | AGTTCCAGTTGCAACCCAGGATGGCCCTGACTATGTGTTCCACAGA |
| | | GCCCATGAGAGAATGCTGTTCCAGACCAGCTACACCCTGGAGAACA |
| | | ATGGCTCTGTGATCTGCATCCCCAACAATGGCCAGTGCTTCTGCCT |
| | | TGCATGGCTGAAGAGTAATGGCACCAATGCTGAGAAGCTGGCTGCC |
| | | AACATCCTGCAGTGGGTGGTGTTTGCACTGTCTGTGGCATGCCTGG |
| | | GCTGGTATGCATATCAGGCATGGAGGGCAACCTGTGGGTGGGAAGA |
| | | AGTGTATGTGGCACTGATTGAAATGATGAAAAGCATTATTGAAGCC |
| | | TTCCATGAATTTGATAGCCCAGCCACCCTGTGGCTGAGCTCTGGCA |
| | | ATGGAGTGGTGTGGATGAGGTATGGAGAATGGCTGCTGACCTGCCC |
| | | TGTGATTCTGATTCATCTGAGCAACCTGACAGGCCTGAAAGATGAT |
| | | TATAGCAAAAGGACCATGGGCCTGCTGGTGTCTGATGTGGGCTGCA |
| | | TTGTGTGGGGAGCAACCTCTGCAATGTGCACAGGCTGGACCAAAAT |
| | | TCTGTTCTTCCTGATTAGCCTGAGCTATGGCATGTATACCTATTTT |
| | | CATGCAGCCAAAGTGTATATTGAAGCATTTCATACAGTGCCAAAAG |
| | | GCCTGTGCAGACAGCTGGTGAGAGCCATGGCCTGGCTGTTCTTTGT |
| | | GAGCTGGGGAATGTTCCCTGTGCTGTTCCTGCTGGGCCCTGAGGGC |
| | | TTTGGCCATATTAGCCCTTATGGCTCTGCAATTGGCCATAGCATTC |
| | | TGGATCTGATTGCAAAGAACATGTGGGGAGTGCTGGGCAACTATCT |
| | | GAGGGTGAAGATTCATGAACACATTCTGCTGTATGGAGATATTAGG |
| | | AAAAAACAGAAAATTACCATTGCAGGCCAGGAAATGGAAGTGGAAA |
| | | CCCTGGTGGCAGAAGAAGAAGATAAGTATGAGAGCAGCCTGGAG |
| 66 | RHd_CGd_coSensorE_ | ATGGTGTCTAGAAGACCTTGGCTGCTGGCCCTGGCTCTGGCTGTGG |
| | NFS-GX-003 | CCCTTGCTGCTGGATCTGCTGGAGCCAGCACAGGCTCAGATGCCAC |
| | | AGTGCCAGTGGCCACACAGGATGGCCCAGATTATGTGTTTCACAGA |
| | | GCCCATGAGAGAATGCTGTTCCAAACAAGCTATACACTGGAAAACA |
| | | ATGGATCTGTGATCTGCATCCCCAACAATGGCCAGTGCTTCTGCCT |
| | | GGCATGGCTTAAGAGCAATGGCACCAATGCAGAGAAGCTGGCTGCC |
| | | AACATCCTGCAGTGGGTTGTGTTTGCCCTGTCTGTTGCTTGCCTGG |
| | | GCTGGTATGCCTACCAAGCCTGGAGGGCTACCTGTGGGTGGGAAGA |
| | | GGTGTATGTGGCCCTGATTGAGATGATGAAAAGCATCATTGAGGCC |
| | | TTCCATGAGTTTGACAGCCCTGCCACCCTGTGGCTGTCCTCTGGCA |
| | | ATGGAGTGGTGTGGATGAGGTATGGAGAATGGCTCCTGACCTGTCC |
| | | TGTGATCCTGATCCACCTGTCTAATCTGACAGGACTGAAGGATGAC |
| | | TACAGCAAAAGGACCATGGGCCTGCTGGTCTCTGATGTGGGATGTA |
| | | TTGTGTGGGGAGCCACCTCTGCCATGTGCACAGGCTGGACCAAGAT |
| | | CCTGTTTTTCCTGATTTCCCTGAGCTATGGAATGTACACCTACTTC |
| | | CATGCTGCAAAAGTGTACATTGAGGCTTTCCACACAGTGCCTAAGG |
| | | GTCTGTGTAGACAGCTGGTGAGGGCTATGGCCTGGCTTTTCTTTGT |
| | | GTCCTGGGGCATGTTCCCTGTGCTGTTTCTGCTGGGCCCTGAGGGC |
| | | TTTGGCCACATCTCTCCTTATGGCTCTGCCATTGGCCACAGCATCC |
| | | TGGACCTGATAGCCAAGAACATGTGGGGAGTGCTGGGAAATTACCT |
| | | GAGAGTGAAGATCCATGAGCACATTCTGCTGTATGGAGACATCAGA |
| | | AAGAAACAGAAGATCACCATTGCAGGCCAGGAGATGGAAGTTGAGA |
| | | CACTGGTGGCTGAAGAAGAAGATAAGTATGAGAGCAGCCTGGAA |
| 67 | RHd_CpG_SensorE_ | GTCCTGCAGGCAGCTGCGCGCTCGCTCGCTCACTGAGGCCGCCCGG |
| | NFS-GX-003_ITR to | GCGTCGGGCGACCTTTGGTCGCCCGGCCTCAGTGAGCGAGCGAGCG |
| | ITR | CGCAGAGAGGGAGTGGCCAACTCCATCACTAGGGGTTCCTGCGGCC |
| | | GCACGCGTAACCCAAGAAATTACAGGCTGAAACCAGAAAAGAACAC |
| | | ATTAAAGCACCAAGAGAAAGTTGGAGTGGGTTGAAGGGAAACAGAT |
| | | TTTTAAAGTTAAGGCTCTGTGAAATGGGTAGAATTAACTACAGGTT |
| | | AAAAATAAAATGTTAACTAAAGGTTGCCTCTGAGTAACAGGATTAT |
| | | GGGTGATTTTAATTGTCTTCTTTGTGTATGTTCAACAGTGACTATA |
| | | ATATGTATTACTTTTGGAATAAAGGAAAACCTGAAAGGTGTGTTGT |
| | | TTTATAAGGGCCCTTAGGTTGCCAAAATTAGAGTCATTGAAATCTA |
| | | AAGCTGATAAAAACTTTAGTGCAAAGATTGTGACATGGGAGACTAC |
| | | ACATACCAGATCCATAATGTACATGAGGACAGTAGGCCGAGGGGCC |
| | | CTGCACATTGAAAGCCCACATGGGAGAAGCCCTTGGGAAGGGGAGT |
| | | GGAAGGATGAGGCAAGGGGCCGGGGGGATGCAGAGGCTGGCAGGCA |
| | | GTCATTTCTCAGCTTCAGCCATTCCCGCCATGGGGGAATGTGGACA |
| | | GAGAAGCCAAACAAATCTCCTAAACAGTAAATGTCAGTCTTCTGTG |
| | | TCAGATATTTAAGAAAACTAACAGAGGTCAGAGAAGACACACCTAC |
| | | AGCAAGTAGACTGTCCCTGTGCTGCCTTTTTGCAACCCCTGCTTTG |
| | | GCAGTGCTCAAGCCCACCTCCTGCTCTGTGCAGACATCTCTTCTTT |
| | | GCTCTTACTAGACCAAGGTGAAAGAAAACTCTCACCTTCTCCCATC |
| | | TGGCCCCACAGCATCTGGAACACACTGATCCTCATAATCCTTGTTC |
| | | TTGAGAAATATTAATGACTTAATCTCCCAAGCTTGCTCCCTCTCCT |
| | | GTGCAGGCCATCTCAGTATGTTTTGCAGACAAGACCCAGAGAAGTC |
| | | CAGACTGGACTTGTTGCAGACTGCAAAACTGCCATTGGAAGGCCTC |
| | | CGTCCCAGTCCTTCTACAGAGTAGCCAGTGGGATTCCCAGCCGTTA |
| | | GTCTTTGCCCTTTATCTGCCTGGCAAAGCTTTTAATTGGCTTGATC |
| | | TGTCATTCCGCTAGACATAAAGGGGACAATCCCCGGATTAGGAAGG |
| | | AGCTCTCCAGCTCGGGTAAGGAGTCTCAAGGCAAGGTAGGCAAGCA |
| | | CCACCGGTCCGCACTCTCGCCCAGCTTTTACGGGAAGAAGAGAGGA |
| | | TCCGCCACCATGGTGAGCAGAAGACCCTGGCTTTTGGCCTTGGCCC |
| | | TGGCTGTGGCCCTTGCTGCTGGATCTGCTGGGGCATCTACAGGCTC |
| | | TGATGCTACAGTTCCAGTTGCAACCCAGGATGGCCCTGACTATGTG |
| | | TTCCACAGAGCCCATGAGAGAATGCTGTTCCAGACCAGCTACACCC |
| | | TGGAGAACAATGGCTCTGTGATCTGCATCCCCAACAATGGCCAGTG |
| | | CTTCTGCCTTGCATGGCTGAAGAGTAATGGCACCAATGCTGAGAAG |
| | | CTGGCTGCCAACATCCTGCAGTGGGTGGTGTTTGCACTGTCTGTGG |
| | | CATGCCTGGGCTGGTATGCATATCAGGCATGGAGGGCAACCTGTGG |
| | | GTGGGAAGAAGTGTATGTGGCACTGATTGAAATGATGAAAAGCATT |
| | | ATTGAAGCCTTCCATGAATTTGATAGCCCAGCCACCCTGTGGCTGA |
| | | GCTCTGGCAATGGAGTGGTGTGGATGAGGTATGGAGAATGGCTGCT |
| | | GACCTGCCCTGTGATTCTGATTCATCTGAGCAACCTGACAGGCCTG |
| | | AAAGATGATTATAGCAAAAGGACCATGGGCCTGCTGGTGTCTGATG |
| | | TGGGCTGCATTGTGTGGGGAGCAACCTCTGCAATGTGCACAGGCTG |
| | | GACCAAAATTCTGTTCTTCCTGATTAGCCTGAGCTATGGCATGTAT |
| | | ACCTATTTTCATGCAGCCAAAGTGTATATTGAAGCATTTCATACAG |
| | | TGCCAAAAGGCCTGTGCAGACAGCTGGTGAGAGCCATGGCCTGGCT |
| | | GTTCTTTGTGAGCTGGGGAATGTTCCCTGTGCTGTTCCTGCTGGGC |
| | | CCTGAGGGCTTTGGCCATATTAGCCCTTATGGCTCTGCAATTGGCC |
| | | ATAGCATTCTGGATCTGATTGCAAAGAACATGTGGGGAGTGCTGGG |
| | | CAACTATCTGAGGGTGAAGATTCATGAACACATTCTGCTGTATGGA |
| | | GATATTAGGAAAAAACAGAAAATTACCATTGCAGGCCAGGAAATGG |
| | | AAGTGGAAACCCTGGTGGCAGAAGAAGAAGATAAGTATGAGAGCAG |
| | | CCTGGAGTAAAGCGGCCGCCGGCAAGTAAGCTTATCGATAATCAAC |
| | | CTCTGGATTACAAAATTTGTGAAAGATTGACTGGTATTCTTAACTA |
| | | TGTTGCTCCTTTTACGCTATGTGGATACGCTGCTTTAATGCCTTTG |
| | | TATCATGCTATTGCTTCCCGTATGGCTTTCATTTTCTCCTCCTTGT |
| | | ATAAATCCTGGTTGCTGTCTCTTTATGAGGAGTTGTGGCCCGTTGT |
| | | CAGGCAACGTGGCGTGGTGTGCACTGTGTTTGCTGACGCAACCCCC |
| | | ACTGGTTGGGGCATTGCCACCACCTGTCAGCTCCTTTCCGGGACTT |
| | | TCGCTTTCCCCCTCCCTATTGCCACGGCGGAACTCATCGCCGCCTG |
| | | CCTTGCCCGCTGCTGGACAGGGGCTCGGCTGTTGGGCACTGACAAT |
| | | TCCGTGGTGTTGTCGGGGAAATCATCGTCCTTTCCTTGGCTGCTCG |
| | | CCTATGTTGCCACCTGGATTCTGCGCGGGACGTCCTTCTGCTACGT |
| | | CCCTTCGGCCCTCAATCCAGCGGACCTTCCTTCCCGCGGCCTGCTG |
| | | CCGGCTCTGCGGCCTCTTCCGCGTCTTCGCCTTCGCCCTCAGACGA |
| | | GTCGGATCTCCCTTTGGGCCGCCTCCCCGCATCGATACCGAGCGCT |
| | | GCTCGAGAGATCTACGGGTGGCATCCCTGTGACCCCTCCCCAGTGC |
| | | CTCTCCTGGCCCTGGAAGTTGCCACTCCAGTGCCCACCAGCCTTGT |
| | | CCTAATAAAATTAAGTTGCATCATTTTGTCTGACTAGGTGTCCTTC |
| | | TATAATATTATGGGGTGGAGGGGGGTGGTATGGAGCAAGGGGCAAG |
| | | TTGGGAAGACAACCTGTAGGGCCTGCGGGGTCTATTGGGAACCAAG |
| | | CTGGAGTGCAGTGGCACAATCTTGGCTCACTGCAATCTCCGCCTCC |
| | | TGGGTTCAAGCGATTCTCCTGCCTCAGCCTCCCGAGTTGTTGGGAT |
| | | TCCAGGCATGCATGACCAGGCTCAGCTAATTTTTGTTTTTTTGGTA |
| | | GAGACGGGGTTTCACCATATTGGCCAGGCTGGTCTCCAACTCCTAA |
| | | TCTCAGGTGATCTACCCACCTTGGCCTCCCAAATTGCTGGGATTAC |
| | | AGGCGTGAACCACTGCTCCCTTCCCTGTCCTTCTGATTTTGTAGGT |
| | | AACCACGTGCGGACCGAGCGGCCGCAGGAACCCCTAGTGATGGAGT |
| | | TGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTG |
| | | AGCGAGCGAGCGCGCAGCTGCCTGCAGGGGC |
| 68 | ARFe_RHd_CpG_ | TGTCCTGCAGGCAGCTGCGCGCTCGCTCGCTCACTGAGGCCGCCCG |
| | SensorE_NFS-GX- | GGCGTCGGGCGACCTTTGGTCGCCCGGCCTCAGTGAGCGAGCGAGC |
| | 003_ITR to ITR | GCGCAGAGAGGGAGTGGCCAACTCCATCACTAGGGGTTCCTGCGGC |
| | | CGCACGCGTAACCCAAGAAATTACAGGCTGAAACCAGAAAAGAACA |
| | | CATTAAAGCACCAAGAGAAAGTTGGAGTGGGTTGAAGGGAAACAGA |
| | | TTTTTAAAGTTAAGGCTCTGTGAAATGGGTAGAATTAACTACAGGT |
| | | TAAAAATAAAATGTTAACTAAAGGTTGCCTCTGAGTAACAGGATTA |
| | | TGGGTGATTTTAATTGTCTTCTTTGTGTATGTTCAACAGTGACTAT |
| | | AATATGTATTACTTTTGGAATAAAGGAAAACCTGAAAGGTGTGTTG |
| | | TTTTATAAGGGCCCTTAGGTTGCCAAAATTAGAGTCATTGAAATCT |
| | | AAAGCTGATAAAAACTTTAGTGCAAAGATTGTGACATGGGAGACTA |
| | | CACATACCAGATCCATAATGTACATGAGGACAGTAGGCCGAGGGGC |
| | | CCTGCACATTGAAAGCCCACATGGGAGAAGCCCTTGGGAAGGGGAG |
| | | TGGAAGGATGAGGCAAGGGGCCGGGGGGATGCAGAGGCTGGCAGGC |
| | | AGTCATTTCTCAGCTTCAGCCATTCCCGCCATGGGGGAATGTGGAC |
| | | AGAGAAGCCAAACAAATCTCCTAAACAGTAAATGTCAGTCTTCTGT |
| | | GTCAGATATTTAAGAAAACTAACAGAGGTCAGAGAAGACACACCTA |
| | | CAGCAAGTAGACTGTCCCTGTGCTGCCTTTTTGCAACCCCTGCTTT |
| | | GGCAGTGCTCAAGCCCACCTCCTGCTCTGTGCAGACATCTCTTCTT |
| | | TGCTCTTACTAGACCAAGGTGAAAGAAAACTCTCACCTTCTCCCAT |
| | | CTGGCCCCACAGCATCTGGAACACACTGATCCTCATAATCCTTGTT |
| | | CTTGAGAAATATTAATGACTTAATCTCCCAAGCTTGCTCCCTCTCC |
| | | TGTGCAGGCCATCTCAGTATGTTTTGCAGACAAGACCCAGAGAAGT |
| | | CCAGACTGGACTTGTTGCAGACTGCAAAACTGCCATTGGAAGGCCT |
| | | CCGTCCCAGTCCTTCTACAGAGTAGCCAGTGGGATTCCCAGCCGTT |
| | | AGTCTTTGCCCTTTATCTGCCTGGCAAAGCTTTTAATTGGCTTGAT |
| | | CTGTCATTCCGCTAGACATAAAGGGGACAATCCCCGGATTAGGAAG |
| | | GAGCTCTCCAGCTCGGGTAAGGAGTCTCAAGGCAAGGTAGGCAAGC |
| | | ACCACCGGTCCGCACTCTCGCCCAGCTTTTACGGGAAGAAGAGAGG |
| | | ATCCGCCACCATGGTGAGCAGAAGACCCTGGCTTTTGGCCTTGGCC |
| | | CTGGCTGTGGCCCTTGCTGCTGGATCTGCTGGGGCATCTACAGGCT |
| | | CTGATGCTACAGTTCCAGTTGCAACCCAGGACGGCCCTGACTATGT |
| | | GTTCCACAGAGCCCATGAGAGAATGCTGTTCCAGACCAGCTACACC |
| | | CTGGAGAACAACGGCTCTGTGATCTGCATCCCCAACAACGGCCAGT |
| | | GCTTCTGCCTTGCATGGCTGAAGAGTAATGGCACCAATGCTGAGAA |
| | | GCTGGCTGCCAACATCCTGCAGTGGGTGGTGTTTGCACTGTCTGTG |
| | | GCATGCCTGGGCTGGTATGCATATCAGGCTTGGAGGGCAACCTGTG |
| | | GGTGGGAAGAAGTGTATGTGGCACTGATTGAAATGATGAAAAGCAT |
| | | TATTGAAGCCTTCCATGAATTTGATAGCCCAGCCACCCTGTGGCTG |
| | | AGCTCTGGCAACGGAGTGGTGTGGATGAGGTATGGAGAGTGGCTGC |
| | | TGACCTGCCCTGTGATTCTGATTCATCTGAGCAACCTGACAGGCCT |
| | | GAAAGATGATTATAGCAAAAGGACTATGGGCCTGCTGGTGTCTGAT |
| | | GTGGGCTGCATTGTGTGGGGAGCAACCTCTGCAATGTGCACAGGCT |
| | | GGACCAAAATTCTGTTCTTCCTGATTAGCCTGTCTTATGGCATGTA |
| | | TACCTATTTTCATGCAGCCAAAGTGTATATTGAAGCATTTCATACA |
| | | GTGCCAAAAGGCCTGTGCAGACAGCTGGTGAGAGCTATGGCCTGGC |
| | | TGTTCTTTGTGAGCTGGGGAATGTTCCCTGTGCTGTTCCTGCTGGG |
| | | CCCTGAGGGCTTTGGCCATATTAGCCCTTATGGCTCTGCAATTGGC |
| | | CATAGCATTCTGGATCTGATTGCAAAGAATATGTGGGGAGTGCTGG |
| | | GCAACTATCTGAGGGTGAAGATTCATGAACACATTCTGCTGTATGG |
| | | AGATATTAGGAAAAAACAGAAAATTACCATTGCAGGCCAGGAGATG |
| | | GAAGTGGAAACCCTGGTGGCAGAAGAAGAAGATAAGTATGAGAGCA |
| | | GCCTGGAGTAAAGCGGCCGCCGGCAAGTAAGCTTATCGATAATCAA |
| | | CCTCTGGATTACAAAATTTGTGAAAGATTGACTGGTATTCTTAACT |
| | | ATGTTGCTCCTTTTACGCTATGTGGATACGCTGCTTTAATGCCTTT |
| | | GTATCATGCTATTGCTTCCCGTATGGCTTTCATTTTCTCCTCCTTG |
| | | TATAAATCCTGGTTGCTGTCTCTTTATGAGGAGTTGTGGCCCGTTG |
| | | TCAGGCAACGTGGCGTGGTGTGCACTGTGTTTGCTGACGCAACCCC |
| | | CACTGGTTGGGGCATTGCCACCACCTGTCAGCTCCTTTCCGGGACT |
| | | TTCGCTTTCCCCCTCCCTATTGCCACGGCGGAACTCATCGCCGCCT |
| | | GCCTTGCCCGCTGCTGGACAGGGGCTCGGCTGTTGGGCACTGACAA |
| | | TTCCGTGGTGTTGTCGGGGAAATCATCGTCCTTTCCTTGGCTGCTC |
| | | GCCTATGTTGCCACCTGGATTCTGCGCGGGACGTCCTTCTGCTACG |
| | | TCCCTTCGGCCCTCAATCCAGCGGACCTTCCTTCCCGCGGCCTGCT |
| | | GCCGGCTCTGCGGCCTCTTCCGCGTCTTCGCCTTCGCCCTCAGACG |
| | | AGTCGGATCTCCCTTTGGGCCGCCTCCCCGCATCGATACCGAGCGC |
| | | TGCTCGAGAGATCTACGGGTGGCATCCCTGTGACCCCTCCCCAGTG |
| | | CCTCTCCTGGCCCTGGAAGTTGCCACTCCAGTGCCCACCAGCCTTG |
| | | TCCTAATAAAATTAAGTTGCATCATTTTGTCTGACTAGGTGTCCTT |
| | | CTATAATATTATGGGGTGGAGGGGGGTGGTATGGAGCAAGGGGCAA |
| | | GTTGGGAAGACAACCTGTAGGGCCTGCGGGGTCTATTGGGAACCAA |
| | | GCTGGAGTGCAGTGGCACAATCTTGGCTCACTGCAATCTCCGCCTC |
| | | CTGGGTTCAAGCGATTCTCCTGCCTCAGCCTCCCGAGTTGTTGGGA |
| | | TTCCAGGCATGCATGACCAGGCTCAGCTAATTTTTGTTTTTTTGGT |
| | | AGAGACGGGGTTTCACCATATTGGCCAGGCTGGTCTCCAACTCCTA |
| | | ATCTCAGGTGATCTACCCACCTTGGCCTCCCAAATTGCTGGGATTA |
| | | CAGGCGTGAACCACTGCTCCCTTCCCTGTCCTTCTGATTTTGTAGG |
| | | TAACCACGTGCGGACCGAGCGGCCGCAGGAACCCCTAGTGATGGAG |
| | | TTGGCCACTCCCTCTCTGCGCGCTCGCTCGCTCACTGAGGCCGGGC |
| | | GACCAAAGGTCGCCCGACGCCCGGGCTTTGCCCGGGCGGCCTCAGT |
| | | GAGCGAGCGAGCGCGCAGCTGCCTGC |
| 69 | ARFe_RHd_CpG_SensorE | ATGGTGAGCAGAAGACCCTGGCTTTTGGCCTTGGCCCTGGCT |
| | (NFS-GX-004) | GTGGCCCTTGCTGCTGGATCTGCTGGGGCATCTACAGGCTCT |
| | | GATGCTACAGTTCCAGTTGCAACCCAGGACGGCCCTGACTAT |
| | | GTGTTCCACAGAGCCCATGAGAGAATGCTGTTCCAGACCAGC |
| | | TACACCCTGGAGAACAACGGCTCTGTGATCTGCATCCCCAAC |
| | | AACGGCCAGTGCTTCTGCCTTGCATGGCTGAAGAGTAATGGC |
| | | ACCAATGCTGAGAAGCTGGCTGCCAACATCCTGCAGTGGGTG |
| | | GTGTTTGCACTGTCTGTGGCATGCCTGGGCTGGTATGCATAT |
| | | CAGGCTTGGAGGGCAACCTGTGGGTGGGAAGAAGTGTATGTG |
| | | GCACTGATTGAAATGATGAAAAGCATTATTGAAGCCTTCCAT |
| | | GAATTTGATAGCCCAGCCACCCTGTGGCTGAGCTCTGGCAAC |
| | | GGAGTGGTGTGGATGAGGTATGGAGAGTGGCTGCTGACCTGC |
| | | CCTGTGATTCTGATTCATCTGAGCAACCTGACAGGCCTGAAA |
| | | GATGATTATAGCAAAAGGACTATGGGCCTGCTGGTGTCTGAT |
| | | GTGGGCTGCATTGTGTGGGGAGCAACCTCTGCAATGTGCACA |
| | | GGCTGGACCAAAATTCTGTTCTTCCTGATTAGCCTGTCTTAT |
| | | GGCATGTATACCTATTTTCATGCAGCCAAAGTGTATATTGAA |
| | | GCATTTCATACAGTGCCAAAAGGCCTGTGCAGACAGCTGGTG |
| | | AGAGCTATGGCCTGGCTGTTCTTTGTGAGCTGGGGAATGTTC |
| | | CCTGTGCTGTTCCTGCTGGGCCCTGAGGGCTTTGGCCATATT |
| | | AGCCCTTATGGCTCTGCAATTGGCCATAGCATTCTGGATCTG |
| | | ATTGCAAAGAATATGTGGGGAGTGCTGGGCAACTATCTGAGG |
| | | GTGAAGATTCATGAACACATTCTGCTGTATGGAGATATTAGG |
| | | AAAAAACAGAAAATTACCATTGCAGGCCAGGAGATGGAAGTG |
| | | GAAACCCTGGTGGCAGAAGAAGAAGATAAGTATGAGAGCAGC |
| | | CTGGAG |
| 70 | ARFe_RHd_CpG_ | MVSRRPWLLALALAVALAAGSAGASTGSDATVPVATQDGPDYVFHR |
| | SensorE (NSF-GX-004) | AHERMLFQTSYTLENNGSVICIPNNGQCFCLAWLKSNGTNAEKLAA |
| | | NILQWVVFALSVACLGWYAYQAWRATCGWEEVYVALIEMMKSIIEA |
| | | FHEFDSPATLWLSSGNGVVWMRYGEWLLTCPVILIHLSNLTGLKDD |
| | | YSKRTMGLLVSDVGCIVWGATSAMCTGWTKILFFLISLSYGMYTYF |
| | | HAAKVYIEAFHTVPKGLCRQLVRAMAWLFFVSWGMFPVLFLLGPEG |
| | | FGHISPYGSAIGHSILDLIAKNMWGVLGNYLRVKIHEHILLYGDIR |
| | | KKQKITIAGQEMEVETLVAEEEDKYESSLE |
| 71 | RHd_CpG_coSensorE_ | GCAGGCAGCTGCGCGCTCGCTCGCTCACTGAGGCCGCCCGGGCGTC |
| | NFS-GX-003_ITR to | GGGCGACCTTTGGTCGCCCGGCCTCAGTGAGCGAGCGAGCGCGCAG |
| | ITR | AGAGGGAGTGGCCAACTCCATCACTAGGGGTTCCTGCGGCCGCACG |
| | | CGTAACCCAAGAAATTACAGGCTGAAACCAGAAAAGAACACATTAA |
| | | AGCACCAAGAGAAAGTTGGAGTGGGTTGAAGGGAAACAGATTTTTA |
| | | AAGTTAAGGCTCTGTGAAATGGGTAGAATTAACTACAGGTTAAAAA |
| | | TAAAATGTTAACTAAAGGTTGCCTCTGAGTAACAGGATTATGGGTG |
| | | ATTTTAATTGTCTTCTTTGTGTATGTTCAACAGTGACTATAATATG |
| | | TATTACTTTTGGAATAAAGGAAAACCTGAAAGGTGTGTTGTTTTAT |
| | | AAGGGCCCTTAGGTTGCCAAAATTAGAGTCATTGAAATCTAAAGCT |
| | | GATAAAAACTTTAGTGCAAAGATTGTGACATGGGAGACTACACATA |
| | | CCAGATCCATAATGTACATGAGGACAGTAGGCCGAGGGGCCCTGCA |
| | | CATTGAAAGCCCACATGGGAGAAGCCCTTGGGAAGGGGAGTGGAAG |
| | | GATGAGGCAAGGGGCCGGGGGGATGCAGAGGCTGGCAGGCAGTCAT |
| | | TTCTCAGCTTCAGCCATTCCCGCCATGGGGGAATGTGGACAGAGAA |
| | | GCCAAACAAATCTCCTAAACAGTAAATGTCAGTCTTCTGTGTCAGA |
| | | TATTTAAGAAAACTAACAGAGGTCAGAGAAGACACACCTACAGCAA |
| | | GTAGACTGTCCCTGTGCTGCCTTTTTGCAACCCCTGCTTTGGCAGT |
| | | GCTCAAGCCCACCTCCTGCTCTGTGCAGACATCTCTTCTTTGCTCT |
| | | TACTAGACCAAGGTGAAAGAAAACTCTCACCTTCTCCCATCTGGCC |
| | | CCACAGCATCTGGAACACACTGATCCTCATAATCCTTGTTCTTGAG |
| | | AAATATTAATGACTTAATCTCCCAAGCTTGCTCCCTCTCCTGTGCA |
| | | GGCCATCTCAGTATGTTTTGCAGACAAGACCCAGAGAAGTCCAGAC |
| | | TGGACTTGTTGCAGACTGCAAAACTGCCATTGGAAGGCCTCCGTCC |
| | | CAGTCCTTCTACAGAGTAGCCAGTGGGATTCCCAGCCGTTAGTCTT |
| | | TGCCCTTTATCTGCCTGGCAAAGCTTTTAATTGGCTTGATCTGTCA |
| | | TTCCGCTAGACATAAAGGGGACAATCCCCGGATTAGGAAGGAGCTC |
| | | TCCAGCTCGGGTAAGGAGTCTCAAGGCAAGGTAGGCAAGCACCACC |
| | | GGTCCGCACTCTCGCCCAGCTTTTACGGGAAGAAGAGAGGATCCGC |
| | | CACCATGGTGTCTAGAAGACCTTGGCTGCTGGCCCTGGCTCTGGCT |
| | | GTGGCCCTTGCTGCTGGATCTGCTGGAGCCAGCACAGGCTCAGATG |
| | | CCACAGTGCCAGTGGCCACACAGGATGGCCCAGATTATGTGTTTCA |
| | | CAGAGCCCATGAGAGAATGCTGTTCCAAACAAGCTATACACTGGAA |
| | | AACAATGGATCTGTGATCTGCATCCCCAACAATGGCCAGTGCTTCT |
| | | GCCTGGCATGGCTTAAGAGCAATGGCACCAATGCAGAGAAGCTGGC |
| | | TGCCAACATCCTGCAGTGGGTTGTGTTTGCCCTGTCTGTTGCTTGC |
| | | CTGGGCTGGTATGCCTACCAAGCCTGGAGGGCTACCTGTGGGTGGG |
| | | AAGAGGTGTATGTGGCCCTGATTGAGATGATGAAAAGCATCATTGA |
| | | GGCCTTCCATGAGTTTGACAGCCCTGCCACCCTGTGGCTGTCCTCT |
| | | GGCAATGGAGTGGTGTGGATGAGGTATGGAGAATGGCTCCTGACCT |
| | | GTCCTGTGATCCTGATCCACCTGTCTAATCTGACAGGACTGAAGGA |
| | | TGACTACAGCAAAAGGACCATGGGCCTGCTGGTCTCTGATGTGGGA |
| | | TGTATTGTGTGGGGAGCCACCTCTGCCATGTGCACAGGCTGGACCA |
| | | AGATCCTGTTTTTCCTGATTTCCCTGAGCTATGGAATGTACACCTA |
| | | CTTCCATGCTGCAAAAGTGTACATTGAGGCTTTCCACACAGTGCCT |
| | | AAGGGTCTGTGTAGACAGCTGGTGAGGGCTATGGCCTGGCTTTTCT |
| | | TTGTGTCCTGGGGCATGTTCCCTGTGCTGTTTCTGCTGGGCCCTGA |
| | | GGGCTTTGGCCACATCTCTCCTTATGGCTCTGCCATTGGCCACAGC |
| | | ATCCTGGACCTGATAGCCAAGAACATGTGGGGAGTGCTGGGAAATT |
| | | ACCTGAGAGTGAAGATCCATGAGCACATTCTGCTGTATGGAGACAT |
| | | CAGAAAGAAACAGAAGATCACCATTGCAGGCCAGGAGATGGAAGTT |
| | | GAGACACTGGTGGCTGAAGAAGAAGATAAGTATGAGAGCAGCCTGG |
| | | AATAAAGCGGCCGCCGGCAAGTAAGCTTATCGATAATCAACCTCTG |
| | | GATTACAAAATTTGTGAAAGATTGACTGGTATTCTTAACTATGTTG |
| | | CTCCTTTTACGCTATGTGGATACGCTGCTTTAATGCCTTTGTATCA |
| | | TGCTATTGCTTCCCGTATGGCTTTCATTTTCTCCTCCTTGTATAAA |
| | | TCCTGGTTGCTGTCTCTTTATGAGGAGTTGTGGCCCGTTGTCAGGC |
| | | AACGTGGCGTGGTGTGCACTGTGTTTGCTGACGCAACCCCCACTGG |
| | | TTGGGGCATTGCCACCACCTGTCAGCTCCTTTCCGGGACTTTCGCT |
| | | TTCCCCCTCCCTATTGCCACGGCGGAACTCATCGCCGCCTGCCTTG |
| | | CCCGCTGCTGGACAGGGGCTCGGCTGTTGGGCACTGACAATTCCGT |
| | | GGTGTTGTCGGGGAAATCATCGTCCTTTCCTTGGCTGCTCGCCTAT |
| | | GTTGCCACCTGGATTCTGCGCGGGACGTCCTTCTGCTACGTCCCTT |
| | | CGGCCCTCAATCCAGCGGACCTTCCTTCCCGCGGCCTGCTGCCGGC |
| | | TCTGCGGCCTCTTCCGCGTCTTCGCCTTCGCCCTCAGACGAGTCGG |
| | | ATCTCCCTTTGGGCCGCCTCCCCGCATCGATACCGAGCGCTGCTCG |
| | | AGAGATCTACGGGTGGCATCCCTGTGACCCCTCCCCAGTGCCTCTC |
| | | CTGGCCCTGGAAGTTGCCACTCCAGTGCCCACCAGCCTTGTCCTAA |
| | | TAAAATTAAGTTGCATCATTTTGTCTGACTAGGTGTCCTTCTATAA |
| | | TATTATGGGGTGGAGGGGGGTGGTATGGAGCAAGGGGCAAGTTGGG |
| | | AAGACAACCTGTAGGGCCTGCGGGGTCTATTGGGAACCAAGCTGGA |
| | | GTGCAGTGGCACAATCTTGGCTCACTGCAATCTCCGCCTCCTGGGT |
| | | TCAAGCGATTCTCCTGCCTCAGCCTCCCGAGTTGTTGGGATTCCAG |
| | | GCATGCATGACCAGGCTCAGCTAATTTTTGTTTTTTTGGTAGAGAC |
| | | GGGGTTTCACCATATTGGCCAGGCTGGTCTCCAACTCCTAATCTCA |
| | | GGTGATCTACCCACCTTGGCCTCCCAAATTGCTGGGATTACAGGCG |
| | | TGAACCACTGCTCCCTTCCCTGTCCTTCTGATTTTGTAGGTAACCA |
| | | CGTGCGGACCGAGCGGCCGCAGGAACCCCTAGTGATGGAGTTGGCC |
| | | ACTCCCTCTCTGCGCGCTCGCTCGCTCACTGAGGCCGGGCGACCAA |
| | | AGGTCGCCCGACGCCCGGGCTTTGCCCGGGCGGCCTCAGTGAGCGA |
| | | GCGAGCGCGCAGCT |
| 72 | ARFe_RHd_CpG_ | GCAGGCAGCTGCGCGCTCGCTCGCTCACTGAGGCCGCCCGGGCGTC |
| | coSensorE_NFS-GX-003 | GGGCGACCTTTGGTCGCCCGGCCTCAGTGAGCGAGCGAGCGCGCAG |
| | | AGAGGGAGTGGCCAACTCCATCACTAGGGGTTCCTGCGGCCGCACG |
| | | CGTAACCCAAGAAATTACAGGCTGAAACCAGAAAAGAACACATTAA |
| | | AGCACCAAGAGAAAGTTGGAGTGGGTTGAAGGGAAACAGATTTTTA |
| | | AAGTTAAGGCTCTGTGAAATGGGTAGAATTAACTACAGGTTAAAAA |
| | | TAAAATGTTAACTAAAGGTTGCCTCTGAGTAACAGGATTATGGGTG |
| | | ATTTTAATTGTCTTCTTTGTGTATGTTCAACAGTGACTATAATATG |
| | | TATTACTTTTGGAATAAAGGAAAACCTGAAAGGTGTGTTGTTTTAT |
| | | AAGGGCCCTTAGGTTGCCAAAATTAGAGTCATTGAAATCTAAAGCT |
| | | GATAAAAACTTTAGTGCAAAGATTGTGACATGGGAGACTACACATA |
| | | CCAGATCCATAATGTACATGAGGACAGTAGGCCGAGGGGCCCTGCA |
| | | CATTGAAAGCCCACATGGGAGAAGCCCTTGGGAAGGGGAGTGGAAG |
| | | GATGAGGCAAGGGGCCGGGGGGATGCAGAGGCTGGCAGGCAGTCAT |
| | | TTCTCAGCTTCAGCCATTCCCGCCATGGGGGAATGTGGACAGAGAA |
| | | GCCAAACAAATCTCCTAAACAGTAAATGTCAGTCTTCTGTGTCAGA |
| | | TATTTAAGAAAACTAACAGAGGTCAGAGAAGACACACCTACAGCAA |
| | | GTAGACTGTCCCTGTGCTGCCTTTTTGCAACCCCTGCTTTGGCAGT |
| | | GCTCAAGCCCACCTCCTGCTCTGTGCAGACATCTCTTCTTTGCTCT |
| | | TACTAGACCAAGGTGAAAGAAAACTCTCACCTTCTCCCATCTGGCC |
| | | CCACAGCATCTGGAACACACTGATCCTCATAATCCTTGTTCTTGAG |
| | | AAATATTAATGACTTAATCTCCCAAGCTTGCTCCCTCTCCTGTGCA |
| | | GGCCATCTCAGTATGTTTTGCAGACAAGACCCAGAGAAGTCCAGAC |
| | | TGGACTTGTTGCAGACTGCAAAACTGCCATTGGAAGGCCTCCGTCC |
| | | CAGTCCTTCTACAGAGTAGCCAGTGGGATTCCCAGCCGTTAGTCTT |
| | | TGCCCTTTATCTGCCTGGCAAAGCTTTTAATTGGCTTGATCTGTCA |
| | | TTCCGCTAGACATAAAGGGGACAATCCCCGGATTAGGAAGGAGCTC |
| | | TCCAGCTCGGGTAAGGAGTCTCAAGGCAAGGTAGGCAAGCACCACC |
| | | GGTCCGCACTCTCGCCCAGCTTTTACGGGAAGAAGAGAGGATCCGC |
| | | CACCATGGTGTCTAGAAGACCTTGGCTGCTGGCCCTGGCTCTGGCT |
| | | GTGGCCCTTGCTGCTGGATCTGCTGGAGCCAGCACAGGCTCAGATG |
| | | CCACAGTGCCAGTGGCCACACAGGACGGCCCAGATTATGTGTTTCA |
| | | CAGAGCCCATGAGAGAATGCTGTTCCAAACAAGCTATACACTGGAA |
| | | AACAACGGATCTGTGATCTGCATCCCCAACAACGGCCAGTGCTTCT |
| | | GCCTGGCTTGGCTTAAGAGTAATGGCACCAATGCAGAGAAGCTGGC |
| | | TGCCAACATCCTGCAGTGGGTTGTGTTTGCCCTGTCTGTTGCTTGC |
| | | CTGGGCTGGTATGCCTACCAAGCCTGGAGGGCTACCTGTGGGTGGG |
| | | AAGAGGTGTATGTGGCCCTGATTGAGATGATGAAAAGCATCATTGA |
| | | GGCCTTCCATGAGTTTGACAGCCCTGCCACCCTGTGGCTGTCCTCT |
| | | GGGAATGGAGTGGTGTGGATGAGGTATGGAGAGTGGCTCCTGACCT |
| | | GTCCTGTGATCCTGATCCACCTGTCTAATCTGACAGGACTGAAAGA |
| | | CGACTACAGCAAAAGGACTATGGGCCTGCTGGTCTCTGATGTGGGA |
| | | TGTATTGTGTGGGGAGCCACCTCTGCCATGTGCACAGGCTGGACCA |
| | | AGATCCTGTTTTTCCTGATTTCCCTGAGCTATGGAATGTACACCTA |
| | | CTTCCATGCTGCAAAAGTGTACATTGAGGCTTTCCACACAGTGCCT |
| | | AAGGGTCTGTGTAGACAGCTGGTGAGGGCTATGGCCTGGCTTTTCT |
| | | TTGTGTCCTGGGGCATGTTCCCTGTGCTGTTTCTGCTGGGCCCTGA |
| | | GGGCTTTGGCCACATCTCTCCTTATGGCTCTGCCATTGGCCACAGC |
| | | ATCCTGGACCTGATAGCCAAAAACATGTGGGGAGTGCTGGGAAATT |
| | | ACCTGAGAGTGAAGATCCATGAGCACATTCTGCTGTATGGAGACAT |
| | | CAGAAAGAAACAGAAGATCACCATTGCAGGCCAGGAAATGGAAGTT |
| | | GAGACACTGGTGGCTGAAGAAGAAGATAAGTATGAGAGCAGCCTGG |
| | | AATAAAGCGGCCGCCGGCAAGTAAGCTTATCGATAATCAACCTCTG |
| | | GATTACAAAATTTGTGAAAGATTGACTGGTATTCTTAACTATGTTG |
| | | CTCCTTTTACGCTATGTGGATACGCTGCTTTAATGCCTTTGTATCA |
| | | TGCTATTGCTTCCCGTATGGCTTTCATTTTCTCCTCCTTGTATAAA |
| | | TCCTGGTTGCTGTCTCTTTATGAGGAGTTGTGGCCCGTTGTCAGGC |
| | | AACGTGGCGTGGTGTGCACTGTGTTTGCTGACGCAACCCCCACTGG |
| | | TTGGGGCATTGCCACCACCTGTCAGCTCCTTTCCGGGACTTTCGCT |
| | | TTCCCCCTCCCTATTGCCACGGCGGAACTCATCGCCGCCTGCCTTG |
| | | CCCGCTGCTGGACAGGGGCTCGGCTGTTGGGCACTGACAATTCCGT |
| | | GGTGTTGTCGGGGAAATCATCGTCCTTTCCTTGGCTGCTCGCCTAT |
| | | GTTGCCACCTGGATTCTGCGCGGGACGTCCTTCTGCTACGTCCCTT |
| | | CGGCCCTCAATCCAGCGGACCTTCCTTCCCGCGGCCTGCTGCCGGC |
| | | TCTGCGGCCTCTTCCGCGTCTTCGCCTTCGCCCTCAGACGAGTCGG |
| | | ATCTCCCTTTGGGCCGCCTCCCCGCATCGATACCGAGCGCTGCTCG |
| | | AGAGATCTACGGGTGGCATCCCTGTGACCCCTCCCCAGTGCCTCTC |
| | | CTGGCCCTGGAAGTTGCCACTCCAGTGCCCACCAGCCTTGTCCTAA |
| | | TAAAATTAAGTTGCATCATTTTGTCTGACTAGGTGTCCTTCTATAA |
| | | TATTATGGGGTGGAGGGGGGTGGTATGGAGCAAGGGGCAAGTTGGG |
| | | AAGACAACCTGTAGGGCCTGCGGGGTCTATTGGGAACCAAGCTGGA |
| | | GTGCAGTGGCACAATCTTGGCTCACTGCAATCTCCGCCTCCTGGGT |
| | | TCAAGCGATTCTCCTGCCTCAGCCTCCCGAGTTGTTGGGATTCCAG |
| | | GCATGCATGACCAGGCTCAGCTAATTTTTGTTTTTTTGGTAGAGAC |
| | | GGGGTTTCACCATATTGGCCAGGCTGGTCTCCAACTCCTAATCTCA |
| | | GGTGATCTACCCACCTTGGCCTCCCAAATTGCTGGGATTACAGGCG |
| | | TGAACCACTGCTCCCTTCCCTGTCCTTCTGATTTTGTAGGTAACCA |
| | | CGTGCGGACCGAGCGGCCGCAGGAACCCCTAGTGATGGAGTTGGCC |
| | | ACTCCCTCTCTGCGCGCTCGCTCGCTCACTGAGGCCGGGCGACCAA |
| | | AGGTCGCCCGACGCCCGGGCTTTGCCCGGGCGGCCTCAGTGAGCGA |
| | | GCGAGCGCGCAGCTGCCTGCAGGGGCG |
| 73 | ARFe_RHd_CpG_coSensorE | ATGGTGTCTAGAAGACCTTGGCTGCTGGCCCTGGCTCTGGCTGTGG |
| | (NFS-GX-004) | CCCTTGCTGCTGGATCTGCTGGAGCCAGCACAGGCTCAGATGCCAC |
| | | AGTGCCAGTGGCCACACAGGACGGCCCAGATTATGTGTTTCACAGA |
| | | GCCCATGAGAGAATGCTGTTCCAAACAAGCTATACACTGGAAAACA |
| | | ACGGATCTGTGATCTGCATCCCCAACAACGGCCAGTGCTTCTGCCT |
| | | GGCTTGGCTTAAGAGTAATGGCACCAATGCAGAGAAGCTGGCTGCC |
| | | AACATCCTGCAGTGGGTTGTGTTTGCCCTGTCTGTTGCTTGCCTGG |
| | | GCTGGTATGCCTACCAAGCCTGGAGGGCTACCTGTGGGTGGGAAGA |
| | | GGTGTATGTGGCCCTGATTGAGATGATGAAAAGCATCATTGAGGCC |
| | | TTCCATGAGTTTGACAGCCCTGCCACCCTGTGGCTGTCCTCTGGGA |
| | | ATGGAGTGGTGTGGATGAGGTATGGAGAGTGGCTCCTGACCTGTCC |
| | | TGTGATCCTGATCCACCTGTCTAATCTGACAGGACTGAAAGACGAC |
| | | TACAGCAAAAGGACTATGGGCCTGCTGGTCTCTGATGTGGGATGTA |
| | | TTGTGTGGGGAGCCACCTCTGCCATGTGCACAGGCTGGACCAAGAT |
| | | CCTGTTTTTCCTGATTTCCCTGAGCTATGGAATGTACACCTACTTC |
| | | CATGCTGCAAAAGTGTACATTGAGGCTTTCCACACAGTGCCTAAGG |
| | | GTCTGTGTAGACAGCTGGTGAGGGCTATGGCCTGGCTTTTCTTTGT |
| | | GTCCTGGGGCATGTTCCCTGTGCTGTTTCTGCTGGGCCCTGAGGGC |
| | | TTTGGCCACATCTCTCCTTATGGCTCTGCCATTGGCCACAGCATCC |
| | | TGGACCTGATAGCCAAAAACATGTGGGGAGTGCTGGGAAATTACCT |
| | | GAGAGTGAAGATCCATGAGCACATTCTGCTGTATGGAGACATCAGA |
| | | AAGAAACAGAAGATCACCATTGCAGGCCAGGAAATGGAAGTTGAGA |
| | | CACTGGTGGCTGAAGAAGAAGATAAGTATGAGAGCAGCCTGGAA |
| 74 | ARFe_RHd_CpG_coSensorE | MVSRRPWLLALALAVALAAGSAGASTGSDATVPVATQDGPDYVFHR |
| | (NFS-GX-004) | AHERMLFQTSYTLENNGSVICIPNNGQCFCLAWLKSNGTNAEKLAA |
| | | NILQWVVFALSVACLGWYAYQAWRATCGWEEVYVALIEMMKSIIEA |
| | | FHEFDSPATLWLSSGNGVVWMRYGEWLLTCPVILIHLSNLTGLKDD |
| | | YSKRTMGLLVSDVGCIVWGATSAMCTGWTKILFFLISLSYGMYTYF |
| | | HAAKVYIEAFHTVPKGLCRQLVRAMAWLFFVSWGMFPVLFLLGPEG |
| | | FGHISPYGSAIGHSILDLIAKNMWGVLGNYLRVKIHEHILLYGDIR |
| | | KKQKITIAGQEMEVETLVAEEEDKYESSLE |
| 75 | NFS-GX-010, AAV- | TTGGCCACTCCCTCTCTGCGCGCTCGCTCGCTCACTGAGGCCGCCC |
| | ProX573.2-(T528)- | GGGCAAAGCCCGGGCGTCGGGCGACCTTTGGTCGCCCGGCCTCAGT |
| | ReaChr, Codon- | GAGCGAGCGAGCGCGCAGAGAGGGAGTGGCCAACTCCATCACTAGG |
| | optimized version 2, | GGTTCCTTTGTCGACTCAACCCAAGAAATTACAGGCTGAAACCAGA |
| | CpG-depleted, | AAAGAACACATTAAAGCACCAAGAGAAAGTTGGAGTGGGTTGAAGG |
| | alternative reading | GAAACAGATTTTTAAAGTTAAGGCTCTGTGAAATGGGTAGAATTAA |
| | frame eliminated | CTACAGGTTAAAAATAAAATGTTAACTAAAGGTTGCCTCTGAGTAA |
| | sequence-WPRE-hGH | CAGGATTATGGGTGATTTTAATTGTCTTCTTTGTGTATGTTCAACA |
| | poly A | GTGACTATAATATGTATTACTTTTGGAATAAAGGAAAACCTGAAAG |
| | | GTGTGTTGTTTTATAAGGGCCCTTAGGTTGCCAAAATTAGAGTCAT |
| | | TGAAATCTAAAGCTGATAAAAACTTTAGTGCAAAGATTGTGACATG |
| | | GGAGACTACACATACCAGATCCATAATGTACATGAGGACAGTAGGC |
| | | CGAGGGGCCCTGCACATTGAAAGCCCACATGGGAGAAGCCCTTGGG |
| | | AAGGGGAGTGGAAGGATGAGGCAAGGGGCCGGGGGGATGCAGAGGC |
| | | TGGCAGGCAGTCATTTCTCAGCTTCAGCCATTCCCGCCTTGGGGGA |
| | | ATGTGGACAGAGAAGCCAAACAAATCTCCTAAACAGTAAATGTCAG |
| | | TCTTCTGTGTCAGATATTTAAGAAAACTAACAGAGGTCAGAGAAGA |
| | | CACACCTACAGCAAGTAGACTGTCCCTGTGCTGCCTTTTTGCAACC |
| | | CCTGCTTTGGCAGTGCTCAAGCCCACCTCCTGCTCTGTGCAGACAT |
| | | CTCTTCTTTGCTCTTACTAGACCAAGGTGAAAGAAAACTCTCACCT |
| | | TCTCCCATCTGGCCCCACAGCATCTGGAACACACTGATCCTCATAA |
| | | TCCTTGTTCTTGAGAAATATTAATGACTTAATCTCCCAAGCTTGCT |
| | | CCCTCTCCTGTGCAGGCCATCTCAGTATGTTTTGCAGACAAGACCC |
| | | AGAGAAGTCCAGACTGGACTTGTTGCAGACTGCAAAACTGCCATTG |
| | | GAAGGCCTCCGTCCCAGTCCTTCTACAGAGTAGCCAGTGGGATTCC |
| | | CAGCCGTTAGTCTTTGCCCTTTATCTGCCTGGCAAAGCTTTTAATT |
| | | GGCTTGATCTGTCATTCCGCTAGACATAAAGGGGACAATCCCCGGA |
| | | TTAGGAAGGAGCTCTCCAGCTCGGGTAAGGAGTCTCAAGGCAAGGT |
| | | AGGCAAGCACCACCGGTCCGCACTCTCGCCCAGCTTTTACGGGAAG |
| | | AAGAGACCGCCACCATGGTGAGCAGAAGACCCTGGCTTTTGGCCTT |
| | | GGCCCTGGCTGTGGCCCTTGCTGCTGGATCTGCTGGGGCATCTACA |
| | | GGCTCTGATGCTACAGTTCCAGTTGCAACCCAGGACGGCCCTGACT |
| | | ATGTGTTCCACAGAGCCCATGAGAGAATGCTGTTCCAGACCAGCTA |
| | | CACCCTGGAGAACAACGGCTCTGTGATCTGCATCCCCAACAACGGC |
| | | CAGTGCTTCTGCCTTGCATGGCTGAAGAGTAATGGCACCAATGCTG |
| | | AGAAGCTGGCTGCCAACATCCTGCAGTGGGTGGTGTTTGCACTGTC |
| | | TGTGGCATGCCTGGGCTGGTATGCATATCAGGCTTGGAGGGCAACC |
| | | TGTGGGTGGGAAGAAGTGTATGTGGCACTGATTGAAATGATGAAAA |
| | | GCATTATTGAAGCCTTCCATGAATTTGATAGCCCAGCCACCCTGTG |
| | | GCTGAGCTCTGGCAACGGAGTGGTGTGGATGAGGTATGGAGAGTGG |
| | | CTGCTGACCTGCCCTGTGATTCTGATTCATCTGAGCAACCTGACAG |
| | | GCCTGAAAGATGATTATAGCAAAAGGACTATGGGCCTGCTGGTGTC |
| | | TGATGTGGGCTGCATTGTGTGGGGAGCAACCTCTGCAATGTGCACA |
| | | GGCTGGACCAAAATTCTGTTCTTCCTGATTAGCCTGTCTTATGGCA |
| | | TGTATACCTATTTTCATGCAGCCAAAGTGTATATTGAAGCATTTCA |
| | | TACAGTGCCAAAAGGCCTGTGCAGACAGCTGGTGAGAGCTATGGCC |
| | | TGGCTGTTCTTTGTGAGCTGGGGAATGTTCCCTGTGCTGTTCCTGC |
| | | TGGGCCCTGAGGGCTTTGGCCATATTAGCCCTTATGGCTCTGCAAT |
| | | TGGCCATAGCATTCTGGATCTGATTGCAAAGAATATGTGGGGAGTG |
| | | CTGGGCAACTATCTGAGGGTGAAGATTCATGAACACATTCTGCTGT |
| | | ATGGAGATATTAGGAAAAAACAGAAAATTACCATTGCAGGCCAGGA |
| | | GATGGAAGTGGAAACCCTGGTGGCAGAAGAAGAAGATAAGTATGAG |
| | | AGCAGCCTGGAGTAATCAACCTCTGGATTACAAAATTTGTGAAAGA |
| | | TTGACTGGTATTCTTAACTATGTTGCTCCTTTTACGCTATGTGGAT |
| | | ACGCTGCTTTAATGCCTTTGTATCATGCTATTGCTTCCCGTATGGC |
| | | TTTCATTTTCTCCTCCTTGTATAAATCCTGGTTGCTGTCTCTTTAT |
| | | GAGGAGTTGTGGCCCGTTGTCAGGCAACGTGGCGTGGTGTGCACTG |
| | | TGTTTGCTGACGCAACCCCCACTGGTTGGGGCATTGCCACCACCTG |
| | | TCAGCTCCTTTCCGGGACTTTCGCTTTCCCCCTCCCTATTGCCACG |
| | | GCGGAACTCATCGCCGCCTGCCTTGCCCGCTGCTGGACAGGGGCTC |
| | | GGCTGTTGGGCACTGACAATTCCGTGGTGTTGTCGGGGAAATCATC |
| | | GTCCTTTCCTTGGCTGCTCGCCTATGTTGCCACCTGGATTCTGCGC |
| | | GGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCT |
| | | TCGCCTTCGCCCTCAGACGAGTCGGATCTCCCTTTGGGCCGCCTCC |
| | | CCGCGGGTGGCATCCCTGTGACCCCTCCCCAGTGCCTCTCCTGGCC |
| | | CTGGAAGTTGCCACTCCAGTGCCCACCAGCCTTGTCCTAATAAAAT |
| | | TAAGTTGCATCATTTTGTCTGACTAGGTGTCCTTCTATAATATTAT |
| | | GGGGTGGAGGGGGGTGGTATGGAGCAAGGGGCAAGTTGGGAAGACA |
| | | ACCTGTAGGGCCTGCGGGGTCTATTGGGAACCAAGCTGGAGTGCAG |
| | | TGGCACAATCTTGGCTCACTGCAATCTCCGCCTCCTGGGTTCAAGC |
| | | GATTCTCCTGCCTCAGCCTCCCGAGTTGTTGGGATTCCAGGCATGC |
| | | ATGACCAGGCTCAGCTAATTTTTGTTTTTTTGGTAGAGACGGGGTT |
| | | TCACCATATTGGCCAGGCTGGTCTCCAACTCCTAATCTCAGGTGAT |
| | | CTACCCACCTTGGCCTCCCAAATTGCTGGGATTACAGGCGTGAACC |
| | | ACTGCTCCCTTCCCTGTCCTTCTGATTTTGAGTCCTAGGAGGAACC |
| | | CCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCC |
| | | GGGCGGCCTCAGTGAGCGAGCGAGCGCGCAGAGAGGGAGTGGCCAA |
| 76 | NFS-GX-011, AAV- | TTGGCCACTCCCTCTCTGCGCGCTCGCTCGCTCACTGAGGCCGCCC |
| | ProX573.2- ReaChr, | GGGCAAAGCCCGGGCGTCGGGCGACCTTTGGTCGCCCGGCCTCAGT |
| | Codon-optimized | GAGCGAGCGAGCGCGCAGAGAGGGAGTGGCCAACTCCATCACTAGG |
| | version 1, CpG- | GGTTCCTTTGTCGACTCAACCCAAGAAATTACAGGCTGAAACCAGA |
| | depleted, alternative | AAAGAACACATTAAAGCACCAAGAGAAAGTTGGAGTGGGTTGAAGG |
| | reading frame | GAAACAGATTTTTAAAGTTAAGGCTCTGTGAAATGGGTAGAATTAA |
| | eliminated sequence- | CTACAGGTTAAAAATAAAATGTTAACTAAAGGTTGCCTCTGAGTAA |
| | WPRE-hGH poly A | CAGGATTATGGGTGATTTTAATTGTCTTCTTTGTGTATGTTCAACA |
| | | GTGACTATAATATGTATTACTTTTGGAATAAAGGAAAACCTGAAAG |
| | | GTGTGTTGTTTTATAAGGGCCCTTAGGTTGCCAAAATTAGAGTCAT |
| | | TGAAATCTAAAGCTGATAAAAACTTTAGTGCAAAGATTGTGACATG |
| | | GGAGACTACACATACCAGATCCATAATGTACATGAGGACAGTAGGC |
| | | CGAGGGGCCCTGCACATTGAAAGCCCACATGGGAGAAGCCCTTGGG |
| | | AAGGGGAGTGGAAGGATGAGGCAAGGGGCCGGGGGGATGCAGAGGC |
| | | TGGCAGGCAGTCATTTCTCAGCTTCAGCCATTCCCGCCATGGGGGA |
| | | ATGTGGACAGAGAAGCCAAACAAATCTCCTAAACAGTAAATGTCAG |
| | | TCTTCTGTGTCAGATATTTAAGAAAACTAACAGAGGTCAGAGAAGA |
| | | CACACCTACAGCAAGTAGACTGTCCCTGTGCTGCCTTTTTGCAACC |
| | | CCTGCTTTGGCAGTGCTCAAGCCCACCTCCTGCTCTGTGCAGACAT |
| | | CTCTTCTTTGCTCTTACTAGACCAAGGTGAAAGAAAACTCTCACCT |
| | | TCTCCCATCTGGCCCCACAGCATCTGGAACACACTGATCCTCATAA |
| | | TCCTTGTTCTTGAGAAATATTAATGACTTAATCTCCCAAGCTTGCT |
| | | CCCTCTCCTGTGCAGGCCATCTCAGTATGTTTTGCAGACAAGACCC |
| | | AGAGAAGTCCAGACTGGACTTGTTGCAGACTGCAAAACTGCCATTG |
| | | GAAGGCCTCCGTCCCAGTCCTTCTACAGAGTAGCCAGTGGGATTCC |
| | | CAGCCGTTAGTCTTTGCCCTTTATCTGCCTGGCAAAGCTTTTAATT |
| | | GGCTTGATCTGTCATTCCGCTAGACATAAAGGGGACAATCCCCGGA |
| | | TTAGGAAGGAGCTCTCCAGCTCGGGTAAGGAGTCTCAAGGCAAGGT |
| | | AGGCAAGCACCACCGGTCCGCACTCTCGCCCAGCTTTTACGGGAAG |
| | | AAGAGACCGCCACCATGGTGTCTAGAAGACCTTGGCTGCTGGCCCT |
| | | GGCTCTGGCTGTGGCCCTTGCTGCTGGATCTGCTGGAGCCAGCACA |
| | | GGCTCAGATGCCACAGTGCCAGTGGCCACACAGGACGGCCCAGATT |
| | | ATGTGTTTCACAGAGCCCATGAGAGAATGCTGTTCCAAACAAGCTA |
| | | TACACTGGAAAACAACGGATCTGTGATCTGCATCCCCAACAACGGC |
| | | CAGTGCTTCTGCCTGGCTTGGCTTAAGAGTAATGGCACCAATGCAG |
| | | AGAAGCTGGCTGCCAACATCCTGCAGTGGGTTGTGTTTGCCCTGTC |
| | | TGTTGCTTGCCTGGGCTGGTATGCCTACCAAGCCTGGAGGGCTACC |
| | | TGTGGGTGGGAAGAGGTGTATGTGGCCCTGATTGAGATGATGAAAA |
| | | GCATCATTGAGGCCTTCCATGAGTTTGACAGCCCTGCCACCCTGTG |
| | | GCTGTCCTCTGGGAATGGAGTGGTGTGGATGAGGTATGGAGAGTGG |
| | | CTCCTGACCTGTCCTGTGATCCTGATCCACCTGTCTAATCTGACAG |
| | | GACTGAAAGACGACTACAGCAAAAGGACTATGGGCCTGCTGGTCTC |
| | | TGATGTGGGATGTATTGTGTGGGGAGCCACCTCTGCCATGTGCACA |
| | | GGCTGGACCAAGATCCTGTTTTTCCTGATTTCCCTGAGCTATGGAA |
| | | TGTACACCTACTTCCATGCTGCAAAAGTGTACATTGAGGCTTTCCA |
| | | CACAGTGCCTAAGGGTCTGTGTAGACAGCTGGTGAGGGCTATGGCC |
| | | TGGCTTTTCTTTGTGTCCTGGGGCATGTTCCCTGTGCTGTTTCTGC |
| | | TGGGCCCTGAGGGCTTTGGCCACATCTCTCCTTATGGCTCTGCCAT |
| | | TGGCCACAGCATCCTGGACCTGATAGCCAAAAACATGTGGGGAGTG |
| | | CTGGGAAATTACCTGAGAGTGAAGATCCATGAGCACATTCTGCTGT |
| | | ATGGAGACATCAGAAAGAAACAGAAGATCACCATTGCAGGCCAGGA |
| | | AATGGAAGTTGAGACACTGGTGGCTGAAGAAGAAGATAAGTATGAG |
| | | AGCAGCCTGGAATAATCAACCTCTGGATTACAAAATTTGTGAAAGA |
| | | TTGACTGGTATTCTTAACTATGTTGCTCCTTTTACGCTATGTGGAT |
| | | ACGCTGCTTTAATGCCTTTGTATCATGCTATTGCTTCCCGTATGGC |
| | | TTTCATTTTCTCCTCCTTGTATAAATCCTGGTTGCTGTCTCTTTAT |
| | | GAGGAGTTGTGGCCCGTTGTCAGGCAACGTGGCGTGGTGTGCACTG |
| | | TGTTTGCTGACGCAACCCCCACTGGTTGGGGCATTGCCACCACCTG |
| | | TCAGCTCCTTTCCGGGACTTTCGCTTTCCCCCTCCCTATTGCCACG |
| | | GCGGAACTCATCGCCGCCTGCCTTGCCCGCTGCTGGACAGGGGCTC |
| | | GGCTGTTGGGCACTGACAATTCCGTGGTGTTGTCGGGGAAATCATC |
| | | GTCCTTTCCTTGGCTGCTCGCCTATGTTGCCACCTGGATTCTGCGC |
| | | GGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCT |
| | | TCGCCTTCGCCCTCAGACGAGTCGGATCTCCCTTTGGGCCGCCTCC |
| | | CCGCGGGTGGCATCCCTGTGACCCCTCCCCAGTGCCTCTCCTGGCC |
| | | CTGGAAGTTGCCACTCCAGTGCCCACCAGCCTTGTCCTAATAAAAT |
| | | TAAGTTGCATCATTTTGTCTGACTAGGTGTCCTTCTATAATATTAT |
| | | GGGGTGGAGGGGGGTGGTATGGAGCAAGGGGCAAGTTGGGAAGACA |
| | | ACCTGTAGGGCCTGCGGGGTCTATTGGGAACCAAGCTGGAGTGCAG |
| | | TGGCACAATCTTGGCTCACTGCAATCTCCGCCTCCTGGGTTCAAGC |
| | | GATTCTCCTGCCTCAGCCTCCCGAGTTGTTGGGATTCCAGGCATGC |
| | | ATGACCAGGCTCAGCTAATTTTTGTTTTTTTGGTAGAGACGGGGTT |
| | | TCACCATATTGGCCAGGCTGGTCTCCAACTCCTAATCTCAGGTGAT |
| | | CTACCCACCTTGGCCTCCCAAATTGCTGGGATTACAGGCGTGAACC |
| | | ACTGCTCCCTTCCCTGTCCTTCTGATTTTGAGTCCTAGGAGGAACC |
| | | CCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCC |
| | | GGGCGGCCTCAGTGAGCGAGCGAGCGCGCAGAGAGGGAGTGGCCAA |
| 77 | NFS-GX-012, AAV- | TTGGCCACTCCCTCTCTGCGCGCTCGCTCGCTCACTGAGGCCGCCC |
| | ProX573.2- ReaChr, | GGGCAAAGCCCGGGCGTCGGGCGACCTTTGGTCGCCCGGCCTCAGT |
| | Codon-optimized | GAGCGAGCGAGCGCGCAGAGAGGGAGTGGCCAACTCCATCACTAGG |
| | version 2, CpG- | GGTTCCTTTGTCGACTCAACCCAAGAAATTACAGGCTGAAACCAGA |
| | depleted, alternative | AAAGAACACATTAAAGCACCAAGAGAAAGTTGGAGTGGGTTGAAGG |
| | reading frame | GAAACAGATTTTTAAAGTTAAGGCTCTGTGAAATGGGTAGAATTAA |
| | eliminated sequence- | CTACAGGTTAAAAATAAAATGTTAACTAAAGGTTGCCTCTGAGTAA |
| | WPRE-hGH polyA | CAGGATTATGGGTGATTTTAATTGTCTTCTTTGTGTATGTTCAACA |
| | | GTGACTATAATATGTATTACTTTTGGAATAAAGGAAAACCTGAAAG |
| | | GTGTGTTGTTTTATAAGGGCCCTTAGGTTGCCAAAATTAGAGTCAT |
| | | TGAAATCTAAAGCTGATAAAAACTTTAGTGCAAAGATTGTGACATG |
| | | GGAGACTACACATACCAGATCCATAATGTACATGAGGACAGTAGGC |
| | | CGAGGGGCCCTGCACATTGAAAGCCCACATGGGAGAAGCCCTTGGG |
| | | AAGGGGAGTGGAAGGATGAGGCAAGGGGCCGGGGGGATGCAGAGGC |
| | | TGGCAGGCAGTCATTTCTCAGCTTCAGCCATTCCCGCCATGGGGGA |
| | | ATGTGGACAGAGAAGCCAAACAAATCTCCTAAACAGTAAATGTCAG |
| | | TCTTCTGTGTCAGATATTTAAGAAAACTAACAGAGGTCAGAGAAGA |
| | | CACACCTACAGCAAGTAGACTGTCCCTGTGCTGCCTTTTTGCAACC |
| | | CCTGCTTTGGCAGTGCTCAAGCCCACCTCCTGCTCTGTGCAGACAT |
| | | CTCTTCTTTGCTCTTACTAGACCAAGGTGAAAGAAAACTCTCACCT |
| | | TCTCCCATCTGGCCCCACAGCATCTGGAACACACTGATCCTCATAA |
| | | TCCTTGTTCTTGAGAAATATTAATGACTTAATCTCCCAAGCTTGCT |
| | | CCCTCTCCTGTGCAGGCCATCTCAGTATGTTTTGCAGACAAGACCC |
| | | AGAGAAGTCCAGACTGGACTTGTTGCAGACTGCAAAACTGCCATTG |
| | | GAAGGCCTCCGTCCCAGTCCTTCTACAGAGTAGCCAGTGGGATTCC |
| | | CAGCCGTTAGTCTTTGCCCTTTATCTGCCTGGCAAAGCTTTTAATT |
| | | GGCTTGATCTGTCATTCCGCTAGACATAAAGGGGACAATCCCCGGA |
| | | TTAGGAAGGAGCTCTCCAGCTCGGGTAAGGAGTCTCAAGGCAAGGT |
| | | AGGCAAGCACCACCGGTCCGCACTCTCGCCCAGCTTTTACGGGAAG |
| | | AAGAGACCGCCACCATGGTGAGCAGAAGACCCTGGCTTTTGGCCTT |
| | | GGCCCTGGCTGTGGCCCTTGCTGCTGGATCTGCTGGGGCATCTACA |
| | | GGCTCTGATGCTACAGTTCCAGTTGCAACCCAGGACGGCCCTGACT |
| | | ATGTGTTCCACAGAGCCCATGAGAGAATGCTGTTCCAGACCAGCTA |
| | | CACCCTGGAGAACAACGGCTCTGTGATCTGCATCCCCAACAACGGC |
| | | CAGTGCTTCTGCCTTGCATGGCTGAAGAGTAATGGCACCAATGCTG |
| | | AGAAGCTGGCTGCCAACATCCTGCAGTGGGTGGTGTTTGCACTGTC |
| | | TGTGGCATGCCTGGGCTGGTATGCATATCAGGCTTGGAGGGCAACC |
| | | TGTGGGTGGGAAGAAGTGTATGTGGCACTGATTGAAATGATGAAAA |
| | | GCATTATTGAAGCCTTCCATGAATTTGATAGCCCAGCCACCCTGTG |
| | | GCTGAGCTCTGGCAACGGAGTGGTGTGGATGAGGTATGGAGAGTGG |
| | | CTGCTGACCTGCCCTGTGATTCTGATTCATCTGAGCAACCTGACAG |
| | | GCCTGAAAGATGATTATAGCAAAAGGACTATGGGCCTGCTGGTGTC |
| | | TGATGTGGGCTGCATTGTGTGGGGAGCAACCTCTGCAATGTGCACA |
| | | GGCTGGACCAAAATTCTGTTCTTCCTGATTAGCCTGTCTTATGGCA |
| | | TGTATACCTATTTTCATGCAGCCAAAGTGTATATTGAAGCATTTCA |
| | | TACAGTGCCAAAAGGCCTGTGCAGACAGCTGGTGAGAGCTATGGCC |
| | | TGGCTGTTCTTTGTGAGCTGGGGAATGTTCCCTGTGCTGTTCCTGC |
| | | TGGGCCCTGAGGGCTTTGGCCATATTAGCCCTTATGGCTCTGCAAT |
| | | TGGCCATAGCATTCTGGATCTGATTGCAAAGAATATGTGGGGAGTG |
| | | CTGGGCAACTATCTGAGGGTGAAGATTCATGAACACATTCTGCTGT |
| | | ATGGAGATATTAGGAAAAAACAGAAAATTACCATTGCAGGCCAGGA |
| | | GATGGAAGTGGAAACCCTGGTGGCAGAAGAAGAAGATAAGTATGAG |
| | | AGCAGCCTGGAGTAATCAACCTCTGGATTACAAAATTTGTGAAAGA |
| | | TTGACTGGTATTCTTAACTATGTTGCTCCTTTTACGCTATGTGGAT |
| | | ACGCTGCTTTAATGCCTTTGTATCATGCTATTGCTTCCCGTATGGC |
| | | TTTCATTTTCTCCTCCTTGTATAAATCCTGGTTGCTGTCTCTTTAT |
| | | GAGGAGTTGTGGCCCGTTGTCAGGCAACGTGGCGTGGTGTGCACTG |
| | | TGTTTGCTGACGCAACCCCCACTGGTTGGGGCATTGCCACCACCTG |
| | | TCAGCTCCTTTCCGGGACTTTCGCTTTCCCCCTCCCTATTGCCACG |
| | | GCGGAACTCATCGCCGCCTGCCTTGCCCGCTGCTGGACAGGGGCTC |
| | | GGCTGTTGGGCACTGACAATTCCGTGGTGTTGTCGGGGAAATCATC |
| | | GTCCTTTCCTTGGCTGCTCGCCTATGTTGCCACCTGGATTCTGCGC |
| | | GGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCT |
| | | TCGCCTTCGCCCTCAGACGAGTCGGATCTCCCTTTGGGCCGCCTCC |
| | | CCGCGGGTGGCATCCCTGTGACCCCTCCCCAGTGCCTCTCCTGGCC |
| | | CTGGAAGTTGCCACTCCAGTGCCCACCAGCCTTGTCCTAATAAAAT |
| | | TAAGTTGCATCATTTTGTCTGACTAGGTGTCCTTCTATAATATTAT |
| | | GGGGTGGAGGGGGGTGGTATGGAGCAAGGGGCAAGTTGGGAAGACA |
| | | ACCTGTAGGGCCTGCGGGGTCTATTGGGAACCAAGCTGGAGTGCAG |
| | | TGGCACAATCTTGGCTCACTGCAATCTCCGCCTCCTGGGTTCAAGC |
| | | GATTCTCCTGCCTCAGCCTCCCGAGTTGTTGGGATTCCAGGCATGC |
| | | ATGACCAGGCTCAGCTAATTTTTGTTTTTTTGGTAGAGACGGGGTT |
| | | TCACCATATTGGCCAGGCTGGTCTCCAACTCCTAATCTCAGGTGAT |
| | | CTACCCACCTTGGCCTCCCAAATTGCTGGGATTACAGGCGTGAACC |
| | | ACTGCTCCCTTCCCTGTCCTTCTGATTTTGAGTCCTAGGAGGAACC |
| | | CCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCC |
| | | GGGCGGCCTCAGTGAGCGAGCGAGCGCGCAGAGAGGGAGTGGCCAA |
| 78 | AAV WT Seq 1 | GGAGGGGTGGAGTCGTGACGTGAATTACGTCATAGGGTTAGGGAGG |
| 79 | AAV WT Seq 2 | GTAGATAAGTAGCATGGCGGGTTAATCATTAACTACA |
| 80 | NFS-GX-017 | TTGGCCACTCCCTCTCTGCGCGCTCGCTCGCTCACTGAGGCCGCCC |
| | AAV-ProA7- | GGGCAAAGCCCGGGCGTCGGGCGACCTTTGGTCGCCCGGCCTCAGT |
| | ReaChR, Codon- | GAGCGAGCGAGCGCGCAGAGAGGGAGTGGCCAACTCCATCACTAGG |
| | optimized version 1, | GGTTCCTTTGTCGACTCCATCCTGAGAGATGAGCCAGGACAAAGAA |
| | CpG-depleted, | CCAGTAATAGCTCCTGGAGCAGCACATCTGTTTTGCCAGGATTATC |
| | alternative reading | CCTTGGATCTCTTAAAACCGAGACCTTGTAATCTGAAGACTCAACT |
| | frame eliminated | TGGGCTGTACCCTTAACCTTCAGCTCTATGATGCAAGTGAGTCCAC |
| | sequence-WPRE-hGH | AGGACCGGAGGCTTTGAGATGAGCTTTTCAGAAGGGAGGAGTTGGC |
| | poly A | CGCTTGCTCCCAGAGCTCCAGCACCTGCATTCTTCTGGCTATGTCA |
| | | GAAGCCAGATCATTTCCCTCGTTAAAAACAAAAACAAAAAAACAAA |
| | | CAAACAAAATGTTAGTCTTTGCCCTTTATCTGCCTGGCAAAGCTTT |
| | | TAATTGGCTTGATCTGTCATTCCGCTAGACATAAAGGGGACAATCC |
| | | CCGGATTAGGAAGGAGCTCTCCAGCTCGGGTAAGGAGTCTCAAGGC |
| | | AAGGTAGGCAAGCACCACCGGTCCGCACTCTCGCCCAGCTTTTACG |
| | | GGAAGAAGAGACCGCCACCATGGTGTCTAGAAGACCTTGGCTGCTG |
| | | GCCCTGGCTCTGGCTGTGGCCCTTGCTGCTGGATCTGCTGGAGCCA |
| | | GCACAGGCTCAGATGCCACAGTGCCAGTGGCCACACAGGACGGCCC |
| | | AGATTATGTGTTTCACAGAGCCCATGAGAGAATGCTGTTCCAAACA |
| | | AGCTATACACTGGAAAACAACGGATCTGTGATCTGCATCCCCAACA |
| | | ACGGCCAGTGCTTCTGCCTGGCTTGGCTTAAGAGTAATGGCACCAA |
| | | TGCAGAGAAGCTGGCTGCCAACATCCTGCAGTGGGTTGTGTTTGCC |
| | | CTGTCTGTTGCTTGCCTGGGCTGGTATGCCTACCAAGCCTGGAGGG |
| | | CTACCTGTGGGTGGGAAGAGGTGTATGTGGCCCTGATTGAGATGAT |
| | | GAAAAGCATCATTGAGGCCTTCCATGAGTTTGACAGCCCTGCCACC |
| | | CTGTGGCTGTCCTCTGGGAATGGAGTGGTGTGGATGAGGTATGGAG |
| | | AGTGGCTCCTGACCTGTCCTGTGATCCTGATCCACCTGTCTAATCT |
| | | GACAGGACTGAAAGACGACTACAGCAAAAGGACTATGGGCCTGCTG |
| | | GTCTCTGATGTGGGATGTATTGTGTGGGGAGCCACCTCTGCCATGT |
| | | GCACAGGCTGGACCAAGATCCTGTTTTTCCTGATTTCCCTGAGCTA |
| | | TGGAATGTACACCTACTTCCATGCTGCAAAAGTGTACATTGAGGCT |
| | | TTCCACACAGTGCCTAAGGGTCTGTGTAGACAGCTGGTGAGGGCTA |
| | | TGGCCTGGCTTTTCTTTGTGTCCTGGGGCATGTTCCCTGTGCTGTT |
| | | TCTGCTGGGCCCTGAGGGCTTTGGCCACATCTCTCCTTATGGCTCT |
| | | GCCATTGGCCACAGCATCCTGGACCTGATAGCCAAAAACATGTGGG |
| | | GAGTGCTGGGAAATTACCTGAGAGTGAAGATCCATGAGCACATTCT |
| | | GCTGTATGGAGACATCAGAAAGAAACAGAAGATCACCATTGCAGGC |
| | | CAGGAAATGGAAGTTGAGACACTGGTGGCTGAAGAAGAAGATAAGT |
| | | ATGAGAGCAGCCTGGAATAATCAACCTCTGGATTACAAAATTTGTG |
| | | AAAGATTGACTGGTATTCTTAACTATGTTGCTCCTTTTACGCTATG |
| | | TGGATACGCTGCTTTAATGCCTTTGTATCATGCTATTGCTTCCCGT |
| | | ATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTG |
| | | CACTGTGTTTGCTGACGCAACCCCCACTGGTTGGGGCATTGCCACC |
| | | ACCTGTCAGCTCCTTTCCGGGACTTTCGCTTTCCCCCTCCCTATTG |
| | | CCACGGCGGAACTCATCGCCGCCTGCCTTGCCCGCTGCTGGACAGG |
| | | GGCTCGGCTGTTGGGCACTGACAATTCCGTGGTGTTGTCGGGGAAA |
| | | TCATCGTCCTTTCCTTGGCTGCTCGCCTATGTTGCCACCTGGATTC |
| | | TGCGCGGGACGTCCTTCTGCTACGTCCCTTCGGCCCTCAATCCAGC |
| | | GGACCTTCCTTCCCGCGGCCTGCTGCCGGCTCTGCGGCCTCTTCCG |
| | | CGTCTTCGCCTTCGCCCTCAGACGAGTCGGATCTCCCTTTGGGCCG |
| | | CCTCCCCGCGGGTGGCATCCCTGTGACCCCTCCCCAGTGCCTCTCC |
| | | TGGCCCTGGAAGTTGCCACTCCAGTGCCCACCAGCCTTGTCCTAAT |
| | | AAAATTAAGTTGCATCATTTTGTCTGACTAGGTGTCCTTCTATAAT |
| | | ATTATGGGGTGGAGGGGGGTGGTATGGAGCAAGGGGCAAGTTGGGA |
| | | AGACAACCTGTAGGGCCTGCGGGGTCTATTGGGAACCAAGCTGGAG |
| | | TGCAGTGGCACAATCTTGGCTCACTGCAATCTCCGCCTCCTGGGTT |
| | | CAAGCGATTCTCCTGCCTCAGCCTCCCGAGTTGTTGGGATTCCAGG |
| | | CATGCATGACCAGGCTCAGCTAATTTTTGTTTTTTTGGTAGAGACG |
| | | GGGTTTCACCATATTGGCCAGGCTGGTCTCCAACTCCTAATCTCAG |
| | | GTGATCTACCCACCTTGGCCTCCCAAATTGCTGGGATTACAGGCGT |
| | | GAACCACTGCTCCCTTCCCTGTCCTTCTGATTTTGAGTCCTAGGAG |
| | | GAACCCCTAGTGATGGAGTTGGCCACTCCCTCTCTGCGCGCTCGCT |
| | | CGCTCACTGAGGCCGGGCGACCAAAGGTCGCCCGACGCCCGGGCTT |
| | | TGCCCGGGCGGCCTCAGTGAGCGAGCGAGCGCGCAGAGAGGGAGTG |
| | | GCCAA |
| 81 | NFS-GX-018, AAV- | TTGGCCACTCCCTCTCTGCGCGCTCGCTCGCTCACTGAGGCCGCCC |
| | ProA7- ReaChR, | GGGCAAAGCCCGGGCGTCGGGCGACCTTTGGTCGCCCGGCCTCAGT |
| | Codon-optimized | GAGCGAGCGAGCGCGCAGAGAGGGAGTGGCCAACTCCATCACTAGG |
| | version 2, CpG- | GGTTCCTTTGTCGACTCCATCCTGAGAGATGAGCCAGGACAAAGAA |
| | depleted, alternative | CCAGTAATAGCTCCTGGAGCAGCACATCTGTTTTGCCAGGATTATC |
| | reading frame | CCTTGGATCTCTTAAAACCGAGACCTTGTAATCTGAAGACTCAACT |
| | eliminated sequence- | TGGGCTGTACCCTTAACCTTCAGCTCTATGATGCAAGTGAGTCCAC |
| | WPRE-hGH polyA | AGGACCGGAGGCTTTGAGATGAGCTTTTCAGAAGGGAGGAGTTGGC |
| | | CGCTTGCTCCCAGAGCTCCAGCACCTGCATTCTTCTGGCTATGTCA |
| | | GAAGCCAGATCATTTCCCTCGTTAAAAACAAAAACAAAAAAACAAA |
| | | CAAACAAAATGTTAGTCTTTGCCCTTTATCTGCCTGGCAAAGCTTT |
| | | TAATTGGCTTGATCTGTCATTCCGCTAGACATAAAGGGGACAATCC |
| | | CCGGATTAGGAAGGAGCTCTCCAGCTCGGGTAAGGAGTCTCAAGGC |
| | | AAGGTAGGCAAGCACCACCGGTCCGCACTCTCGCCCAGCTTTTACG |
| | | GGAAGAAGAGACCGCCACCATGGTGAGCAGAAGACCCTGGCTTTTG |
| | | GCCTTGGCCCTGGCTGTGGCCCTTGCTGCTGGATCTGCTGGGGCAT |
| | | CTACAGGCTCTGATGCTACAGTTCCAGTTGCAACCCAGGACGGCCC |
| | | TGACTATGTGTTCCACAGAGCCCATGAGAGAATGCTGTTCCAGACC |
| | | AGCTACACCCTGGAGAACAACGGCTCTGTGATCTGCATCCCCAACA |
| | | ACGGCCAGTGCTTCTGCCTTGCATGGCTGAAGAGTAATGGCACCAA |
| | | TGCTGAGAAGCTGGCTGCCAACATCCTGCAGTGGGTGGTGTTTGCA |
| | | CTGTCTGTGGCATGCCTGGGCTGGTATGCATATCAGGCTTGGAGGG |
| | | CAACCTGTGGGTGGGAAGAAGTGTATGTGGCACTGATTGAAATGAT |
| | | GAAAAGCATTATTGAAGCCTTCCATGAATTTGATAGCCCAGCCACC |
| | | CTGTGGCTGAGCTCTGGCAACGGAGTGGTGTGGATGAGGTATGGAG |
| | | AGTGGCTGCTGACCTGCCCTGTGATTCTGATTCATCTGAGCAACCT |
| | | GACAGGCCTGAAAGATGATTATAGCAAAAGGACTATGGGCCTGCTG |
| | | GTGTCTGATGTGGGCTGCATTGTGTGGGGAGCAACCTCTGCAATGT |
| | | GCACAGGCTGGACCAAAATTCTGTTCTTCCTGATTAGCCTGTCTTA |
| | | TGGCATGTATACCTATTTTCATGCAGCCAAAGTGTATATTGAAGCA |
| | | TTTCATACAGTGCCAAAAGGCCTGTGCAGACAGCTGGTGAGAGCTA |
| | | TGGCCTGGCTGTTCTTTGTGAGCTGGGGAATGTTCCCTGTGCTGTT |
| | | CCTGCTGGGCCCTGAGGGCTTTGGCCATATTAGCCCTTATGGCTCT |
| | | GCAATTGGCCATAGCATTCTGGATCTGATTGCAAAGAATATGTGGG |
| | | GAGTGCTGGGCAACTATCTGAGGGTGAAGATTCATGAACACATTCT |
| | | GCTGTATGGAGATATTAGGAAAAAACAGAAAATTACCATTGCAGGC |
| | | CAGGAGATGGAAGTGGAAACCCTGGTGGCAGAAGAAGAAGATAAGT |
| | | ATGAGAGCAGCCTGGAGTAATCAACCTCTGGATTACAAAATTTGTG |
| | | AAAGATTGACTGGTATTCTTAACTATGTTGCTCCTTTTACGCTATG |
| | | TGGATACGCTGCTTTAATGCCTTTGTATCATGCTATTGCTTCCCGT |
| | | ATGGCTTTCATTTTCTCCTCCTTGTATAAATCCTGGTTGCTGTCTC |
| | | TTTATGAGGAGTTGTGGCCCGTTGTCAGGCAACGTGGCGTGGTGTG |
| | | CACTGTGTTTGCTGACGCAACCCCCACTGGTTGGGGCATTGCCACC |
| | | ACCTGTCAGCTCCTTTCCGGGACTTTCGCTTTCCCCCTCCCTATTG |
| | | CCACGGCGGAACTCATCGCCGCCTGCCTTGCCCGCTGCTGGACAGG |
| | | GGCTCGGCTGTTGGGCACTGACAATTCCGTGGTGTTGTCGGGGAAA |
| | | TCATCGTCCTTTCCTTGGCTGCTCGCCTATGTTGCCACCTGGATTC |
| | | TGCGCGGGACGTCCTTCTGCTACGTCCCTTCGGCCCTCAATCCAGC |
| | | GGACCTTCCTTCCCGCGGCCTGCTGCCGGCTCTGCGGCCTCTTCCG |
| | | CGTCTTCGCCTTCGCCCTCAGACGAGTCGGATCTCCCTTTGGGCCG |
| | | CCTCCCCGCGGGTGGCATCCCTGTGACCCCTCCCCAGTGCCTCTCC |
| | | TGGCCCTGGAAGTTGCCACTCCAGTGCCCACCAGCCTTGTCCTAAT |
| | | AAAATTAAGTTGCATCATTTTGTCTGACTAGGTGTCCTTCTATAAT |
| | | ATTATGGGGTGGAGGGGGGTGGTATGGAGCAAGGGGCAAGTTGGGA |
| | | AGACAACCTGTAGGGCCTGCGGGGTCTATTGGGAACCAAGCTGGAG |
| | | TGCAGTGGCACAATCTTGGCTCACTGCAATCTCCGCCTCCTGGGTT |
| | | CAAGCGATTCTCCTGCCTCAGCCTCCCGAGTTGTTGGGATTCCAGG |
| | | CATGCATGACCAGGCTCAGCTAATTTTTGTTTTTTTGGTAGAGACG |
| | | GGGTTTCACCATATTGGCCAGGCTGGTCTCCAACTCCTAATCTCAG |
| | | GTGATCTACCCACCTTGGCCTCCCAAATTGCTGGGATTACAGGCGT |
| | | GAACCACTGCTCCCTTCCCTGTCCTTCTGATTTTGAGTCCTAGGAG |
| | | GAACCCCTAGTGATGGAGTTGGCCACTCCCTCTCTGCGCGCTCGCT |
| | | CGCTCACTGAGGCCGGGCGACCAAAGGTCGCCCGACGCCCGGGCTT |
| | | TGCCCGGGCGGCCTCAGTGAGCGAGCGAGCGCGCAGAGAGGGAGTG |
| | | GCCAA |
| 82 | NFS-GX-019, AAV- | TTGGCCACTCCCTCTCTGCGCGCTCGCTCGCTCACTGAGGCCGCCC |
| | 4xProSC- ReaChR, | GGGCAAAGCCCGGGCGTCGGGCGACCTTTGGTCGCCCGGCCTCAGT |
| | Codon-optimized | GAGCGAGCGAGCGCGCAGAGAGGGAGTGGCCAACTCCATCACTAGG |
| | version 1, CpG- | GGTTCCTTTGTCGACTCGTTAGTCTTTGCCCTTTATCTGCCTGGCA |
| | depleted, alternative | AAGCTTTTAATTGGCTTGATCTGTCATTCCGCTAGACATAAAGGGG |
| | reading frame | ACAATCCCCGGATTAGGAAGGAGCTCTCCAGCTCGGGTAAGGAGTC |
| | eliminated sequence- | TCAAGGCAAGGTAGGCAAGCACCACCGGTCCGCACTCTCGCCCAGC |
| | WPRE-hGH polyA | TTTTACGGGAAGAAGAGAGTTAGTCTTTGCCCTTTATCTGCCTGGC |
| | | AAAGCTTTTAATTGGCTTGATCTGTCATTCCGCTAGACATAAAGGG |
| | | GACAATCCCCGGATTAGGAAGGAGCTCTCCAGCTCGGGTAAGGAGT |
| | | CTCAAGGCAAGGTAGGCAAGCACCACCGGTCCGCACTCTCGCCCAG |
| | | CTTTTACGGGAAGAAGAGAGTTAGTCTTTGCCCTTTATCTGCCTGG |
| | | CAAAGCTTTTAATTGGCTTGATCTGTCATTCCGCTAGACATAAAGG |
| | | GGACAATCCCCGGATTAGGAAGGAGCTCTCCAGCTCGGGTAAGGAG |
| | | TCTCAAGGCAAGGTAGGCAAGCACCACCGGTCCGCACTCTCGCCCA |
| | | GCTTTTACGGGAAGAAGAGAGTTAGTCTTTGCCCTTTATCTGCCTG |
| | | GCAAAGCTTTTAATTGGCTTGATCTGTCATTCCGCTAGACATAAAG |
| | | GGGACAATCCCCGGATTAGGAAGGAGCTCTCCAGCTCGGGTAAGGA |
| | | GTCTCAAGGCAAGGTAGGCAAGCACCACCGGTCCGCACTCTCGCCC |
| | | AGCTTTTACGGGAAGAAGAGAGCCGCCACCATGGTGTCTAGAAGAC |
| | | CTTGGCTGCTGGCCCTGGCTCTGGCTGTGGCCCTTGCTGCTGGATC |
| | | TGCTGGAGCCAGCACAGGCTCAGATGCCACAGTGCCAGTGGCCACA |
| | | CAGGACGGCCCAGATTATGTGTTTCACAGAGCCCATGAGAGAATGC |
| | | TGTTCCAAACAAGCTATACACTGGAAAACAACGGATCTGTGATCTG |
| | | CATCCCCAACAACGGCCAGTGCTTCTGCCTGGCTTGGCTTAAGAGT |
| | | AATGGCACCAATGCAGAGAAGCTGGCTGCCAACATCCTGCAGTGGG |
| | | TTGTGTTTGCCCTGTCTGTTGCTTGCCTGGGCTGGTATGCCTACCA |
| | | AGCCTGGAGGGCTACCTGTGGGTGGGAAGAGGTGTATGTGGCCCTG |
| | | ATTGAGATGATGAAAAGCATCATTGAGGCCTTCCATGAGTTTGACA |
| | | GCCCTGCCACCCTGTGGCTGTCCTCTGGGAATGGAGTGGTGTGGAT |
| | | GAGGTATGGAGAGTGGCTCCTGACCTGTCCTGTGATCCTGATCCAC |
| | | CTGTCTAATCTGACAGGACTGAAAGACGACTACAGCAAAAGGACTA |
| | | TGGGCCTGCTGGTCTCTGATGTGGGATGTATTGTGTGGGGAGCCAC |
| | | CTCTGCCATGTGCACAGGCTGGACCAAGATCCTGTTTTTCCTGATT |
| | | TCCCTGAGCTATGGAATGTACACCTACTTCCATGCTGCAAAAGTGT |
| | | ACATTGAGGCTTTCCACACAGTGCCTAAGGGTCTGTGTAGACAGCT |
| | | GGTGAGGGCTATGGCCTGGCTTTTCTTTGTGTCCTGGGGCATGTTC |
| | | CCTGTGCTGTTTCTGCTGGGCCCTGAGGGCTTTGGCCACATCTCTC |
| | | CTTATGGCTCTGCCATTGGCCACAGCATCCTGGACCTGATAGCCAA |
| | | AAACATGTGGGGAGTGCTGGGAAATTACCTGAGAGTGAAGATCCAT |
| | | GAGCACATTCTGCTGTATGGAGACATCAGAAAGAAACAGAAGATCA |
| | | CCATTGCAGGCCAGGAAATGGAAGTTGAGACACTGGTGGCTGAAGA |
| | | AGAAGATAAGTATGAGAGCAGCCTGGAATAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTA |
| | | TTGCTTCCCGTATGGCTTTCATTTTCTCCTCCTTGTATAAATCCTG |
| | | GTTGCTGTCTCTTTATGAGGAGTTGTGGCCCGTTGTCAGGCAACGT |
| | | GGCGTGGTGTGCACTGTGTTTGCTGACGCAACCCCCACTGGTTGGG |
| | | GCATTGCCACCACCTGTCAGCTCCTTTCCGGGACTTTCGCTTTCCC |
| | | CCTCCCTATTGCCACGGCGGAACTCATCGCCGCCTGCCTTGCCCGC |
| | | TGCTGGACAGGGGCTCGGCTGTTGGGCACTGACAATTCCGTGGTGT |
| | | TGTCGGGGAAATCATCGTCCTTTCCTTGGCTGCTCGCCTATGTTGC |
| | | CACCTGGATTCTGCGCGGGACGTCCTTCTGCTACGTCCCTTCGGCC |
| | | CTCAATCCAGCGGACCTTCCTTCCCGCGGCCTGCTGCCGGCTCTGC |
| | | GGCCTCTTCCGCGTCTTCGCCTTCGCCCTCAGACGAGTCGGATCTC |
| | | CCTTTGGGCCGCCTCCCCGCGGGTGGCATCCCTGTGACCCCTCCCC |
| | | AGTGCCTCTCCTGGCCCTGGAAGTTGCCACTCCAGTGCCCACCAGC |
| | | CTTGTCCTAATAAAATTAAGTTGCATCATTTTGTCTGACTAGGTGT |
| | | CCTTCTATAATATTATGGGGTGGAGGGGGGTGGTATGGAGCAAGGG |
| | | GCAAGTTGGGAAGACAACCTGTAGGGCCTGCGGGGTCTATTGGGAA |
| | | CCAAGCTGGAGTGCAGTGGCACAATCTTGGCTCACTGCAATCTCCG |
| | | CCTCCTGGGTTCAAGCGATTCTCCTGCCTCAGCCTCCCGAGTTGTT |
| | | GGGATTCCAGGCATGCATGACCAGGCTCAGCTAATTTTTGTTTTTT |
| | | TGGTAGAGACGGGGTTTCACCATATTGGCCAGGCTGGTCTCCAACT |
| | | CCTAATCTCAGGTGATCTACCCACCTTGGCCTCCCAAATTGCTGGG |
| | | ATTACAGGCGTGAACCACTGCTCCCTTCCCTGTCCTTCTGATTTTG |
| | | AGTCCTAGGAGGAACCCCTAGTGATGGAGTTGGCCACTCCCTCTCT |
| | | GCGCGCTCGCTCGCTCACTGAGGCCGGGCGACCAAAGGTCGCCCGA |
| | | CGCCCGGGCTTTGCCCGGGCGGCCTCAGTGAGCGAGCGAGCGCGCA |
| | | GAGAGGGAGTGGCCAA |
| 83 | NFS-GX-020, AAV- | TTGGCCACTCCCTCTCTGCGCGCTCGCTCGCTCACTGAGGCCGCCC |
| | 4xProSC- ReaChR, | GGGCAAAGCCCGGGCGTCGGGCGACCTTTGGTCGCCCGGCCTCAGT |
| | Codon-optimized | GAGCGAGCGAGCGCGCAGAGAGGGAGTGGCCAACTCCATCACTAGG |
| | version 2, CpG- | GGTTCCTTTGTCGACTCGTTAGTCTTTGCCCTTTATCTGCCTGGCA |
| | depleted, alternative | AAGCTTTTAATTGGCTTGATCTGTCATTCCGCTAGACATAAAGGGG |
| | reading frame | ACAATCCCCGGATTAGGAAGGAGCTCTCCAGCTCGGGTAAGGAGTC |
| | eliminated sequence- | TCAAGGCAAGGTAGGCAAGCACCACCGGTCCGCACTCTCGCCCAGC |
| | WPRE-hGH polyA | TTTTACGGGAAGAAGAGAGTTAGTCTTTGCCCTTTATCTGCCTGGC |
| | | AAAGCTTTTAATTGGCTTGATCTGTCATTCCGCTAGACATAAAGGG |
| | | GACAATCCCCGGATTAGGAAGGAGCTCTCCAGCTCGGGTAAGGAGT |
| | | CTCAAGGCAAGGTAGGCAAGCACCACCGGTCCGCACTCTCGCCCAG |
| | | CTTTTACGGGAAGAAGAGAGTTAGTCTTTGCCCTTTATCTGCCTGG |
| | | CAAAGCTTTTAATTGGCTTGATCTGTCATTCCGCTAGACATAAAGG |
| | | GGACAATCCCCGGATTAGGAAGGAGCTCTCCAGCTCGGGTAAGGAG |
| | | TCTCAAGGCAAGGTAGGCAAGCACCACCGGTCCGCACTCTCGCCCA |
| | | GCTTTTACGGGAAGAAGAGAGTTAGTCTTTGCCCTTTATCTGCCTG |
| | | GCAAAGCTTTTAATTGGCTTGATCTGTCATTCCGCTAGACATAAAG |
| | | GGGACAATCCCCGGATTAGGAAGGAGCTCTCCAGCTCGGGTAAGGA |
| | | GTCTCAAGGCAAGGTAGGCAAGCACCACCGGTCCGCACTCTCGCCC |
| | | AGCTTTTACGGGAAGAAGAGAGCCGCCACCATGGTGAGCAGAAGAC |
| | | CCTGGCTTTTGGCCTTGGCCCTGGCTGTGGCCCTTGCTGCTGGATC |
| | | TGCTGGGGCATCTACAGGCTCTGATGCTACAGTTCCAGTTGCAACC |
| | | CAGGACGGCCCTGACTATGTGTTCCACAGAGCCCATGAGAGAATGC |
| | | TGTTCCAGACCAGCTACACCCTGGAGAACAACGGCTCTGTGATCTG |
| | | CATCCCCAACAACGGCCAGTGCTTCTGCCTTGCATGGCTGAAGAGT |
| | | AATGGCACCAATGCTGAGAAGCTGGCTGCCAACATCCTGCAGTGGG |
| | | TGGTGTTTGCACTGTCTGTGGCATGCCTGGGCTGGTATGCATATCA |
| | | GGCTTGGAGGGCAACCTGTGGGTGGGAAGAAGTGTATGTGGCACTG |
| | | ATTGAAATGATGAAAAGCATTATTGAAGCCTTCCATGAATTTGATA |
| | | GCCCAGCCACCCTGTGGCTGAGCTCTGGCAACGGAGTGGTGTGGAT |
| | | GAGGTATGGAGAGTGGCTGCTGACCTGCCCTGTGATTCTGATTCAT |
| | | CTGAGCAACCTGACAGGCCTGAAAGATGATTATAGCAAAAGGACTA |
| | | TGGGCCTGCTGGTGTCTGATGTGGGCTGCATTGTGTGGGGAGCAAC |
| | | CTCTGCAATGTGCACAGGCTGGACCAAAATTCTGTTCTTCCTGATT |
| | | AGCCTGTCTTATGGCATGTATACCTATTTTCATGCAGCCAAAGTGT |
| | | ATATTGAAGCATTTCATACAGTGCCAAAAGGCCTGTGCAGACAGCT |
| | | GGTGAGAGCTATGGCCTGGCTGTTCTTTGTGAGCTGGGGAATGTTC |
| | | CCTGTGCTGTTCCTGCTGGGCCCTGAGGGCTTTGGCCATATTAGCC |
| | | CTTATGGCTCTGCAATTGGCCATAGCATTCTGGATCTGATTGCAAA |
| | | GAATATGTGGGGAGTGCTGGGCAACTATCTGAGGGTGAAGATTCAT |
| | | GAACACATTCTGCTGTATGGAGATATTAGGAAAAAACAGAAAATTA |
| | | CCATTGCAGGCCAGGAGATGGAAGTGGAAACCCTGGTGGCAGAAGA |
| | | AGAAGATAAGTATGAGAGCAGCCTGGAGTAATCAACCTCTGGATTA |
| | | CAAAATTTGTGAAAGATTGACTGGTATTCTTAACTATGTTGCTCCT |
| | | TTTACGCTATGTGGATACGCTGCTTTAATGCCTTTGTATCATGCTA |
| | | TTGCTTCCCGTATGGCTTTCATTTTCTCCTCCTTGTATAAATCCTG |
| | | GTTGCTGTCTCTTTATGAGGAGTTGTGGCCCGTTGTCAGGCAACGT |
| | | GGCGTGGTGTGCACTGTGTTTGCTGACGCAACCCCCACTGGTTGGG |
| | | GCATTGCCACCACCTGTCAGCTCCTTTCCGGGACTTTCGCTTTCCC |
| | | CCTCCCTATTGCCACGGCGGAACTCATCGCCGCCTGCCTTGCCCGC |
| | | TGCTGGACAGGGGCTCGGCTGTTGGGCACTGACAATTCCGTGGTGT |
| | | TGTCGGGGAAATCATCGTCCTTTCCTTGGCTGCTCGCCTATGTTGC |
| | | CACCTGGATTCTGCGCGGGACGTCCTTCTGCTACGTCCCTTCGGCC |
| | | CTCAATCCAGCGGACCTTCCTTCCCGCGGCCTGCTGCCGGCTCTGC |
| | | GGCCTCTTCCGCGTCTTCGCCTTCGCCCTCAGACGAGTCGGATCTC |
| | | CCTTTGGGCCGCCTCCCCGCGGGTGGCATCCCTGTGACCCCTCCCC |
| | | AGTGCCTCTCCTGGCCCTGGAAGTTGCCACTCCAGTGCCCACCAGC |
| | | CTTGTCCTAATAAAATTAAGTTGCATCATTTTGTCTGACTAGGTGT |
| | | CCTTCTATAATATTATGGGGTGGAGGGGGGTGGTATGGAGCAAGGG |
| | | GCAAGTTGGGAAGACAACCTGTAGGGCCTGCGGGGTCTATTGGGAA |
| | | CCAAGCTGGAGTGCAGTGGCACAATCTTGGCTCACTGCAATCTCCG |
| | | CCTCCTGGGTTCAAGCGATTCTCCTGCCTCAGCCTCCCGAGTTGTT |
| | | GGGATTCCAGGCATGCATGACCAGGCTCAGCTAATTTTTGTTTTTT |
| | | TGGTAGAGACGGGGTTTCACCATATTGGCCAGGCTGGTCTCCAACT |
| | | CCTAATCTCAGGTGATCTACCCACCTTGGCCTCCCAAATTGCTGGG |
| | | ATTACAGGCGTGAACCACTGCTCCCTTCCCTGTCCTTCTGATTTTG |
| | | AGTCCTAGGAGGAACCCCTAGTGATGGAGTTGGCCACTCCCTCTCT |
| | | GCGCGCTCGCTCGCTCACTGAGGCCGGGCGACCAAAGGTCGCCCGA |
| | | CGCCCGGGCTTTGCCCGGGCGGCCTCAGTGAGCGAGCGAGCGCGCA |
| | | GAGAGGGAGTGGCCAA |
| 84 | AAV5 cap | ATGTCTTTTGTTGATCACCCTCCAGATTGGTTGGAAGAAGTTGGTG |
| | | AAGGTCTTCGCGAGTTTTTGGGCCTTGAAGCGGGCCCACCGAAACC |
| | | AAAACCCAATCAGCAGCATCAAGATCAAGCCCGTGGTCTTGTGCTG |
| | | CCTGGTTATAACTATCTCGGACCCGGAAACGGTCTCGATCGAGGAG |
| | | AGCCTGTCAACAGGGCAGACGAGGTCGCGCGAGAGCACGACATCTC |
| | | GTACAACGAGCAGCTTGAGGCGGGAGACAACCCCTACCTCAAGTAC |
| | | AACCACGCGGACGCCGAGTTTCAGGAGAAGCTCGCCGACGACACAT |
| | | CCTTCGGGGGAAACCTCGGAAAGGCAGTCTTTCAGGCCAAGAAAAG |
| | | GGTTCTCGAACCTTTTGGCCTGGTTGAAGAGGGTGCTAAGACGGCC |
| | | CCTACCGGAAAGCGGATAGACGACCACTTTCCAAAAAGAAAGAAGG |
| | | CTCGGACCGAAGAGGACTCCAAGCCTTCCACCTCGTCAGACGCCGA |
| | | AGCTGGACCCAGCGGATCCCAGCAGCTGCAAATCCCAGCCCAACCA |
| | | GCCTCAAGTTTGGGAGCTGATACAATGTCTGCGGGAGGTGGCGGCC |
| | | CATTGGGCGACAATAACCAAGGTGCCGATGGAGTGGGCAATGCCTC |
| | | GGGAGATTGGCATTGCGATTCCACGTGGATGGGGGACAGAGTCGTC |
| | | ACCAAGTCCACCCGAACCTGGGTGCTGCCCAGCTACAACAACCACC |
| | | AGTACCGAGAGATCAAAAGCGGCTCCGTCGACGGAAGCAACGCCAA |
| | | CGCCTACTTTGGATACAGCACCCCCTGGGGGTACTTTGACTTTAAC |
| | | CGCTTCCACAGCCACTGGAGCCCCCGAGACTGGCAAAGACTCATCA |
| | | ACAACTACTGGGGCTTCAGACCCCGGTCCCTCAGAGTCAAAATCTT |
| | | CAACATTCAAGTCAAAGAGGTCACGGTGCAGGACTCCACCACCACC |
| | | ATCGCCAACAACCTCACCTCCACCGTCCAAGTGTTTACGGACGACG |
| | | ACTACCAGCTGCCCTACGTCGTCGGCAACGGGACCGAGGGATGCCT |
| | | GCCGGCCTTCCCTCCGCAGGTCTTTACGCTGCCGCAGTACGGTTAC |
| | | GCGACGCTGAACCGCGACAACACAGAAAATCCCACCGAGAGGAGCA |
| | | GCTTCTTCTGCCTAGAGTACTTTCCCAGCAAGATGCTGAGAACGGG |
| | | CAACAACTTTGAGTTTACCTACAACTTTGAGGAGGTGCCCTTCCAC |
| | | TCCAGCTTCGCTCCCAGTCAGAACCTGTTCAAGCTGGCCAACCCGC |
| | | TGGTGGACCAGTACTTGTACCGCTTCGTGAGCACAAATAACACTGG |
| | | CGGAGTCCAGTTCAACAAGAACCTGGCCGGGAGATACGCCAACACC |
| | | TACAAAAACTGGTTCCCGGGGCCCATGGGCCGAACCCAGGGCTGGA |
| | | ACCTGGGCTCCGGGGTCAACCGCGCCAGTGTCAGCGCCTTCGCCAC |
| | | GACCAATAGGATGGAGCTCGAGGGCGCGAGTTACCAGGTGCCCCCG |
| | | CAGCCGAACGGCATGACCAACAACCTCCAGGGCAGCAACACCTATG |
| | | CCCTGGAGAACACTATGATCTTCAACAGCCAGCCGGCGAACCCGGG |
| | | CACCACCGCCACGTACCTCGAGGGCAACATGCTCATCACCAGCGAG |
| | | AGCGAGACGCAGCCGGTGAACCGCGTGGCGTACAACGTCGGCGGGC |
| | | AGATGGCCACCAACAACCAGAGCTCCACCACTGCCCCCGCGACCGG |
| | | CACGTACAACCTCCAGGAAATCGTGCCCGGCAGCGTGTGGATGGAG |
| | | AGGGACGTGTACCTCCAAGGACCCATCTGGGCCAAGATCCCAGAGA |
| | | CGGGGGCGCACTTTCACCCCTCTCCGGCCATGGGCGGATTCGGACT |
| | | CAAACACCCACCGCCCATGATGCTCATCAAGAACACGCCTGTGCCC |
| | | GGAAATATCACCAGCTTCTCGGACGTGCCCGTCAGCAGCTTCATCA |
| | | CCCAGTACAGCACCGGGCAGGTCACCGTGGAGATGGAGTGGGAGCT |
| | | CAAGAAGGAAAACTCCAAGAGGTGGAACCCAGAGATCCAGTACACA |
| | | AACAACTACAACGACCCCCAGTTTGTGGACTTTGCCCCGGACAGCA |
| | | CCGGGGAATACAGAACCACCAGACCTATCGGAACCCGATACCTTAC |
| | | CCGACCCCTTTAA |
| 85 | 4xProSC | GTTAGTCTTTGCCCTTTATCTGCCTGGCAAAGCTTTTAATTGGCTT |
| | | GATCTGTCATTCCGCTAGACATAAAGGGGACAATCCCCGGATTAGG |
| | | AAGGAGCTCTCCAGCTCGGGTAAGGAGTCTCAAGGCAAGGTAGGCA |
| | | AGCACCACCGGTCCGCACTCTCGCCCAGCTTTTACGGGAAGAAGAG |
| | | AGTTAGTCTTTGCCCTTTATCTGCCTGGCAAAGCTTTTAATTGGCT |
| | | TGATCTGTCATTCCGCTAGACATAAAGGGGACAATCCCCGGATTAG |
| | | GAAGGAGCTCTCCAGCTCGGGTAAGGAGTCTCAAGGCAAGGTAGGC |
| | | AAGCACCACCGGTCCGCACTCTCGCCCAGCTTTTACGGGAAGAAGA |
| | | GAGTTAGTCTTTGCCCTTTATCTGCCTGGCAAAGCTTTTAATTGGC |
| | | TTGATCTGTCATTCCGCTAGACATAAAGGGGACAATCCCCGGATTA |
| | | GGAAGGAGCTCTCCAGCTCGGGTAAGGAGTCTCAAGGCAAGGTAGG |
| | | CAAGCACCACCGGTCCGCACTCTCGCCCAGCTTTTACGGGAAGAAG |
| | | AGAGTTAGTCTTTGCCCTTTATCTGCCTGGCAAAGCTTTTAATTGG |
| | | CTTGATCTGTCATTCCGCTAGACATAAAGGGGACAATCCCCGGATT |
| | | AGGAAGGAGCTCTCCAGCTCGGGTAAGGAGTCTCAAGGCAAGGTAG |
| | | GCAAGCACCACCGGTCCGCACTCTCGCCCAGCTTTTACGGGAAGAA |
| | | GAGAG |
| 86 | WPRE element | TAATCAACCTCTGGATTACAAAATTTGTGAAAGATTGACTGGTATT |
| | (deleted flanking | CTTAACTATGTTGCTCCTTTTACGCTATGTGGATACGCTGCTTTAA |
| | nucleotides) | TGCCTTTGTATCATGCTATTGCTTCCCGTATGGCTTTCATTTTCTC |
| | | CTCCTTGTATAAATCCTGGTTGCTGTCTCTTTATGAGGAGTTGTGG |
| | | CCCGTTGTCAGGCAACGTGGCGTGGTGTGCACTGTGTTTGCTGACG |
| | | CAACCCCCACTGGTTGGGGCATTGCCACCACCTGTCAGCTCCTTTC |
| | | CGGGACTTTCGCTTTCCCCCTCCCTATTGCCACGGCGGAACTCATC |
| | | GCCGCCTGCCTTGCCCGCTGCTGGACAGGGGCTCGGCTGTTGGGCA |
| | | CTGACAATTCCGTGGTGTTGTCGGGGAAATCATCGTCCTTTCCTTG |
| | | GCTGCTCGCCTATGTTGCCACCTGGATTCTGCGCGGGACGTCCTTC |
| | | TGCTACGTCCCTTCGGCCCTCAATCCAGCGGACCTTCCTTCCCGCG |
| | | GCCTGCTGCCGGCTCTGCGGCCTCTTCCGCGTCTTCGCCTTCGCCC |
| | | TCAGACGAGTCGGATCTCCCTTTGGGCCGCCTCCCCGC |
| 87 | Human growth | GGGTGGCATCCCTGTGACCCCTCCCCAGTGCCTCTCCTGGCCCTGG |
| | hormone poly A | AAGTTGCCACTCCAGTGCCCACCAGCCTTGTCCTAATAAAATTAAG |
| | (deleted flanking | TTGCATCATTTTGTCTGACTAGGTGTCCTTCTATAATATTATGGGG |
| | nucleotides) | TGGAGGGGGGTGGTATGGAGCAAGGGGCAAGTTGGGAAGACAACCT |
| | | GTAGGGCCTGCGGGGTCTATTGGGAACCAAGCTGGAGTGCAGTGGC |
| | | ACAATCTTGGCTCACTGCAATCTCCGCCTCCTGGGTTCAAGCGATT |
| | | CTCCTGCCTCAGCCTCCCGAGTTGTTGGGATTCCAGGCATGCATGA |
| | | CCAGGCTCAGCTAATTTTTGTTTTTTTGGTAGAGACGGGGTTTCAC |
| | | CATATTGGCCAGGCTGGTCTCCAACTCCTAATCTCAGGTGATCTAC |
| | | CCACCTTGGCCTCCCAAATTGCTGGGATTACAGGCGTGAACCACTG |
| | | CTCCCTTCCCTGTCCTTCTGATTTT |
| 88 | AAV L-ITR | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAGAGAGGGAG |
| | | TGGCCAA |
| 89 | AAV R-ITR | TTGGCCACTCCCTCTCTGCGCGCTCGCTCGCTCACTGAGGCCGCCC |
| | | GGGCAAAGCCCGGGCGTCGGGCGACCTTTGGTCGCCCGGCCTCAGT |
| | | GAGCGAGCGAGCGCGCAGAGAGGGAGTGGCCAACTCCATCACTAGG |
| | | GGTTCCT |

1. An isolated nucleic acid, comprising:
(i) a promoter that is a hybrid promoter comprising a first nucleotide sequence comprising at least portion of a cone-specific promoter and a second nucleotide sequence comprising at least a portion of a rod specific promoter, where the first and second nucleotide sequences are operably linked and function as a single promoter for expression in cone photoreceptors, or a cone specific promoter, or an active variant, fragment or truncation of either of the foregoing;
(ii) a nucleotide sequence encoding a depolarizing optogenetic protein and optionally a reporter molecule; and
(iii) a Woodchuck Hepatitis Virus Posttranscriptional Regulatory element (WPRE);
wherein the promoter, the nucleotide sequence encoding a depolarizing optogenetic protein and optionally a reporter molecule and the WPRE are operably linked.

2. The isolated nucleic acid of claim 1, wherein the promoter is a hybrid promoter comprising SEQ ID NO: 14, SEQ ID NO: 13, SEQ ID NO: 12 or SEQ ID NO: 11 or active variant, fragment or truncation of any of the foregoing.

3. The isolated nucleic acid of claim 1, wherein the promoter is a hybrid promoter and the first nucleotide sequence comprises at least about 150 consecutive nucleotides of SEQ ID NO:2 and the second nucleotide sequence comprises at least about 264 consecutive nucleotides of SEQ ID NO: 10, or an active variant, fragment or truncation of any of the foregoing.

4. The isolated nucleic acid of claim 3, wherein the first nucleotide sequence is selected from the group consisting of SEQ ID NO:24, SEQ ID NO:25, SEQ ID NO:26, SEQ ID NO:27, and a sequence that contains at least about 150 continuous nucleotides from the 3′ terminus of SEQ ID NO:2, and the second nucleic acid comprises SEQ ID NO:54, or an active variant of any of the foregoing.

5. The isolated nucleic acid of claim 4, wherein the second nucleotide comprises SEQ ID NO: 10 or an active variant, fragment or truncation thereof.

6. The isolated nucleic acid of claim 1, wherein the promoter is a cone specific promoter comprising at least about 150 consecutive nucleotides of SEQ ID NO:2 or an active variant thereof.

7. The isolated nucleic acid of claim 6, wherein the promoter is selected from the group consisting of SEQ ID NO:24, SEQ ID NO:25, SEQ ID NO:26, SEQ ID NO:27, and a sequence that contains at least about 150 continuous nucleotides from the 3′ terminus of SEQ ID NO:2, or an active variant thereof.

8. The isolated nucleic acid of claim 1, wherein the hybrid promoter or cone specific promoter comprises one to about ten copies of a sequence selected from the group consisting of SEQ ID NO:24, SEQ ID NO:25, SEQ ID NO:26, SEQ ID NO:27, a sequence that contains at least about 150 continuous nucleotides from the 3′ terminus of SEQ ID NO:2, an active variant of any of the foregoing, and combinations thereof.

9. The isolated nucleic acid of claim 1, wherein the hybrid promoter comprises one to about ten copies of SEQ ID NO:54, or an active variant thereof.

10. The isolated nucleic acid of claim 1, wherein the WPRE comprises a nucleotide sequence having at least 70% identity to SEQ ID NO: 3 or a nucleotide sequence having at least 70% identity to SEQ ID NO: 86.

11. The isolated nucleic acid of claim 1, further comprising a polyadenylation signal (PolyA) that is 3′ of the WPRE.

12. (canceled)

13. The isolated nucleic acid of claim 11, comprising a first AAV ITR that is 5′ of the promoter and a second AAV ITR that is 3′ of the WPRE and optionally 3′ of the PolyA signal.

14. The isolated nucleic acid of claim 1, wherein the depolarizing optogenetic protein is a light responsive polypeptide.

15. The isolated nucleic acid of claim 14, wherein the light responsive polypeptide is a light-gated ion channel polypeptide.

16. The isolated nucleic acid of claim 15, wherein the light-gated ion channel polypeptide is a channelrhodopsin or a functional variant thereof.

17. The isolated nucleic acid of claim 16, wherein the channelrhodopsin is a ReaChR polypeptide or a functional variant thereof.

18. (canceled)

19. The isolated nucleic acid of claim 17, wherein the nucleotide sequence encoding ReaChR or a functional variant thereof is selected from the group consisting of SEQ ID NO: 16, SEQ ID NO:33, SEQ ID NO:34, SEQ ID NO: 58, SEQ ID NO: 59, SEQ ID NO: 61, SEQ ID NO: 62, SEQ ID NO: 63, SEQ ID NO: 64, SEQ ID NO: 65, SEQ ID NO: 66, SEQ ID NO: 69, SEQ ID NO: 73 or an amino acid sequence with at least about 70% identity to any of the foregoing.

20. The isolated nucleic acid of claim 16, wherein the channelrhodopsin or functional variant thereof is engineered to enhance membrane localization when expressed in human cone photoreceptors.

21. The isolated nucleic acid of claim 20, wherein the engineered channelrhodopsin comprises SEQ ID NO:57 or a functional variant thereof.

22. (canceled)

23. The isolated nucleic acid of claim 1, wherein the promoter comprises:
two or more first nucleotide sequences;
two or more second nucleotide sequences;
two or more cone-specific promoters; or
two or more thereof.

24. (canceled)

25. The nucleic acid of claim 1, comprising a nucleotide sequence comprising SEQ ID NO: 4, SEQ ID NO: 5, SEQ ID NO: 6, SEQ ID NO: 7, SEQ ID NO: 17, SEQ ID NO: 18, SEQ ID NO: 19, SEQ ID NO: 20, SEQ ID NO: 21, SEQ ID NO: 35, SEQ ID NO: 55, SEQ ID NO: 56, SEQ ID NO: 60, SEQ ID NO: 67, SEQ ID NO: 68, SEQ ID NO: 71, SEQ ID NO: 72, SEQ ID NO: 75, SEQ ID NO: 76, SEQ ID NO: 77, SEQ ID NO: 80, SEQ ID NO: 81, SEQ ID NO: 82, or SEQ ID NO: 83, or a sequence that has at least about 70% identity to any of the foregoing.

26. (canceled)

27. The nucleic acid of claim 1, wherein the active variant has at least about 70% identity to the corresponding reference sequence.

28. The nucleic acid of claim 27, wherein the active variant contains one or more of codon optimization, CpG-reduction, alternative start site removal, repeat removal, hairpin removal, unwanted splice donor and acceptor site removal, ITR terminal resolution site deletion, stuffer sequence addition, or miRNA addition.

29. A viral particle comprising the nucleic acid of claim 1.

30. A host cell comprising the nucleic acid of claim 1 or the viral particle of claim 29.

31. An AAV vector, comprising:
(i) the nucleic acid of claim 1; and
(ii) an AAV capsid.

32. The AAV vector of claim 31, wherein the AAV capsid is an AAV5 capsid, a PHP.eB capsid, an NHP26 capsid, an AAV8 capsid, an AAV8-BP2 capsid, an AAV9 capsid, or a PHP.B capsid.

33. (canceled)

34. (canceled)

35. A pharmaceutical composition comprising:
(i) a viral particle of claim 29, or an AAV vector of claim 31; and
(ii) a pharmaceutically acceptable excipient.

36. A method of delivering a depolarizing optogenetic protein to a human cone cell in a subject in need thereof, the method comprising administering to the subject a nucleic acid of claim 1, a viral particle of claim 29, an AAV vector of claim 31, or a pharmaceutical composition of claim 35.

37. (canceled)

38. (canceled)

39. A method for restoring sensitivity to light in a human cone photoreceptor cell, the method comprising administering to the human cone photoreceptor cell in need thereof a nucleic acid of any one of claims 1-28, a viral particle of claim 29, an AAV vector of any one of claims 31-33, a host cell of claim 30 or 34 or a pharmaceutical composition of claim 35.

40. A method for treating retinal degeneration in a subject, the method comprising administering to a subject in need thereof an effective amount of a nucleic acid of claim 1, a viral particle of claim 29, an AAV vector of claim 31 or a pharmaceutical composition of claim 35.

41. The method of claim 36, wherein the subject has or is at risk of developing retinitis pigmentosa, rod-cone dystrophy, Leber's congenital amaurosis, Usher's syndrome, Bardet-Biedl Syndrome, Best disease, retinoschisis, Stargardt disease, untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, Choroideremia, a tapetoretinal degeneration retinal vein occlusion or geographic atrophy.

42-54. (canceled)
